(19) United States
(12) Patent Application Publication
(10) Pub. No.: US 2022/0396586 A1
(43) Pub. Date: Dec. 15, 2022

(54) THIAZOLE CARBOXAMIDE COMPOUNDS AND USE THEREOF FOR THE TREATMENT OF MYCOBACTERIAL INFECTIONS

(71) Applicant: THE GLOBAL ALLIANCE FOR TB DRUG DEVELOPMENT, INC., New York, NY (US)

(72) Inventors: Takushi KANEKO, Guilford, CT (US); Nader FOTOUHI, Basking Ridge, NJ (US)

(21) Appl. No.: 17/761,273

(22) PCT Filed: Sep. 25, 2020
(86) PCT No.: PCT/US2020/052937

§ 371 Date: Mar. 17, 2022

Related U.S. Application Data
(60) Provisional application No. 62/906,452, filed on Sep. 26, 2019.

(51) Int. Cl. C07D 513/04; A61K 31/429; C07F 7/08; A61K 31/695; A61K 45/06; A61P 31/06

(52) U.S. Cl. CPC C07D 513/04; A61K 31/429; C07F 7/0812; A61K 31/695; A61K 45/06; A61P 31/06

(57) ABSTRACT

Provided herein are compounds of Formula (I) and Formula (II) as well as pharmaceutically acceptable salts thereof, wherein the substituents are as those disclosed in the specification. These compounds, and the pharmaceutical compositions containing them, are useful for the treatment of tuberculosis.

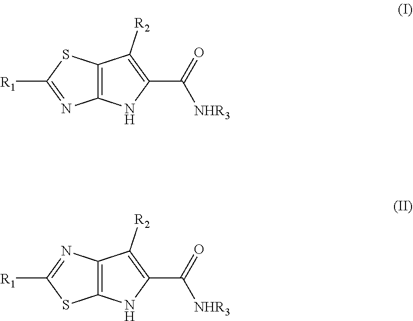

### FIELD OF THE INVENTION

The invention is directed, for example, to compounds of Formula (I) and compounds of Formula (II):

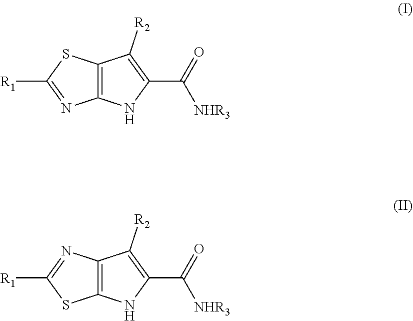

and to pharmaceutical compositions comprising the compounds. The compounds and compositions disclosed herein are antibacterials and are useful for the treatment of tuberculosis and other mycobacterial infections.

All publications, patents, patent applications, and other references cited in this application are incorporated herein by reference in their entirety for all purposes and to the same extent as if each individual publication, patent, patent application or other reference was specifically and individually indicated to be incorporated by reference in its entirety for all purposes. Citation of a reference herein shall not be construed as an admission that such is prior art to the present invention.

### BACKGROUND OF THE INVENTION

Mycobacterium tuberculosis (“M. tb”) is the causative agent of tuberculosis (“TB”), a devastating infectious disease. It is estimated that about 2 million TB patients die each year globally. The treatment of drug-susceptible TB currently centers on four antibiotics, isoniazid, rifampicin, ethambutol, and pyrazinamide which were introduced more than 40 years ago (Franz 2017). Failure to properly treat tuberculosis has caused global drug resistance in M. tb and thus rendering some medications ineffective. A need exists in the art, therefore, to identify new chemical entities to treat TB.

### SUMMARY OF THE INVENTION

The present invention is directed to 4H-pyrrolo[2,3-d]thiazole-5-carboxamides and 4H-pyrrolo[3,2-d]thiazole-5-carboxamides such as compounds of Formula (I) and Formula (II)

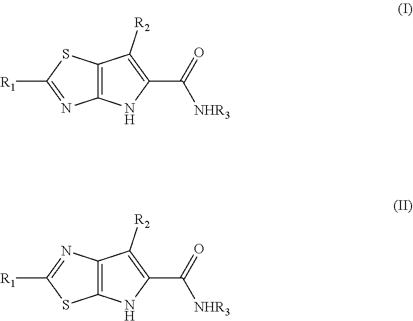

wherein

R₁ is hydrogen, (C₁-C₁₁)alkyl, cycloalkyl, aryl, heteroaryl, alkoxy, or cycloalkoxy;

R₂ is hydrogen, alkyl, cycloalkyl, CN, or halogen;

R₃NH is
(i) (C₄-C₆)alkyl-NH or (C₄-C₇)alkyl-NH;
(ii) (C₅-C₁₀)cycloalkyl-NH;
(iii) —CH₂—(C₅-C₇)cycloalkyl-NH;
(iv) spiro(C₈-C₁₁)cycloalkyl-NH;
(v) phenyl-NH;
(vi)

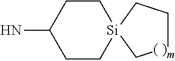

wherein m is 1 or 2; or
(vii)

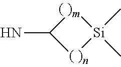

wherein m is 1, 2 or 3 and n is 1, 2, 3, or 4, or a pharmaceutically acceptable salt thereof.

The present invention is also directed to pharmaceutical compositions containing the above compounds and to methods of treating microbial infections such as tuberculosis.

### DETAILED DESCRIPTION OF THE INVENTION

It is to be understood that the terminology employed herein is for the purpose of describing particular embodiments, and is not intended to be limiting. Further, although any methods, devices and materials similar or equivalent to those described herein can be used in the practice or testing of the invention, certain methods, devices and materials are now described.

The present invention relates to novel thiazole carboxamide compounds, their preparations, and to their use as drugs for treating tuberculosis and other mycobacteria infections. The compounds, in certain embodiments, have the following general structures:

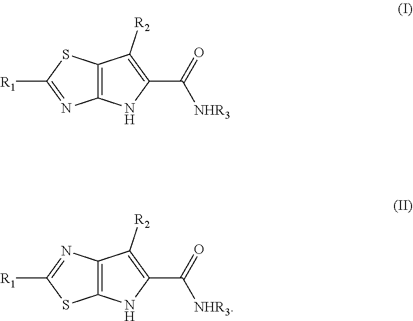

In one embodiment of the invention, the compounds of the invention can treat TB in combination with other anti-TB agents. The anti-TB agents include, but are not limited to, rifampicin, rifabutin, rifapentene, isoniazid, ethambutol, kanamycin, amikacin, capreomycin, clofazimine, cycloserine, para-aminosalicylic acid, linezolid, sutezolid, bedaquiline, delamanid, pretomanid, moxifloxacin, and levofloxacin.

### Definitions

As used herein, the term “alkyl”, alone or in combination with other groups, refers to a branched or straight-chain monovalent saturated aliphatic hydrocarbon radical of one to twenty carbon atoms, in one embodiment one to sixteen carbon atoms, in another embodiment one to ten carbon atoms.

The term “lower alkyl”, alone or in combination with other groups, refers to a branched or straight-chain alkyl radical of one to nine carbon atoms, in one embodiment one to six carbon atoms, in another embodiment one to four carbon atoms, in a further embodiment four to six carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

As used herein, the term “alkoxy” means alkyl-O—; and “alkoyl” means alkyl-CO—. Alkoxy substituent groups or alkoxy-containing substituent groups may be substituted by, for example, one or more alkyl or halo groups.

As used herein, the term “cycloalkoxy” means cycloalkyl-O—. Cycloalkoxy substituent groups may be substituted by, for example, one or more alkyl or halo groups.

As used herein, the term “halogen” means a fluorine, chlorine, bromine or iodine radical, or in some embodiments a fluorine, chlorine or bromine radical.

The term “cycloalkyl” refers to a monovalent mono- or polycarbocyclic radical of three to ten, in one embodiment three to six carbon atoms. This term is further exemplified by radicals such as cyclopropyl, cyclobutyl, cyclopentyl, cyclohexyl, cycloheptyl, norbornyl, adamantyl, indanyl and the like. In one embodiment, the “cycloalkyl” moieties can optionally be substituted with one, two, three or four substituents. Each substituent can independently be alkyl, alkoxy, halogen, amino, hydroxyl, aryl, heteroaryl or oxygen unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl, and the like or those which are specifically exemplified herein.

The term “spirocycloalkyl” refers to two nonaromatic carbocyclic rings that are connected through a common carbon atom. Unless specified otherwise, the individual carbocyclic rings are generally 3- to 6-membered rings or the joined rings are generally an 8- to 11-membered bicyclic ring system. For example, a spiro-(C₈-C₁₁)cycloalkyl group includes groups such as spiro[2.5]octan-6-yl, spiro[3.5]nonan-7-yl, spiro[4.5]decan-8-yl, and spiro[5.5]undecan-3-yl. In one embodiment, the spirocycloalkyl group is spiro[2.5]octan-6-yl.

The term “aryl” refers to an aromatic mono- or polycarbocyclic radical of 6 to 12 carbon atoms having at least one aromatic ring. Examples of such groups include, but are not limited to, phenyl, naphthyl, 1,2,3,4-tetrahydronaphthyl, 1,2-dihydronaphthyl, indanyl, 1H-indenyl and the like.

As used herein, a “ara-alkyl” refers to an alkyl group substituted with at least one aryl group. Similarly, as used herein, “heteroara-alkyl” group refers to an alkyl group substituted with at least one heteroaryl group.

The alkyl, lower alkyl, aryl, and spirocycloalkyl groups, may be substituted or unsubstituted. Additionally, ara-alkyl and heteroara-alkyl groups may be substituted with substituents in addition to aryl or heteroaryl groups. When substituted, there will generally be, for example, 1 to 4 substituents present. These substituents may optionally form a ring with the alkyl, lower alkyl or aryl group with which they are connected. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl, in other embodiments, for example, methoxy and ethoxy), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di-alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl and carbolinyl).

As would be readily understood from the disclosure provided herein, any reference to a group falling within a generic group may be substituted or unsubstituted in the same manner. For example, a phenyl group may be substituted in the same manner as an aryl group. The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. Examples of such groups include, but not limited to, pyridinyl, pyrazinyl, pyridazinyl, 1,2,3-triazinyl, 1,2,4-triazinyl, oxazolyl, thiazolyl, and the like.

The heteroaryl group described above may be substituted independently with one, two, or three substituents. Substituents may include, for example: carbon-containing groups such as alkyl, aryl, arylalkyl (e.g. substituted and unsubstituted phenyl, substituted and unsubstituted benzyl); halogen atoms and halogen-containing groups such as haloalkyl (e.g. trifluoromethyl); oxygen-containing groups such as alcohols (e.g. hydroxyl, hydroxyalkyl, aryl(hydroxyl)alkyl), ethers (e.g. alkoxy, aryloxy, alkoxyalkyl, aryloxyalkyl), aldehydes (e.g. carboxaldehyde), ketones (e.g. alkylcarbonyl, alkylcarbonylalkyl, arylcarbonyl, arylalkylcarbonyl, arycarbonylalkyl), acids (e.g. carboxy, carboxyalkyl), acid derivatives such as esters (e.g. alkoxycarbonyl, alkoxycarbonylalkyl, alkylcarbonyloxy, alkylcarbonyloxyalkyl), amides (e.g. aminocarbonyl, mono- or di-alkylaminocarbonyl, aminocarbonylalkyl, mono- or di-alkylaminocarbonylalkyl, arylaminocarbonyl), carbamates (e.g. alkoxycarbonylamino, aryloxycarbonylamino, aminocarbonyloxy, mono- or di-alkylaminocarbonyloxy, arylminocarbonloxy) and ureas (e.g. mono- or di- alkylaminocarbonylamino or arylaminocarbonylamino); nitrogen-containing groups such as amines (e.g. amino, mono- or di-alkylamino, aminoalkyl, mono- or di-alkylaminoalkyl), azides, nitriles (e.g. cyano, cyanoalkyl), nitro; sulfur-containing groups such as thiols, thioethers, sulfoxides and sulfones (e.g. alkylthio, alkylsulfinyl, alkylsulfonyl, alkylthioalkyl, alkylsulfinylalkyl, alkylsulfonylalkyl, arylthio, arysulfinyl, arysulfonyl, arythioalkyl, arylsulfinylalkyl, arylsulfonylalkyl); and heterocyclic groups containing one or more heteroatoms, (e.g. thienyl, furanyl, pyrrolyl, imidazolyl, pyrazolyl, thiazolyl, isothiazolyl, oxazolyl, oxadiazolyl, thiadiazolyl, aziridinyl, azetidinyl, pyrrolidinyl, pyrrolinyl, imidazolidinyl, imidazolinyl, pyrazolidinyl, tetrahydrofuranyl, pyranyl, pyronyl, pyridyl, pyrazinyl, pyridazinyl, piperidyl, hexahydroazepinyl, piperazinyl, morpholinyl, thianaphthyl, benzofuranyl, isobenzofuranyl, indolyl, oxyindolyl, isoindolyl, indazolyl, indolinyl, 7-azaindolyl, benzopyranyl, coumarinyl, isocoumarinyl, quinolinyl, isoquinolinyl, naphthridinyl, cinnolinyl, quinazolinyl, pyridopyridyl, benzoxazinyl, quinoxalinyl, chromenyl, chromanyl, isochromanyl, phthalazinyl, benzothiazoyl and carbolinyl).

In some instances, a term is preceded by “(C_{#}-C_{#}).” As would be readily understood from the disclosure provided herein, this defines the number of carbon atoms associated with the term. For example, (C₁-C₆)alkyl means an alkyl in which the branched or straight-chain monovalent saturated aliphatic hydrocarbon radical has one to 6 carbon atoms. As would be readily understood from the disclosure provided herein, all substitution definitions apply equally to these structures. For example, (C₁-C₆)alkyl may be substituted in the same manner an alkyl is substituted.

By any range disclosed herein, it is meant that all integer unit amounts within the range are specifically disclosed as part of the invention. Thus, for example, 1 to 12 units means that 1, 2, 3 . . . 12 units are included as embodiments of this invention.

As used herein, multi-drug-resistant tuberculosis (MDR-TB) is a form of TB which has resistance to isoniazid and rifampin, with or without resistance to other drugs. As used herein, pre-extensively drug resistant (Pre-XDR-TB) is a form of TB which has resistance to isoniazid and rifampin and either a fluoroquinolone or an injectable drug but not both. As used herein, extensively drug resistant tuberculosis (XDR-TB) is a form of TB which has resistance to isoniazid, rifampin, fluoroquinolones and at least one injectable drug (e.g., streptomycin, amikacin, kanamycin, capreomycin).

Compounds of the present invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbents or eluant). The invention embraces all of these forms.

In one embodiment, the present invention also provides for combination therapy of the compounds of the present invention with at least one other therapeutic agent. The other agent may be prepared for simultaneous, separate or sequential use in therapy to treat the subject.

In the practice of the method of the present invention, an effective amount of any one of the compounds of this invention, or a combination of any of the compounds of this invention, is administered via any of the usual and acceptable methods known in the art, either singly or in combination. The compounds or compositions can thus be administered, for example, ocularly, orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form or solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases. Thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are representative liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. In one embodiment, the therapeutically effective amount is in an amount of from about 10 mg to about 500 mg per day.

It will be appreciated that the compounds of the invention may be derivatized at functional groups to provide derivatives which are capable of conversion back to the parent compound in vivo. Physiologically acceptable and metabolically labile derivatives, which are capable of producing the parent compounds of general Formulas I and II in vivo are also within the scope of this invention.

Compounds of the present invention can be prepared beginning with commercially available starting materials and utilizing general synthetic techniques and procedures known to those skilled in the art. Chemicals may be purchased from companies such, as for example, Aldrich, Argonaut Technologies, VWR and Lancaster. Chromatography supplies and equipment may be purchased from such companies as for example AnaLogix, Inc., Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are pre-packed silica gel columns used in standard chromatography.

In some embodiments, the compound has one of the following structures:

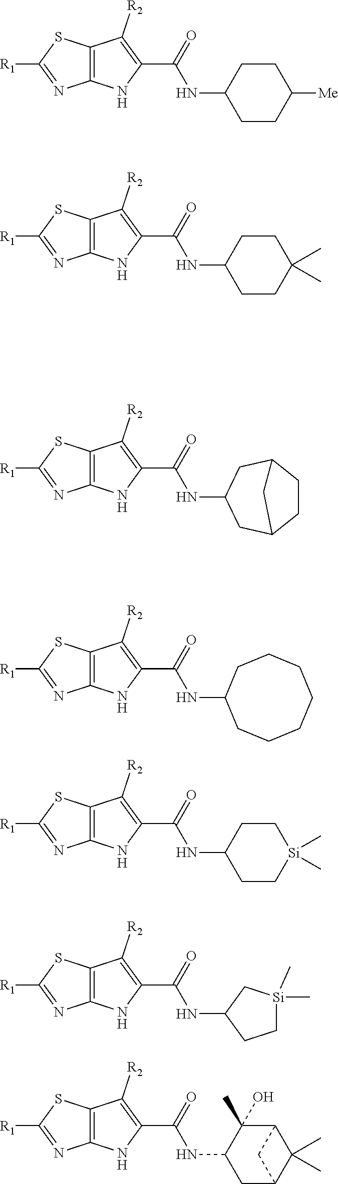

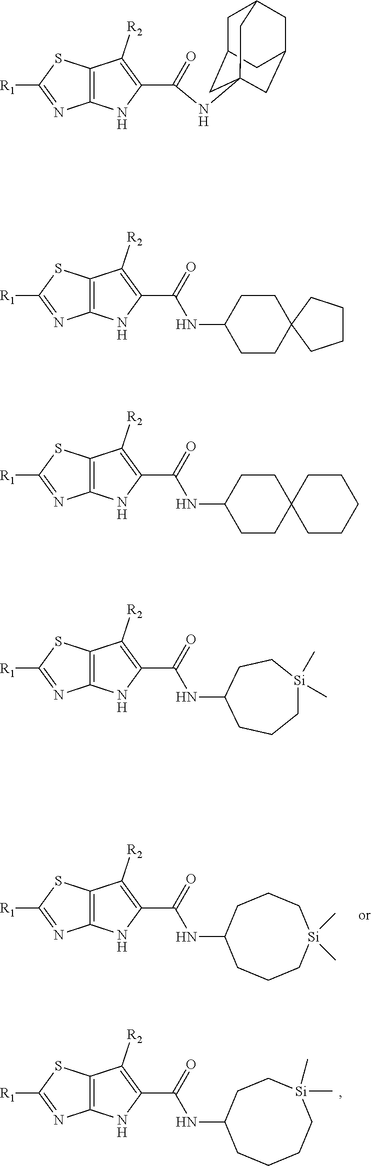

or a pharmaceutically acceptable salt thereof and R₁ and R₂ are as defined as in the paragraphs below.

In some embodiments, the compound has one of the following structures:

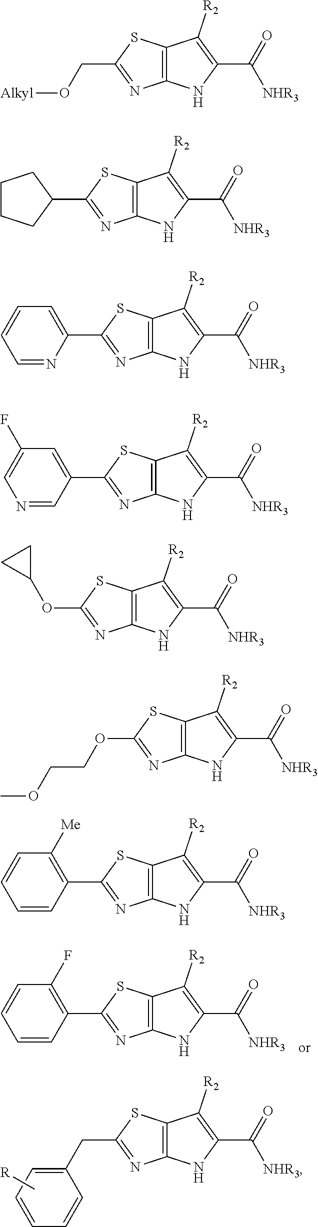

or a pharmaceutically acceptable salt thereof and R₂ and R₃ are as defined as in the paragraphs below.

In some embodiments, R₁ may be an alkoxy group substituted with a cycloalkyl- or cycloalkyl-alkyl-group, for example but not limited to,

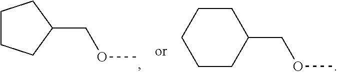

In some embodiments, the compound may have the structure:

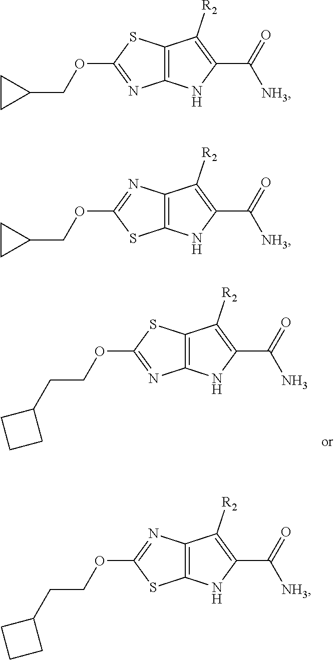

or a pharmaceutically acceptable salt thereof, and R₂ and R₃ are as defined as in the paragraphs below.

In one embodiment, the compound is selected from the group consisting of: MPL-020, MPL-025, MPL-293, MPL-308, MPL-309, MPL-357, MPL-357A, MPL-358, MPL-359, MPL-369, MPL-371, MPL-373, MPL-393, MPL-394, MPL-395, MPL-395A, MPL-403, MPL-404, MPL-426, MPL-427, MPL-431, MPL-458, MPL-459, MPL-472, MPL-474, MPL-475, and MPL-478, or a pharmaceutically acceptable salt thereof.

In some embodiments, R₃NH is

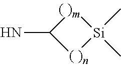

and m is 1-3 and n is 1-4. In another embodiment, m is 1 and n is 1. In another embodiment, m is 1 and n is 2. In another embodiment, m is 1 and n is 3. In another embodiment, m is 1 and n is 4. In another embodiment, m is 2 and n is 1. In another embodiment, m is 2 and n is 2. In another embodiment, m is 2 and n is 3. In another embodiment, m is 2 and n is 4. In another embodiment, m is 3 and n is 1. In another embodiment, m is 3 and n is 2. In another embodiment, m is 3 and n is 3. In another embodiment, m is 3 and n is 4. In the case where m is not equal to n, there exists a stereocenter in the amine and in the resulting amide. The product may be a mixture or it may be resolved individual stereoisomers of the amide although the absolute stereochemical assignments are not made. Under such a case, a number (MPL-xxx) without a suffix A or B is meant for a racemic mixture whereas suffix A and B (such as NUM-xxxA and MPL-xxxB) is meant to indicate resolved enantiomers although no absolute configuration has been assigned to each enantiomer. Separation of stereoisomers are most effectively achieved by the use of Super Fluid Chromatography (SFC) equipped with a chiral column.

Synthesis of Representative Compounds of the Invention

The compounds of the invention can be prepared according to the following two schemes showing general methods A and B:

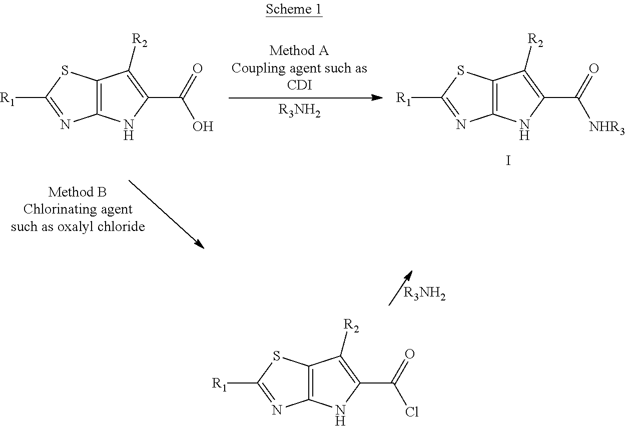

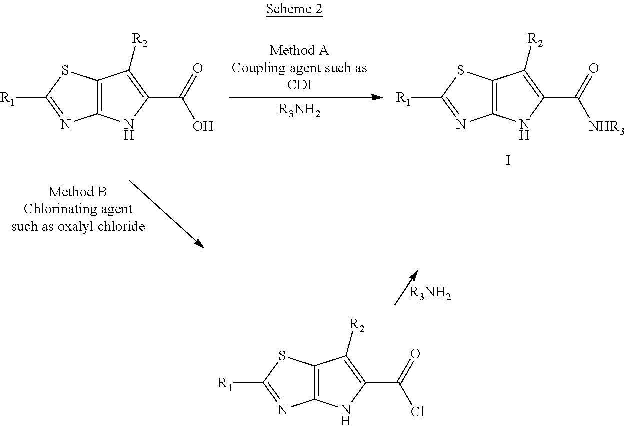

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Abbreviations used: ABPR, automatic back-pressure regulator; ACN, acetonitrile;aq., aqueous

CDI, 1,1′-carbonyl diimidazole; DCM, dichloromethane; DEA, diethylamine; DMF, dimethylformamide; DMSO, dimethylsulfoxide; EtOAc, ethyl acetate; EDCI, 1-ethyl-3-(3-dimethylaminopropyl)carbodiimide; ESI, electrospray ionization; eq, equivalent; FA, formic acid; HOBt, 1-hydroxybenzonitrile; NBS, N-bromosuccinimide; HPLC, high performance liquid chromatography; LAH, lithium aluminium hydride; LCMS or LC-MS, liquid chromatography—mass spectrometry; min, minute; m/z, mass-to-charge ratio; nd, no data; nm, nanometer; NMR, nuclear magnetic resonance; ¹H NMR, proton NMR; Pd(dppf)C12, 1,1′ bis(diphenylphosphino)ferrocene]dichloropalladium(II); Pd₂(dpa)₃, tris(dibenzylideneacetone)dipalladium(0); prep-HPLC, preparative HPLC; prep-TLC, preparative TLC; psi, pound per square inch; sat., saturated; SFC, supercritical fluid chromatography; TEA, triethylamine; THF, tetrahydrofuran; TLC, Thin-layer chromatography; ul, microliter; umol, micromole; XantPhos, 4,5-Bis(diphenylphosphino)-9,9-dimethylxanthene; XPhos, 2-dicyclohexylphosphino-2′,4′,6′-triisopropylbiphenyl; δ, chemical shift in ppm.

Reactions were monitored by TLC or LCMS and compounds were characterized by LCMS and/or NMR. Shimadzu LC20-MS2010 or LC20-MS2020 were used for LC/MS analysis. Varian 400 MHz, Varian 500 MHz or Bruker 500 MHz were used for NMR measurement.

General conditions for prep-HPLC purification: Instrument: Gilson GX281; Flow rate: 25 mL/min; Detector: UV 220 and UV 254 “[water (X)-Y]; B%: J%-K%, Lmin” stands for mobile phase A: X in water; B: Y; gradient J%-K%B over L min. For example, ‘[water(0.225%FA)-ACN];B%: 36%-66%,11min’ means mobile phase: A: 0.025% formic acid in water, B: acetonitrile; gradient: 36%-66%B over 11 min.

### Example 1, MPL-020

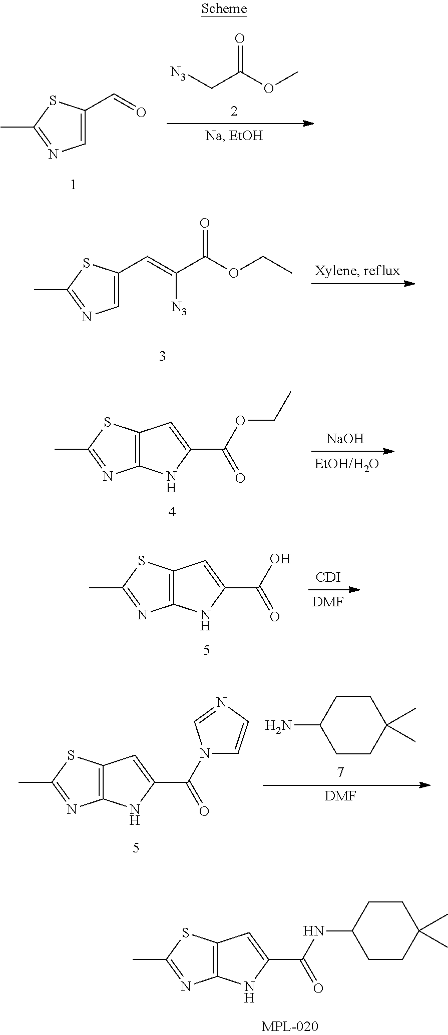

### Step 1. Synthesis of ethyl (Z)-2-azido-3-(2-methylthiazol-5-yl)prop-2-enoate

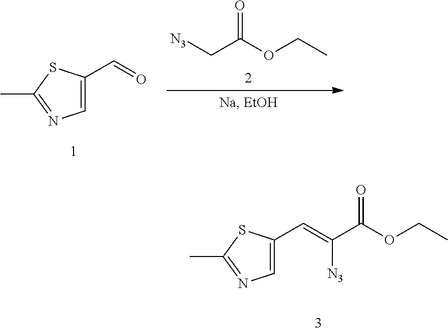

To a stirring solution of Na (723.15 mg, 31.46 mmol, 745.52 μL, 4 eq) in absolute EtOH (46 mL) at 0° C. was added dropwise a solution of 2-methylthiazole-5-carbaldehyde (1 g, 7.86 mmol, 1 eq) and ethyl 2-azidoacetate (4.06 g, 31.46 mmol, 4.41 mL, 4 eq) in EtOH (50 mL). The reaction was stirred at 0° C. for 2 hours. TLC indicated the reaction was finished. The reaction mixture was poured into a saturated solution of ammonium chloride (30 mL) and extracted with EtOAc (40 mL×3). The organic layer was washed once with water and dried over anhydrate sodium sulfate and filtered and concentrated under reduced pressure to give a residue. The residue was purified by column chromatography (SiO₂, petroleum ether/EtOAc=20/1 to 3/1). Compound ethyl (Z)-2-azido-3-(2-methylthiazol-5-yl)prop-2-enoate (780 mg, 3.11 mmol, 39.55% yield, 95% purity) was obtained as a yellow solid.

### Step 2. Synthesis of ethyl 2-methyl-4H-pyrrolo[2,3-d]thiazole-5-carboxylate

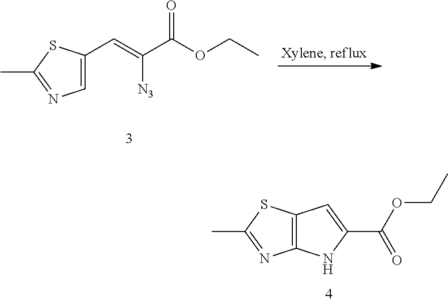 [SMILES: CCOC(=O)/C(=C/c1cnc(C)s1)N=[N+]=[N-] ; CCOC(=O)c1cc2sc(C)nc2[nH]1]

A solution of ethyl (Z)-2-azido-3-(2-methylthiazol-5-yl)prop-2-enoate (780 mg, 3.27 mmol, 1 eq) in xylene (4 mL) was warmed to 150° C. for 1 hr. LCMS (Liquid chromatography—mass spectrometry) showed the desired product was detected. An amount of precipitate was formed in solution and then the suspension solution was filtered and the filter cake was washed with petroleum ether to give a yellow solid. Compound ethyl 2-methyl-4H-pyrrolo[2,3-d]thiazole-5-carboxylate (550 mg, 2.49 mmol, 75.91% yield, 95% purity) was obtained as a yellow solid. LCMS (ESI) m/z 211.0 [M+H]⁺

### Step 3. Synthesis of 2-methyl-4H-pyrrolo[2,3-d]thiazole-5 -carboxylic acid

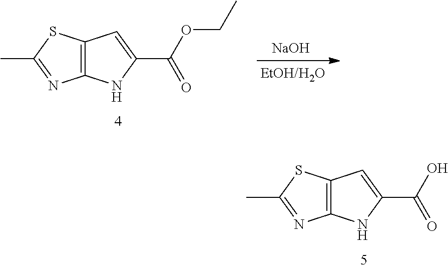

To a solution of ethyl 2-methyl-4H-pyrrolo[2,3-d]thiazole-5-carboxylate (700 mg, 3.33 mmol, 1 eq) in EtOH (20 mL) was added NaOH (2 M, 10 mL, 6.01 eq), the mixture was stirred at 70° C. for 12 hours. LCMS showed the starting material was consumed and the desired product was detected. The mixture was concentrated under reduced pressure to give a residue, then diluted with water (10 mL), acidified with HCl (2 M) to pH=5. The mixture was filtered and the filter cake was washed with 10 mL×3 of Petroleum ether, dried under reduced pressure to 2-methyl-4H-pyrrolo[2,3-d]thiazole-5 -carboxylic acid (500 mg, 2.67 mmol, 80.32% yield, 97.447% purity) was obtained as a yellow solid.

LCMS (ESI) m/z 183.0 [M+H]⁺

### Step 4. Synthesis of N-(4,4-dimethylcyclohexyl)-2-methyl-4H-pyrrolo[2,3-d]thiazole-5-carboxamide

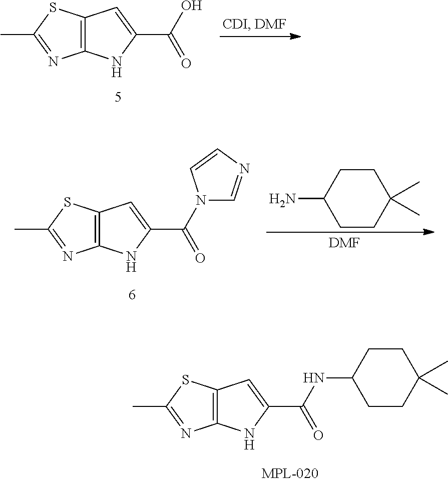

Step 4.1 To a solution of 2-methyl-4H-pyrrolo[2,3-d]thiazole-5-carboxylic acid (400 mg, 2.20 mmol, 1 eq) in DMF (8 mL) was added CDI (391.58 mg, 2.41 mmol, 1.1 eq). Then the resulting solution was stirred at 30° C. for 1 hr.

Step 4.2 A solution of imidazol-1-yl-(2-methyl-4H-pyrrolo[2,3-d]thiazol-5-yl)methanone (509 mg, 2.19 mmol, 1 eq) in DMF (1 mL last step reaction solution) was added dropwise 4,4-dimethylcyclohexanamine (278.82 mg, 2.19 mmol, 1 eq) in DMF (2 mL). The resulting material was stirred at 30° C. for 30 min. LCMS showed the starting material was consumed. The mixture was diluted with EtOAc (100 mL) and washed with LiCl (3% 50 mL×2). The organic layer was dried over Na₂SO₄ and filtered and concentrated under reduced pressure to give a residue. The product was purified by column chromatography (SiO₂, petroleum ether/EtOAc=20/1 to 1/1). Compound N-(4,4-dimethylcyclohexyl)-2-methyl-4H-pyrrolo[2,3-d]thiazole-5-carboxamide (358.8 mg, 1.22 mmol, 55.62% yield, 99% purity) was obtained as a yellow solid.

LCMS (ESI) m/z 292.1 [M+H]⁺; ¹H NMR (500 MHz, CHLOROFORM-d) δ=9.81 (br s, 1H), 6.65 (d, J=2.0 Hz, 1H), 5.77 (br d, J=7.9 Hz, 1H), 4.00-3.83 (m, 1H), 2.87-2.75 (m, 3H), 1.88 (td, J=3.5, 8.6 Hz, 2H), 1.50-1.32 (m, 6H), 0.94 (s, 6H).

### Example 2, MPL-021

Scheme:

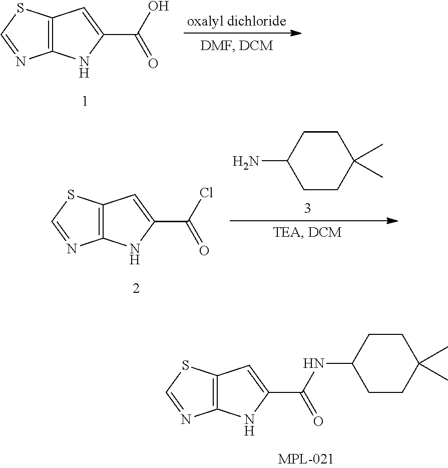

### Step 1. Synthesis of 4H-pyrrolo[2,3-d]thiazole-5-carbonyl chloride

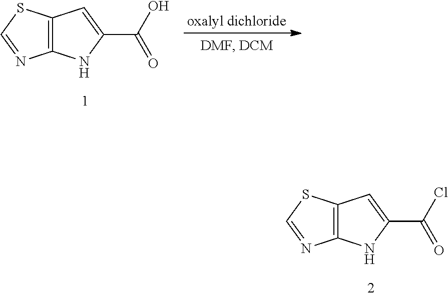

To a solution of (COCl)₂ (2.90 g, 22.85 mmol, 2 mL, 38.42 eq) in DCM (1 mL) was added DMF (2.17 mg, 29.73 μmol, 2.29 μL, 0.05 eq) and 4H-pyrrolo[2,3-d]thiazole-5-carboxylic acid (100 mg, 594.63 μmol, 1 eq) (diluted with DCM 1 mL). The mixture was stirred at 25° C. for 1.5 hours. LCMS (MeOH 0.5 mL) showed the starting material 1 was consumed and desire product formed. The mixture was directly concentrated under reduce pressure to give a residue. The residue was directly used in next step without any purification. Compound 4H-pyrrolo[2,3-d]thiazole-5-carbonyl chloride (100 mg, 428.68 μmol, 72.09% yield, 80% purity) was obtained as a white solid. LCMS (ESI) m/z 182.9 [M+H]⁺

### Step 2. Synthesis of N-(4,4-dimethylcyclohexyl)-4H-pyrrolo[2,3-d]thiazole-5-carboxamide

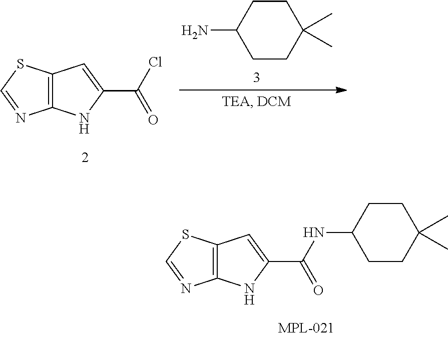

To a solution of 4H-pyrrolo[2,3-d]thiazole-5-carbonyl chloride (100 mg, 535.85 μmol, 1 eq) in DCM (4 mL) was added TEA (108.45 mg, 1.07 mmol, 149.17 μL, 2 eq) and 4,4-dimethylcyclohexanamine (102.26 mg, 803.78 μmol, 1.5 eq). The mixture was stirred at 25° C. for 4 hr. LCMS showed the starting material 2 was consumed and desire product formed. The mixture was directly concentrated under reduce pressure to give a residue. The residue was purified by column chromatography (SiO₂, Petroleum ether: EtOAc=10:1 to 3:1). Compound N-(4,4-dimethylcyclohexyl)-4H-pyrrolo [2,3-d]thiazole-5-carboxamide (35 mg, 122.77 μmol, 22.91% yield, 97.3% purity) was obtained as a white solid.

LCMS (ESI) m/z 278.1 [M+H]⁺; ¹H NMR (400 MHz, CHLOROFORM-d) δ=9.97 (br s, 1H), 8.67 (s, 1H), 6.75 (d, J=1.8 Hz, 1H), 5.85 (br d, J=7.9 Hz, 1H), 4.07-3.83 (m, 1H), 1.95-1.84(m, 2H), 1.52-1.21 (m, 8H), 0.95 (s, 6H).

### Example 3, MPL-025

### Synthesis of 2-methyl-N-[(1S,2S,3S,5R)-2,6,6-trimethylnorpinan-3-yl]-4H-pyrrolo[2,3-d] thiazole-5-carboxamide

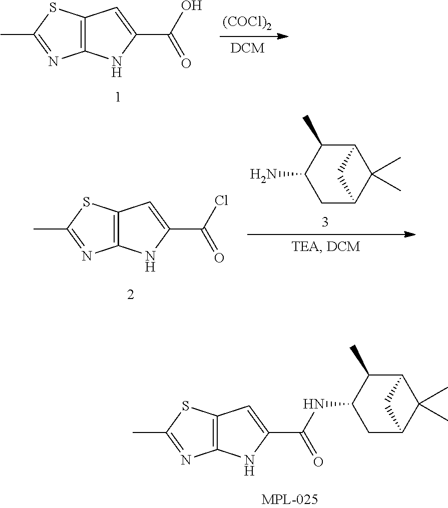

### Step 1. Synthesis of 2-methyl-4H-pyrrolo[2,3-d]thiazole-5-carbonyl chloride

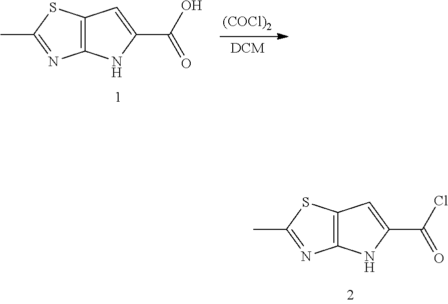

To a solution of 2-methyl-4H-pyrrolo[2,3-d]thiazole-5-carboxylic acid (80 mg, 439.08 umol, 1 eq) in DCM (3 mL) was added oxalyl dichloride (4.35 g, 34.27 mmol, 3 mL, 78.05 eq) and DMF (962.82 ug, 13.17 umol, 1.01 uL, 0.03 eq), the mixture was stireed at 30° C. for 12 hr under N₂. LCMS indicated 10% of reactant 5 was remained, and one main peak with desired MS was detected. The mixture was concentrated under reduced pressure to give a residue. The product 2-methyl-4H-pyrrolo[2,3-d]thiazole-5-carbonyl chloride (88 mg, 438.59 umol, 99.89% yield) was obtained as brown solid and was used directly for the next step without purification.

LCMS (ESI) m/z 197.0 [M−Cl+OMe]⁺

### Step 2. Synthesis of 2-methyl-N-[(1S,2S,3S,5R)-2,6,6-trimethylnorpinan-3-yl]-4H-pyrrolo[2,3-d] thiazole-5-carboxamide

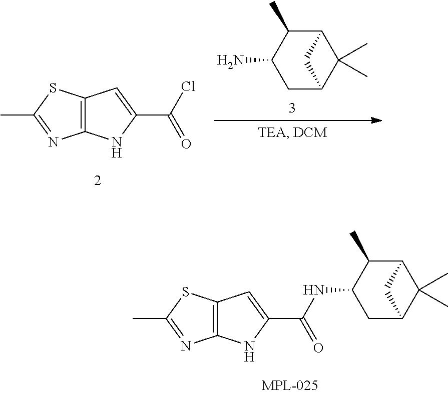

To a solution of 2-methyl-4H-pyrrolo[2,3-d]thiazole-5-carbonyl chloride (88 mg, 438.59 μmol, 1 eq) in DCM (3 mL) was added (1S,2S,3S,5R)-2,6,6-trimethylnorpinan-3-amine (168.05 mg, 1.10 mmol, 2.5 eq) and TEA (110.95 mg, 1.10 mmol, 152.61 uL, 2.5 eq), the mixture was stirred at 25° C. for 0.5 hr under N₂. TLC and LC-MS showed the starting material 1 was consumed completely and one main peak with desired MS was detected. The reaction mixture was diluted with solvent of DCM (40 mL) and washed with brine (20 mL×2), then the organic layers were dried over anhydrous Na₂SO₄, filtered and concentrated under reduced pressure to give a residue. The residue was purified by column chromatography (SiO₂, Petroleum ether: EtOAc=1:0 to 5:1) and prep. HPLC (column: Phenomenex Synergi C18 150*30 mm*4 um;mobile phase: [water(0.225%FA)-ACN];B%: 28%-58%,11 min). The product 2-methyl-N-[(1S,2S,3S,5R)-2,6,6-trimethylnorpinan-3-yl]-4H-pyrrolo[2,3-d]thiazole-5-carboxamide (10.1 mg, 31.45 μmol, 7.17% yield, 98.840% purity) was obtained as white solid.

LCMS (ESI) m/z 318.2 [M+H]⁺; ¹H NMR (400 MHz, CDCl₃) δ=9.73 (br s, 1H), 6.68 (d, J=1.5 Hz, 1H), 5.79 (br d, J=8.6 Hz, 1H), 4.48 (br t, J=8.0 Hz, 1H), 2.80 (s, 3H), 2.75-2.66 (m, 1H), 2.52-2.42 (m, 1H), 2.02 (s, 1H), 1.94-1.85 (m, 2H), 1.69-1.63 (m, 1H), 1.26 (s, 3H), 1.18 (d, J=7.1 Hz, 3H), 1.10 (s, 3H), 0.93 (d, J=9.9 Hz, 1H).

### Example 4, MPL-026

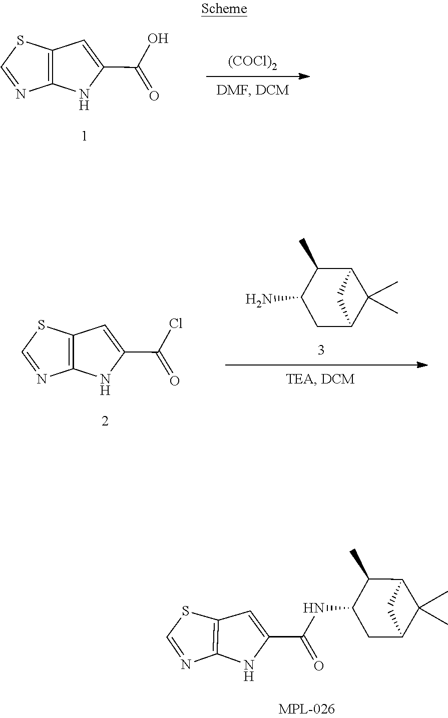 [SMILES: O=C(O)c1cc2scnc2[nH]1 ; "(COCl)2" ; O=C(Cl)c1cc2scnc2[nH]1 ; C[C@@H]1[C@@H](N)C[C@H]2C[C@@H]1C2(C)C ; C[C@@H]1[C@@H](NC(=O)c2cc3scnc3[nH]2)C[C@H]2C[C@@H]1C2(C)C]

### Step 1. Synthesis of 4H-pyrrolo[2,3-d]thiazole-5-carbonyl chloride

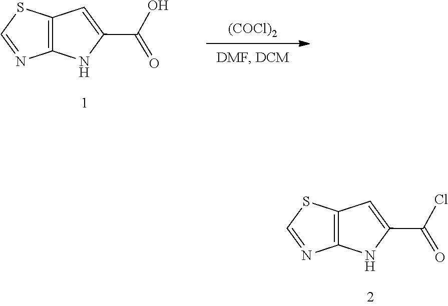 [SMILES: O=C(O)c1cc2scnc2[nH]1 ; "(COCl)2" ; O=C(Cl)c1cc2scnc2[nH]1]

To a solution of (COCl)₂ (2.90 g, 22.85 mmol, 2 mL, 38.42 eq) in DCM (1 mL) was added DMF (2.17 mg, 29.73 μmol, 2.29 μL, 0.05 eq) and 4H-pyrrolo[2,3-d]thiazole-5-carboxylic acid (100 mg, 594.63 μmol, 1 eq) (diluted with DCM 1 mL). The mixture was stirred at 25° C. for 1.5 hr. LCMS (MeOH 0.5 mL) showed the starting material 1 was consumed and desire product formed. The mixture was directly concentrated under reduce pressure to give a residue. The residue was directly used in next step without any purification. Compound 4H-pyrrolo[2,3-d]thiazole-5-carbonyl chloride (100 mg, 428.68 μmol, 72.09% yield, 80% purity) was obtained as a white solid. LCMS (ESI) m/z 182.9 [M+H]⁺

### Step 2. Synthesis of N-[(1S,2S,3S,5R)-2,6,6-trimethylnorpinan-3-yl]-4H-pyrrolo[2,3-d]thiazole-5-carboxamide

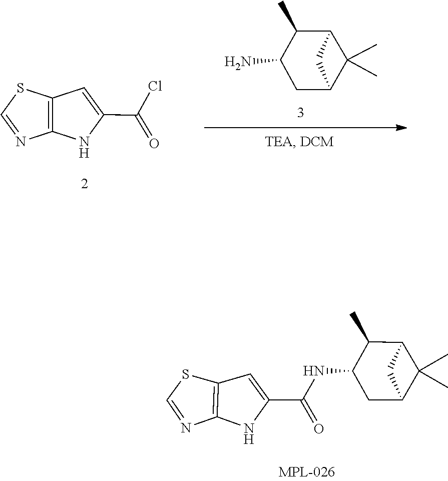 [SMILES: O=C(Cl)c1cc2scnc2[nH]1 ; C[C@@H]1[C@@H](N)C[C@H]2C[C@@H]1C2(C)C ; C[C@@H]1[C@@H](NC(=O)c2cc3scnc3[nH]2)C[C@H]2C[C@@H]1C2(C)C]

To a solution of 4H-pyrrolo[2,3-d]thiazole-5-carbonyl chloride (100 mg, 535.85 μmol, 1 eq) in DCM (4 mL) was added TEA (108.45 mg, 1.07 mmol, 149.17 μL, 2 eq) and (1S,2S,3S,5R)-2,6,6-trimethylnorpinan-3-amine (123.19 mg, 803.78 μmol, 1.5 eq). The mixture was stirred at 25° C. for 4 hours. LCMS showed the starting material 2 was consumed and desire product formed. The mixture was directly concentrated under reduce pressure to give a residue. The residue was purified by column chromatography (SiO₂, Petroleum ether: EtOAc=10:1 to 3:1). Compound N-[(1S,2S,3S,5R)-2,6,6-trimethylnorpinan-3-yl]-4H-pyrrolo[2,3-d]thiazole-5-carboxamide (55 mg, 181.27 μmol, 33.83% yield, 100% purity) was obtained as a white solid.

LCMS (ESI) m/z 304.1 [M+H]⁺; ¹H NMR (400 MHz, METHANOL-d₄) δ=8.81 (s, 1H), 7.16 (s, 1H), 4.61 (s, 1H), 4.53-4.45 (m, 1H), 2.65-2.54 (m, 1H), 2.52-2.41 (m, 1H), 2.12-2.03 (m,1H), 2.02-1.95 (m, 1H), 2.02-1.95 (m, 1H), 1.87 (br t, J=5.7 Hz, 1H), 1.72 (ddd, J=2.0, 6.7, 13.7 Hz, 1H), 1.28 (s, 3H), 1.16-1.10 (m, 6H).

### Example 5, MPL-042

### Synthesis of N-cyclooctyl-2-methyl-4H-pyrrolo[2,3-d]thiazole-5-carboxamide

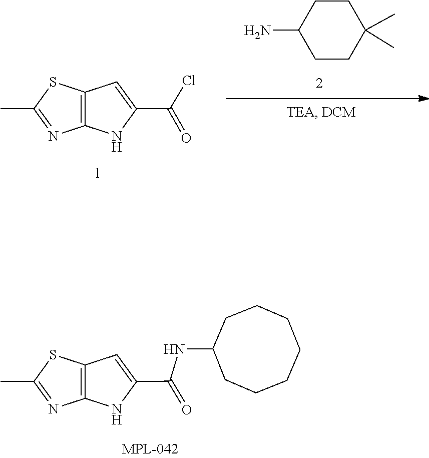

To a solution of 2-methyl-4H-pyrrolo[2,3-d]thiazole-5-carbonyl chloride (88 mg, 438.59 μmol, 1 eq) in DCM (3 mL) was added cyclooctaamine (167.40 mg, 1.32 mmol, 3 eq) and TEA (133.14 mg, 1.32 mmol, 183.14 μL, 3 eq), the mixture was stirred at 25° C. for 0.5 hours under N₂. TLC and LC-MS showed the starting material 1 was consumed completely and one main peak with desired MS was detected. The reaction mixture was diluted with solvent of DCM (40 mL) and washed with brine (20 mL×2). Then the organic layers were dried over anhydrous Na₂SO₄, filtered and concentrated under reduced pressure to give a residue. The residue was purified by column chromatography (SiO2, Petroleum ether:EtOAc=1:0 to 3:1) and prep.HPLC (column: Phenomenex Synergi C18 150*30 mm*4 um;mobile phase: [water (0.225%FA)-ACN];B%: 43%-65%,11 min). The product N-cyclooctyl-2-methyl-4H-pyrrolo[2,3-d]thiazole-5-carboxamide (10 mg, 34.02 μmol, 7.76% yield, 99.127% purity) was obtained as a brown solid. LCMS (ESI) m/z 292.2 [M+H]⁺; ¹H NMR (400 MHz, CDCl₃) δ=9.62 (br s, 1H), 6.64 (d, J=1.8 Hz, 1H), 5.83 (br d, J=7.3 Hz, 1H), 4.20 (br s, 1H), 2.79 (s, 3H), 1.99-1.90 (m, 2H), 1.75-1.60 (m, 12H).

### Example 6, MPL-090

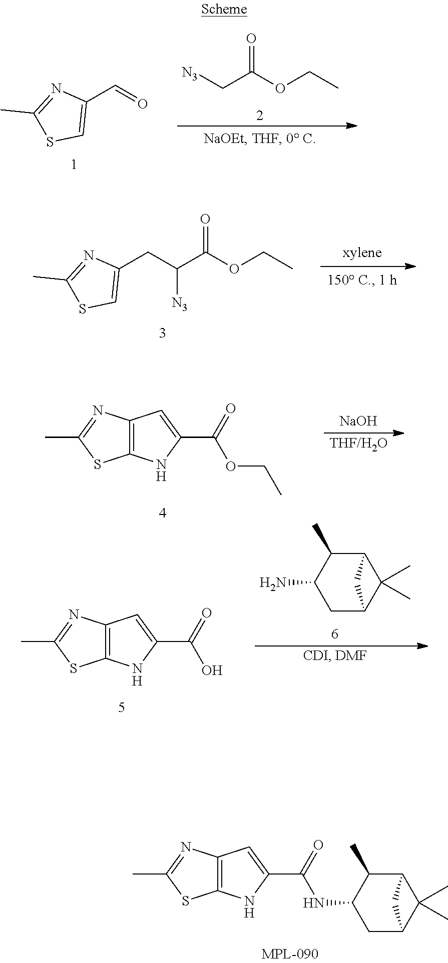

### Step 1. Synthesis of (Z)-ethyl 2-azido-3-(2-methylthiazol-4-yl)acrylate

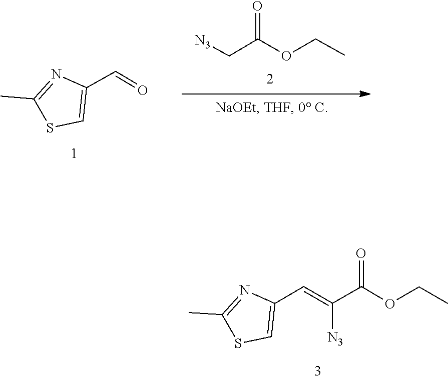

Na (451.98 mg, 19.66 mmol, 465.95 μL, 5 eq) was added to EtOH (20 mL), the mixture was stirred for 1 hr until Na dissolved. Then ethyl 2-azidoacetate (2.03 g, 15.73 mmol, 2.21 mL, 4 eq) and 2-methylthiazole-4-carbaldehyde (0.5 g, 3.93 mmol, 1 eq) in EtOH (20 mL) was added at 0° C. was added, the mixture was stirred at 0° C. for 2 hours under N₂. TLC and LCMS showed the completion of the reaction. The mixture was added to sat. NH₄Cl (40 mL), extracted by EtOAc (50 mL), the organic layer was dried with Na₂SO₄, filtered and concentrated by reduced pressure. The crude product ethyl (Z)-2-azido-3-(2-methylthiazol-4-yl)prop-2-enoate (0.9 g, crude) was obtained as yellow semi-oil. LCMS (ESI), m/z 224.8 [M−N]⁺

### Step 2. Synthesis of ethyl 2-methyl-4H-pyrrolo[3,2-d]thiazole-5-carboxylate

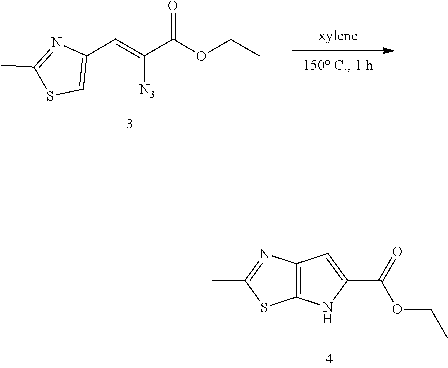 [SMILES: CCOC(=O)/C(=C/c1csc(C)n1)N=[N+]=[N-] ; CCOC(=O)c1cc2nc(C)sc2[nH]1]

The mixture of ethyl (Z)-2-azido-3-(2-methylthiazol-4-yl)prop-2-enoate (0.9 g, 3.78 mmol, 1 eq) in xylene (6 mL) was stirred at 150° C. for 1 hr. LCMS showed the completion of the reaction. The mixture was transferred to silica gel column (petroleum: EtOAc=100:1 to 3:1) directly to afford the product. Ethyl 2-methyl-4H-pyrrolo[3,2-d]thiazole-5-carboxylate (180 mg, 847.55 μmol, 22.44% yield, 99% purity) was obtained as white solid.

### Step 3. Synthesis of 2-methyl-4H-pyrrolo[3,2-d]thiazole-5-carboxylic acid

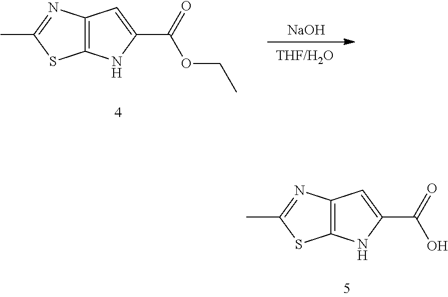

To the solution of ethyl 2-methyl-4H-pyrrolo[3,2-d]thiazole-5-carboxylate (180 mg, 856.11 μmol, 1 eq) in EtOH (4 mL) was added NaOH (2 M, 9.00 mL, 21.03 eq) and the mixture was stirred at 80° C. for 20 hours. LCMS showed the completion of the reaction. The mixture was concentrated under reduced pressure to give a residue, then diluted with water (2 mL), acidified with HCl (2 M) to pH=6. The mixture was filtered and the filter cake was washed with 10 mL H₂O, dried under reduced pressure to give product. The crude product 2-methyl-4H-pyrrolo [3,2-d]thiazole-5-carboxylic acid (100 mg, 521.41 μmol, 60.90% yield, 95% purity) was obtained as a red solid.

### Step 4. Synthesis of 2-methyl-N-((1S,2S,3S,5R)-2,6,6-trimethylbicyclo[3.1.1]heptan-3-yl)-4H-pyrrolo[3,2-d]thiazole-5-carboxamide

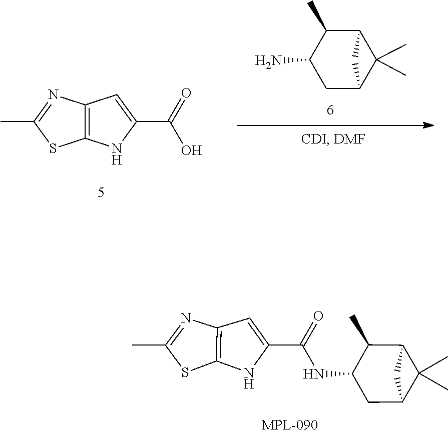

To the solution of 2-methyl-4H-pyrrolo[3,2-d]thiazole-5-carboxylic acid (40 mg, 219.54 μmol, 1 eq) in DMF (1 mL) was added CDI (53.40 mg, 329.31 μmol, 1.5 eq) and the mixture was stirred at 30° C. for 1 hr. Then (1S,2S,3S,5R)-2,6,6-trimethylnorpinan-3-amine (50.47 mg, 329.31 μmol, 1.5 eq) was added and the mixture was stirred at 30° C. for 12 hours under N₂. LCMS showed the completion of the reaction. EtOAc (5 mL) was added, the mixture was washed by LiCl (15%, 5 mL×3), the organic layer was dried with Na₂SO₄, filtered and concentrated by reduced pressure. The residue was purified with pre-HPLC (HCO₂H) (column: Boston Green ODS 150*30 5 u; mobile phase: [water (0.225%FA)-ACN]; B%: 38%-68%,10 min) to afford the product. 2-methyl-N-[(1S,2 S,3 S, 5R)-2, 6, 6-trimethylnorpinan-3 -yl]-4H-pyrrolo[3, 2-d]thiazole-5-carboxamide (30 mg, 94.50 μmol, 43.05% yield, 100% purity) was obtained as a white solid.

LCMS (ESI), m/z 317.9 [M+H]⁺; ¹H NMR (500 MHz, DMSO-d₆) δ=11.85 (s, 1H), 8.04 (d, J=8.5 Hz, 1H), 7.18 (d, J=2.0 Hz, 1H), 4.41-4.28 (m, 1H), 2.68 (s, 3H), 2.42 (br dd, J=10.1, 12.2Hz, 2H), 2.10-2.00 (m, 1H), 1.98-1.92 (m, 1H), 1.81 (t, J=5.0 Hz, 1H), 1.68 (ddd, J=2.1, 6.6, 13.7 Hz, 1H), 1.24 (s, 3H), 1.21 (d, J=9.5 Hz, 1H), 1.09-1.03(m, 6H).

### Example 7, MPL-091

### Step 1. Synthesis of N-cyclooctyl-2-methyl-4H-pyrrolo[3,2-d]thiazole-5-carboxamide

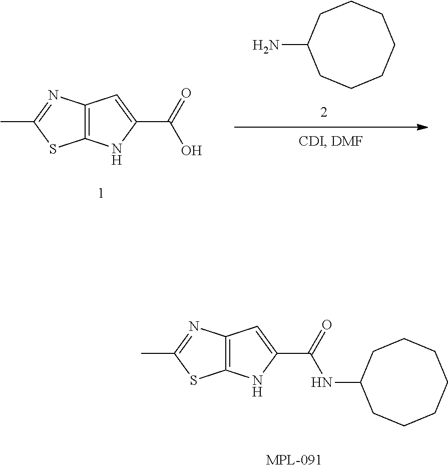 [SMILES: NC1CCCCCCC1 ; Cc1nc2cc(C(=O)O)[nH]c2s1 ; Cc1nc2cc(C(=O)NC3CCCCCCC3)[nH]c2s1]

To the solution of 2-methyl-4H-pyrrolo[3,2-d]thiazole-5-carboxylic acid (60 mg, 329.31 μmol, 1 eq) in DMF (1 mL) was added CDI (80.10 mg, 493.96 μmol, 1.5 eq) and the mixture was stirred at 30° C. for 1 hr. Then cyclooctanamine (62.85 mg, 493.96 μmol, 1.5 eq) was added, the mixture was stirred at 30° C. for 12 hours under N₂. LCMS showed the completion of the reaction. The mixture was filtered and the solid was washed by DMF (2 mL), followed by water (5 mL). Then it was concentrated under reduced pressure to afford N-cyclooctyl-2-methyl-4H-pyrrolo[3,2-d]thiazole-5-carboxamide (60 mg, 205.89 μmol, 62.52% yield, 100% purity) was obtained as a white solid.

LCMS (ESI), m/z 292.1 [M+H]⁺; ¹H NMR (500 MHz, DMSO-d₆) δ=11.81 (br s, 1H), 7.94 (br d, J=7.9 Hz, 1H), 7.16 (s, 1H), 4.06-3.94 (m, 1H), 2.67 (s, 3H), 1.84-1.44 (m, 14H).

### Example 8, MPL-142

### Synthesis of N-cyclooctyl-2-cyclopropyl-4H-pyrrolo[2,3-d]thiazole-5-carboxamide

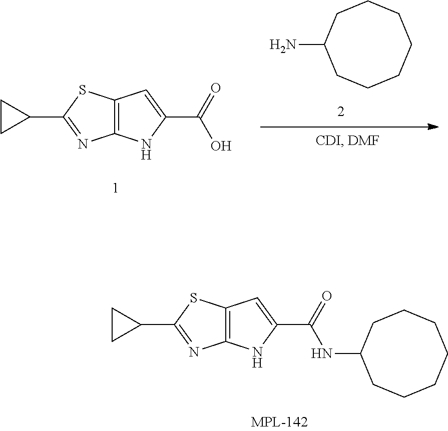 [SMILES: O=C(O)c1cc2sc(C3CC3)nc2[nH]1 ; NC1CCCCCCC1 ; O=C(NC1CCCCCCC1)c1cc2sc(C3CC3)nc2[nH]1]

To a solution of 2-cyclopropyl-4H-pyrrolo[2,3-d]thiazole-5-carboxylic acid (180 mg, 172.88 μmol, 1 eq) in DMF (2 mL) was added CDI (84.10 mg, 518.64 μmol, 3 eq). Compound 1 has been described in the literature (CAS 1379300-94-3). The mixture was stirred at 30° C. for 0.5 h.

Then cyclooctanamine (32.99 mg, 259.32 mol, 1.5 eq) was added and the mixture was stirred for another 12 h at the same temperature. LCMS showed the completion of the reaction. The mixture was dropwise added to water (10 mL), and stirred for 10 min, filtered and the filter cake was dried under reduced pressure to give the crude product. The residue was purified by prep-HPLC (column: YMC-Actus Triart C18 150*30 mm*5 um;mobile phase: [water (0.225%FA)-ACN]; B%: 46%-76%,11 min). The product N-cyclooctyl-2-cyclopropyl-4H-pyrrolo[2,3-d]thiazole-5-carboxamide (9.7 mg, 30.56 μmol, 17.67% yield, 100% purity) was obtained as a white solid.

LCMS (ESI) m/z 318.1 [M+H]⁺; ¹H NMR (400 MHz, DMSO-δ₆) δ=12.10 (s, 1H), 7.86 (d, J=7.8 Hz, 1H), 7.03 (d, J=1.5 Hz, 1H), 3.99 (br d, J=4.4 Hz, 1H), 2.40-2.33 (m, 1H), 1.75-1.47 (m, 14H), 1.16-1.10 (m, 2H), 1.02-0.96 (m, 2H).

### Example 9, MPL-144

### Synthesis of 2-cyclopropyl-N-((1S,2S,3S,5R)-2,6,6-trimethylbicyclo[3.1.1]heptan-3-yl)-4H-pyrrolo[2,3-d]thiazole-5-carboxamide

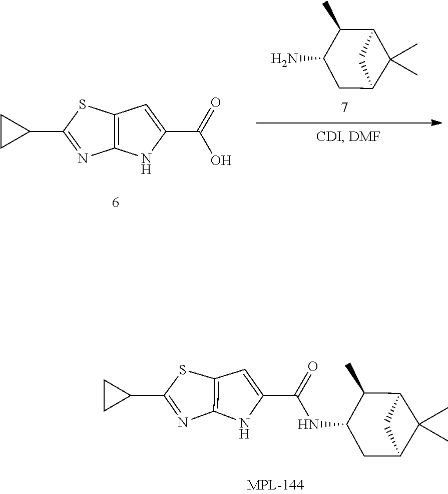 [SMILES: C[C@@H]1[C@@H](N)C[C@H]2C[C@@H]1C2(C)C ; O=C(O)c1cc2sc(C3CC3)nc2[nH]1 ; C[C@@H]1[C@@H](NC(=O)c2cc3sc(C4CC4)nc3[nH]2)C[C@H]2C[C@@H]1C2(C)C]

To a solution of 2-cyclopropyl-4H-pyrrolo[2,3-d]thiazole-5-carboxylic acid (180 mg, 172.88 μmol, 1 eq) in DMF (2 mL) was added CDI (84.10 mg, 518.64 μmol, 3 eq). The mixture was stirred at 30° C. for 0.5 h, then (1S,2S,3S,5R)-2,6,6-trimethylnorpinan-3-amine (79.49 mg, 518.64 μmol, 3 eq) was added and the mixture was stirred for another 23.5 h at the same temperature. LCMS showed the completion of the reaction. The mixture was dropwise added to water (15 mL), and stirred for 10 min, filtered and the filter cake was dried under reduced pressure to give the crude product. The residue was purified by prep-HPLC (column: YMC-Actus Triart C18 150*30 mm*5 um;mobile phase: [water (0.225%FA)-ACN];B%: 58%-85%,11 min). The product 2-cyclopropyl-N-[(1S,2S,3S,5R) -2,6,6-trimethylnorpinan-3-yl]-4H-pyrrolo[3,2-d]thiazole-5-carboxamide (12.2 mg, 34.56 μmol, 19.99% yield, 97.289% purity) was obtained as a white solid.

LCMS (ESI) m/z 344.2 [M+H]⁺; ¹H NMR (400 MHz, DMSO-δ₆) δ=12.11 (s, 1H), 7.97 (br d, J=8.6 Hz, 1H), 7.05 (d, J=1.7 Hz, 1H), 4.32 (br t, J=7.7 Hz, 1H), 2.44-2.33 (m, 3H), 2.02 (br t, J=7.2 Hz, 1H), 1.93 (br s, 1H), 1.84-1.77 (m, 1H), 1.70-1.60 (m, 1H), 1.22 (s, 3H), 1.19-1.11 (m, 3H), 1.07-0.98 (m, 8H).

### Example 10, MPL-150

### Synthesis of 2-cyclopropyl-6-methyl-N-((1S,2S,3S,5R)-2,6,6-trimethylbicyclo[3.1.1]heptan-3-yl)-4H-pyrrolo[2,3-d]thiazole-5-carboxamide

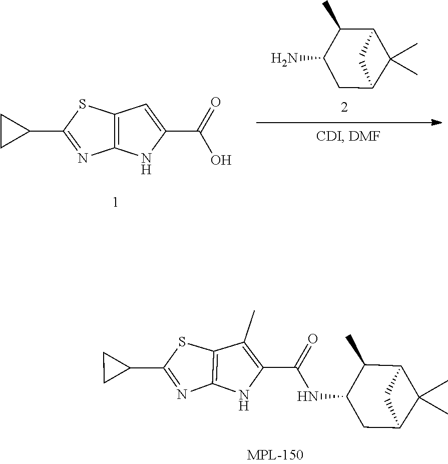 [SMILES: C[C@@H]1[C@@H](N)C[C@H]2C[C@@H]1C2(C)C ; O=C(O)c1cc2sc(C3CC3)nc2[nH]1 ; Cc1c(C(=O)N[C@H]2C[C@H]3C[C@@H]([C@@H]2C)C3(C)C)[nH]c2nc(C3CC3)sc12]

To a solution of 2-cyclopropyl-6-methyl-4H-pyrrolo[2,3-d]thiazole-5-carboxylic acid (compound 1, 60 mg, 269.95 μmol, 1 eq) in DMF (1.5 mL) was added CDI (65.66 mg, 404.93 μmol, 1.5 eq). For the synthesis of compound 1, see Example 14. The mixture was stirred at 25° C. for 0.5 h, then (1S,2S,3S,5R)-2,6,6-trimethylnorpinan-3-amine (62.06 mg, 404.93 μmol, 1.5 eq) was added and the mixture was stirred at the same temperature for 0.5 h. LC-MS showed the starting material 1 was consumed completely and one main peak with desired MS was detected. The mixture was dropwise added to water (50 mL), and stirred for 10 min, filtered and the filter cake was dried under reduced pressure to give the crude product. The residue was purified by prep-HPLC (column: YMC-Actus Triart C18 150*30 mm*5 um;mobile phase: [water (0.225%FA)-ACN];B%: 73%-93%,11 min). The product (39.7 mg, 109.96 μmol, 40.73% yield, 99.020% purity) was obtained as a white solid.

LCMS (ESI) m/z 558.2 [M+H]⁺; ¹H NMR (500 MHz, DMSO-d₆) δ=11.77 (s, 1H), 7.45 (d, J=8.2 Hz, 1H), 4.28 (br s, 1H), 2.47-2.42 (m, 1H), 2.40 (s, 3H), 2.39-2.36 (m, 2H), 2.02-1.91 (m, 2H), 1.81 (t, J=5.2 Hz, 1H), 1.62 (ddd, J=2.3, 6.3, 13.7 Hz, 1H), 1.22 (s, 3H), 1.17-1.12 (m, 2H), 1.09 (s, 1H), 1.07-1.04 (m, 6H), 1.01-0.96 (m, 2H).

### Example 11, MPL-205

### Synthesis of N-(1,1-dimethylsilinan-4-yl)-2-methyl-4H-pyrrolo [2,3-d]thiazole-5-carboxamide

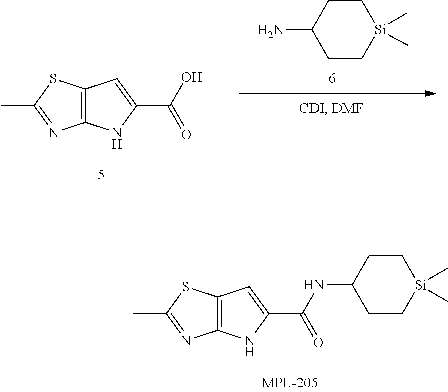

To a solution of 2-methyl-4H-pyrrolo [2,3-d]thiazole-5-carboxylic acid (100 mg, 548.85 μmol, 1 eq) in DMF (3 mL) was added CDI (115.69 mg, 713.50 μmol, 1.3 eq). The mixture was stirred at 25° C. for 0.5 h. Then 1,1-dimethylsilinan-4-amine (102.25 mg, 713.50 μmol, 1.3 eq) was added. The mixture was stirred at 25° C. for 11.5 h. LCMS showed there was no starting material. The reaction was added dropwise to H₂O (20 mL). There was much precipitation which was collected by filter. The cake was diluted with EtOAc (30 mL), dried with anhydrous Na₂SO₄, and filtered. The filtrate was concentrated in vacuo. The crude product was triturated with ACN (2.5 mL) at 30° C. for 45 min, filtered. The cake was transferred in bottom flask. Compound N-(1,1-dimethylsilinan-4-yl)-2-methyl-4H-pyrrolo [2,3-d]thiazole-5-carboxamide (108 mg, 339.69 μmol, 61.89% yield, 96.712% purity) was obtained as a white solid.

LCMS (ESI), m/z 308.1[M+H]⁺; ¹H NMR (500 MHz, DMSO-d₆) δ=12.16 (br s, 1H), 7.89 (br d, J=8.1 Hz, 1H), 7.02 (s, 1H), 3.67 (br d, J=7.9 Hz, 1H), 2.69 (s, 3H), 1.96 (br d, J=9.6 Hz, 2H),1.62-1.50 (m, 2H), 0.76 (br d, J=14.5 Hz, 2H), 0.59 (dt, J=4.5, 14.0 Hz, 2H), 0.10-0.01 (m, 6H).

### Example 12, MPL-206

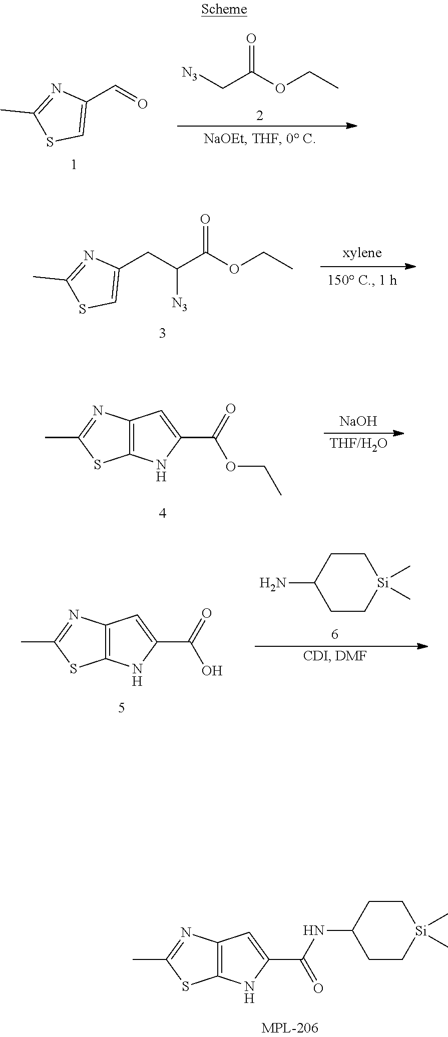

### Step 1. Synthesis of ethyl (Z)-2-azido-3-(2-methylthiazol-4-yl)prop-2-enoate

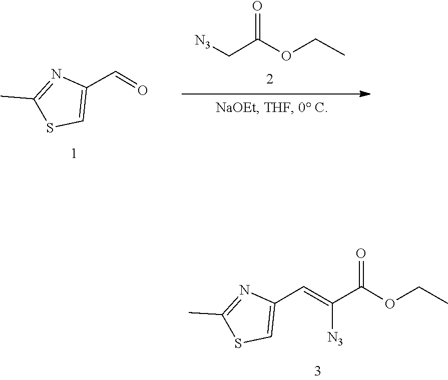

To a solution NaH (4.72 g, 117.96 mmol, 60% purity, 5 eq) in EtOH (90 mL) cooled to 0° C. The mixture was stirred at 0° C. for 0.5 h. Then 2-methylthiazole-4-carbaldehyde (3 g, 23.59 mmol, 1 eq) and ethyl 2-azidoacetate (15.23 g, 117.96 mmol, 16.55 mL, 5 eq) was added. The mixture was stirred at 0° C. for 1.5 h. TLC (Petroleum ether: EtOAc=5:1) and LCMS indicated the reaction was finished. The mixture was adjusted to pH=8 with HCl (2N). The mixture was extracted with EtOAc (400 mL×3). The organic layers were combined and dried over Na₂SO4, filtered and concentrated, leading to the crude product. The residue was purified by column chromatography (SiO₂, petroleum ether/EtOAc=1:0 to 5:1). Compound ethyl (Z)-2-azido-3-(2-methylthiazol-4-yl)prop-2-enoate (1.10 g, 4.14 mmol, 17.56% yield, 90% purity) was obtained as a white solid.

### Step 2. Synthesis of ethyl 2-methyl-4H-pyrrolo[3,2-d]thiazole-5-carboxylate

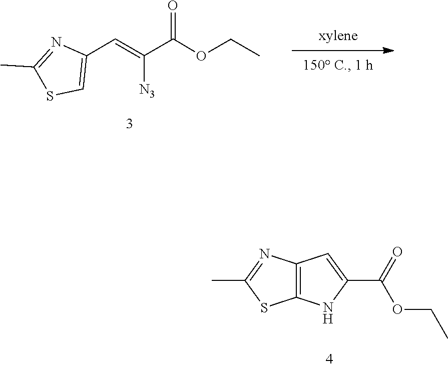 [SMILES: CCOC(=O)/C(=C/c1csc(C)n1)N=[N+]=[N-] ; CCOC(=O)c1cc2nc(C)sc2[nH]1]

The solution of ethyl (Z)-2-azido-3-(2-methylthiazol-4-yl)prop-2-enoate (1.10 g, 4.60 mmol, 1 eq) in xylene (11 mL) was heated to 150° C. for 1 hr. LCMS showed there was no starting material. The reaction mixture was submerged 2 hours and then filtered. The cake was dissolved with EtOAc (20 mL). The mixture was concentrated in vacuo. The filtrate was purified by column chromatography (SiO₂, Petroleum ether EtOAc=1:0 to 3:1). Compound ethyl 2-methyl-4H-pyrrolo[3,2-d]thiazole-5-carboxylate (750 mg, 3.39 mmol, 73.60% yield, 95% purity) was obtained as a white solid.

### Step 3. Synthesis of 2-methyl-4H-pyrrolo[3,2-d]thiazole-5-carboxylic acid

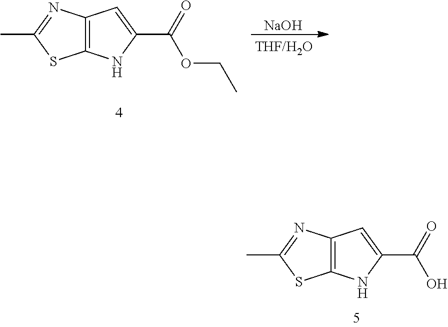

To a solution of ethyl 2-methyl-4H-pyrrolo[3,2-d]thiazole-5-carboxylate (750 mg, 3.57 mmol, 1 eq) in EtOH (1 mL) was added NaOH (2 M, 1 mL, 5.61e-1 eq) the mixture was stirred at 70° C. for 12 hr. LCMS showed the starting material was consumed completely. The mixture was concentrated under reduced pressure to give a residue, then diluted with water (10 mL), acidified with HCl (2 M) to pH=5. The mixture was filtered and the filter cake was dried under reduced pressure to give product. The product 2-methyl-4H-pyrrolo[3,2-d]thiazole-5-carboxylic acid (304 mg, 1.47 mmol, 41.16% yield, 88% purity) was obtained as a brown solid.

### Step 4. Synthesis of N-(1,1-dimethylsilinan-4-yl)-2-methyl-4H-pyrrolo[3,2-d]thiazole-5-carboxamide

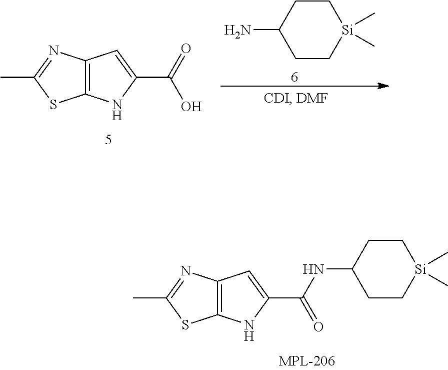

To a solution of 2-methyl-4H-pyrrolo [3,2-d]thiazole-5-carboxylic acid (150 mg, 823.27 μmol, 1 eq) in DMF (4.5 mL) was added CDI (173.54 mg, 1.07 mmol, 1.3 eq). The mixture was stirred at 25° C. for 0.5 h. Then 1,1-dimethylsilinan-4-amine (153.37 mg, 1.07 mmol, 1.3 eq) was added. The mixture was stirred at 10° C. for 11.5 h. LCMS showed there was no starting material. The reaction was added dropwise to H₂O (20 mL). The resulting precipitation was collected by filter.

The cake was diluted with EtOAc (30 mL), dried with anhydrous MgSO₄ and filtered. The filtrate was concentrated in vacuo. The residue was purified by prep-HPLC (column: YMC-Actus Triart C18 100*30 mm*5 um;mobile phase: [water (0.225%FA)-ACN];B%: 46%-76%,11 min). Then residue was diluted in ACN (5 mL) and H₂O (20 mL), then lyophilized. Compound N-(1, 1-dimethylsilinan-4-yl)-2-methyl-4H-pyrrolo [3, 2-d]thiazole-5-carboxamide (61 mg, 189.79 μmol, 23.05% yield, 95.667% purity) was obtained as a brown solid. This material was purified by pre. HPLC (column: Phenomenex Synergi C18 150*30 mm*4 um; mobile phase: [water (0.05%HCl)-ACN]; B%: 42%-72%, 10 min). The compound N-(1, 1-dimethylsilinan-4-yl)-2-methyl-4H-pyrrolo [3, 2-d]thiazole-5-carboxamide (35 mg, 113.83 μmol, 57.38% yield, 100% purity) was obtained as a white solid.

LCMS (ESI), m/z 308.1 [M+H]⁺; ¹H NMR (400 MHz, DMSO-d₆) δ=11.85 (s, 1H), 7.93 (d, J=8.2 Hz, 1H), 7.12 (d, J=2.0 Hz, 1H), 3.66 (br d, J=7.8 Hz, 1H), 2.66 (s, 3H), 1.97 (br d, J=11.3Hz, 2H), 1.63-1.49 (m, 2H), 0.76 (br d, J=14.1 Hz, 2H), 0.65-0.54 (m, 2H), 0.08 (s, 3H), 0.03 (s, 3H).

### Example 13, MPL-224

### Synthesis of N-[(1R,2R,3S,5R)-2-hydroxy-2,6,6-trimethyl-norpinan-3-yl]-2-methyl-4H-pyrrolo[2,3-d]thiazole-5-carboxamide

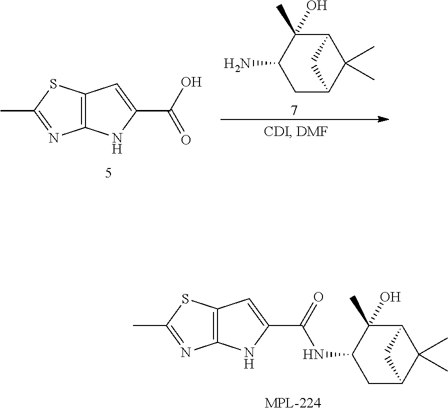

To a solution of 2-methyl-4H-pyrrolo[2,3-d]thiazole-5-carboxylic acid (110 mg, 603.73 μmol, 1 eq) and CDI (127.26 mg, 784.85 μmol, 1.3 eq) in DMF (2 mL). The mixture was stirred at 30° C. for 0.5 h. (1R,2R,3S,5R)-3-amino-2,6,6-trimethyl-norpinan-2-ol (132.85 mg, 784.85 mol, 1.3 eq) was added. The reaction was stirred at 30° C. for 11.5 h. LC-MS showed most of the starting material was consumed. The reaction mixture was added to water (20 mL). Then filtered and the filter cake was washed with 10 mL of water, and dried in vacuo to give product. The residue was diluted in ACN (5 mL) and H₂O (20 mL), then lyophilized. The product N-[(1R,2R,3S,5R)-2-hydroxy-2,6,6-trimethyl-norpinan-3-yl]-2-methyl-4H-pyrrolo[2,3-d]thiazole-5-carboxamide (19.5 mg, 58.48 μmol, 9.69% yield) was obtained as a white solid.

LCMS (ESI) m/z 316.2 [M−OH⁻]⁺; ¹H NMR (400 MHz, DMSO-d6) δ=12.29 (s, 1H), 7.57 (d, J=8.6 Hz, 1H), 7.04 (s, 1H), 4.56-4.43 (m, 2H), 2.71 (s, 3H), 2.31-2.22 (m, 1H), 2.11 (br s, 1H), 1.89 (br d, J=6.3 Hz, 2H), 1.63-1.56 (m, 2H), 1.26 (s, 3H), 1.19 (s, 3H), 1.06 (s, 3H).

### Example 14, MPL-228

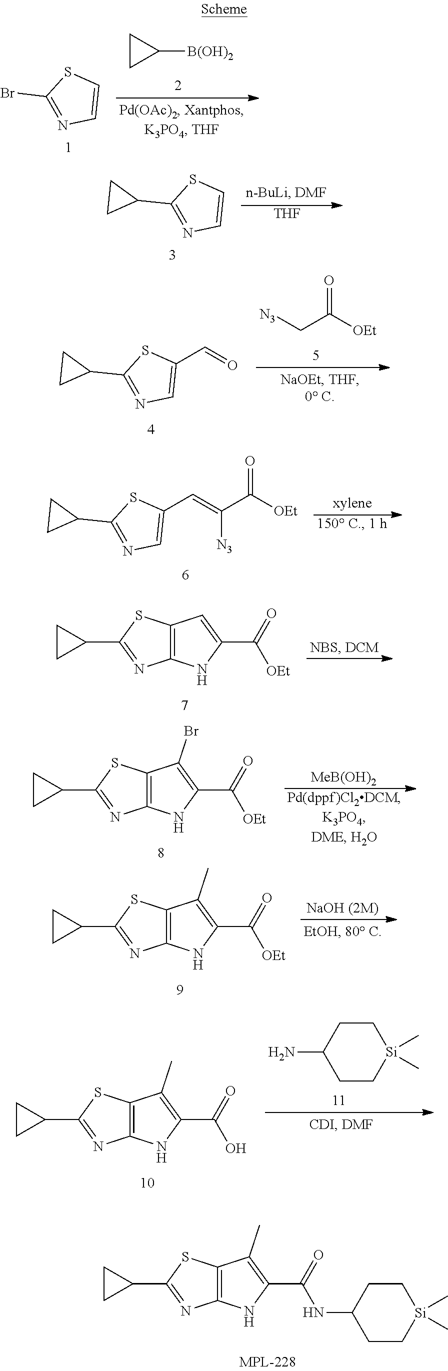

### Step 1. Synthesis of 2-cyclopropylthiazole

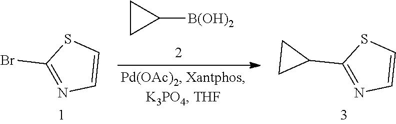

To a solution of 2-bromothiazole (20 g, 121.93 mmol, 10.99 mL, 1 eq) and cyclopropylboronic acid (20.95 g, 243.87 mmol, 2 eq) in THF (250 mL) was added K₃PO₄ (77.65 g, 365.80 mmol, 3 eq), Xantphos (7.06 g, 12.19 mmol, 0.1 eq) and Pd(OAc)₂ (2.74 g, 12.19 mmol, 0.1 eq). The mixture was stirred at 80° C. for 36 hr under N₂. TLC showed the starting material 1 was consumed completely and one new spot formed. The mixture was filtered and distilled under reduced pressure (10 Torr, 100° C.) to give the crude product. The product 2-cyclopropylthiazole (9.3 g, 40.11 mmol, 32.90% yield, 54% purity) was obtained as a colorless solid.

### Step 2. Synthesis of 2-cyclopropylthiazole-5-carbaldehyde

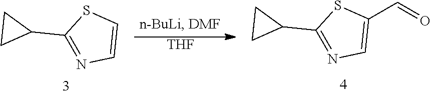 [SMILES: c1csc(C2CC2)n1 ; O=Cc1cnc(C2CC2)s1 ; "THF"]

To a solution of 2-cyclopropylthiazole (8 g, 34.51 mmol, 1 eq) in THF (80 mL) at −78° C. under N₂ was dropwise added n-BuLi (2.5 M, 24.85 mL, 1.8 eq). The mixture was stirred for 0.5 h, then DMF (12.61 g, 172.54 mmol, 13.28 mL, 5 eq) was added dropwise and the mixture was stirred for 1 h. TLC indicated the Reactant 3 was consumed completely. The mixture was quenched by addition of NH₄Cl (50 mL), extracted with EtOAc (200 mL×2). The combined organic layers were dried over Na₂SO₄, filtered and concentrated under reduced pressure to give a residue. The residue was purified by column chromatography (SiO₂, Petroleum ether/EtOAc=1:0 to 3:1). The product 2-cyclopropylthiazole-5-carbaldehyde (3.2 g, 18.80 mmol, 54.48% yield, 90% purity) was obtained as yellow solid.

### Step 3. Synthesis of ethyl (Z)-2-azido-3-(2-cyclopropylthiazol-5-yl)prop-2-enoate

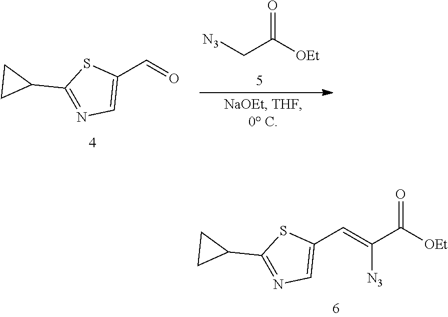 [SMILES: CCOC(=O)CN=[N+]=[N-] ; O=Cc1cnc(C2CC2)s1 ; CCOC(=O)/C(=C/c1cnc(C2CC2)s1)N=[N+]=[N-]]

NaH (2.87 g, 71.80 mmol, 60% purity, 5 eq) was added into EtOH(20 mL), the mixture was stirred at −10° C. for 0.1 h, then the mixture of 2-cyclopropylthiazole-5-carbaldehyde (2.2 g, 14.36 mmol, 1 eq) and ethyl 2-azidoacetate (5.56 g, 43.08 mmol, 6.05 mL, 3 eq) in EtOH(10 mL) was added dropwise at −10° C., the mixture was stirred for 2.9 h at the same temperature TLC showed the starting material 4 was consumed completely. The reaction mixture was quenched by addition of saturated aqueous NH₄Cl (100 mL) 0° C., and then extracted with EtOAc (150 mL×3). The combined organic layers were washed with brine (100 mL), dried over anhydrous Na₂SO₄, filtered and concentrated under reduced pressure to give a residue. The residue was purified by column chromatography (SiO₂, Petroleum ether/EtOAc=1:0 to 1:1). The product ethyl (Z)-2-azido-3-(2-cyclopropylthiazol-5-yl)prop-2-enoate (1.6 g, 5.45 mmol, 37.94% yield, 90% purity) was obtained as yellow solid.

### Step 4. Synthesis of ethyl 2-cyclopropyl-4H-pyrrolo[2,3-d]thiazole-5-carboxylate

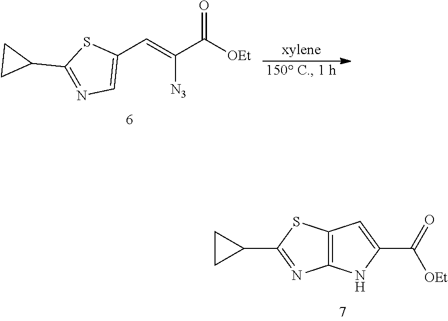

The solution of ethyl (Z)-2-azido-3-(2-cyclopropylthiazol-5-yl)prop-2-enoate (2.5 g, 9.46 mmol, 1 eq) in xylene (25 mL) was stirred at 150° C. for 1 hr. TLC showed the reactant 6 was consumed completely. After cooling to 0° C., a solid separated out and the mixture was filtered. The product ethyl 2-cyclopropyl-4H-pyrrolo[2,3-d]thiazole-5-carboxylate (2 g, 8.46 mmol, 89.48% yield, 100% purity) was obtained as a brown solid.

### Step 5. Synthesis of ethyl 6-bromo-2-cyclopropyl-4H-pyrrolo[2,3-d]thiazole-5-carboxylate

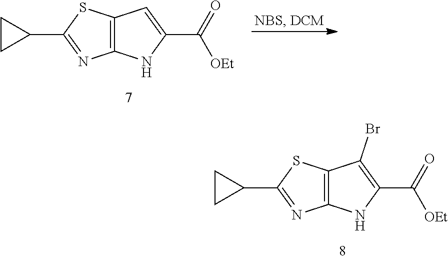

To a solution of ethyl 2-cyclopropyl-4H-pyrrolo[2,3-d]thiazole-5-carboxylate (1.4 g, 5.92 mmol, 1 eq) in DCM (20 mL) at 0° C. was added in portions NBS (1.16 g, 6.52 mmol, 1.1 eq), the mixture was stirred at 0° C. for 1 hr. LCMS showed the reactant 7 was consumed completely. The mixture was quench by addition of water (0.5 mL), then diluted with DCM (30 mL), dried with Na₂SO₄ and concentrated under reduce pressure to give the crude product. The residue was purified by flash silica gel chromatography (EtOAc/Petroleum ether gradient=1:0 to 3:1). The product ethyl 6-bromo-2-cyclopropyl-4H-pyrrolo[2,3-d]thiazole-5-carboxylate (1.7 g, 4.85 mmol, 81.93% yield, 90% purity) was obtained as white solid.

LCMS (ESI) m/z 314.9 [M+H]⁺

### Step 6. Synthesis of ethyl 2-cyclopropyl-6-methyl-4H-pyrrolo[2,3-d]thiazole-5-carboxylate

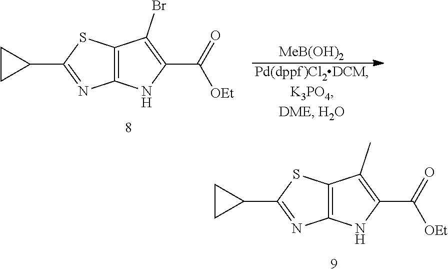 [SMILES: "MeB(OH)2" ; CCOC(=O)c1[nH]c2nc(C3CC3)sc2c1Br ; "Pd(dppf)Cl2·DCM,K3PO4,DME, H2O" ; CCOC(=O)c1[nH]c2nc(C3CC3)sc2c1C]

To a solution of ethyl 6-bromo-2-cyclopropyl-4H-pyrrolo[2,3-d]thiazole-5-carboxylate (1.2 g, 2.74 mmol, 1 eq), methylboronic acid (1.64 g, 27.41 mmol, 10 eq) and K₃PO₄ (1.75 g, 8.22 mmol, 3 eq) and XPhos (130.68 mg, 274.12 μmol, 0.1 eq) in dioxane (15 mL) was added Pd₂(dba)₃ (251.02 mg, 274.12 μmol, 0.1 eq), the mixture was stirred at 110° C. for 12 hr under N₂. LCMS showed the reactant 8 was consumed completely. The mixture was diluted with EtOAc (20 mL), filtered and the filter was concentrated under reduce pressure. The residue was purified by flash silica gel chromatography (EtOAc/Petroleum ether gradient=1:0 to 3:1). The product ethyl 2-cyclopropyl-6-methyl-4H-pyrrolo[2,3-d]thiazole-5-carboxylate (840 mg, 2.68 mmol, 97.93% yield, 80% purity) was obtained as a white solid.

LCMS (ESI) m/z 251.0 [M+H]⁺

### Step 7. Synthesis of 2-cyclopropyl-6-methyl-4H-pyrrolo[2,3-d]thiazole-5-carboxylic acid

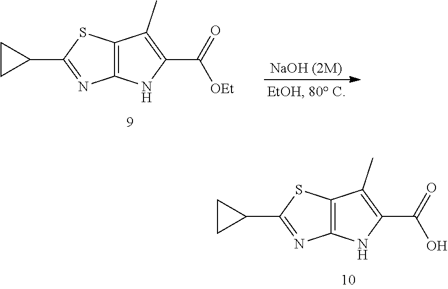 [SMILES: CCOC(=O)c1[nH]c2nc(C3CC3)sc2c1C ; Cc1c(C(=O)O)[nH]c2nc(C3CC3)sc12]

To a solution of ethyl 2-cyclopropyl-6-methyl-4H-pyrrolo[2,3-d]thiazole-5-carboxylate (840 mg, 3.36 mmol, 1 eq) in EtOH (5 mL) was added NaOH (4 M, 5 mL, 5.96 eq), the mixture was stirred at 75° C. for 12 hr. LCMS showed that reactant 9 was consumed completely and the desired MS was detected. The mixture was concentrated under reduced pressure to remove the EtOH, acidified with HCl (6 M) to the pH=4, filtered and the filter cake was dried under reduced pressure. The product 2-cyclopropyl-6-methyl-4H-pyrrolo[2,3-d]thiazole-5-carboxylic acid (550 mg, 2.10 mmol, 62.68% yield, 85% purity) was obtained as a brown solid.

LCMS (ESI) m/z 223.0 [M+H]⁺

### Step 8. Synthesis of 2-cyclopropyl-N-(1,1-dimethylsilinan-4-yl)-6-methyl-4H-pyrrolo [2,3-d]thiazole-5-carboxamide

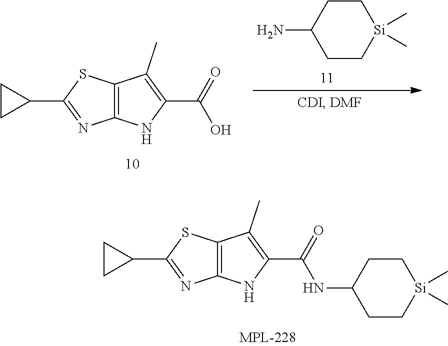

To a solution of 2-cyclopropyl-6-methyl-4H-pyrrolo[2,3-d]thiazole-5-carboxylic acid (300 mg, 1.35 mmol, 1 eq) in DMF (3 mL) was added CDI (328.29 mg, 2.02 mmol, 1.5 eq), the mixture was stirred at 30° C. for 0.5 h, then the 1,1-dimethylsilinan-4-amine (290.13 mg, 2.02 mmol, 1.5 eq) was added and the mixture was stirred for another 0.5 h under the same conditions. LC-MS showed the reaction was consumed completely. The mixture was diluted with DMF (1.5 mL) then purified by prep-HPLC (column: YMC-Actus Triart C18 150*30 mm*5 um;mobile phase: [water (0.225%FA)-ACN];B%: 65%-95%,10 min) and recrystallization in EtOAc (10 mL) at 100° C. under 1 atm. The product 2-cyclopropyl-N-(1,1-dimethylsilinan-4-yl)-6-methyl-4H-pyrrolo[2,3-d]thiazole-5-carboxamide (111.8 mg, 321.51 μmol, 23.82% yield, 99.946% purity) was obtained as a white solid.

LCMS (ESI) m/z 348.1 [M+H]⁺; ¹H NMR (400 MHz, DMSO-d₆) δ=11.77 (s, 1H), 7.26 (br d, J=7.8 Hz, 1H), 3.66 (br d, J=8.2 Hz, 1H), 2.40-2.31 (m, 4H), 1.98 (br d, J=10.2 Hz, 2H), 1.62-1.48 (m, 2H), 1.19-1.11 (m, 2H), 1.02-0.95 (m, 2H), 0.76 (br d, J=14.1 Hz, 2H), 0.63-0.52 (m, 2H), 0.07 (s, 3H), 0.03 (s, 3H).

### Example 15, MPL-240

### Synthesis of 2-cyclopropyl-N-(1,1-dimethylsilinan-4-yl)-4H-pyrrolo[2,3-d]thiazole-5-carboxamide

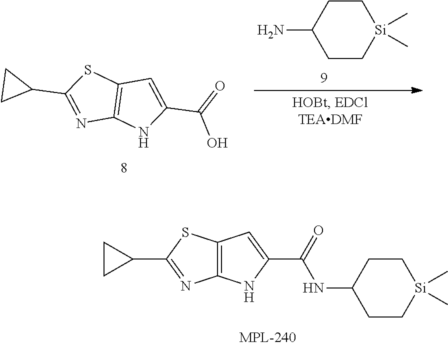

To a solution of 2-cyclopropyl-4H-pyrrolo[2,3-d]thiazole-5-carboxylic acid (1 g, 4.80 mmol, 1 eq) and 1,1-dimethylsilinan-4-amine (1.12 g, 6.24 mmol, 1.3 eq, HCl salt) in DMF (10 mL) was added a solution of HOBt (1.30 g, 9.60 mmol, 2 eq) and EDCI (1.84 g, 9.60 mmol, 2 eq) in DMF (10 mL). TEA (1.94 g, 19.21 mmol, 2.67 mL, 4 eq) was then added to the mixture. The mixture was stirred at 20° C. for 2 hr. LC-MS showed most of the starting material was consumed, desired mass was detected. The reaction mixture was mixed into NaHCO₃ (Sat. 300 mL). Filtered, the cake was washed with water (50 mL×2). The crude was purified by flash silica gel chromatography (ISCO®; 20 g SepaFlash® Silica Flash Column, Eluent of 0˜30% EtOAc/Petroleum ether gradient at35 mL/min). Compound 2-cyclopropyl-N-(1,1-dimethylsilinan-4-yl)-4H-pyrrolo[2,3-d]thiazole-5-carboxamide (1.02 g, 3.06 mmol, 63.82% yield, 100% purity) was obtained as a light yellow solid.

MS (ESI) m/z 334.0 [M+H]⁺; ¹H NMR (400 MHz, DMSO-d₆) δ=12.08 (s, 1 H) 7.82 (d, J=7.83 Hz, 1 H) 6.99 (d, J=1.96 Hz, 1 H) 3.58-3.71 (m, 1 H) 2.29-2.39 (m, 1 H) 1.95 (br d, J=10.56 Hz, 2 H) 1.46-1.62 (m, 2 H) 1.09-1.16 (m, 2 H) 0.95-1.01 (m, 2 H) 0.74 (br d, J=14.48 Hz, 2 H) 0.57 (td, J=14.09, 4.70 Hz, 2 H) 0.06 (s, 3 H) 0.01 (s, 3 H).

### Example 16, MPL-291

### Synthesis of N-cyclooctyl-2-cyclopropyl-6-methyl-4H-pyrrolo[2,3-d]thiazole-5-carboxamide

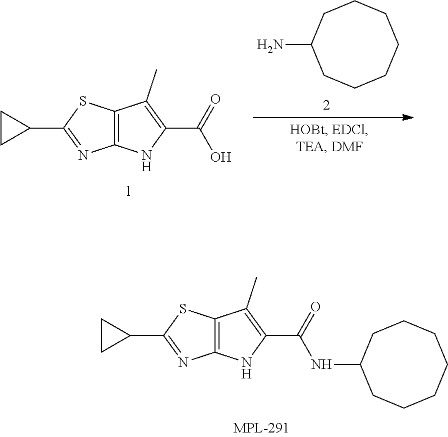 [SMILES: Cc1c(C(=O)O)[nH]c2nc(C3CC3)sc12 ; NC1CCCCCCC1 ; Cc1c(C(=O)NC2CCCCCCC2)[nH]c2nc(C3CC3)sc12]

To a solution of 2-cyclopropyl-6-methyl-4H-pyrrolo[2,3-d]thiazole-5-carboxylic acid (100 mg, 449.92 umol, 1 eq) and cyclooctanamine (57.24 mg, 449.92 umol, 1 eq) in DMF (1 mL) was added HOBt (182.38 mg, 1.35 mmol, 3 eq) and EDCI (258.75 mg, 1.35 mmol, 3 eq), followed by TEA (273.16 mg, 2.70 mmol, 375.74 uL, 6 eq). The mixture was stirred at 30° C. for 1 hr. LC-MS showed desired compound was detected. The reaction mixture was diluted with H₂O (10 mL) and extracted with EtOAc (10 mL×2). The combined organic layer was washed with 5% LiCl in water (10 mL×2), dried over Na₂SO₄, and filtered and concentrated under reduced pressure. The residue was purified by prep-HPLC (column: Welch Xtimate C18 150* 25 mm*5 um; mobile phase: A: 0.225% formic acid in water, B: CH₃CN, gradient: 46%-76% B over 10 min). Compound N-cyclooctyl-2-cyclopropyl-6 -methyl-4H-pyrrolo[2,3-d]thiazole-5-carboxamide (39.8 mg, 120.07 umol, 26.69% yield) was obtained as a white solid.

LCMS (ESI) m/z: 332.0 [M+H]⁺; ¹H NMR (400 MHz, METHANOL-d₄) δ=4.10 (td, J=4.5, 8.7 Hz, 1H), 2.45 (s, 3H), 2.37-2.29 (m, 1H), 1.88 (br d, J=11.0 Hz, 2H), 1.73(br dd, J=6.1, 14.7 Hz, 4H), 1.61 (br s, 8H), 1.21-1.14 (m, 2H), 1.11-1.03 (m, 2H).

### Example 17, MPL-293

### Synthesis of 2-cyclopropyl-N-(1,1-dimethylsilocan-4-yl)-6-methyl-4H-pyrrolo [2,3-d]thiazole-5-carboxamide

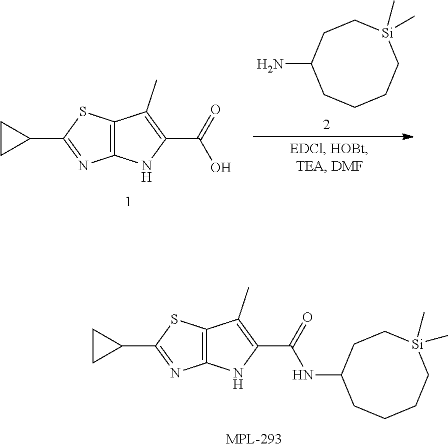 [SMILES: Cc1c(C(=O)O)[nH]c2nc(C3CC3)sc12 ; C[Si]1(C)CCCCC(N)CC1 ; Cc1c(C(=O)NC2CCCC[Si](C)(C)CC2)[nH]c2nc(C3CC3)sc12]

To a solution of 2-cyclopropyl-6-methyl-4H-pyrrolo[2,3-d]thiazole-5-carboxylic acid (25 mg, 112.48 umol, 1.2 eq) and 1,1-dimethylsilocan-4-amine (19.48 mg, 93.73 umol, 1 eq, HCl salt) in DMF (0.5 mL) was added a solution of EDCI (35.94 mg, 187.47 umol, 2 eq) and HOBt (25.33 mg, 187.47 umol, 2 eq) in DMF (0.5 mL), followed by TEA (37.94 mg, 374.93 umol, 52.19 uL, 4 eq). The mixture was stirred at 20° C. for 2 hr. LCMS showed reactant 1 was consumed completely and desired mass was detected. The mixture was diluted with MeOH (2 mL) and filtered to remove insoluble matter. The filtrate was purified by prep-HPLC (column: YMC-Actus Triart C18 150*30 mm*5 um; mobile phase: A: 0.225% formic acid in water, B: CH₃CN, gradient: 70%-100% B over 11 min). Compound 2-cyclopropyl-N-(1,1-dimethylsilocan-4-yl)-6-methyl-4H-pyrrolo [2,3-d]thiazole-5-carboxamide (16 mg, 42.60 umol, 45.45% yield, 100% purity) was obtained as a light brown solid.

LCMS (ESI) m/z 376.1 [M+H]⁺; ¹H NMR (500 MHz, DMSO-d₆) δ=11.80 (s, 1H), 7.27 (d, J=7.8 Hz, 1H), 3.92 (br s, 1H), 2.38 (s, 3H), 2.37-2.33 (m, 1H), 1.86-1.75 (m, 1H), 1.72-1.50 (m, 6H), 1.48-1.36 (m, 1H), 1.18-1.11 (m, 2H), 1.01-0.93 (m, 2H), 0.81-0.63 (m, 3H), 0.59-0.45 (m, 1H), 0.01 (d, J=8.5 Hz, 6H).

### Example 18, MPL-297

### Synthesis of 2-cyclopropyl-N-(1,1-dimethylsilolan-3-yl)-4H-pyrrolo[2,3-d]thiazole-5-carboxamide

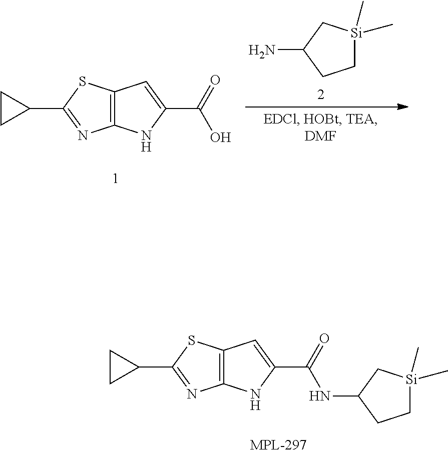

To a solution of 2-cyclopropyl-4H-pyrrolo[2,3-d]thiazole-5-carboxylic acid (30 mg, 144.07 umol, 1 eq), 1,1-dimethylsilolan-3-amine (23.88 mg, 144.07 umol, 1 eq, HCl salt) in DMF (1 mL) was added HOBt (58.40 mg, 432.20 umol, 3 eq) and EDCI (82.85 mg, 432.20 umol, 3 eq), followed by TEA (87.47 mg, 864.40 umol, 120.31 uL, 6 eq). The mixture was stirred at 20° C. for 1 hr. LCMS showed that desired compound was detected. The reaction mixture was filtered and the filtrate was purified by prep-HPLC (column: YMC-Actus Triart C18 150*30 mm*5 um; mobile phase: A: 0.225% formic acid in water, B: CH₃CN; gradient: 49%-79% B over 11 min). Compound 2-cyclopropyl-N-(1,1-dimethylsilolan-3-yl)-4H-pyrrolo[2,3-d]thiazole-5-carboxamide (24 mg, 72.11 umol, 50.06% yield, 96% purity) was obtained as a white solid.

LCMS (ESI) m/z: 320.1[M+H]⁺; ¹H NMR (400 MHz, METHANOL-d₄) δ=6.98 (s, 1H), 4.13-4.02 (m, 1H), 2.37-2.28 (m, 1H), 2.22-2.11 (m, 1H), 1.52-1.40 (m, 1H),1.26-1.14 (m, 3H), 1.11-1.04 (m, 2H), 0.87 (dd, J=6.1, 14.7 Hz, 1H), 0.67-0.56 (m, 2H), 0.19 (s, 6H).

### Example 19, MPL-304

### Synthesis of 2-cyclopropyl-N-(1,1-dimethylsilocan-5-yl) -4H-pyrrolo[2,3-d]thiazole-5-carboxamide

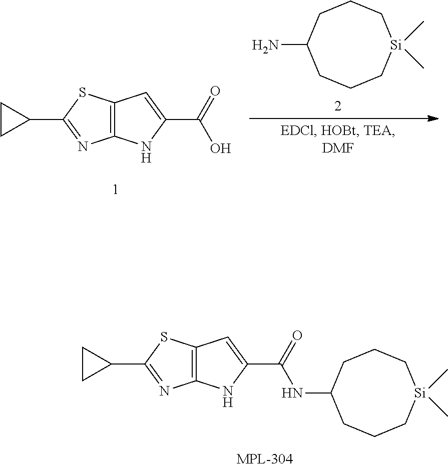 [SMILES: C[Si]1(C)CCCC(N)CCC1 ; O=C(O)c1cc2sc(C3CC3)nc2[nH]1 ; C[Si]1(C)CCCC(NC(=O)c2cc3sc(C4CC4)nc3[nH]2)CCC1]

To a solution of 2-cyclopropyl-4H-pyrrolo[2,3-d]thiazole-5-carboxylic acid (30 mg, 144.07 umol, 1 eq), 1,1-dimethylsilocan-5-amine (29.94 mg, 144.07 umol, 1 eq, HCl salt) in DMF (0.5 mL) was added a solution of HOBt (58.40 mg, 432.20 umol, 3 eq) and EDCI (82.85 mg, 432.20 umol, 3 eq) in DMF (0.5 mL), followed by TEA (87.47 mg, 864.40 umol, 120.31 uL, 6 eq). The mixture was stirred at 20° C. for 1 hr. LCMS showed that desired compound was detected. The reaction mixture was filtered. The filtrate was purified by prep-HPLC (column: YMC-Actus Triart C18 150*30 mm*5 um; mobile phase: A: 0.225% formic acid in water, B: CH₃CN; gradient: 50%-80% B over 11 min). The product from prep-HPLC purification was further purified by SFC (Waters Prep SFC 80Q; column: DAICEL CHIRALPAK IG (250 mm*30 mm,10 um); mobile phase: A: 0.1%NH₃H₂O in EtOH, B: CO₂; gradient: 60%B, isocratic; flow rate: 60 mL/min). 2-cyclopropyl-N-(1,1-dimethylsilocan-5-yl) -4H-pyrrolo[2,3-d]thiazole-5-carboxamide (11 mg, 30.34 umol, 36.57% yield, 99.739% purity) was obtained as a white solid.

LCMS m/z: 362.1 [M+1]⁺; ¹H NMR (400 MHz, METHANOL-d₄) δ=6.97 (s, 1H), 4.20-4.13 (m, 1H), 2.37-2.28 (m, 1H), 1.85-1.73 (m, 4H), 1.71 (br t, J=5.3 Hz, 4H), 1.21-1.15 (m, 2H), 1.11-1.06 (m, 2H), 0.86-0.76 (m, 4H), 0.05 (s, 3H), 0.03 (s, 3H).

### Example 20, MPL-308

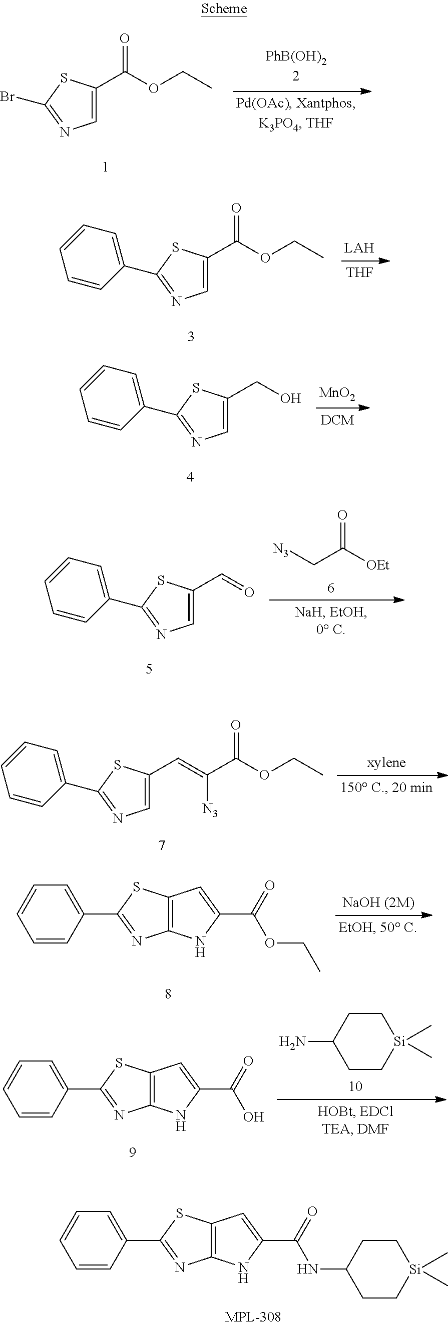

### Step 1. Synthesis of ethyl 2-phenylthiazole-5-carboxylate

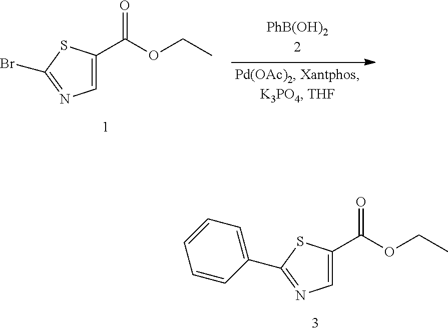 [SMILES: CCOC(=O)c1cnc(Br)s1 ; CCOC(=O)c1cnc(-c2ccccc2)s1]

To a mixture of ethyl 2-bromothiazole-5-carboxylate (7.7 g, 32.62 mmol, 1 eq), phenylboronic acid (19.88 g, 163.08 mmol, 5 eq), K₃PO₄ (10.38 g, 48.92 mmol, 1.5 eq) and Xantphos (3.77 g, 6.52 mmol, 0.2 eq) was added THF (100 mL). The mixture was purged with N₂, Pd(OAc)₂ (732.24 mg, 3.26 mmol, 0.1 eq) was then added. The mixture was stirred at 80° C. for 24 hr. TLC showed two new spots. The mixture was filtered. The cake was washed with EtOAc (10 mL×2). The combined filtrate was dried over Na₂SO₄, and then filtered and concentrated under reduced pressure. The residue was purified by column chromatography (SiO₂, 0-1% Ethyl acetate in petroleum ether). Compound ethyl 2-phenylthiazole-5-carboxylate (4.78 g, 19.47 mmol, 59.68% yield, 95% purity) was obtained as a white solid. ¹H NMR was recorded.

### Step 2. Synthesis of (2-phenylthiazol-5-yl)methanol

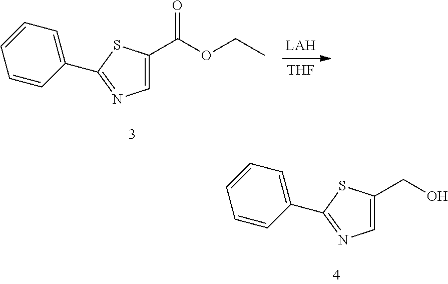 [SMILES: CCOC(=O)c1cnc(-c2ccccc2)s1 ; OCc1cnc(-c2ccccc2)s1 ; "THF"]

LAH (1 g, 26.35 mmol, 1.12 eq) was added to THF (10 mL). A solution of ethyl 2-phenylthiazole-5-carboxylate (5.5 g, 23.58 mmol, 1 eq) in THF (50 mL) was then added under stirring at 0° C. The reaction mixture was stirred at 0-25° C. for 30 min. TLC (Petroleum ether: EtOAc=3:1) showed one spot with higher polarity. The reaction mixture was quenched with water (1 mL), NaOH (15% in water, 1 mL) and water (3 mL). The mixture was then filtered. The filter cake was washed with EtOAc (10 mL×5). The combined filtrate was dried over Na₂SO₄, and then filtered and concentrated under reduced pressure. Compound (2-phenylthiazol-5-yl)methanol (4.5 g, 21.18 mmol, 89.82% yield, 90% purity) was obtained as a yellow oil, which was used for the next step without purification. ¹H NMR was recorded.

### Step 3. Synthesis of 2-phenylthiazole-5-carbaldehyde

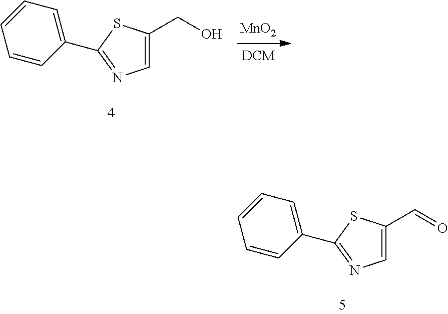

To a solution of (2-phenylthiazol-5-yl)methanol (4.5 g, 23.53 mmol, 1 eq) in DCM (50 mL) was added MnO₂ (20.46 g, 235.30 mmol, 10 eq). The mixture was stirred at 25° C. for 5 hr. TLC (Petroleum ether:EtOAc=10:1) showed the starting material was consumed completely. The mixture was then filtered. The filter cake was washed with EtOAc (20 mL×3). The combined filtrate was dried over Na₂SO₄, and then filtered and concentrated under reduced pressure. Compound 2-phenylthiazole-5-carbaldehyde (3.48 g, 16.57 mmol, 70.42% yield, 90% purity) was obtained as a yellow solid. ¹H NMR was recorded. The crude product was used for the next step without further purification.

### Step 4. Synthesis of ethyl (Z)-2-azido-3-(2-phenylthiazol-5-yl)prop-2-enoate

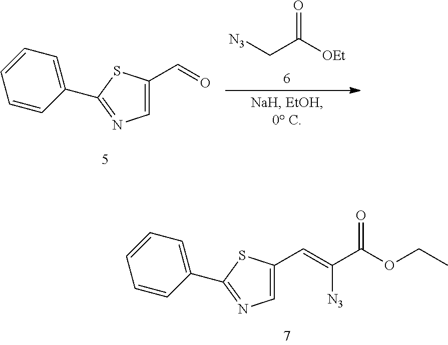

NaH (528.40 mg, 13.21 mmol, 60% purity, 5 eq) was added to EtOH (5 mL) in batches. The mixture was stirred at 30° C. until a clear solution was formed, and then cooled to −10° C. A solution of 2-phenylthiazole-5-carbaldehyde (500 mg, 2.64 mmol, 1 eq) and ethyl 2-azidoacetate (1.71 g, 13.21 mmol, 1.85 mL, 5 eq) in EtOH (2 mL) was added dropwise. The mixture was stirred at −10° C.˜0° C. for 2 hr. TLC (Petroleum ether: EtOAc=5:1) indicated the starting material was consumed completely. The reaction was quenched with HCl (3M in water, about 5 eq) until pH turned to 6, and then concentrated under reduced pressure until ⅕ of the original volume left, and then extracted with EtOAc (100 mL×2). The combined organic layer was washed with brine (50 mL×2), dried over Na₂SO₄, and filtered and concentrated under reduced pressure. The residue was purified by column chromatography (SiO₂, 0-100% Ethyl acetate in petroleum ether). Compound ethyl (Z)-2-azido-3-(2-phenylthiazol-5-yl)prop-2-enoate (220 mg, 586.01 umol, 22.18% yield, 80% purity) was obtained as a yellow oil.

LCMS (ESI) m/z 301.1 [M+H]⁺; ¹H NMR was recorded.

### Step 5. Synthesis of ethyl 2-phenyl-4H-pyrrolo[2,3-d]thiazole-5-carboxylate

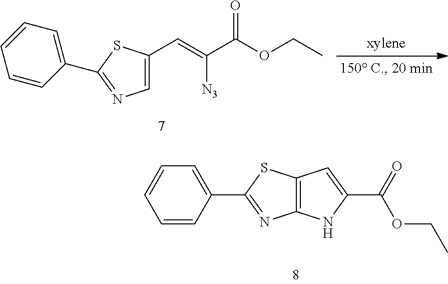 [SMILES: CCOC(=O)/C(=C/c1cnc(-c2ccccc2)s1)N=[N+]=[N-] ; CCOC(=O)c1cc2sc(-c3ccccc3)nc2[nH]1]

Ethyl (Z)-2-azido-3-(2-phenylthiazol-5-yl)prop-2-enoate (220 mg, 732.52 umol, 1 eq) in xylene (2 mL) was stirred at 150° C. for 20 min. LCMS showed desire product was detected. The mixture was cooled to room temperature and filtered. The cake was washed with a mixed solvent of petroleum ether and EtOAc (10:1, 5 mL×4) and collected. Compound ethyl 2-phenyl-4H-pyrrolo[2,3-d]thiazole-5-carboxylate (99.6 mg, 329.17 umol, 44.94% yield, 90% purity) was obtained as a white solid.

LCMS (ESI) m/z 273.0 [M+H]⁺; ¹H NMR was recorded.

### Step 6. Synthesis of 2-phenyl-4H-pyrrolo[2,3-d]thiazole-5-carboxylic acid

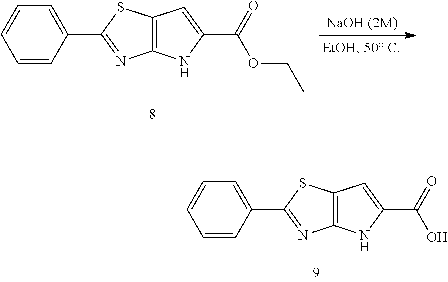 [SMILES: CCOC(=O)c1cc2sc(-c3ccccc3)nc2[nH]1 ; O=C(O)c1cc2sc(-c3ccccc3)nc2[nH]1]

To a solution of ethyl 2-phenyl-4H-pyrrolo[2,3-d]thiazole-5-carboxylate (99.6 mg, 365.74 umol, 1 eq) in EtOH (1 mL) was added NaOH (4 M in water, 1 mL, 10.94 eq). The mixture was stirred at 25° C. for 12 hr, and then stirred at 50° C. for 12 hr. LCMS showed desired product was detected. The reaction mixture was concentrated under reduced pressure to remove EtOH, and then adjusted to pH 3 with HCl (6 M in water) and filtered. The cake was washed with water (2 mL×3), and then diluted with a mixed solvent of water (5 mL) and CH₃CN (5 mL) and lyophilized. Compound 2-phenyl-4H-pyrrolo[2,3-d]thiazole-5-carboxylic acid (68 mg, 278.38 umol, 76.11% yield) was obtained as a brown solid, which was used for the next step without further purification.

LCMS (ESI) m/z 245.1 [M+H]⁺; ¹H NMR (500 MHz, DMSO-d₆) δ=12.72 (br s, 1H), 7.96 (dd, J=1.4, 8.0 Hz, 2H), 7.55-7.49 (m, 3H), 7.10 (s, 1H).

### Step 7. N-(1,1-dimethylsilinan-4-yl)-2-phenyl-4H-pyrrolo[2,3-d]thiazole-5-carboxamide

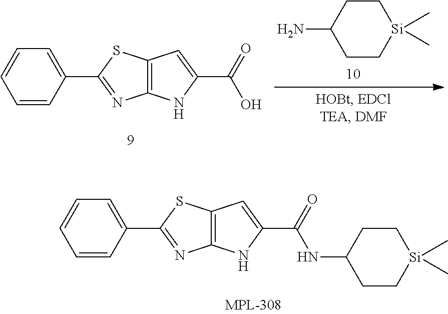

To a solution of 2-phenyl-4H-pyrrolo[2,3-d]thiazole-5-carboxylic acid (68 mg, 278.38 umol, 1 eq) and 1,1-dimethylsilinan-4-amine (55.05 mg, 306.22 umol, 1.1 eq, HCl salt) in DMF (1 mL) at 25° C., a solution of HOBt (112.84 mg, 835.14 umol, 3 eq) and EDCI (160.10 mg, 835.14 umol, 3 eq) in DMF (1 mL) was added, followed by TEA (140.85 mg, 1.39 mmol, 193.74 uL, 5 eq). The reaction mixture was stirred at 25° C. for 2 hr. LCMS showed the desired product was detected. The mixture was poured into NaHCO₃ solution (saturated NaHCO₃: H₂O=2:1, 10 mL). Precipitates formed. The mixture was stirred at 25° C. for 10 min and then filtered. The cake was washed with water (5 mL×3) and collected. The crude product was triturated with CH₃CN (5 mL) at 25° C. for 20 min. The solid was collected by filtration. Compound N-(1,1-dimethylsilinan-4-yl)-2-phenyl-4H -pyrrolo[2,3-d]thiazole-5-carboxamide (31.1 mg, 84.16 umol, 30.23% yield, 100% purity) was obtained as a brown solid.

LCMS (ESI) m/z 370.1 [M+H]⁺; ¹H NMR (500 MHz, DMSO-d₆) δ=7.99-7.76 (m, 3H), 7.53-7.39 (m, 3H), 6.99 (br s, 1H), 3.73-3.63 (m, 1H), 1.97 (br d, J=8.9 Hz, 2H), 1.63-1.54 (m, 2H), 0.76 (br d, J=14.5 Hz, 2H), 0.60 (dt, J=4.7, 13.9 Hz, 2H), 0.08 (s, 3H), 0.03 (s, 3H).

### Example 21, MPL-309

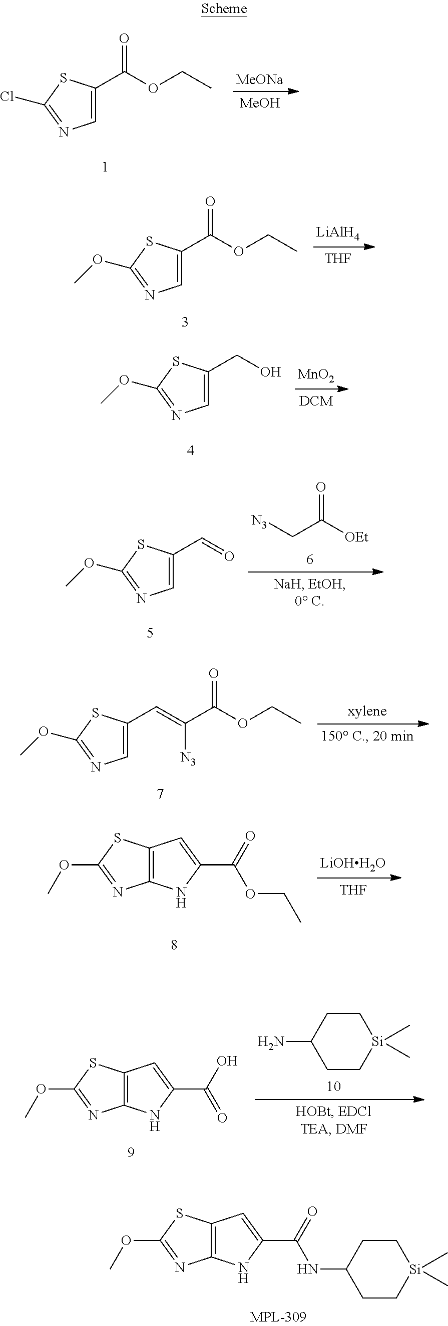

### Step 1. Synthesis of methyl 2-methoxythiazole-5-carboxylate

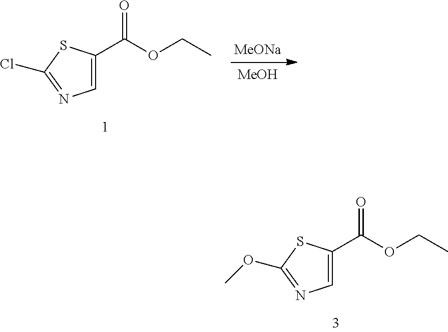

A solution of ethyl 2-chlorothiazole-5-carboxylate (15 g, 78.27 mmol, 1 eq) and NaOMe (70.48 g, 391.37 mmol, 30% in MeOH, 5 eq) in MeOH (100 mL) was stirred at 25° C. for 30 min. TLC showed the starting material was consumed completely. The reaction was quenched with aqueous HCl (1 M, 400 mL). The mixture was diluted with water (300 mL), and then extracted with EtOAc (200 mL×4). The combined organic layer was dried over Na₂SO₄, and then filtered and concentrated in vacuo. Compound methyl 2-methoxythiazole-5-carboxylate (13 g, 60.05 mmol, 76.72% yield, 80% purity) was obtained as a yellow solid, which was used for the next step without further purification. ¹H NMR was recorded.

### Step 2. Synthesis of (2-methoxythiazol-5-yl)methanol

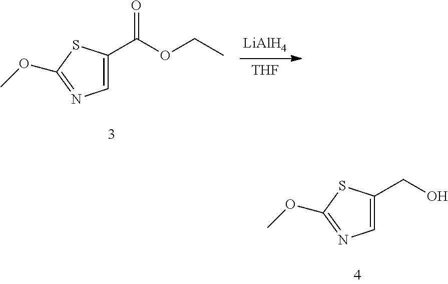

To an ice-cooled solution of methyl 2-methoxythiazole-5-carboxylate (13 g, 75.06 mmol, 1 eq) in dried THF (100 mL) was added LiAlH₄ (4.30 g, 113.31 mmol, 1.51 eq) in batches. The mixture was stirred at 0-20° C. for 30 min. LCMS indicated reactant 3 was consumed completely and desired compound was detected. The reaction was quenched with water (4.3 mL), NaOH (15%, 4.3 mL) and water (12.9 mL). The mixture was then filtered. The filter cake was washed with EtOAc (100 mL×5). The combined filtrate was dried over Na₂SO₄, and then filtered and concentrated under reduced pressure. Compound (2-methoxythiazol-5-yl)methanol (10 g, 58.55 mmol, 78.00% yield, 85% purity) was obtained as a yellow solid, which was used for the next step without further purification.

LCMS (ESI) m/z 146.1 [M+H]⁺; ¹H NMR was recorded.

### Step 3. Synthesis of 2-methoxythiazole-5-carbaldehyde

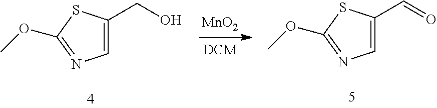

To a solution of (2-methoxythiazol-5-yl)methanol (20 g, 137.76 mmol, 1 eq) in DCM (200 mL) was added MnO₂ (119.77 g, 1.38 mol, 10 eq). The mixture was stirred at 25° C. for 2 hr. LC-MS showed desired mass. The mixture was filtered. The filter cake was washed with EtOAc (100 mL×5). The combined filtrate was dried over Na₂SO₄, and then filtered and concentrated under reduced pressure. The residue was purified by column chromatography (SiO₂, 0-10% Ethyl acetate in petroleum ether). Compound 2-methoxythiazole-5-carbaldehyde (15.33 g, 96.37 mmol, 69.96% yield, 90% purity) was obtained as a yellow oil.

LCMS (ESI) m/z 144.3 [M+H]⁺; ¹H NMR was recorded.

### Step 4. Synthesis of ethyl (Z)-2-azido-3-(2-methoxythiazol-5-yl)prop-2-enoate

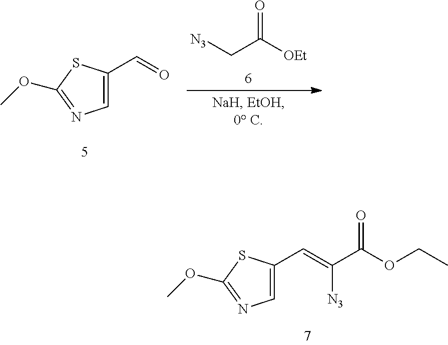

NaH (12.99 g, 324.80 mmol, 60% purity, 5 eq) was added to EtOH (150 mL) in batches. The mixture was stirred at 30° C. until a clear solution formed, and then cooled to −10° C. A solution of 2-methoxythiazole-5-carbaldehyde (9.3 g, 64.96 mmol, 1 eq) and ethyl 2-azidoacetate (41.94 g, 324.80 mmol, 45.58 mL, 5 eq) in EtOH (50 mL) was then added dropwise. The reaction mixture was stirred at −10° C.˜0° C. for 2 hr. TLC (Petroleum ether: EtOAc=5:1) indicated reactant 5 was consumed completely. The reaction was quenched with HCl (3 M in water, about 5 eq) until pH turned to 6, concentrated under reduced pressure until ⅕ of the original volume left, and then extracted with EtOAc (200 mL×2). The combined organic layer was washed with brine (200 mL×2), dried over Na₂SO₄, and then filtered and concentrated under reduced pressure. The residue was purified by column chromatography (SiO₂, 0-10% Ethyl acetate in petroleum ether). Compound ethyl (Z)-2-azido-3-(2-methoxythiazol-5-yl)prop-2-enoate (10 g, 31.46 mmol, 48.43% yield, 80% purity) was obtained as a yellow solid. ¹H NMR was recorded.

### Step 5. Synthesis of ethyl 2-methoxy-4H-pyrrolo[2,3-d]thiazole-5-carboxylate

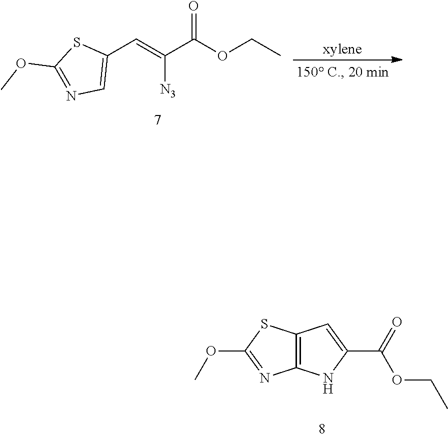

Ethyl (Z)-2-azido-3-(2-methoxythiazol-5-yl)prop-2-enoate (10 g, 39.33 mmol, 1 eq) in xylene (20 mL) was stirred at 150° C. for 20 min. TLC (Petroleum ether: EtOAc=3:1) indicated reactant 7 was consumed completely. The mixture was filtered. The cake was washed with petroleum ether (20 mL×3). The collected filter cake was purified by column chromatography (SiO₂, 0-20% Ethyl acetate in petroleum ether). Compound ethyl 2-methoxy-4H-pyrrolo[2,3-d]thiazole-5-carboxylate (3.34 g, 14.01 mmol, 35.62% yield, 95% purity) was obtained as a yellow solid. ¹H NMR was recorded.

### Step 6. Synthesis of 2-methoxy-4H-pyrrolo[2,3-d]thiazole-5-carboxylic acid

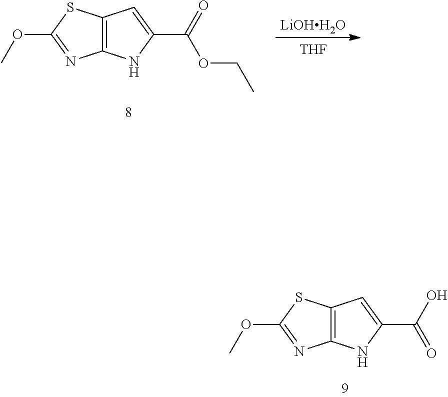

To a solution of ethyl 2-methoxy-4H-pyrrolo[2,3-d]thiazole-5-carboxylate (6.77 g, 29.92 mmol, 1 eq) in THF (30 mL) was added a solution of LiOH.H₂O (7.53 g, 179.53 mmol, 6 eq) in H₂O (30 mL). The reaction mixture was stirred at 80° C. for 12 hr. LC-MS showed desired compound was detected. The reaction mixture was concentrated under reduced pressure to remove THF, and then adjusted to pH 2 using HCl (6 M in water), and then filtered and concentrated under reduced pressure to give 2-methoxy-4H-pyrrolo[2,3-d]thiazole-5-carboxylic acid (2.3 g, 10.44 mmol, 34.90% yield, 90% purity) was obtained as a brown solid, which was used for the next step without further purification.

LCMS (ESI) m/z 199.2 [M+H]⁺; ¹H NMR (400 MHz, DMSO-d₆) δ=12.39 (br s, 2H), 6.93 (d, J=2.0 Hz, 1H), 4.09 (s, 3H).

### Step 7. Synthesis of N-(1,1-dimethylsilinan-4-yl)-2-methoxy-4H-pyrrolo[2,3-d]thiazole-5-carboxamide

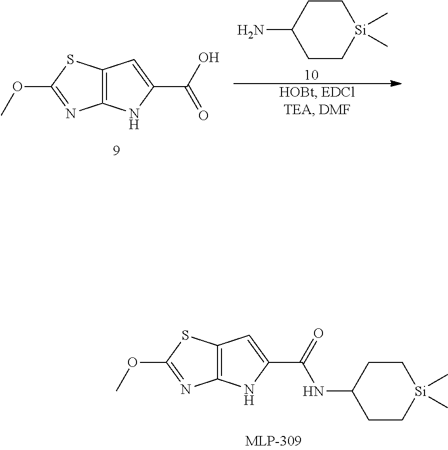

To a solution of 2-methoxy-4H-pyrrolo[2,3-d]thiazole-5-carboxylic acid (1.9 g, 9.59 mmol, 1 eq) and 1,1-dimethylsilinan-4-amine (2.07 g, 11.50 mmol, 1.2 eq, HCl salt) in DMF (15 mL) was added a solution of HOBt (3.89 g, 28.76 mmol, 3 eq) and EDCI (5.51 g, 28.76 mmol, 3 eq) in DMF (15 mL), followed by TEA (4.85 g, 47.93 mmol, 6.67 mL, 5 eq). The reaction mixture was stirred at 25° C. for 2 hr. LC-MS showed desired compound was detected. The reaction mixture was quenched with NaHCO3 solution (saturated NaHCO₃: H₂O=2:1, 100 mL) at 25° C., and then filtered. The filter cake was collected and dried under reduced pressure. The residue was purified by column chromatography (SiO₂, 0-33% Ethyl acetate in petroleum ether). The isolated product was triturated with MTBE 50 mL at 25° C. for 30 min. The solid was collected by filtration. Compound N-(1,1-dimethylsilinan-4-yl)-2-methoxy-4H-pyrrolo[2,3-d]thiazole-5-carboxamide (2.19 g, 6.48 mmol, 61.44% yield, 95.7% purity) was obtained as a yellow solid.

LCMS (ESI) m/z 324.0 [M+H] ; ¹H NMR (400 MHz, CHLOROFORM-d) δ=9.48 (br s, 1H), 6.57 (d, J=2.0 Hz, 1H), 5.71 (br d, J=8.2 Hz, 1H), 3.94-3.80 (m, 1H), 2.23-2.09 (m, 2H), 1.60-1.45 (m, 2H), 0.85-0.61 (m, 4H), 0.06 (d, J=13.7 Hz, 6H).

### Example 22, MPL-357, MPL-357A and MPL-357B

### Synthesis of N-(1,1-dimethylsilepan-4-yl)-2-phenyl-4H-pyrrolo[2,3-d]thiazole-5-carboxamide, (R)-N-(1,1-dimethylsilepan-4-yl)-2-phenyl-4H-pyrrolo[2,3-d]thiazole-5-carboxamide and (5)-N-(1,1-dimethylsilepan-4-yl)-2-phenyl-4H-pyrrolo[2,3-d]thiazole-5-carboxamide

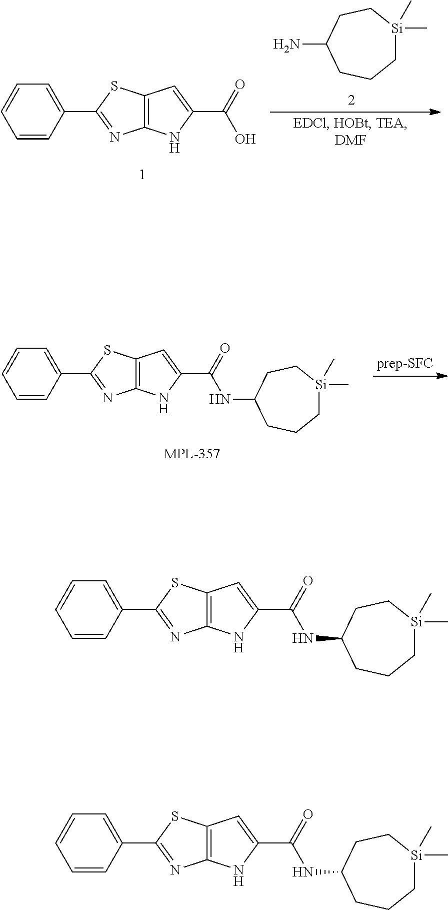

Compound 1 (990 mg, 3.65 mmol, 90% purity) was made from ethyl 2-bromothiazole-5-carboxylate (5 g, 21.18 mmol) using the same procedures described for the synthesis of compound 9 in Example 20.

To a solution of 2-phenyl-4H-pyrrolo[2,3-d]thiazole-5-carboxylic acid (495 mg, 2.03 mmol, 1 eq) and 1,1-dimethylsilepan-4-amine (471.25 mg, 2.43 mmol, 1.2 eq, HCl salt) in DMF (3 mL) was added a solution of HOBt (821.46 mg, 6.08 mmol, 3 eq) and EDCI (1.17 g, 6.08 mmol, 3 eq) in DMF (2 mL), followed by TEA (1.03 g, 10.13 mmol, 1.41 mL, 5 eq). The mixture was stirred at 25° C. for 2 hr. LCMS showed desired compound was detected. The reaction mixture was quenched with aqueous NaHCO₃ (saturated NaHCO₃: H₂O=1:2, 100 mL) at 25° C., and filtered. The filter cake was collected and dried under reduced pressure and then purified by column chromatography (SiO₂, 0-20% Ethyl acetate in petroleum ether). Compound N-(1,1-dimethylsilepan-4-yl)-2-phenyl-4H-pyrrolo[2,3-d]thiazole-5-carboxamide (595 mg, 1.55 mmol, 76.55% yield, 100% purity) was obtained as a yellow solid.

LCMS (ESI) m/z 384.2 [M+H]⁺; ¹H NMR (500 MHz, DMSO-d₆) δ=12.42 (s, 1H), 8.03 (d, J=8.1 Hz, 1H), 7.96-7.88 (m, 2H), 7.59-7.38 (m, 3H), 7.16 (d, J=1.7 Hz, 1H), 3.87 (br d, J=8.4 Hz, 1H), 2.08-1.32 (m, 6H), 0.92-0.48 (m, 4H), 0.04 (d, J=8.5 Hz, 6H).

MPL-357 (130 mg, 338.91 umol) was separated by SFC (Waters Prep SFC 80Q, DAICEL CHIRALCEL OJ-H(250 mm*30 mm,5 um); mobile phase: A: 0.1%NH₃H₂O in MeOH, B: CO₂; gradient: 40%B isocratic; flow rate: 70 mL/min) to afford two peaks (two enantiomers), (R)-N-(1, 1-dimethylsilepan-4-yl)-2-phenyl-4H-pyrrolo [2,3 -d]thiazole-5 -carboxamide and (S)-N-(1,1-dimethylsilepan-4-yl)-2-phenyl-4H-pyrrolo [2,3 -d]thiazole-5 -carboxamide.

Peak1 (MPL-357A): 41.3 mg, 107.67 umol, 31.77% yield, 100% purity, white solid

LCMS (ESI) m/z 384.2 [M+H]⁺; ¹H NMR (400 MHz, DMSO-d₆) δ=12.40 (br s, 1H), 8.11-7.85 (m, 3H), 7.55-7.44 (m, 2H), 7.16 (s, 1H), 3.88 (br s, 1H), 1.93-1.45 (m, 6H), 0.83-0.53 (m, 4H), 0.04 (d, J=6.7 Hz, 6H).

Peak2 (MPL-357B): 44.3 mg, 115.49 umol, 34.08% yield, 100% purity, white solid

LCMS (ESI) m/z 384.2 [M+H]⁺; ¹H NMR (400 MHz, DMSO-d₆) δ=12.40 (br s, 1H), 8.08-7.88 (m, 3H), 7.53-7.44 (m, 2H), 7.16 (s, 1H), 3.88 (br s, 1H), 1.91-1.48 (m, 6H), 0.82-0.57 (m, 4H), 0.04 (d, J=7.0 Hz, 6H).

MPL-357A and MPL-357B were also analyzed by analytical SFC.

Conditions:

Instrument: Agilent 1260 with DAD detector

Column: ChiralCel OJ-H 150×4.6 mm 5 um particle size

Mobile phase: A: CO₂, B: 0.05% DEA in MeOH

Gradient: 40% B, isocratic

Flow rate: 2.5 mL/min

Column temp.: 35° C.

ABPR: 100 bar

MPL-357A: retention time 6.09 min; 100% ee; MPL-357B: 7.56 min; 97.8% ee

### Example 23, MPL-358

### Synthesis of 2-phenyl-N-(5-silaspiro[4.5]decan-8-yl)-4H-pyrrolo[2,3-d]thiazole-5-carboxamide

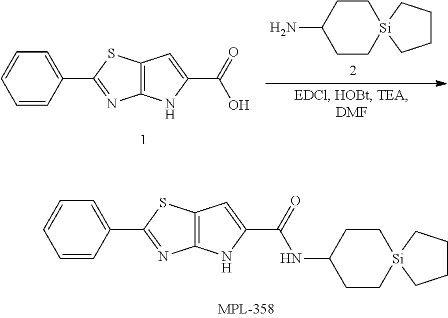 [SMILES: O=C(O)c1cc2sc(-c3ccccc3)nc2[nH]1 ; NC1CC[Si]2(CCCC2)CC1 ; O=C(NC1CC[Si]2(CCCC2)CC1)c1cc2sc(-c3ccccc3)nc2[nH]1]

To a solution of 2-phenyl-4H-pyrrolo[2,3-d]thiazole-5-carboxylic acid (495 mg, 2.03 mmol, 1 eq) and 5-silaspiro[4.5]decan-8-amine (458.75 mg, 2.23 mmol, 1.1 eq, HCl salt) in DMF (3 mL) at 25° C., a solution of HOBt (821.46 mg, 6.08 mmol, 3 eq) and EDCI (1.17 g, 6.08 mmol, 3 eq) in DMF (2 mL) was added, followed by TEA (1.03 g, 10.13 mmol, 1.41 mL, 5 eq). The reaction mixture was stirred at 25° C. for 2 hr. LC-MS showed desired compound was detected. The reaction mixture was quenched by aqueous NaHCO₃ (saturated NaHCO₃: H₂O=1:2, 100 mL at 25° C., and filtered. The filter cake was dried under reduced pressure and then purified by column chromatography (SiO₂, 0-33% Ethyl acetate in petroleum ether). Compound 2-phenyl-N-(5-silaspiro[4.5]decan-8-yl)-4H-pyrrolo[2,3 -d]thiazole-5-carboxamide (623.8 mg, 1.58 mmol, 77.81% yield, 100% purity) was obtained as a yellow solid.

LCMS (ESI) m/z 396.2 [M+H]⁺; ¹H NMR (500 MHz, DMSO-d₆) δ=12.43 (d, J=1.2 Hz, 1H), 8.01 (d, J=8.2 Hz, 1H), 7.98-7.89 (m, 2H), 7.60-7.40 (m, 3H), 7.15 (d, J=1.8 Hz, 1H), 3.82-3.68 (m, 1H), 2.11-2.01 (m, 2H), 1.69-1.45 (m, 6H), 0.89-0.43 (m, 8H).

### Example 24, MPL-359

### Synthesis of 2-phenyl-N-(6-silaspiro[5.5]undecan-3-yl)-4H-pyrrolo[2,3-d]thiazole-5-carboxamide

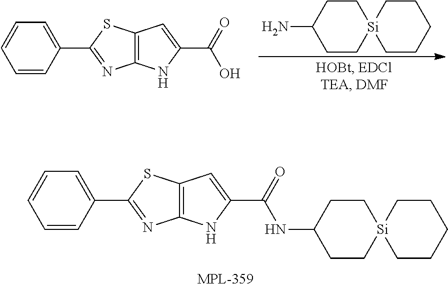 [SMILES: O=C(O)c1cc2sc(-c3ccccc3)nc2[nH]1 ; NC1CC[Si]2(CCCCC2)CC1 ; O=C(NC1CC[Si]2(CCCCC2)CC1)c1cc2sc(-c3ccccc3)nc2[nH]1]

To a solution of 2-phenyl-4H-pyrrolo[2,3-d]thiazole-5-carboxylic acid (20 mg, 81.88 umol, 1 eq) and 6-silaspiro[5.5]undecan-3-amine (21.60 mg, 98.25 umol, 1.2 eq, HCl salt) in DMF (1 mL) at 25° C. was added a solution of HOBt (33.19 mg, 245.63 umol, 3 eq) and EDCI (47.09 mg, 245.63 umol, 3 eq) in DMF (1 mL), followed by TEA (41.43 mg, 409.39 umol, 56.98 uL, 5 eq). The mixture was stirred at 25° C. for 1 hr. LC-MS showed desired compound was detected. The mixture was purified by prep-HPLC (YMC-Actus Triart C18 150*30 mm*5 um; mobile phase: A: 0.225% formic acid in water, B: CH₃CN; gradient: 75%-100% B over 11 min). Compound 2-phenyl-N-(6-silaspiro[5.5]undecan-3-yl)-4H-pyrrolo[2,3-d]thiazole-5-carboxamide (5.3 mg, 12.94 umol, 15.80% yield, 100% purity) was obtained as a yellow solid.

LCMS (ESI) m/z 410.2 [M+H]⁺; ¹H NMR (500 MHz, DMSO-d₆) δ=12.42 (s, 1H), 8.05-7.88 (m, 3H), 7.55-7.42 (m, 3H), 7.14 (s, 1H), 3.82-3.61 (m, 1H), 2.06-1.93 (m, 2H), 1.69-1.49 (m, 6H), 1.39 (br s, 2H), 0.94-0.82 (m, 2H), 0.75-0.65 (m, 2H), 0.65-0.53 (m, 4H).

### Example 25, MPL-364

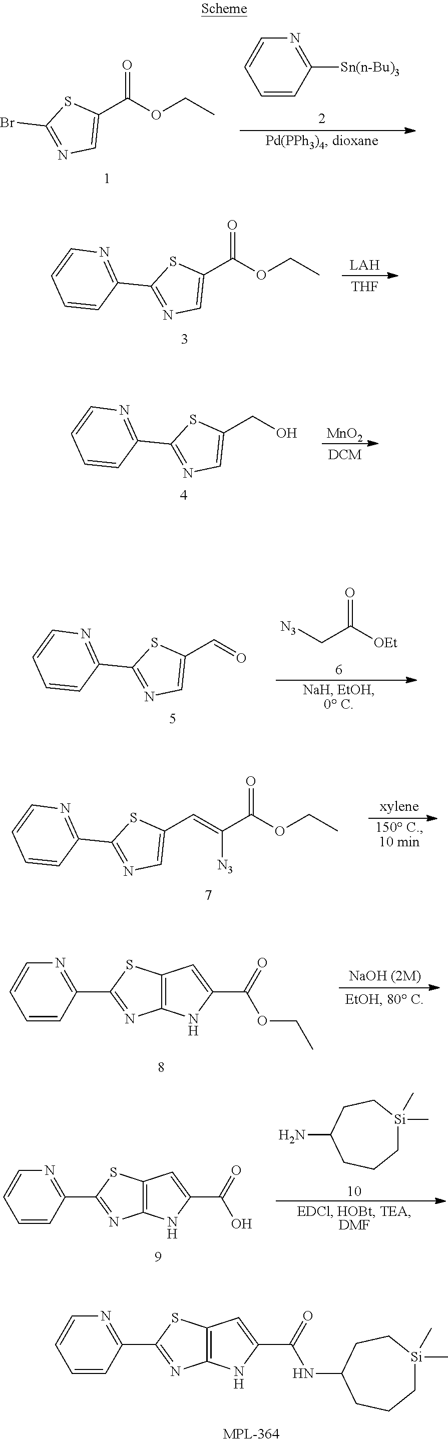

### Step 1. Synthesis of ethyl 2-(pyridin-2-yl)thiazole-5-carboxylate

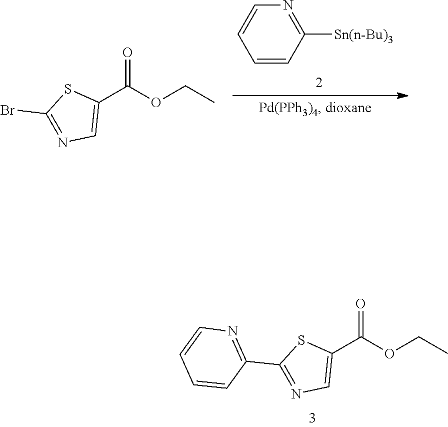

To a solution of ethyl 2-bromothiazole-5-carboxylate (1 g, 4.24 mmol, 1 eq) and 2-(tributylstannyl)pyridine (3.12 g, 8.47 mmol, 2 eq) in dioxane (50 mL) was added Pd(PPh₃)₄ (250.04 mg, 216.38 umol, 5.11e-2 eq) and CuI (80.67 mg, 423.57 umol, 0.1 eq). The mixture was stirred at 120° C. for 12 hr under N₂ atmosphere. TLC indicated one major new spot with higher polarity. The reaction mixture was diluted with H₂O (50 mL) and extracted with EtOAc 150 mL (50 mL×3). The combined organic layer was dried over Na₂SO₄, and then filtered and concentrated under reduced pressure to give a residue which was purified by flash silica gel chromatography (0-10% Ethyl acetate in petroleum ether). Compound ethyl 2-(2-pyridyl)thiazole-5-carboxylate (0.8 g, 3.24 mmol, 76.59% yield, 95% purity) was obtained as a colorless oil. ¹H NMR was recorded.

### Step 2. Synthesis of [2-(2-pyridyl)thiazol-5-y]methanol

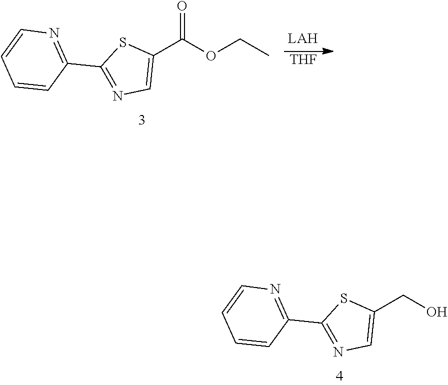 [SMILES: CCOC(=O)c1cnc(-c2ccccn2)s1 ; OCc1cnc(-c2ccccn2)s1 ; "THF"]

To a solution of ethyl 2-(2-pyridyl)thiazole-5-carboxylate (0.8 g, 3.41 mmol, 1 eq) in THF (10 mL) was added LiAlH₄ (194.41 mg, 5.12 mmol, 1.5 eq). The mixture was stirred at 0° C. for 1 hr. LC-MS indicated desired mass was detected. The reaction was quenched with 0.2 mL of H₂O, 0.2 mL of NaOH (3 M in water) and 0.6 mL of H₂O. and then filtered. The filtrate was concentrated under reduced pressure to afford [2-(2-pyridyl)thiazol-5-yl]methanol (370 mg, crude) as a red solid. The crude product was used for the next step without purification.

LCMS (ESI) m/z:193.1[M+H]⁺

### Step 3. Synthesis of 2-(2-pyridyl)thiazole-5-carbaldehyde

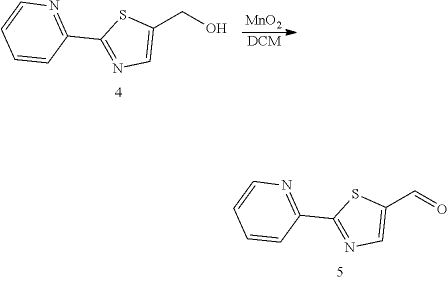

To a solution of [2-(2-pyridyl)thiazol-5-yl]methanol (370 mg, 1.92 mmol, 1 eq) in DCM (20 mL) was added MnO₂ (2.51 g, 28.87 mmol, 15 eq). The mixture was stirred at 25° C. for 12 hr. LC-MS indicated desired mass was detected. The reaction mixture was filtered and concentrated under reduced pressure to give a residue which was purified by flash silica gel chromatography (0-30% Ethyl acetate in petroleum ether). Compound 2-(2-pyridyl)thiazole-5-carbaldehyde (200 mg, 1.05 mmol, 54.63% yield, 100% purity) was obtained as a yellow solid.

LCMS (ESI) m/z 191.0[M+H]⁺; ¹H NMR was recorded.

### Step 4. Synthesis of ethyl (Z)-2-azido-3-[2-(2-pyridyl)thiazol-5-yl]prop-2-enoate

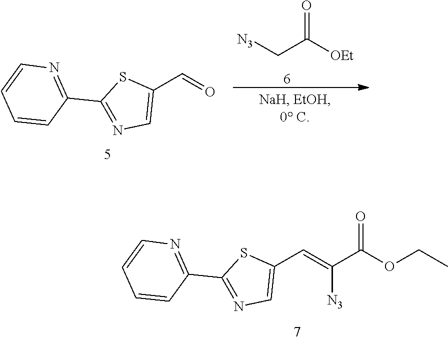

To a solution of EtOH (5 mL) was added NaH (210.26 mg, 5.26 mmol, 60% purity, 5 eq) at 0° C. The reaction mixture was stirred at 0° C. for 0.5 hr. Then a solution of 2-(2-pyridyl)thiazole-5-carbaldehyde (200 mg, 1.05 mmol, 1 eq) and ethyl 2-azidoacetate (678.77 mg, 5.26 mmol, 737.79 uL, 5 eq) in EtOH (3 mL) was added. The reaction mixture was stirred at 0° C. for 1 hr. TLC indicated one major new spot with lower polarity. The reaction mixture was quenched with saturated NH₄Cl (20 mL) at 0° C., and then extracted with petroleum ether (20 mL×2). The combined organic layer was dried over Na₂SO₄, and filtered and concentrated under reduced pressure to give a residue which was purified by flash silica gel chromatography (0-30% Ethyl acetate in petroleum ether). Compound ethyl (Z)-2-azido-3-[2-(2-pyridyl)thiazol-5-yl]prop-2-enoate (300 mg, 945.83 umol, 89.96% yield, 95% purity) was obtained as a colorless oil.

LCMS (ESI) m/z 302.1[M+H]⁺

### Step 5. Synthesis of ethyl 2-(2-pyridyl)-4H-pyrrolo[2,3-d]thiazole-5-carboxylate

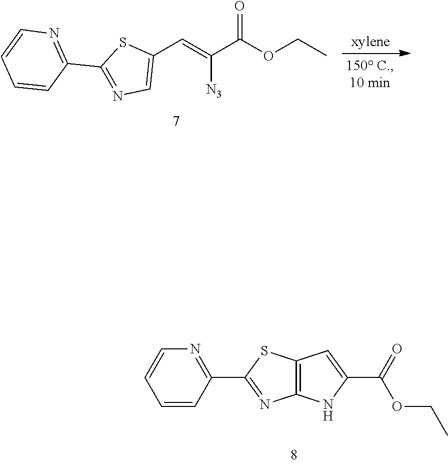 [SMILES: CCOC(=O)/C(=C/c1cnc(-c2ccccn2)s1)N=[N+]=[N-] ; CCOC(=O)c1cc2sc(-c3ccccn3)nc2[nH]1]

A mixture of ethyl (Z)-2-azido-3-[2-(2-pyridyl)thiazol-5-yl]prop-2-enoate (300 mg, 995.61 umol, 1 eq) in xylene (6 mL) was degassed and purged with N₂ for 3 times, and then stirred at 150° C. for 1 hr under N₂ atmosphere. The reaction mixture was cooled to 25° C., and then filtered to collect solid. Compound ethyl 2-(2-pyridyl)-4H-pyrrolo[2,3-d]thiazole-5-carboxylate (150 mg, 521.39 umol, 52.37% yield, 95% purity) was obtained as a yellow solid. ¹H NMR was recorded.

### Step 6. Synthesis of 2-(2-pyridyl)-4H-pyrrolo[2,3-d]thiazole-5-carboxylic acid

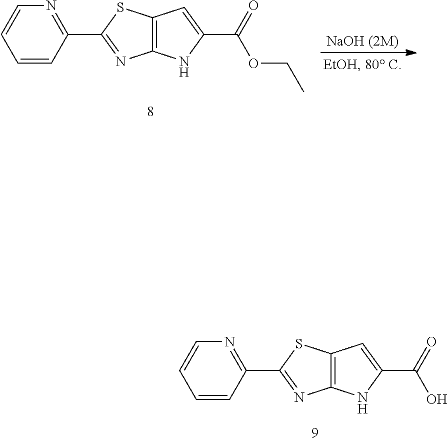 [SMILES: CCOC(=O)c1cc2sc(-c3ccccn3)nc2[nH]1 ; O=C(O)c1cc2sc(-c3ccccn3)nc2[nH]1]

To a solution of ethyl 2-(2-pyridyl)-4H-pyrrolo[2,3-d]thiazole-5-carboxylate (150 mg, 548.83 umol, 1 eq) in THF (2 mL) was added LiOH (4 M in water, 1.00 mL, 7.29 eq). The mixture was stirred at 80° C. for 16 hr. LC-MS indicated desired mass was detected. The reaction mixture was concentrated under reduced pressure to remove solvent. The residue was diluted with H₂O (10 mL) and acidified to pH 3 with 6 M HCl in water, and then extracted with EtOAc (30 mL×4). The combined organic layer was dried over Na₂SO₄, and then filtered and concentrated under reduced pressure to afford 2-(2-pyridyl)-4H-pyrrolo[2,3-d]thiazole-5-carboxylic acid (50 mg, crude) was obtained as a yellow oil. The crude product was used for the next step without further purification.

LCMS (ESI) m/z: 246.1 [M+H]⁺; ¹H NMR (500 MHz, METHANOL-d₄) 8.57 (d, J=4.9 Hz, 1H), 8.20 (d, J=7.9 Hz, 1H), 7.91 (t, J=8.5 Hz, 1H), 7.45-7.39 (m, 1H), 7.13 (s, 1H).

### Step 7. Synthesis of N-(1,1-dimethylsilepan-4-yl)-2-(2-pyridyl)-4H-pyrrolo[2,3-d]thiazole-5-carboxamide

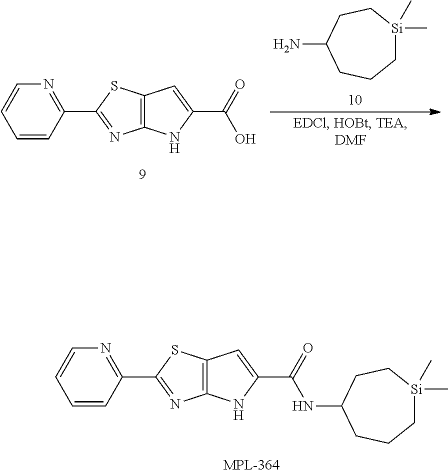

To a solution of 2-(2-pyridyl)-4H-pyrrolo[2,3-d]thiazole-5-carboxylic acid (50.00 mg, 203.87 umol, 1 eq) and 1,1-dimethylsilepan-4-amine (47.41 mg, 244.64 umol, 1.2 eq, HCl salt) in DMF (5 mL), was added HOBt (82.64 mg, 611.61 umol, 3 eq), EDCI (117.25 mg, 611.61 umol, 3 eq) and TEA (123.78 mg, 1.22 mmol, 170.25 uL, 6 eq). The mixture was stirred at 25° C. for 16 hr. LC-MS indicated desired mass was detected. The reaction mixture was concentrated under reduced pressure to remove solvent. The residue was diluted with H₂O (20 mL) and extracted with EtOAc (30 mL×2). The combined organic layer was washed with saturated NaHCO₃ (30 mL×2) and 5% LiCl in water (30 mL×2), dried over Na₂SO₄, filtered and concentrated under reduced pressure to give a residue which was purified by flash silica gel chromatography (0-40% Ethyl acetate in petroleum ether). Compound N-(1,1-dimethylsilepan-4-yl)-2-(2-pyridyl)-4H-pyrrolo [2,3-d]thiazole-5-carboxamide (28.6 mg, 70.74 umol, 34.70% yield, 95.12% purity) was obtained as a white solid.

LCMS (ESI) m/z: 385.2 [M+H]⁺; ¹H NMR (400 MHz, METHANOL-d₄) 8.56 (d, J=4.3 Hz, 1H), 8.19 (d, J=8.2 Hz, 1H), 7.90 (dt, J=1.6, 7.8 Hz, 1H), 7.41 (dd, J=5.5, 7.0 Hz, 1H), 7.11 (s,1H), 3.95 (br s, 1H), 2.10-1.90 (m, 3H), 1.82-1.69 (m, 1H), 1.65-1.50 (m, 2H), 0.89-0.79 (m, 2H), 0.78-0.64 (m, 2H), 0.06 (d, J=7.0 Hz, 6H).

### Example 26, MPL-365

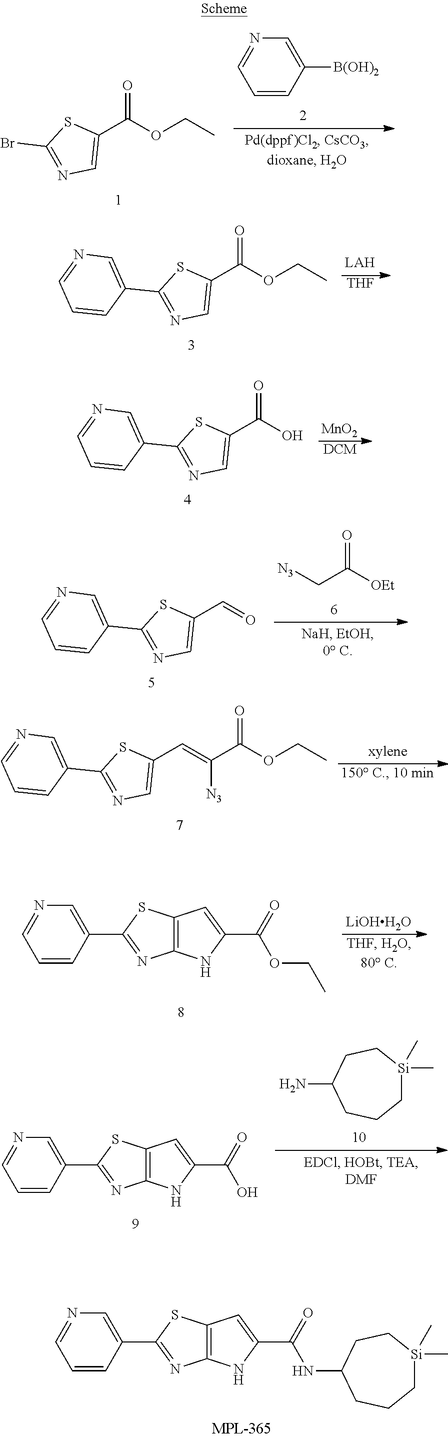

### Step 1. Synthesis oft ethyl 2-(3-pyridyl)thiazole-5-carboxylate

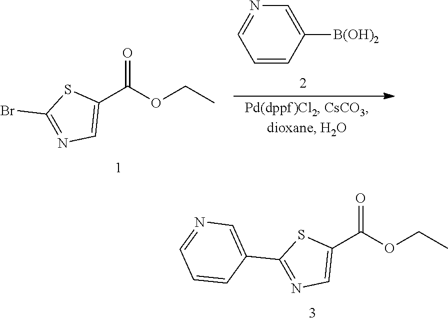 [SMILES: CCOC(=O)c1cnc(Br)s1 ; OB(O)c1cccnc1 ; CCOC(=O)c1cnc(-c2cccnc2)s1]

To a mixture of ethyl 2-bromothiazole-5-carboxylate (2 g, 8.47 mmol, 1 eq), 3-pyridylboronic acid (1.25 g, 10.17 mmol, 1.2 eq) and Cs₂CO₃ (5.52 g, 16.94 mmol, 2 eq) in H₂O (0.2 mL) and dioxane (20 mL) was added Pd(dppf)Cl₂ (619.87 mg, 847.15 umol, 0.1 eq) under N₂. The mixture was heated to 110° C. for 12 hr. LCMS indicated desired mass was detected. The reaction mixture was diluted with EtOAc (40 mL) and filtered to remove the insoluble solid. The filtrate was concentrated in vacuo. The residue was purified by column chromatography (SiO₂, 0-30% EtOAc in petroleum ether). Compound ethyl 2-(3-pyridyl)thiazole-5-carboxylate (931 mg, 3.97 mmol, 46.91% yield) was obtained as a brown solid.

LCMS (ESI) m/z: 235.1 [M+H]⁺; ¹H NMR was recorded.

### Step 2. Synthesis of [2-(3-pyridyl)thiazol-5-yl]methanol

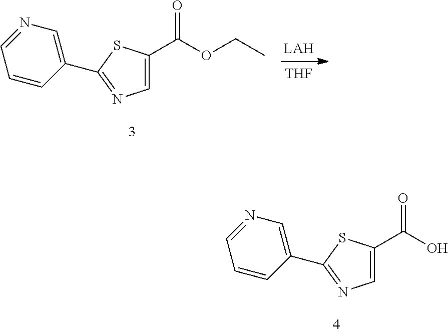 [SMILES: CCOC(=O)c1cnc(-c2cccnc2)s1 ; O=C(O)c1cnc(-c2cccnc2)s1 ; "THF"]

To an ice-cooled solution of ethyl 2-(3-pyridyl)thiazole-5-carboxylate (931 mg, 3.97 mmol, 1 eq) in dry THF (5 mL) was added LAH (226.24 mg, 5.96 mmol, 1.5 eq) in batches. The mixture was stirred at 0-20° C. for 1 hr. TLC (Petroleum ether: Ethyl acetate=1:2) indicated the starting material was consumed completely and two new spots formed. The reaction was quenched with water (0.2 mL), NaOH (15%, 0.2 mL) and water (0.6 mL), and filtered. The filter cake was washed with dichloromethane (30 mL×10). The combined filtrate was dried over Na₂SO₄, and then filtered and concentrated under reduced pressure. The residue was purified by column chromatography (SiO₂, 0-100% Ethyl acetate in petroleum ether). Compound [2-(3-pyridyl)thiazol-5-yl]methanol (293 mg, 1.45 mmol, 36.44% yield, 95% purity) was obtained as a yellow solid. ¹H NMR was recorded.

### Step 3. Synthesis of 2-(3-pyridyl)thiazole-5-carbaldehyde

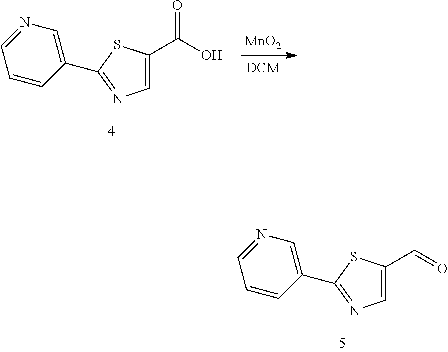

To a solution of [2-(3-pyridyl)thiazol-5-yl]methanol (293 mg, 1.52 mmol, 1 eq) in DCM (20 mL) was added MnO₂ (1.33 g, 15.24 mmol, 10 eq). The mixture was stirred at 25° C. for 12 hr. TLC (Petroleum ether: Ethyl acetate=3:1) indicated the reactant was consumed completely and one new spot formed. The mixture was filtered. The filtrate was concentrated under reduced pressure to afford 2-(3-pyridyl)thiazole-5-carbaldehyde (231 mg, 1.15 mmol, 75.69% yield, 95% purity) as a yellow solid. ¹H NMR was recorded.

### Step 4. Synthesis of ethyl (Z)-2-azido-3-[2-(3-pyridyl)thiazol-5-yl]prop-2-enoate

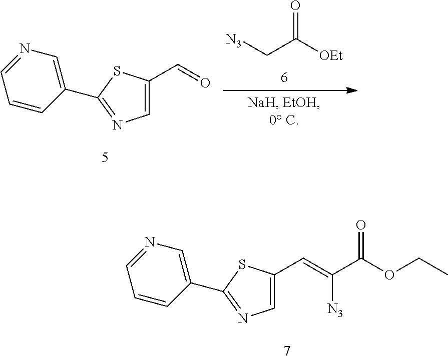 [SMILES: O=Cc1cnc(-c2cccnc2)s1 ; CCOC(=O)CN=[N+]=[N-] ; CCOC(=O)/C(=C/c1cnc(-c2cccnc2)s1)N=[N+]=[N-]]

NaH (145.71 mg, 3.64 mmol, 60% purity, 3 eq) was added to EtOH (7 mL) in batches. The mixture was stirred at 20° C. until a clear solution formed, and then cooled to −10° C. A solution of 2-(3-pyridyl)thiazole-5-carbaldehyde (231 mg, 1.21 mmol, 1 eq) and ethyl 2-azidoacetate (470.39 mg, 3.64 mmol, 511.29 uL, 3 eq) in EtOH (8 mL) and THF (4 mL) was added dropwise. The mixture was stirred at −10° C.˜0° C. for 2 hr. TLC (Petroleum ether: Ethyl acetate=1:1) indicated the aldehyde was consumed completely and many new spots formed. The reaction was quenched with HCl (1M in water) until pH turned to 6, and then extracted with EtOAc (20 mL×2). The combined organic layer was washed with brine (20 mL×2), dried over Na₂SO₄, and then filtered and concentrated under reduced pressure to give a residue which was purified by column chromatography (SiO₂, 0-40% Ethyl acetate in petroleum ether). Compound ethyl (Z)-2-azido-3-[2-(3-pyridyl)thiazol-5-yl]prop-2-enoate (100 mg, 331.87 umol, 27.33% yield, 100% purity) was obtained as a yellow oil.

LCMS (ESI) m/z: 302.1 [M+H]⁺

### Step 5. Synthesis of ethyl 2-(3-pyridyl)-4H-pyrrolo[2,3-d]thiazole-5-carboxylate

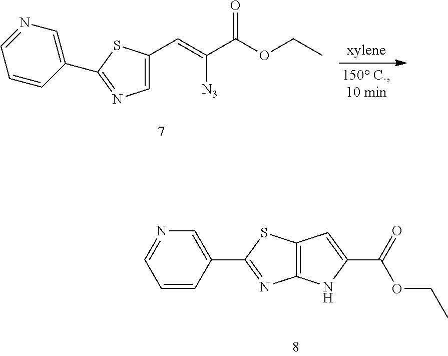 [SMILES: CCOC(=O)/C(=C/c1cnc(-c2cccnc2)s1)N=[N+]=[N-] ; CCOC(=O)c1cc2sc(-c3cccnc3)nc2[nH]1]

A solution of ethyl (Z)-2-azido-3-[2-(3-pyridyl)thiazol-5-yl]prop-2-enoate (100 mg, 331.87 umol, 1 eq) in xylene (5 mL) was stirred at 150° C. for 10 min. LCMS showed desired mass was detected. The reaction mixture was concentrated under reduced pressure to remove solvent. Compound ethyl 2-(3-pyridyl)-4H-pyrrolo[2,3-d]thiazole-5-carboxylate (65 mg, 190.26 umol, 57.33% yield, 80% purity) was obtained as a yellow solid.

LCMS (ESI) m/z: 274.1 [M+H]⁺; ¹H NMR was recorded.

### Step 6. Synthesis of 2-(3-pyridyl)-4H-pyrrolo[2,3-d]thiazole-5-carboxylic acid

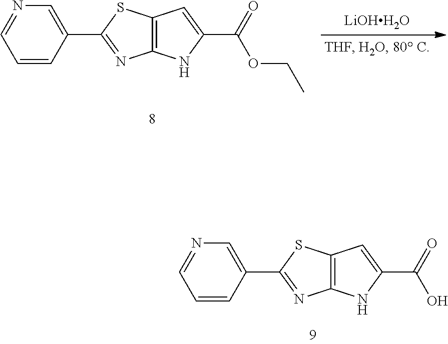 [SMILES: CCOC(=O)c1cc2sc(-c3cccnc3)nc2[nH]1 ; "LiOH·H2O" ; O=C(O)c1cc2sc(-c3cccnc3)nc2[nH]1]

To a solution of ethyl 2-(3-pyridyl)-4H-pyrrolo[2,3-d]thiazole-5-carboxylate (60 mg, 219.53 umol, 1 eq) in THF (1.5 mL) was added a solution of LiOH.H₂O (55.27 mg, 1.32 mmol, 6 eq) in H₂O (1.5 mL). The mixture was stirred at 80° C. for 16 hr. LCMS showed desired compound was detected. The reaction mixture was concentrated under reduced pressure to remove THF, and then acidified to pH 6-7 with HCl (1 N in water) and lyophilized. The residue was diluted with a mixed solution of dichloromethane and methanol (10:1, 5 mL) and filtered. The filtrate was concentrated under reduced pressure to give 2-(3-pyridyl)-4H-pyrrolo[2,3-d]thiazole-5-carboxylic acid (53 mg, crude, HCl salt) as a yellow solid.

LCMS (ESI) m/z: 246.0 [M+H]⁺

### Step 7. Synthesis of N-(1,1-dimethylsilepan-4-yl)-2-(3-pyridyl)-4H-pyrrolo[2,3-d]thiazole-5-carboxamide

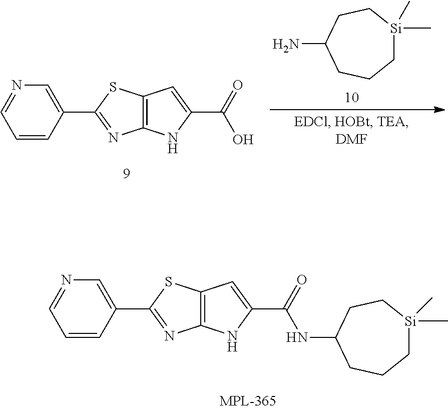

To a solution of 2-(3-pyridyl)-4H-pyrrolo[2,3-d]thiazole-5-carboxylic acid (35 mg, 142.71 umol, 1 eq) and 1,1-dimethylsilepan-4-amine (33.19 mg, 171.25 umol, 1.2 eq, HCl salt) in DMF (1.5 mL) was added a solution of EDCI (82.07 mg, 428.12 umol, 3 eq) and HOBt (57.85 mg, 428.12 umol, 3 eq) in DMF (0.5 mL), followed by TEA (86.64 mg, 856.25 umol, 119.18 uL, 6 eq). The mixture was stirred at 20° C. for 1 hr. LCMS showed desired mass was detected. The mixture was filtered to obtain filtrate which was purified by prep-HPLC (column: Phenomenex Synergi C18 150*30 mm*4 um; mobile phase: A: 0.05% HCl in water, B: CH₃CN, gradient: 50%-70% B over 9 min). Compound N-(1,1-dimethylsilepan-4-yl)-2-(3-pyridyl)-4H-pyrrolo[2,3-d]thiazole-5-carboxamide (12.5 mg, 32.50 umol, 22.78% yield, 100% purity) was obtained as a yellow solid.

LCMS (ESI) m/z: 385.2 [M+H]⁺; ¹H NMR (400 MHz, DMSO-d₆) δ=12.55 (br s, 1H), 9.15 (br s, 1H), 8.67 (br s, 1H), 8.35 (br s, 1H), 8.11 (br s, 1H), 7.61 (br s, 1H), 7.20 (s, 1H), 3.87 (br s, 1H), 1.97-1.80 (m, 3H), 1.74-1.61 (m, 1H), 1.57-1.44 (m, 2H), 0.83-0.69 (m, 2H), 0.67-0.57 (m, 2H), 0.04 (d, J=6.8 Hz, 6H).

### Example 27, MPL-369

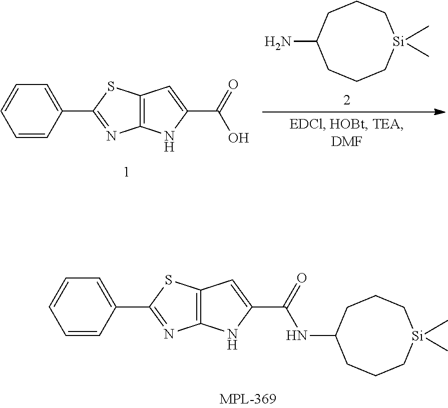

To a solution of 2-phenyl-4H-pyrrolo[2,3-d]thiazole-5-carboxylic acid (53 mg, 216.97 umol, 1 eq) and 1,1-dimethylsilocan-5-amine (45.09 mg, 216.97 umol, 1 eq, HCl salt) in DMF (1 mL) at 25° C. was added a solution of HOBt (87.95 mg, 650.92 umol, 3 eq) and EDCI (124.78 mg, 650.92 umol, 3 eq) in DMF (1 mL), followed by TEA (109.78 mg, 1.08 mmol, 151.00 uL, 5 eq). The mixture was stirred at 25° C. for 12 hr. LC-MS showed desired compound was detected. The mixture was purified by prep-HPLC (column: YMC-Actus Triart C18 150*30 mm*5 um; mobile phase: A: 0.225% formic acid in water, B: CH₃CN, gradient: 70%-100% B over 11 min). The residue from prep-HPLC was further purified by SFC (Berger MG II; column: DAICEL CHIRALPAK AS (250 mm*30 mm, 10 um); mobile phase: A: 0.1%NH₃H₂O in EtOH; B: CO₂; 30% B isocratic, flow rate: 60 mL/min). Compound N-(1,1-dimethylsilocan-5-yl)-2-phenyl-4H-pyrrolo[2,3-d]thiazole-5-carboxamide (24.5 mg, 59.15 umol, 27.26% yield, 96% purity) was obtained as a white solid.

LCMS (ESI) m/z 398.1 [M+H]⁺; ¹H NMR (500 MHz, CHLOROFORM-d) δ=9.56 (br s, 1H), 7.97 (dd, J=1.4, 8.0 Hz, 2H), 7.51-7.39 (m, 3H), 6.72 (d, J=1.8 Hz, 1H), 5.84 (br d, J=8.5 Hz, 1H), 4.32-4.05 (m, 1H), 1.89-1.61 (m, 8H), 0.87-0.73 (m, 4H), 0.11-−0.01 (m, 6H).

### Example 28, MPL-370

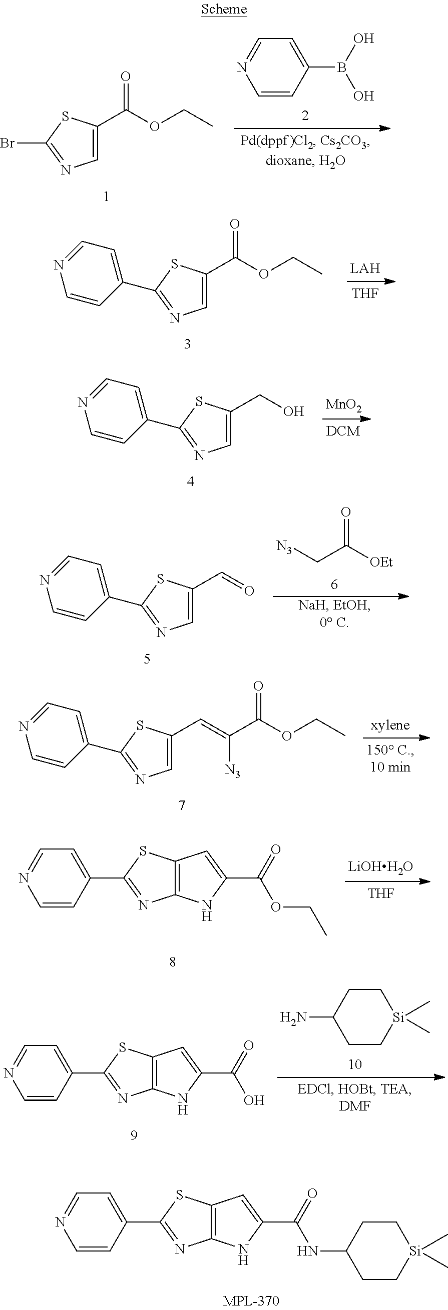

### Step 1. Synthesis of ethyl 2-(4-pyridyl)thiazole-5-carboxylate

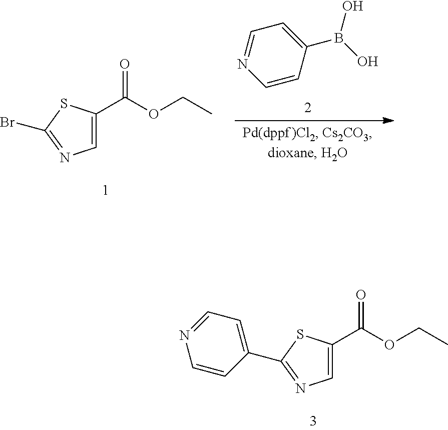 [SMILES: CCOC(=O)c1cnc(Br)s1 ; OB(O)c1ccncc1 ; CCOC(=O)c1cnc(-c2ccncc2)s1]

To a mixture of ethyl 2-bromothiazole-5-carboxylate (2 g, 8.47 mmol, 1 eq), 4-pyridylboronic acid (1.56 g, 12.71 mmol, 1.5 eq), Cs₂CO₃ (5.52 g, 16.94 mmol, 2 eq) was added dioxane (50 mL) and H₂O (0.5 mL). The mixture was purged with N₂, then Pd(dppf)Cl₂ (619.86 mg, 847.15 umol, 0.1 eq) was added. The mixture was stirred at 110° C. for 12 hr. LC-MS showed desired compound was detected. The mixture was filtered. The cake was washed with EtOAc (50 mL×2). The combined filtrate was dried over Na₂SO₄. The solvent was removed in vacuo. The residue was purified by column chromatography (SiO₂, 0-25% Ethyl acetate in petroleum ether). Compound ethyl 2-(4-pyridyl)thiazole-5-carboxylate (1.42 g, 5.77 mmol, 68.07% yield, 95% purity) was obtained as a black solid.

LCMS (ESI) m/z 235.0 [M+H]⁺; ¹H NMR was recorded.

### Step 2. Synthesis of [2-(4-pyridyl)thiazol-5-yl]methanol

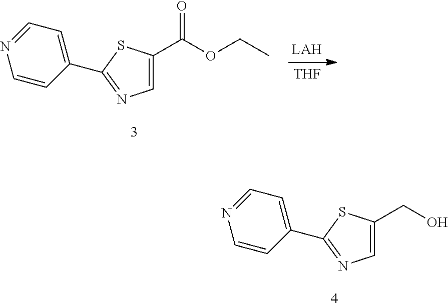 [SMILES: CCOC(=O)c1cnc(-c2ccncc2)s1 ; OCc1cnc(-c2ccncc2)s1 ; "THF"]

To an ice-cooled solution of ethyl 2-(4-pyridyl)thiazole-5-carboxylate (1.42 g, 6.06 mmol, 1 eq) in dried THF (30 mL) was added LAH (350 mg, 9.22 mmol, 1.52 eq) in batches. The mixture was stirred at 0-20° C. for 1 hr. TLC indicated reactant 3 was consumed completely. The reaction was quenched with water (0.35 mL), NaOH (15%, 0.35 mL) and water (1.05 mL), and then filtered. The filter cake was washed with EtOAc (50 mL×3). The combined filtrate was dried over Na₂SO₄, and then filtered and concentrated under reduced pressure. Compound [2-(4-pyridyl)thiazol-5-yl]methanol (730 mg, 3.04 mmol, 50.12% yield, 80% purity) was obtained as a yellow solid. ¹H NMR was recorded. The crude product was used for next step without further purification.

### Step 3. Synthesis of 2-(4-pyridyl)thiazole-5-carbaldehyde

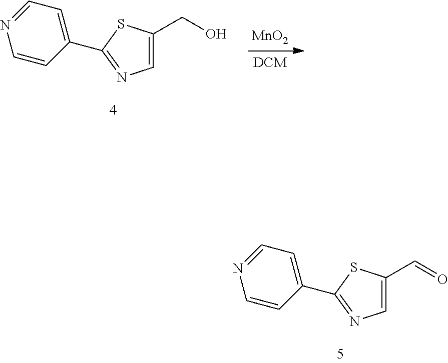

To a solution of [2-(4-pyridyl)thiazol-5-yl]methanol (730 mg, 3.80 mmol, 1 eq) in DCM (10 mL) was added MnO₂ (3.30 g, 37.97 mmol, 10 eq). The mixture was stirred at 25° C. for 2 hr. TLC showed one major new spot formed. The mixture was then filtered. The filter cake was washed with EtOAc (20 mL×5). The combined filtrate was dried over Na₂SO₄, and then filtered and concentrated under reduced pressure. The residue was purified by column chromatography (SiO₂, 0-25% Ethyl acetate in petroleum ether). Compound 2-(4-pyridyl)thiazole-5-carbaldehyde (294 mg, 1.39 mmol, 36.63% yield, 90% purity) was obtained as a white solid. ¹H NMR was recorded.

### Step 4. Synthesis of ethyl (Z)-2-azido-3-[2-(4-pyridyl)thiazol-5-yl]prop-2-enoate

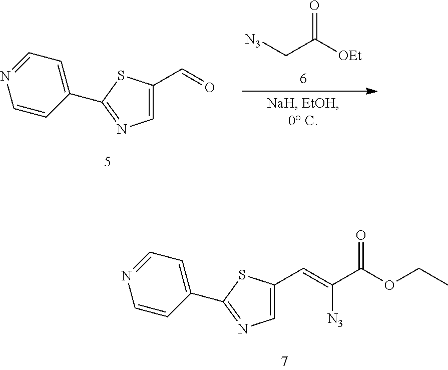 [SMILES: O=Cc1cnc(-c2ccncc2)s1 ; CCOC(=O)CN=[N+]=[N-] ; CCOC(=O)/C(=C/c1cnc(-c2ccncc2)s1)N=[N+]=[N-]]

NaH (185.45 mg, 4.64 mmol, 60% purity, 3 eq) was added to EtOH (2 mL) in batches. The mixture was stirred at 30° C. until a clear solution formed, and then cooled to −10° C. Then a solution of 2-(4-pyridyl)thiazole-5-carbaldehyde (294 mg, 1.55 mmol, 1 eq) and ethyl 2-azidoacetate (598.68 mg, 4.64 mmol, 650.74 uL, 3 eq) in EtOH (2 mL) was added to the mixture dropwise. The mixture was stirred at −10° C.˜0° C. for 2 hr. LC-MS showed desired compound was detected. The reaction was quenched with saturated NH₄Cl (5 mL), and then extracted with EtOAc (10 mL×2). The combined organic layer was washed with brine (5 mL×2), dried over Na₂SO₄, and then filtered and concentrated under reduced pressure to give a residue which was purified by column chromatography (SiO₂, 0-25% Ethyl acetate in petroleum ether). Compound ethyl (Z)-2-azido-3-[2-(4-pyridyl)thiazol-5-yl]prop-2-enoate (100 mg, 232.31 umol, 15.03% yield, 70% purity) was obtained as a yellow solid.

LCMS (ESI) m/z 273.9 [M+H]⁺

### Step 5. Synthesis of ethyl 2-(4-pyridyl)-4H-pyrrolo[2,3-d]thiazole-5-carboxylate

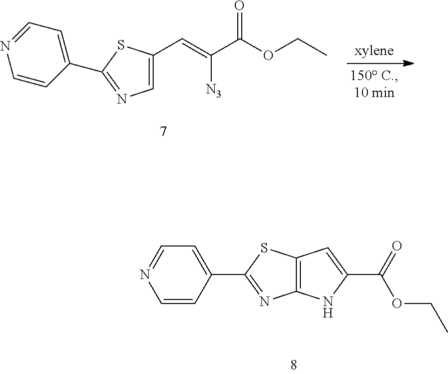 [SMILES: CCOC(=O)/C(=C/c1cnc(-c2ccncc2)s1)N=[N+]=[N-] ; CCOC(=O)c1cc2sc(-c3ccncc3)nc2[nH]1]

Ethyl (Z)-2-azido-3-[2-(4-pyridyl)thiazol-5-yl]prop-2-enoate (100 mg, 331.87 umol, 1 eq) in xylene (3 mL) was stirred at 150° C. for 10 min. LC-MS showed product was detected. The rmixture was purified by column chromatography (SiO₂, 0-25% Ethyl acetate in petroleum ether). Compound ethyl 2-(4-pyridyl)-4H-pyrrolo[2,3-d]thiazole-5-carboxylate (50 mg, 54.88 umol, 16.54% yield, 30% purity) was obtained as a yellow solid.

LCMS (ESI) m/z 274.2 [M+H]⁺; ¹H NMR was recorded.

### Step 6. Synthesis of 2-(4-pyridyl)-4H-pyrrolo[2,3-d]thiazole-5-carboxylic acid

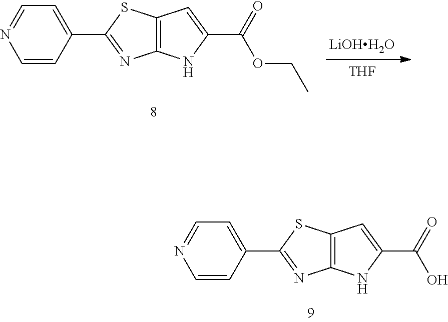

To a solution of ethyl 2-(4-pyridyl)-4H-pyrrolo[2,3-d]thiazole-5-carboxylate (50 mg, 182.94 umol, 1 eq) in THF (2 mL) was added a solution of LiOH.H₂O (30.71 mg, 731.77 umol, 4 eq) in H₂O (2 mL). The mixture was stirred at 80° C. for 2 hr. LC-MS showed the starting material remained. The mixture was stirred at 80° C. for additional 12 hr. LC-MS showed the starting material was consumed completely and desired compound was detected. The reaction mixture was concentrated under reduced pressure to remove THF, and then extracted with EtOAc (10 mL×3). The aqueous phase was acidified to pH 6 with HCl (6 M in water), diluted with water (10 mL) and extracted with EtOAc (20 mL×2). The combined organic layer was washed with brine (20 mL×2), dried over Na₂SO₄, and filtered and concentrated under reduced pressure to give 2-(4-pyridyl)-4H-pyrrolo[2,3-d]thiazole-5-carboxylic acid (15 mg, 48.93 umol, 26.75% yield, 80% purity) as a yellow solid. The crude product was used for the next step without further purification.

LCMS (ESI) m/z 246.0 [M+H]⁺; ¹H NMR (500 MHz, METHANOL-d₄) δ=8.58-8.53 (m, 2H), 7.87-7.81 (m, 2H), 7.79-7.70 (m, 1H).

### Step 7. Synthesis of N-(1,1-dimethylsilinan-4-yl)-2-(4-pyridyl)-4H-pyrrolo[2,3-d]thiazole-5-carboxamide

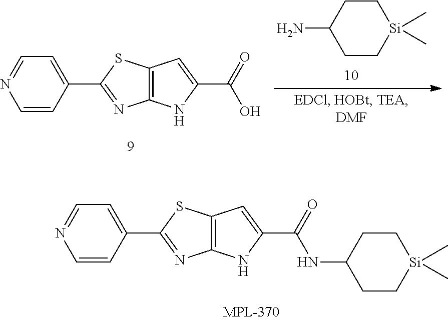

To a solution of 2-(4-pyridyl)-4H-pyrrolo[2,3-d]thiazole-5-carboxylic acid (15 mg, 61.16 umol, 1 eq) and 1,1-dimethylsilinan-4-amine (10.52 mg, 73.39 umol, 1.2 eq, HCl salt) in DMF (1 mL) at 25° C. was added a solution of HOBt (24.79 mg, 183.48 umol, 3 eq) and EDCI (35.17 mg, 183.48 umol, 3 eq) in DMF (1 mL), followed by TEA (30.94 mg, 305.80 umol, 42.56 uL, 5 eq). The reaction was stirred at 25° C. for 2 hr. LC-MS showed desired compound was detected. The mixture was purified by prep-HPLC (column: YMC-Actus Triart C18 150*30 mm*5 um; mobile phase: A: 0.225% formic acid in water, B: CH₃CN; gradient: 30%-60% B over 11 min). Compound N-(1,1-dimethylsilinan-4-yl)-2-(4-pyridyl)-4H-pyrrolo [2,3 -d]thiazole-5 -carboxamide (2.8 mg, 7.56 umol, 12.36% yield, 100% purity) was obtained as a yellow solid.

LCMS (ESI) m/z 371.1 [M+H]⁺; ¹H NMR (500 MHz, CHLOROFORM-d) δ=9.83 (br s, 1H), 8.72 (br d, J=5.0 Hz, 2H), 7.84 (d, J=6.1 Hz, 2H), 6.76 (d, J=1.8 Hz, 1H), 5.90 (br d, J=8.1 Hz, 1H), 3.99-3.85 (m, 1H), 2.25-2.11 (m, 2H), 1.62-1.54 (m, 3H), 0.86-0.66 (m, 4H), 0.08 (d, J=17.9 Hz, 6H).

### Example 29, MPL-371

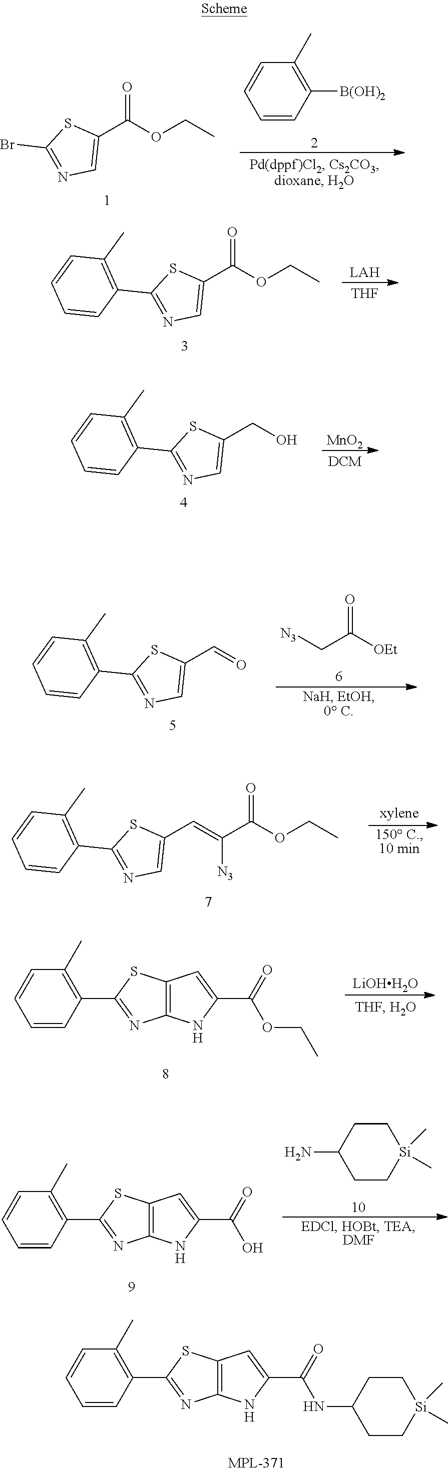

### Step 1. Synthesis of ethyl 2-(o-tolyl)thiazole-5-carboxylate

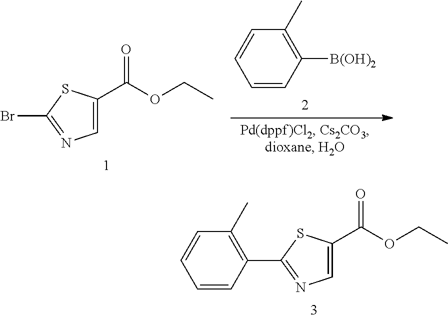

A mixture of ethyl 2-bromothiazole-5-carboxylate (2 g, 8.47 mmol, 1 eq), o-tolylboronic acid (1.73 g, 12.71 mmol, 1.5 eq) and Cs₂CO₃ (5.52 g, 16.94 mmol, 2 eq) in H₂O (0.3 mL) and dioxane (30 mL) was de-gassed, Pd(dppf)Cl₂.CH₂Cl₂ (691.81 mg, 847.15 umol, 0.1 eq) was then added. The mixture was heated at 100° C. for 12 hours under N₂. LCMS indicated desired mass was detected. The mixture was filtered to obtain filtrate, which was purified by column chromatography (SiO₂, 0-4% Ethyl acetate in petroleum ether). Compound ethyl 2-(o-tolyl)thiazole-5-carboxylate (1.64 g, 5.98 mmol, 70.54% yield, 90% purity) was obtained as a green oil.

LCMS (ESI) m/z: 248.1 [M+H]⁺; ¹H NMR was recorded.

### Step 2. Synthesis of [2-(o-tolyl)thiazol-5-yl]methanol

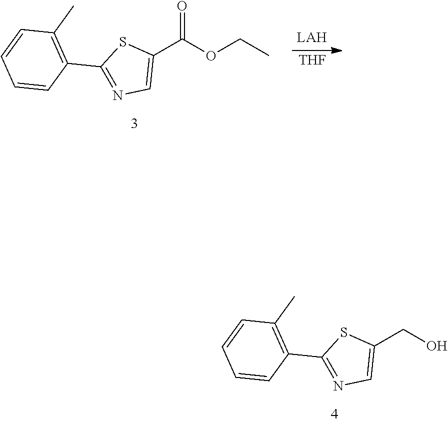 [SMILES: CCOC(=O)c1cnc(-c2ccccc2C)s1 ; Cc1ccccc1-c1ncc(CO)s1 ; "THF"]

To an ice-cooled solution of ethyl 2-(o-tolyl)thiazole-5-carboxylate (1.64 g, 6.64 mmol, 1 eq) in dry THF (5 mL) was added LAH (377.95 mg, 9.96 mmol, 1.5 eq) in batches. The mixture was stirred at 0-20° C. for 1 hr. TLC (Petroleum ether: Ethyl acetate=3:1) indicated compound 3 was consumed completely and two new spots formed. The reaction was quenched with water (0.38 mL), NaOH (15%, 0.38 mL) and water (1.14 mL). The mixture was then filtered. The filter cake was washed with dichloromethane (30 mL×3). The combined filtrate was dried over Na₂SO₄, filtered and concentrated under reduced pressure. The residue was purified by column chromatography (SiO₂, 0-100% Ethyl acetate in petroleum ether). Compound [2-(o-tolyl)thiazol-5-yl]methanol (1.3 g, 5.70 mmol, 85.85% yield, 90% purity) was obtained as a green oil. ¹H NMR was recorded.

### Step 3. Synthesis of 2-(o-tolyl)thiazole-5-carbaldehyde

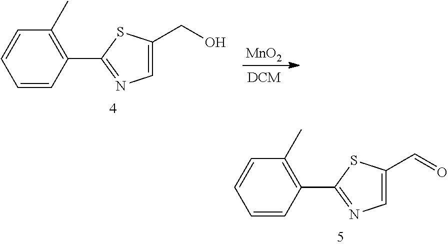

To a solution of [2-(o-tolyl)thiazol-5-yl]methanol (1.3 g, 6.33 mmol, 1 eq) in DCM (20 mL) was added MnO₂ (5.51 g, 63.33 mmol, 10 eq). The mixture was stirred at 20° C. for 2 hr. TLC (Petroleum ether: Ethyl acetate=3:1) indicated compound 4 was consumed completely and many new spots formed. The mixture was filtered to obtain filtrate which was purified by column chromatography (SiO₂, 0-30% Ethyl acetate in petroleum ether). Compound 2-(o-tolyl)thiazole-5-carbaldehyde (1.1 g, 5.14 mmol, 81.18% yield, 95% purity) was obtained as a brown solid. ¹H NMR was recorded.

### Step 4. Synthesis of ethyl (Z)-2-azido-3-[2-(o-tolyl)thiazol-5-yl]prop-2-enoate

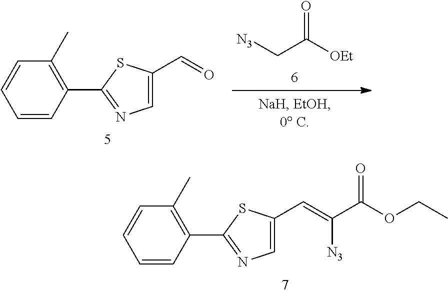 [SMILES: Cc1ccccc1-c1ncc(C=O)s1 ; CCOC(=O)CN=[N+]=[N-] ; CCOC(=O)/C(=C/c1cnc(-c2ccccc2C)s1)N=[N+]=[N-]]

NaH (295.16 mg, 7.38 mmol, 60% purity, 3 eq) was added to EtOH (8 mL) in batches. The mixture was stirred at 20° C. until a clear solution formed, and then cooled to −10° C. Then a solution of 2-(o-tolyl)thiazole-5-carbaldehyde (500 mg, 2.46 mmol, 1 eq) and ethyl 2-azidoacetate (952.85 mg, 7.38 mmol, 1.04 mL, 3 eq) in THF (5 mL)was added dropwise. The mixture was stirred at −10° C.˜0° C. for 2 hr. TLC (Petroleum ether: Ethyl acetate=3:1) indicated compound 5 was consumed completely and many new spots formed. The reaction was quenched with saturated NH₄Cl (40 mL), and then extracted with EtOAc (20 mL×2). The combined organic layer was washed with brine (20 mL×2), dried over Na₂SO₄, filtered and concentrated under reduced pressure to give a residue which was purified by column chromatography (SiO2, 0-20% Ethyl acetate in petroleum ether). Compound ethyl (Z)-2-azido-3-[2-(o-tolyl) thiazol-5-yl] prop-2-enoate (500 mg, 1.10 mmol, 44.61% yield, 69% purity) was obtained as a yellow oil.

LCMS (ESI) m/z: 315.1 [M+H]⁺

### Step 5. Synthesis of ethyl 2-(o-tolyl)-4H-pyrrolo[2,3-d]thiazole-5-carboxylate

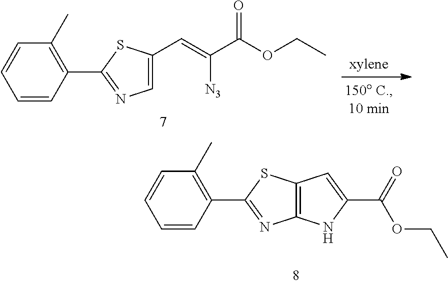 [SMILES: CCOC(=O)/C(=C/c1cnc(-c2ccccc2C)s1)N=[N+]=[N-] ; CCOC(=O)c1cc2sc(-c3ccccc3C)nc2[nH]1]

A solution of ethyl (Z)-2-azido-3-[2-(o-tolyl) thiazol-5-yl]prop-2-enoate (500 mg, 1.59 mmol, 1 eq) in xylene (3 mL) was stirred at 150° C. for 20 min. LC-MS showed desired mass was detected. The reaction mixture was concentrated under reduced pressure to remove solvent. The residue was purified by column chromatography (SiO₂, 0-20% Ethyl acetate in petroleum ether). Compound ethyl 2-(o-tolyl)-4H-pyrrolo[2,3-d] thiazole-5-carboxylate (213 mg, 706.66 umol, 44.43% yield, 95% purity) was obtained as a yellow solid.

LCMS (ESI) m/z: 287.1 [M+H]⁺; ¹H NMR was recorded.

### Step 6. Synthesis of 2-(o-tolyl)-4H-pyrrolo[2,3-d]thiazole-5-carboxylic acid

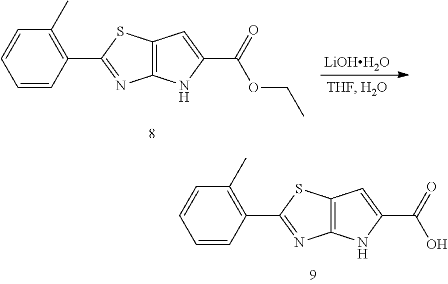

To a solution of ethyl 2-(o-tolyl)-4H-pyrrolo[2,3-d]thiazole-5-carboxylate (213 mg, 743.85 umol, 1 eq) in THF (4 mL) was added a solution of LiOH.H₂O (187.29 mg, 4.46 mmol, 6 eq) in H₂O (4 mL). The mixture was stirred at 80° C. for 12 hr. LC-MS showed desired mass was detected. The reaction mixture was concentrated under reduced pressure to remove THF. The aqueous phase was acidified to pH 3-4 with HCl (1 N in water) and then filtered. The cake was washed with petroleum ether (15 mL) and dried under reduced pressure. Compound 2-(o-tolyl)-4H-pyrrolo [2,3-d]thiazole-5-carboxylic acid (180 mg, 662.03 umol, 89.00% yield, 95% purity) was obtained as a white solid.

LCMS (ESI) m/z: 259.0 [M+H]⁺; ¹H NMR (500 MHz, DMSO-d₆) δ=12.72 (br s, 2H), 7.74 (d, J=7.5 Hz, 1H), 7.43-7.38 (m, 2H), 7.38-7.32 (m, 1H), 7.12 (d, J=1.5 Hz, 1H), 2.59 (s, 3H).

### Step 7. Synthesis of N-(1,1-dimethylsilinan-4-yl)-2-(o-tolyl)-4H-pyrrolo[2,3-d]thiazole-5-carboxamide

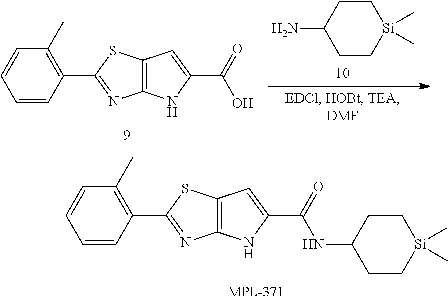

To a solution of 2-(o-tolyl)-4H-pyrrolo[2,3-d]thiazole-5-carboxylic acid (50 mg, 193.58 umol, 1 eq) and 1,1-dimethylsilinan-4-amine (41.76 mg, 232.29 umol, 1.2 eq, HCl) in DMF (2 mL) was added a solution of EDCI (111.33 mg, 580.73 umol, 3 eq) and HOBt (78.47 mg, 580.73 umol, 3 eq) in DMF (0.5 mL), followed by TEA (117.53 mg, 1.16 mmol, 161.66 uL, 6 eq). The mixture was stirred at 20° C. for 1 hr. LC-MS showed desired mass. The reaction mixture was filtered to obtain filtrate which was purified by prep-HPLC (column: YMC-Actus Triart C18 150*30 mm*5 um; mobile phase: A: 0.225% formic acid in water, B: CH₃CN, gradient: 80%-100% B over 11 min). Compound N-(1,1-dimethylsilinan-4-yl)-2-(o-tolyl)-4H-pyrrolo[2,3-d]thiazole-5-carboxamide (41.2 mg, 106.95 umol, 55.25% yield, 99.57% purity) was obtained as a white solid.

LCMS (ESI) m/z: 384.1 [M+H]⁺; ¹H NMR (400 MHz, DMSO-d₆) δ=12.42 (br s, 1H), 7.97 (d, J=8.2 Hz, 1H), 7.73 (d, J=7.0 Hz, 1H), 7.41-7.29 (m, 3H), 7.14 (s, 1H), 3.69 (br d, J=7.8 Hz,1H), 2.59 (s, 3H), 1.99 (br d, J=9.4 Hz, 2H), 1.68-1.47 (m, 2H), 0.78 (br d, J=14.9 Hz, 2H), 0.66-0.55 (m, 2H), 0.11-0.01 (m, 6H).

### Example 30, MPL-372

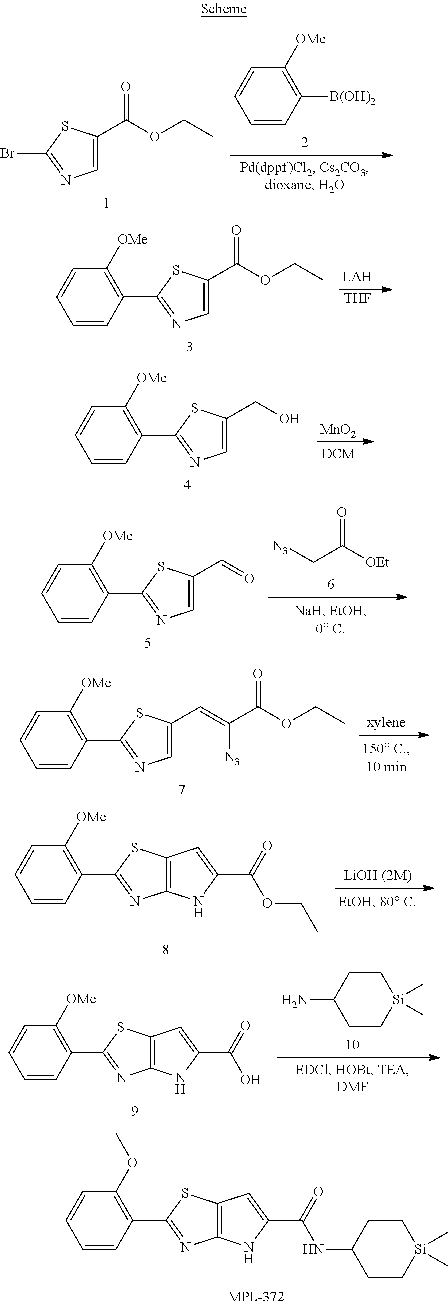

### Step 1. Synthesis of ethyl 2-(2-methoxyphenyl)thiazole-5-carboxylate

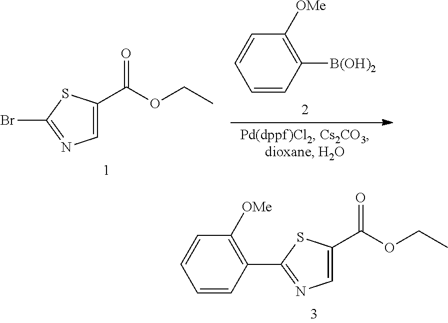

To a solution of ethyl 2-bromothiazole-5-carboxylate (2 g, 8.47 mmol, 1 eq) and (2-methoxyphenyl)boronic acid (3.86 g, 25.41 mmol, 3 eq) in dioxane (20 mL) and H₂O (2 mL) was added Pd(dppf)Cl₂ (309.88 mg, 423.50 umol, 0.05 eq) and Cs₂CO₃ (13.80 g, 42.35 mmol, 5 eq). The mixture was stirred at 120° C. for 12 hr under N₂ atmosphere. LC-MS indicated desired mass was detected. The reaction mixture was concentrated under reduced pressure to remove solvent, then diluted with H₂O (50 mL) and extracted with EtOAc 150 mL (50 mL×3). The combined organic layer was dried over Na₂SO₄, and then filtered and concentrated under reduced pressure to give a residue which was purified by flash silica gel chromatography (0-10% Ethyl acetate in petroleum ether). Compound ethyl 2-(2-methoxyphenyl)thiazole-5-carboxylate (1.4 g, 5.05 mmol, 59.63% yield, 95% purity) was obtained as a white solid. ¹H NMR was recorded.

### Step 2. Synthesis of [2-(2-methoxyphenyl)thiazol-5-yl]methanol

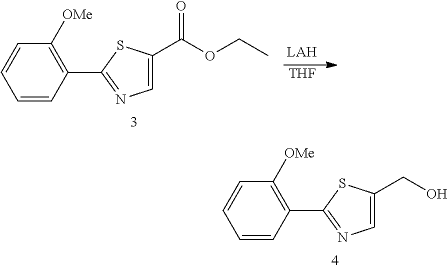 [SMILES: CCOC(=O)c1cnc(-c2ccccc2OC)s1 ; COc1ccccc1-c1ncc(CO)s1 ; "THF"]

To a solution of ethyl 2-(2-methoxyphenyl)thiazole-5-carboxylate (1.4 g, 5.32 mmol, 1 eq) in THF (10 mL) was added LiAlH₄ (302.70 mg, 7.98 mmol, 1.5 eq). The mixture was stirred at 0° C. for 1 hr. TLC indicated one major new spot with higher polarity. The reaction mixture was quenched with H₂O (0.3 mL) at 0° C., followed by addition of 0.3 mL of NaOH in water (3M) and 0.9 mL of water. The mixture was filtered. The filtrate was concentered under reduce pressure. Compound [2-(2-methoxyphenyl) thiazol-5-yl] methanol (1.05 g, crude) was obtained as a yellow solid. The crude product was used for the next step without purification.

### Step 3. Synthesis of 2-(2-methoxyphenyl) thiazole-5-carbaldehyde

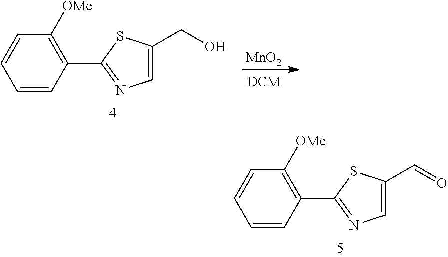

To a solution of [2-(2-methoxyphenyl)thiazol-5-yl]methanol (1.05 g, 4.75 mmol, 1 eq) in DCM (10 mL) was added MnO₂ (7.43 g, 85.41 mmol, 18 eq). The mixture was stirred at 30° C. for 12 hr. TLC indicated one major new spot with lower polarity. The reaction mixture was filtered and concentrated under reduced pressure to give a residue which was purified by flash silica gel chromatography (0-10% Ethyl acetate in petroleum ether). Compound 2-(2-methoxyphenyl) thiazole-5-carbaldehyde (0.8 g, 3.47 mmol, 73.05% yield, 95% purity) was obtained as a white solid. ¹H NMR was recorded.

### Step 4. Synthesis of ethyl (Z)-2-azido-3-[2-(2-methoxyphenyl)thiazol-5-yl]prop-2-enoate

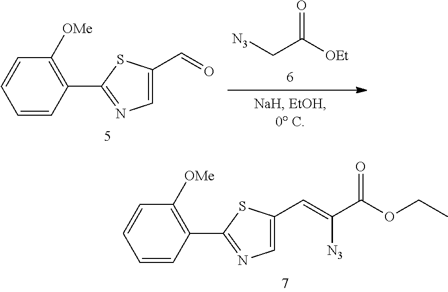

To EtOH (10 mL) was added NaH (729.73 mg, 18.24 mmol, 60% purity, 5 eq) at 0° C. The reaction mixture was stirred at 0° C. for 0.5 hr. Then a solution of 2-(2-methoxyphenyl)thiazole-5-carbaldehyde (0.8 g, 3.65 mmol, 1 eq) and ethyl 2-azidoacetate (2.36 g, 18.24 mmol, 2.56 mL, 5 eq) in THF (3 mL) was added. The reaction mixture was stirred at 0° C. for 1 hr. TLC indicated one major new spot with lower polarity. The reaction mixture was quenched by addition of saturated NH₄Cl (20 m) at 0° C., and then extracted with EtOAc (20 mL×2). The combined organic layer was dried over Na₂SO₄, and then filtered and concentrated under reduced pressure to give a residue which was purified by flash silica gel chromatography (0-30% Ethyl acetate in petroleum ether). Compound ethyl (Z)-2-azido-3-[2-(2-methoxyphenyl)thiazol-5-yl]prop-2-enoate (1.2 g, crude) in 5 mL of xylene was obtained as a colorless oil.

LCMS (ESI) m/z 331.1[M+H]⁺

### Step 5. Synthesis of ethyl 2-(2-methoxyphenyl)-4H-pyrrolo[2,3-d]thiazole-5-carboxylate

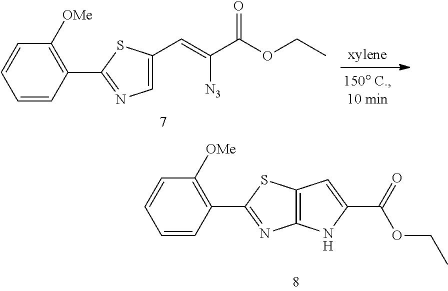 [SMILES: CCOC(=O)/C(=C/c1cnc(-c2ccccc2OC)s1)N=[N+]=[N-] ; CCOC(=O)c1cc2sc(-c3ccccc3OC)nc2[nH]1]

A mixture of ethyl (Z)-2-azido-3-[2-(2-methoxyphenyl)thiazol-5-yl]prop-2-enoate (1.2 g, 3.63 mmol, 1 eq) in xylene (10 mL) was degassed and purged with N₂ for 3 times, and then stirred at 150° C. for 0.5 hr under N₂ atmosphere. TLC indicated one major new spot with higher polarity formed. The reaction mixture was concentrated under reduced pressure to give a residue which was purified by flash silica gel chromatography (0-30% Ethyl acetate in petroleum ether). Compound ethyl 2-(2-methoxyphenyl)-4H-pyrrolo[2,3-d]thiazole-5-carboxylate (250 mg, 785.52 umol, 21.63% yield, 95% purity) was obtained as a yellow oil.

LCMS (ESI) m/z: 303.1 [M+H]⁺

### Step 6. Synthesis of 2-(2-methoxyphenyl)-4H-pyrrolo[2,3-d]thiazole-5-carboxylic acid

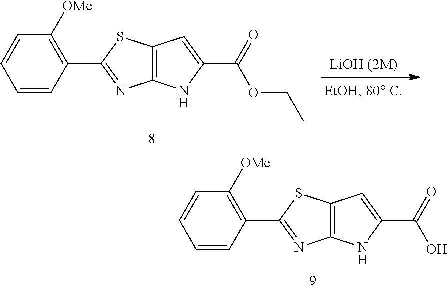 [SMILES: CCOC(=O)c1cc2sc(-c3ccccc3OC)nc2[nH]1 ; COc1ccccc1-c1nc2[nH]c(C(=O)O)cc2s1]

To a solution of ethyl 2-(2-methoxyphenyl)-4H-pyrrolo[2,3-d]thiazole-5-carboxylate (250 mg, 826.86 umol, 1 eq) in MeOH (10 mL) was added LiOH (2 M in water, 4.13 mL, 10 eq). The mixture was stirred at 80° C. for 5 hr. LC-MS indicated desired mass was detected. The reaction mixture was concentrated under reduced pressure to remove solvent. The residue was diluted with 1M HCl (20 mL) and filtered to afford 2-(2-methoxyphenyl)-4H-pyrrolo[2,3-d]thiazole-5-carboxylic acid (160 mg, 554.15 umol, 67.02% yield, 95% purity) as a yellow solid. The product was used for the next step without further purification.

LCMS (ESI) m/z: 275.1 [M+H]⁺; ¹H NMR (500 MHz, DMSO-d6) δ=8.27 (dd, J=1.4, 7.9 Hz, 1H), 7.50-7.42 (m, 1H), 7.25 (d, J=8.2 Hz, 1H), 7.12 (t, J=7.5 Hz, 1H), 7.05 (s, 1H), 4.03 (s, 3H).

### Step 7. Synthesis of N-(1,1-dimethylsilinan-4-yl)-2-(2-methoxyphenyl)-4H-pyrrolo[2,3-d]thiazole-5-carboxamide

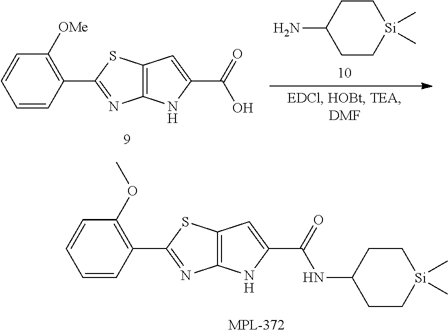

A mixture of 2-(2-methoxyphenyl)-4H-pyrrolo[2,3-d]thiazole-5-carboxylic acid (50 mg, 182.29 umol, 1 eq), 1,1-dimethylsilinan-4-amine (50 mg, 278.14 umol, 1.53 eq, HCl salt), HOBt (73.89 mg, 546.86 umol, 3 eq), EDCI (104.83 mg, 546.86 umol, 3 eq) and TEA (110.67 mg, 1.09 mmol, 152.23 uL, 6 eq) in DMF (5 mL) was degassed and purged with N₂ for 3 times, and then stirred at 25° C. for 16 hr under N₂ atmosphere. LC-MS indicated desired mass was detected. The reaction mixture was diluted with H₂O (20 mL) and extracted with EtOAc (20 mL×3). The combined organic layer was washed with saturated NaHCO₃ (30 mL×2) and 5% LiCl in water (30 mL×2), dried over Na₂SO₄, filtered and concentrated under reduced pressure to give a residue which was purified by flash silica gel chromatography (0-50% Ethyl acetate in petroleum ether). Compound N-(1,1-dimethylsilinan-4-yl)-2-(2-methoxyphenyl)-4H-pyrrolo[2,3-d]thiazole-5-carboxamide (30.1 mg, 74.88 umol, 41.08% yield, 99.41% purity) was obtained as a white solid.

LCMS (ESI) m/z: 400.1 [M+H]⁺; ¹H NMR (400 MHz, METHANOL-d₄) δ=8.27 (br d, J=7.8 Hz, 1H), 7.42-7.31 (m, 1H), 7.13 (br d, J=8.1 Hz, 1H), 7.07-6.96 (m, 2H), 4.00 (s, 3H), 3.72 (br t, J=11.4 Hz, 1H), 2.09 (br d, J=11.7 Hz, 2H), 1.68-1.52 (m, 2H), 0.85-0.75 (m, 2H), 0.73-0.59 (m, 2H), 0.08 (s, 3H), 0.00 (s, 3H).

### Example 31, MPL-373

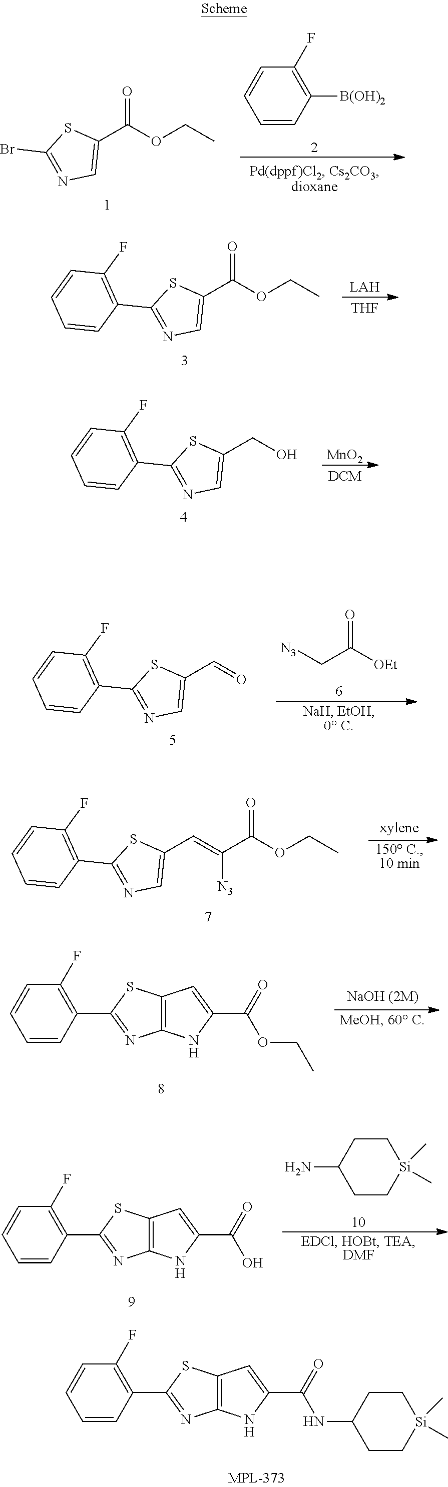

### Step 1. Synthesis of ethyl 2-(2-fluorophenyl)thiazole-5-carboxylate

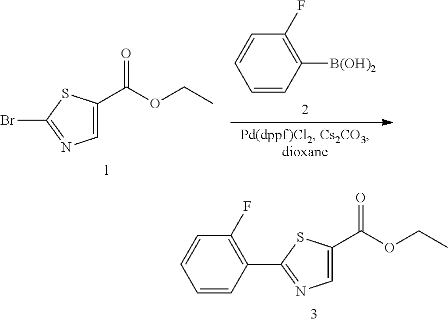

To a solution of ethyl 2-bromothiazole-5-carboxylate (2 g, 8.47 mmol, 1 eq) in dioxane (50 mL) was added (2-fluorophenyl)boronic acid (5.93 g, 42.36 mmol, 5 eq) and Cs₂CO₃ (4.14 g, 12.71 mmol, 1.5 eq). Then Pd(dppf)Cl₂.CH₂Cl₂ (69.18 mg, 84.71 umol, 0.01 eq) was added under N₂. The mixture was stirred at 110° C. for 12 hr. LCMS showed the starting material was consumed completely and desired mass was detected. The mixture was filtered, the filtrate was concentrated under reduced pressure to give a residue which was purified by flash silica gel chromatography (0-5% Ethyl acetate in petroleum ether). Compound ethyl 2-(2-fluorophenyl)thiazole-5-carboxylate (1.6 g, 6.37 mmol, 75.16% yield) was obtained as a colorless oil. ¹H NMR was recorded.

### Step 2. Synthesis of [2-(2-fluorophenyl)thiazol-5-yl]methanol

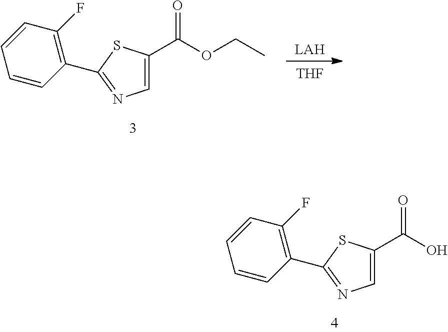 [SMILES: CCOC(=O)c1cnc(-c2ccccc2F)s1 ; O=C(O)c1cnc(-c2ccccc2F)s1 ; "THF"]

To a solution of ethyl 2-(2-fluorophenyl)thiazole-5-carboxylate (1.6 g, 6.37 mmol, 1 eq) in THF (30 mL) was added LAH (241.67 mg, 6.37 mmol, 1 eq) in batches at 0° C. The mixture was stirred at 0° C. for 1 hr. TLC (Petroleum ether: EtOAc=3:1) showed the starting material was consumed completely and one new spot formed. The reaction was quenched with water (0.25 mL), NaOH (15%, 0.25 mL) and water (0.75 mL) and filtered. The filter cake was washed with EtOAc (30 mL×5). The combined filtrate was dried over Na₂SO₄, and then filtered and concentrated under reduced pressure. Compound [2-(2-fluorophenyl)thiazol-5-yl]methanol (1.3 g, 5.59 mmol, 87.82% yield, 90% purity) was obtained as a white solid. ¹H NMR was recorded.

### Step 3. Synthesis of 2-(2-fluorophenyl)thiazole-5-carbaldehyde

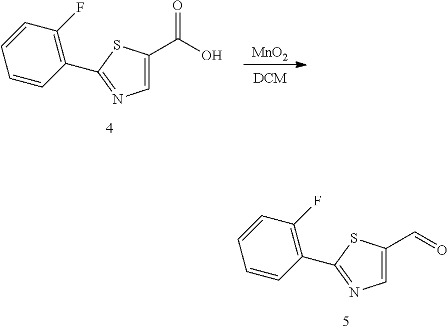

To a solution of [2-(2-fluorophenyl)thiazol-5-yl]methanol (1.3 g, 6.21 mmol, 1 eq) in DCM (20 mL) was added MnO₂ (10.80 g, 124.26 mmol, 20 eq). The mixture was stirred at 20° C. for 12 hr. TLC (Petroleum ether: EtOAc=10:1) showed the starting material was consumed completely and one new spot formed. The mixture was filtered. The cake was washed with EtOAc (10 mL×5). The combined filtrate was concentrated under reduced pressure to give a residue which was purified by flash silica gel chromatography (0-30% Ethyl acetate in petroleum ether). Compound 2-(2-fluorophenyl)thiazole-5-carbaldehyde (1.2 g, 5.21 mmol, 83.89% yield, 90% purity) was obtained as a white solid. ¹H NMR was recorded.

### Step 4. Synthesis of ethyl (Z)-2-azido-3-[2-(2-fluorophenyl)thiazol-5-yl]prop-2-enoate

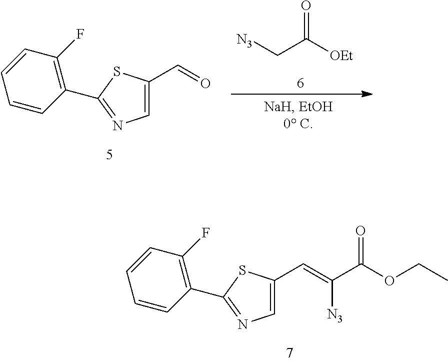 [SMILES: O=Cc1cnc(-c2ccccc2F)s1 ; CCOC(=O)CN=[N+]=[N-] ; CCOC(=O)/C(=C/c1cnc(-c2ccccc2F)s1)N=[N+]=[N-]]

NaH (1.16 g, 28.95 mmol, 60% purity, 5 eq) was added to EtOH (20 mL) in batches. The mixture was stirred at 30° C. until a clear solution formed, and then cooled to −10° C. Then a solution of 2-(2-fluorophenyl)thiazole-5-carbaldehyde (1.2 g, 5.79 mmol, 1 eq) and ethyl 2-azidoacetate (3.74 g, 28.95 mmol, 4.06 mL, 5 eq) in EtOH (2 mL) was added to the mixture dropwise. The mixture was stirred at −10° C.˜0° C. for 2 hr. TLC (Petroleum ether: EtOAc=5:1) indicated reactant 5 was consumed completely, and one major new spot with lower polarity formed. The reaction mixture was poured into HCl (0.2 M in water, 80 mL), and then extracted with EtOAc (30 mL). The organic layer was washed with brine (50 mL), dried over Na₂SO₄, and then filtered and concentrated. The residue was purified by flash silica gel chromatography (0-21% Ethyl acetate in petroleum ether). Compound ethyl (Z)-2-azido-3-[2-(2-fluorophenyl)thiazol-5-yl]prop-2-enoate (700 mg, 2.09 mmol, 36.08% yield, 95% purity) was obtained as a yellow solid. ¹H NMR was recorded.

### Step 5. Synthesis of ethyl 2-(2-fluorophenyl)-4H-pyrrolo[2,3-d]thiazole-5-carboxylate

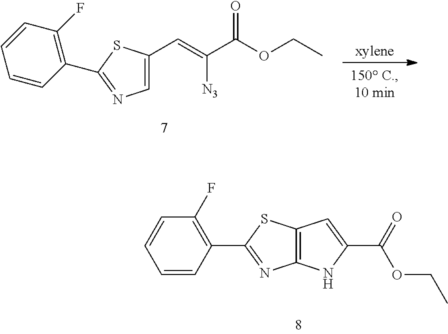 [SMILES: CCOC(=O)/C(=C/c1cnc(-c2ccccc2F)s1)N=[N+]=[N-] ; CCOC(=O)c1cc2sc(-c3ccccc3F)nc2[nH]1]

A solution of ethyl (Z)-2-azido-3-[2-(2-fluorophenyl)thiazol-5-yl]prop-2-enoate (700 mg, 2.20 mmol, 1 eq) in xylene (7 mL) was stirred and refluxed at 150° C. for 0.2 hr. TLC (Petroleum ether: EtOAc=5:1) indicated reactant 7 was consumed completely, and one major new spot with lower polarity formed. The mixture was cooled to 10° C. gradually. The product was crystallized from reaction solution after 12 hr and was collected by filtration. Compound ethyl 2-(2-fluorophenyl)-4H-pyrrolo[2,3-d]thiazole-5-carboxylate (500 mg, 1.64 mmol, 74.41% yield, 95% purity) was obtained as a yellow solid. ¹H NMR was recorded.

### Step 6. Synthesis of 2-(2-fluorophenyl)-4H-pyrrolo[2,3-d]thiazole-5-carboxylic acid

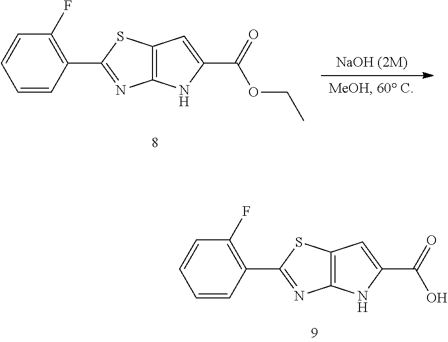 [SMILES: CCOC(=O)c1cc2sc(-c3ccccc3F)nc2[nH]1 ; O=C(O)c1cc2sc(-c3ccccc3F)nc2[nH]1]

To a solution of ethyl 2-(2-fluorophenyl)-4H-pyrrolo[2,3-d]thiazole-5-carboxylate (500 mg, 1.72 mmol, 1 eq) in MeOH (5 mL) was added NaOH (4 M, 5 mL, 11.61 eq). The mixture was stirred at 20° C. for 60 hr. TLC (Petroleum ether: EtOAc=3:1) showed starting material remained and one new spot formed. The mixture was stirred at 60° C. for additional 12 hr. TLC (Petroleum ether: EtOAc=3:1) showed the reaction completed. The mixture was concentrated under reduced pressure to remove MeOH, and then acidified with HCl (3N, in water) to pH 3 and filtered. The solid was collected and washed by water (5 mL×2) and petroleum ether (5 mL×2) and dried by lyophilization. Compound 2-(2-fluorophenyl)-4H-pyrrolo[2,3-d]thiazole-5-carboxylic acid (450 mg, 1.63 mmol, 94.65% yield, 95% purity) was obtained as a gray solid.

¹H NMR (500 MHz, DMSO-d₆) δ=12.90-11.97 (m, 1H), 8.24-8.18 (m, 1H), 7.55-7.48 (m, 1H), 7.45-7.35 (m, 1H), 7.45-7.35 (m, 2H), 6.99 (s, 1H).

### Step 7. Synthesis of N-(1,1-dimethylsilinan-4-yl)-2-(2-fluorophenyl)-4H-pyrrolo[2,3-d]thiazole-5-carboxamide

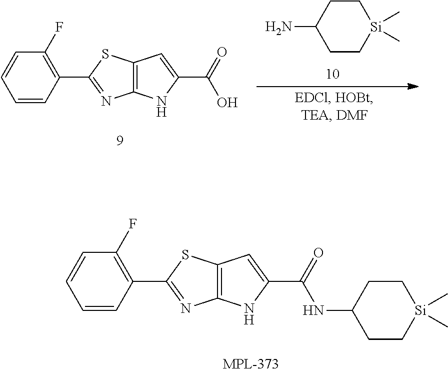

To a solution of 2-(2-fluorophenyl)-4H-pyrrolo[2,3-d]thiazole-5-carboxylic acid (50 mg, 190.65 umol, 1 eq) and 1,1-dimethylsilinan-4-amine (41.13 mg, 228.78 umol, 1.2 eq, HCl salt) in DMF (1 mL) was added a solution of EDCI (73.10 mg, 381.30 umol, 2 eq) and HOBt (51.52 mg, 381.30 umol, 2 eq) in DMF (1 mL), followed by TEA (77.17 mg, 762.60 umol, 106.14 uL, 4 eq). The mixture was stirred at 20° C. for 2 hr. LC-MS showed reactant 9 was consumed completely and one main peak with desired mass was detected. The mixture was filtered to remove insoluble matter and purified by prep-HPLC (column: YMC-Actus Triart C18 150*30 mm*5 um; mobile phase: A: 0.225% formic acid in water, B: CH₃CN; gradient:64%-94% B over 11 min).

Compound N-(1,1-dimethylsilinan-4-yl)-2-(2-fluorophenyl)-4H-pyrrolo [2,3 -d]thiazole-5-carboxamide (47.6 mg, 119.85 umol, 62.86% yield, 97.58% purity) was obtained as a white solid.

LCMS (ESI) m/z 388.0 [M+H]⁺; ¹H NMR (500 MHz, DMSO-d₆) δ=12.40 (br s, 1H), 8.12 (dt, J=1.7, 7.9 Hz, 1H), 7.96 (d, J=8.1 Hz, 1H), 7.46-7.40 (m, 1H), 7.36-7.26 (m, 2H), 7.11-7.04 (m, 1H), 3.68-3.55 (m, 1H), 1.98-1.84 (m, 2H), 1.56-1.42 (m, 2H), 0.69 (br d, J=14.6 Hz, 2H), 0.52 (dt, J=4.8, 14.1 Hz, 2H), 0.03-0.09 (m, 6H).

### Example 32, MPL-393

### Synthesis of 2-methoxy-N-(5-silaspiro[4.5]decan-8-yl)-4H-pyrrolo[2,3-d]thiazole-5-carboxamide

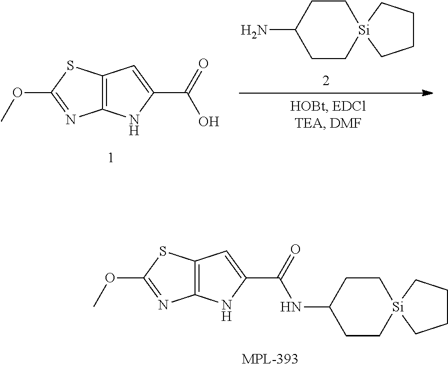 [SMILES: NC1CC[Si]2(CCCC2)CC1 ; COc1nc2[nH]c(C(=O)O)cc2s1 ; COc1nc2[nH]c(C(=O)NC3CC[Si]4(CCCC4)CC3)cc2s1]

To a solution of 2-methoxy-4H-pyrrolo[2,3-d]thiazole-5-carboxylic acid (40 mg, 201.82 umol, 1 eq) and 5-silaspiro[4.5]decan-8-amine (45.69 mg, 222.00 umol, 1.1 eq, HCl salt) in DMF (1 mL) was added a solution of HOBt (81.81 mg, 605.45 umol, 3 eq) and EDCI (116.07 mg, 605.45 umol, 3 eq) in DMF (2 mL) , followed by TEA (102.11 mg, 1.01 mmol, 140.45 uL, 5 eq). The reaction mixture was stirred at 25° C. for 12 hr. LC-MS showed desired compound was detected. The mixture was purified by prep-HPLC (column: Phenomenex Synergi C18 150*30 mm*4 um; mobile phase: A: 0.225% formic acid in water, B: CH₃CN; gradient: 59%-89% B over 11 min). Compound 2-methoxy-N-(5-silaspiro[4.5]decan-8-yl)-4H-pyrrolo[2,3-d]thiazole-5-carboxamide (18.8 mg, 51.80 umol, 25.67% yield, 96.3% purity) was obtained as a brown solid.

LCMS (ESI) m/z 350.1 [M+H]⁺; ¹H NMR (500 MHz, DMSO-d₆) δ=12.08 (br s, 1H), 7.76 (d, J=8.2 Hz, 1H), 6.97 (s, 1H), 4.14-3.99 (m, 3H), 3.79-3.60 (m, 1H), 2.10-1.93 (m, 2H), 1.64-1.47 (m, 6H), 0.86-0.44 (m, 8H).

### Example 33, MPL-394

### Synthesis of 2-methoxy-N-(6-silaspiro[5.5]undecan-3-yl)-4H-pyrrolo[2,3-d]thiazole-5-carboxamide

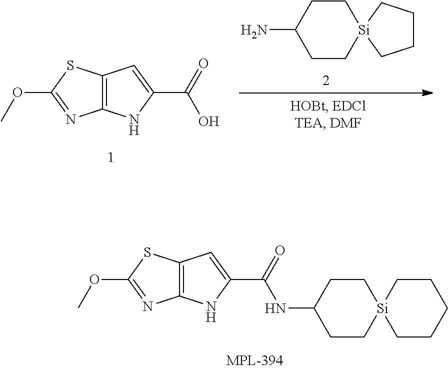 [SMILES: NC1CC[Si]2(CCCC2)CC1 ; COc1nc2[nH]c(C(=O)O)cc2s1 ; COc1nc2[nH]c(C(=O)NC3CC[Si]4(CCCCC4)CC3)cc2s1]

To a solution of 2-methoxy-4H-pyrrolo[2,3-d]thiazole-5-carboxylic acid (45 mg, 227.04 umol, 1 eq) and 6-silaspiro[5.5]undecan-3-amine (54.90 mg, 249.75 umol, 1.1 eq, HCl salt) in DMF (1 mL) was added a solution of HOBt (92.04 mg, 681.13 umol, 3 eq) and EDCI (130.57 mg, 681.13 umol, 3 eq) in DMF (1 mL), followed by TEA (114.87 mg, 1.14 mmol, 158.01 uL, 5 eq). The mixture was stirred at 25° C. for 12 hr. LC-MS showed desired compound was detected. The mixture was purified by prep-HPLC (column: YMC-Actus Triart C18 150*30 mm*5 um; mobile phase: A: 0.225% formic acid in water, B: CH₃CN; gradient: 65%-95% B over 11 min). Compound 2-methoxy-N-(6-silaspiro[5.5]undecan-3-yl)-4H-pyrrolo[2,3-d]thiazole-5-carboxamide (7.2 mg, 19.80 umol, 8.72% yield, 100% purity) was obtained as a yellow solid.

LCMS (ESI) m/z 364.0 [M+H]⁺; ¹H NMR (500 MHz, DMSO-d₆) δ=12.08 (s, 1H), 7.75 (d, J=8.2 Hz, 1H), 6.95 (s, 1H), 4.06 (s, 3H), 3.73-3.59 (m, 1H), 2.04-1.88 (m, 2H), 1.73-1.30 (m, 8H), 0.96-0.47 (m, 8H).

### Example 34, MPL-395, MPL-395A and MPL-395B

### Synthesis of N-(1,1-dimethylsilepan-4-yl)-2-methoxy-4H-pyrrolo[2,3-d]thiazole-5-carboxamide, (R)-N-(1,1-dimethylsilepan-4-yl)-2-methoxy-4H-pyrrolo[2,3-d]thiazole-5-carboxamide and (S)-N-(1,1-dimethylsilepan-4-yl)-2-methoxy-4H-pyrrolo[2,3-d]thiazole-5-carboxamide

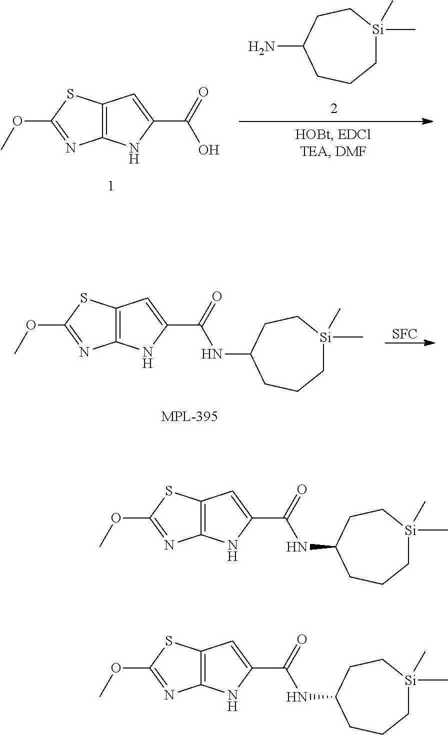

To a solution of 2-methoxy-4H-pyrrolo[2,3-d]thiazole-5-carboxylic acid (40 mg, 201.82 umol, 1 eq) and 1,1-dimethylsilepan-4-amine (46.93 mg, 242.18 umol, 1.2 eq, HCl salt) in DMF (1 mL) was added a solution of HOBt (81.81 mg, 605.45 umol, 3 eq) and EDCI (116.07 mg, 605.45 umol, 3 eq) in DMF (2 mL), followed by TEA (102.11 mg, 1.01 mmol, 140.45 uL, 5 eq). The reaction mixture was stirred at 25° C. for 12 hr. LCMS showed desired compound was detected. The mixture was purified by prep-HPLC (column: Phenomenex Synergi C18 150*30 mm*4 um; mobile phase: A: 0.225% formic acid in water, B: CH₃CN; gradient: 57%-87% B over 11 min). Compound N-(1,1-dimethylsilepan-4-yl)-2-methoxy-4H-pyrrolo[2,3-d]thiazole-5-carboxamide (17.6 mg, 52.15 umol, 25.84% yield, 100% purity) was obtained as a yellow solid.

LCMS (ESI) m/z 338.1 [M+H]⁺; ¹H NMR (500 MHz, DMSO-d₆) δ=12.07 (s, 1H), 7.78 (d, J=8.1 Hz, 1H), 6.97 (d, J=1.5 Hz, 1H), 4.07 (s, 3H), 3.83 (br d, J=8.4 Hz, 1H), 1.98-1.36 (m, 6H), 0.81-0.50 (m, 4H), 0.03 (d, J=7.6 Hz, 6H).

Racemic MPL-395 was also prepared at 1.01 mmol scale. The product isolated from prep-HPLC was separated by SFC (waters SFC Prep 80; column: DAICEL CHIRALCEL OD(250 mm*30 mm,10 um); mobile phase: A: 0.1%NH₃H₂O in EtOH, B: CO₂; 25%B isocratic; flow rate: 70 mL/min) to afford two peaks (two enantiomers), (R)-N-(1,1-dimethylsilepan-4-yl)-2-methoxy-4H-pyrrolo[2,3-d]thiazole-5-carboxamide and (S)-N-(1,1-dimethylsilepan-4-yl)-2-methoxy-4H-pyrrolo[2,3-d]thiazole-5-carboxamide.

Peak 1 (MPL-395A): 49.8 mg, 145.78 umol, 16.4% yield, 98.8% purity, yellow solid.

LCMS (ESI) m/z 338.3 [M+H]⁺; ¹H NMR (400 MHz, DMSO-d6) δ=12.03 (s, 1H), 7.77-7.72 (m, 1H), 6.96-6.91 (m, 1H), 4.03 (s, 3H), 3.83-3.75 (m, 1H), 1.89-1.74 (m, 3H), 1.64-1.55 (m, 1H), 1.47-1.37 (m, 2H), 0.77-0.53 (m, 4H), 0.02-−0.04 (m, 6H).

Peak2 (MPL-395B): 87.3 mg, 258.66 umol, 29.1% yield, 100% purity, yellow solid.

LCMS (ESI) m/z 338.3 [M+H]⁺; ¹H NMR (400 MHz, DMSO-d6) δ=12.03 (s, 1H), 7.75 (d, J=8.0 Hz, 1H), 6.94 (d, J=1.9 Hz, 1H), 4.06-4.01 (m, 3H), 3.84-3.77 (m, 1H), 1.90-1.74 (m, 3H), 1.67-1.57 (m, 1H), 1.47-1.37 (m, 2H), 0.77-0.52 (m, 4H), −0.01 (d, J=6.1 Hz, 6H).

MPL-395A and MPL-395B were also analyzed by analytical SFC.

Conditions:

Instrument: Waters UPCC with PDA Detector and QDa Detector

Column: Chiral MD-3 100*4.6 mm, 3 um particle size

Mobile phase: A: CO₂, B: 0.05% DEA in ethanol

Gradient: 5% to 40%B in 4.5 min and hold 40%B for 0.5 min, then 5%B for 1 min

Flow rate: 2.8 mL/min

Column temp.: 35° C.

ABPR: 1500 psi

MPL-395A: retention time 3.29 min, 99.64% ee; MPL-395B: 3.42 min; 98.94% ee

### Example 35, MPL-396

### Synthesis of N-(1,1-dimethylsilocan-5-yl)-2-methoxy-4H-pyrrolo[2,3-d]thiazole-5-carboxamide

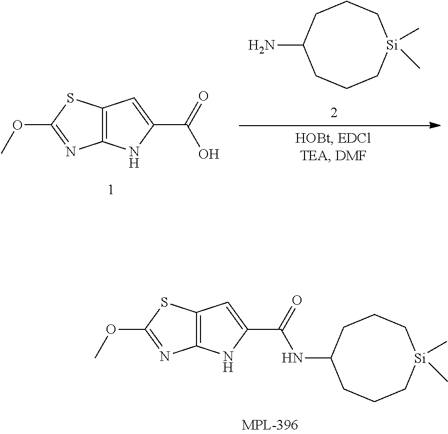

To a solution of 2-methoxy-4H-pyrrolo[2,3-d]thiazole-5-carboxylic acid (30 mg, 151.36 umol, 1 eq) and1,1-dimethylsilocan-5-amine (28.53 mg, 166.50 umol, 1.1 eq, HCl) in DMF (1 mL) was added a solution of HOBt (61.36 mg, 454.09 umol, 3 eq) and EDCI (87.05 mg, 454.09 umol, 3 eq) in DMF (1 mL), followed by TEA (76.58 mg, 756.82 umol, 105.34 uL, 5 eq). The mixture was stirred at 25° C. for 2 hr. LC-MS showed desired compound was detected. The mixture was purified by prep-HPLC (column: YMC-Actus Triart C18 150*30 mm*5 um; mobile phase: A: 0.225% formic acid in water, B: CH₃CN, gradient: 60%-90% B over 11 min). The product from prep-HPLC was further purified by prep-SFC (Berger MG II; column: Phenomenex-Cellulose-2 (250 mm*30 mm, 10 um); mobile phase: 0.1%NH₃H₂O in EtOH; B: CO₂; gradient: 40%B, isocratic, flow rate: 60 mL/min). Compound N-(1,1-dimethylsilocan-5-yl)-2-methoxy-4H-pyrrolo[2,3-d]thiazole-5-carboxamide (4 mg, 10.58 umol, 6.99% yield, 93% purity) was obtained as a white solid.

LCMS (ESI) m/z 352.2 [M+H]⁺; ¹H NMR (500 MHz, DMSO-d₆) δ=12.06 (s, 1H), 7.84 (d, J=8.1 Hz, 1H), 6.97 (d, J=1.8 Hz, 1H), 4.07 (s, 3H), 4.04-3.93 (m, 1H), 1.77-1.45 (m, 8H), 0.88-0.53 (m, 4H), 0.10-0.04 (m, 6H).

### Example 36, MPL-403

### Synthesis of N-(1,1-dimethylsilolan-3-yl)-2-methoxy-4H-pyrrolo[2,3-d]thiazole-5-carboxamide

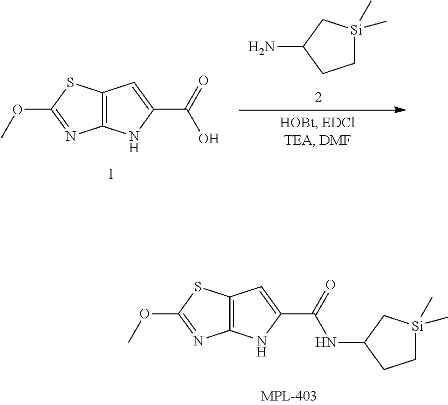

To a solution of 2-methoxy-4H-pyrrolo[2,3-d]thiazole-5-carboxylic acid (40 mg, 201.82 umol, 1 eq) and 1,1-dimethylsilolan-3-amine (36.79 mg, 222.00 umol, 1.1 eq, HCl) in DMF (1 mL) was added a solution of HOBt (81.81 mg, 605.45 umol, 3 eq) and EDCI (116.07 mg, 605.45 umol, 3 eq) in DMF (1 mL), followed by TEA (102.11 mg, 1.01 mmol, 140.45 uL, 5 eq). The mixture was stirred at 25° C. for 12 hr. LC-MS showed desired compound was detected. The mixture was purified by prep-HPLC (column: YMC-Actus Triart C18 150*30 mm*5 um; mobile phase: A: 0.225% formic acid in water, B: CH₃CN; gradient:57%-87% B over 11 min). Compound N-(1,1-dimethylsilolan-3-yl)-2-methoxy-4H-pyrrolo[2,3-d]thiazole-5-carboxamide (17.6 mg, 56.30 umol, 27.90% yield, 99% purity) was obtained as a yellow solid.

LCMS (ESI) m/z 309.9 [M+H]⁺; ¹H NMR (500 MHz, DMSO-d₆) δ=12.07 (s, 1H), 7.80 (d, J=7.6 Hz, 1H), 6.96 (d, J=1.8 Hz, 1H), 4.07 (s, 3H), 4.03-3.87 (m, 1H), 2.00 (br s, 1H), 1.40 (dq, J=6.9, 12.1 Hz, 1H), 1.06 (dd, J=4.9, 14.2 Hz, 1H), 0.79 (br dd, J=5.4, 14.7 Hz, 1H), 0.66-0.44 (m, 2H), 0.16 (d, J=1.2 Hz, 6H).

### Example 37, MPL-404

### Synthesis of N-(1,1-dimethylsilolan-3-yl)-2-phenyl-4H-pyrrolo[2,3-d]thiazole-5-carboxamide

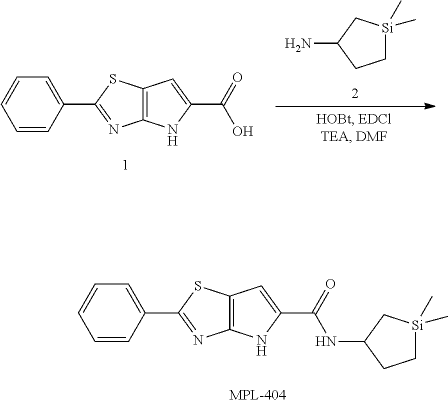

To a solution of 2-phenyl-4H-pyrrolo[2,3-d]thiazole-5-carboxylic acid (29.75 mg, 121.81 umol, 1 eq) and 1,1-dimethylsilolan-3-amine (22.21 mg, 133.99 umol, 1.1 eq, HCl salt) in DMF (1 mL) was added a solution of EDCI (46.70 mg, 243.62 umol, 2 eq) and HOBt (32.92 mg, 243.62 umol, 2 eq), followed by TEA (49.30 mg, 487.24 umol, 67.82 uL, 4 eq). The mixture was stirred at 20° C. for 2 hr. LC-MS showed reactant 1 was consumed completely and one main peak with desired mass was detected. The mixture was diluted with MeOH (2 mL) and filtered to remove insoluble matter. The filtrate was purified by prep-HPLC (column: YMC-Actus Triart C18 150*30 mm*5 um; mobile phase: A: 0.225% formic acid in water, B: CH₃CN; gradient: 61%-91% B over 11 min). Compound N-(1,1-dimethylsilolan-3-yl)-2-phenyl-4H-pyrrolo[2,3-d]thiazole-5-carboxamide (27 mg, 74.31 umol, 61.00% yield, 97.843% purity) was obtained as a white solid.

LCMS (ESI) m/z 356.1 [M+H]⁺; ¹H NMR (400 MHz, DMSO-d₆) δ=12.24 (s, 1H), 7.87 (d, J=7.6 Hz, 1H), 7.78-7.72 (m, 2H), 7.38-7.26 (m, 3H), 6.97 (d, J=2.0 Hz, 1H), 3.84 (br dd, J=6.7, 11.9 Hz, 1H), 1.93-1.80 (m, 1H), 1.26 (dq, J=7.0, 12.0 Hz, 1H), 0.93 (br dd, J=5.1, 14.2 Hz, 1H), 0.69-0.58 (m, 1H), 0.47 (dd, J=11.2, 13.9 Hz, 1H), 0.41-0.29 (m, 1H), 0.00 (d, J=1.7 Hz, 6H).

### Example 38, MPL-426

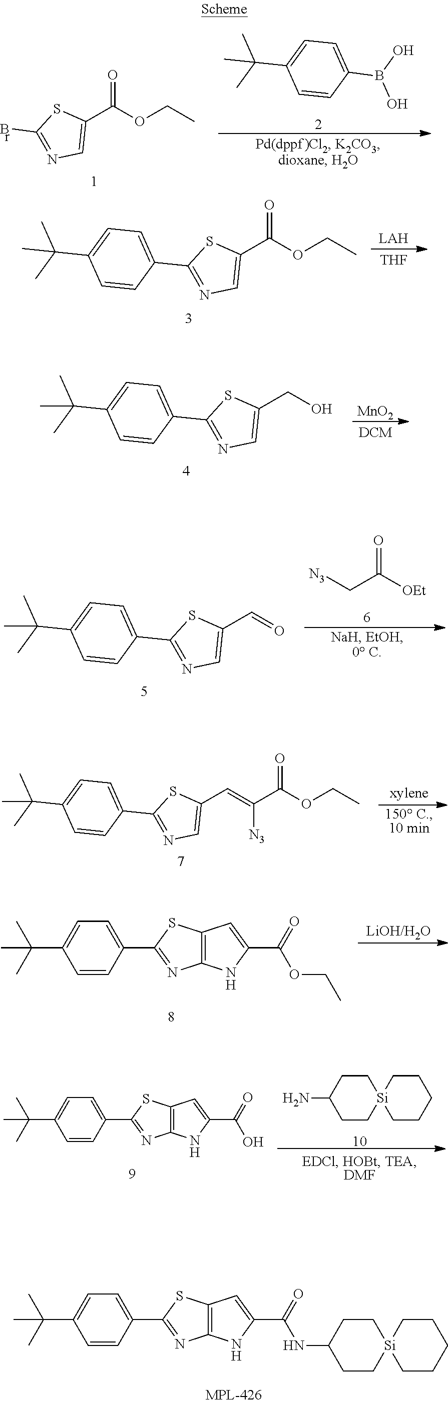

### Step 1. Synthesis of ethyl 2-(4-tert-butylphenyl)thiazole-5-carboxylate

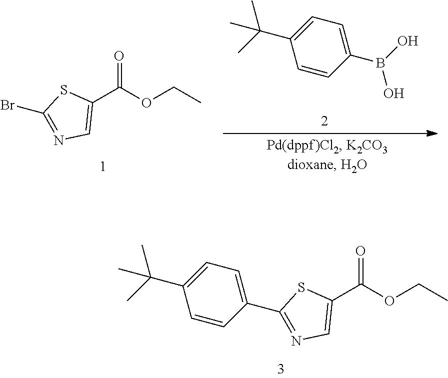 [SMILES: CCOC(=O)c1cnc(Br)s1 ; CC(C)(C)c1ccc(B(O)O)cc1 ; CCOC(=O)c1cnc(-c2ccc(C(C)(C)C)cc2)s1]

To a mixture of ethyl 2-bromothiazole-5-carboxylate (200 mg, 847.15 umol, 1 eq), (4-tert-butylphenyl) boronic acid (452.47 mg, 2.54 mmol, 3 eq) and K₂CO₃ (351.24 mg, 2.54 mmol, 3 eq) in dioxane (3 mL) was added H₂O (30 uL). The mixture was purged with N₂, then Pd(dppf)Cl₂ (61.99 mg, 84.71 umol, 0.1 eq) was added. The mixture was stirred at 110° C. for 12 hr under N₂. TLC showed that reactant 1 was consumed and new spot formed. The mixture was filtered. The cake was washed with EtOAc (50 mL×3). The combined filtrate was dried over Na₂SO₄. The solvent was removed in vacuo. The residue was purified by column chromatography (SiO₂, 0-20% Ethyl acetate in petroleum ether). Compound ethyl 2-(4-tert-butylphenyl)thiazole-5-carboxylate (220 mg, 681.27 umol, 80.42% yield, 89.6% purity) was obtained as a yellow oil. ¹H NMR was recorded.

### Step 2. Synthesis of [2-(4-tert-butylphenyl)thiazol-5-yl]methanol

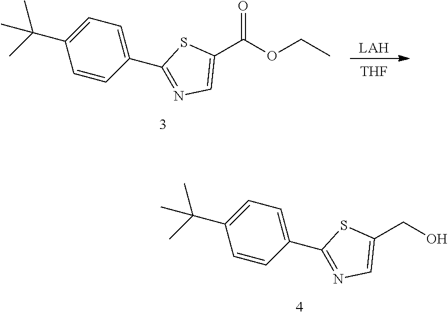

To the mixture of ethyl 2-(4-tert-butylphenyl)thiazole-5-carboxylate (1.7 g, 5.87 mmol, 1 eq) in THF (20 mL) was added LAH (668.87 mg, 17.62 mmol, 3 eq) at 0° C. The mixture was stirred at 0° C. for 20 min. TLC showed that desired compound was detected. The reaction was quenched by addition of H₂O (0.085 mL), NaOH (15% in water, 0.0855 mL) and H₂O (0.255 mL), and filtered. The filter cake was washed with EtOAC (5 mL). The combined filtrate was dried over by Na₂SO₄ and concentrated in vacuo. Compound [2-(4-tert-butylphenyl)thiazol-5-yl]methanol (1.4 g, crude) was obtained as a yellow oil. The crude product was used for the next step directly.

### Step 3. Synthesis of 2-(4-tert-butylphenyl)thiazole-5-carbaldehyde

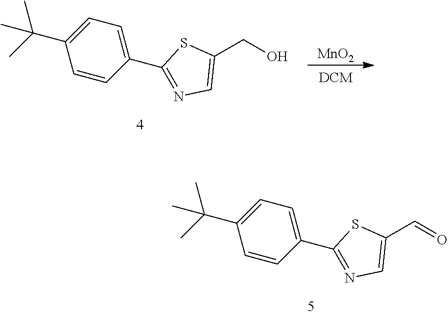

To a solution of [2-(4-tert-butylphenyl)thiazol-5-yl]methanol (1.4 g, 5.66 mmol, 1 eq) in DCM (50 mL) was added MnO₂ (9.84 g, 113.20 mmol, 20 eq). The mixture was stirred at 30° C. for 4 hr. TLC indicated that the reactant 4 was consumed and one new spot formed. The mixture was filtered. The filtrate was concentrated under reduce pressure. The residue was purified by column chromatography (SiO₂, 0-20% Ethyl acetate in petroleum ether). Compound 2-(4-tert-butylphenyl)thiazole-5-carbaldehyde (1 g, 3.26 mmol, 52.04% yield, 80% purity) was obtained as a yellow solid. ¹H NMR was recorded.

### Step 4. Synthesis of ethyl (Z)-2-azido-3-[2-(4-tert-butylphenyl)thiazol-5-yl]prop-2-enoate

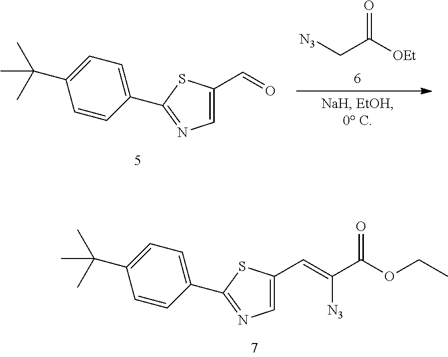 [SMILES: CC(C)(C)c1ccc(-c2ncc(C=O)s2)cc1 ; CCOC(=O)CN=[N+]=[N-] ; CCOC(=O)/C(=C/c1cnc(-c2ccc(C(C)(C)C)cc2)s1)N=[N+]=[N-]]

NaH (163.02 mg, 4.08 mmol, 60% purity, 5 eq) was added to EtOH (2 mL) in batches. The mixture was stirred at 30° C. until a clear solution formed, and then cooled to −10° C. Then a solution of 2-(4-tert-butylphenyl)thiazole-5-carbaldehyde (200 mg, 815.20 umol, 1 eq) and ethyl 2-azidoacetate (315.77 mg, 2.45 mmol, 343.22 uL, 3 eq) in EtOH (5 mL) was added to the mixture slowly. The mixture was stirred at −10° C.˜0° C. for 2 hr. The reaction mixture was poured into saturated NH₄Cl (20 mL), and then extracted with EtOAc (10 mL×2). The combined organic layer was washed with brine (30 mL×2), dried over Na₂SO₄, filtered and concentrated under reduced pressure to give a residue which was purified by column chromatography (SiO₂, 0-10% Ethyl acetate in petroleum ether). Compound ethyl (Z)-2-azido-3-[2-(4-tert-butylphenyl)thiazol-5-yl]prop-2-enoate (130 mg, 145.89 umol, 17.90% yield, 40% purity) was obtained as a yellow solid.

LCMS (ESI) m/z: 357.2 [M+H]⁺

### Step 5. Synthesis of ethyl 2-(4-tert-butylphenyl)-4H-pyrrolo[2,3-d]thiazole-5-carboxylate

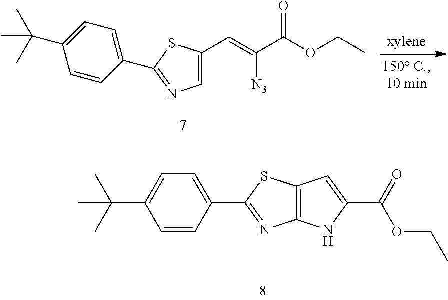

A solution of ethyl (Z)-2-azido-3-[2-(4-tert-butylphenyl)thiazol-5-yl]prop-2-enoate (130 mg, 364.72 umol, 1 eq) in xylene (5 mL) was stirred at 150° C. for 10 min. TLC showed one new spot formed. The mixture was concentrated under reduce pressure. The residue was purified by column chromatography (SiO₂, 0-30% Ethyl acetate in petroleum ether). Ethyl 2-(4-tert-butylphenyl)-4H-pyrrolo[2,3-d]thiazole-5-carboxylate (100 mg, crude) was obtained as a yellow solid.

### Step 6. Synthesis of 2-(4-tent-butylphenyl)-4H-pyrrolo[2,3-d]thiazole-5-carboxylic acid

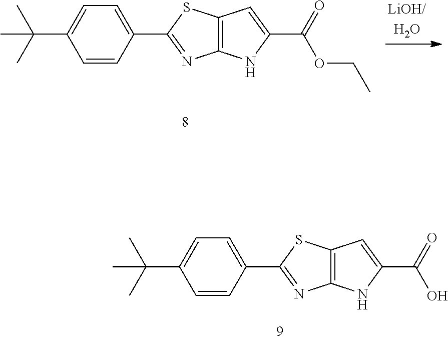

To a solution of ethyl 2-(4-tert-butylphenyl)-4H-pyrrolo[2,3-d]thiazole-5-carboxylate (550 mg, 1.67 mmol, 1 eq) in EtOH (10 mL) was added a solution of LiOH.H₂O (1.41 g, 33.49 mmol, 20 eq) in H₂O (5 mL). The mixture was stirred at 80° C. for 1 hr. TLC showed that reactant 8 was consumed and new spot formed. The mixture was concentrated under reduced pressure to remove EtOH, and then diluted with water (20 mL) and acidified to pH 4 with 1 N HCl in water. The acidic solution was extracted with EtOAc (30 mL×2). The combined organic layer was dried over Na₂SO₄, filtered and concentrated under reduced pressure to afford 2-(4-tert-butylphenyl)-4H-pyrrolo[2,3-d]thiazole-5-carboxylic acid (450 mg, 1.42 mmol, 84.99% yield, 95% purity) as a yellow solid.

¹H NMR (400 MHz, DMSO-d₆) δ=7.84 (d, J=8.6 Hz, 2H), 7.50 (d, J=8.6 Hz, 2H), 7.05 (d, J=1.7 Hz, 1H), 1.32-1.21 (m, 9H).

### Step 6. Synthesis of 2-(4-tert-butylphenyl)-N-(6-silaspiro[5.5]undecan-3-yl)-4H-pyrrolo[2,3-d]thiazole-5-carboxamide

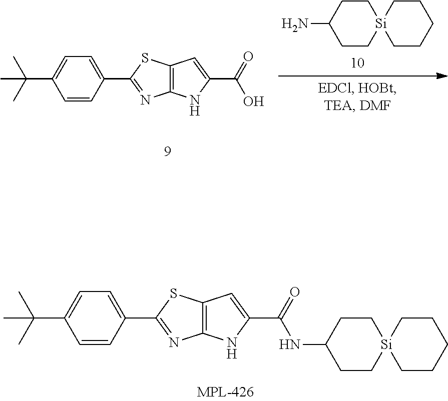 [SMILES: NC1CC[Si]2(CCCCC2)CC1 ; CC(C)(C)c1ccc(-c2nc3[nH]c(C(=O)O)cc3s2)cc1 ; CC(C)(C)c1ccc(-c2nc3[nH]c(C(=O)NC4CC[Si]5(CCCCC5)CC4)cc3s2)cc1]

To a solution of 2-(4-tert-butylphenyl)-4H-pyrrolo[2,3-d]thiazole-5-carboxylic acid (80 mg, 266.33 umol, 1 eq), 6-silaspiro[5.5]undecan-3-amine (58.55 mg, 266.33 umol, 1 eq, HCl salt) in DMF (0.5 mL) was added a solution of HOBt (107.96 mg, 799.00 umol, 3 eq) and EDCI (153.17 mg, 799.00 umol, 3 eq) in DMF (0.5 mL), followed by TEA (161.70 mg, 1.60 mmol, 222.42 uL, 6 eq). The mixture was stirred at 25° C. for 1 hr. LCMS showed that desired compound was detected. The reaction mixture was filtered and the filtrate was purified by prep-HPLC (column: YMC-Actus Triart C18 150*30 mm*5 um; mobile phase: A: 0.225% formic acid in water, B: CH₃CN; gradient: 90%-100% B over 11 min). Compound 2-(4-tert-butylphenyl)-N-(6-silaspiro[5.5]undecan-3-yl)-4H-pyrrolo[2,3-d]thiazole-5-carboxamide (14.1 mg, 29.13 umol, 10.94% yield, 96.215% purity) was obtained as a white solid.

LCMS (ESI) m/z: 466.3 [M+H]⁺; ¹H NMR (400 MHz, METHANOL-d₄) δ=7.90 (d, J=8.4 Hz, 2H), 7.52 (d, J=8.5 Hz, 2H), 7.07 (s, 1H), 3.78 (br t, J=11.4 Hz, 1H),2.14 (br d, J=10.8 Hz, 2H), 1.78-1.61 (m, 6H), 1.49-1.43 (m, 2H), 1.37 (s, 9H), 0.97 (br d, J=14.8 Hz, 2H), 0.80-0.76 (m, 2H), 0.72-0.63 (m, 4H).

### Example 39, MPL-427

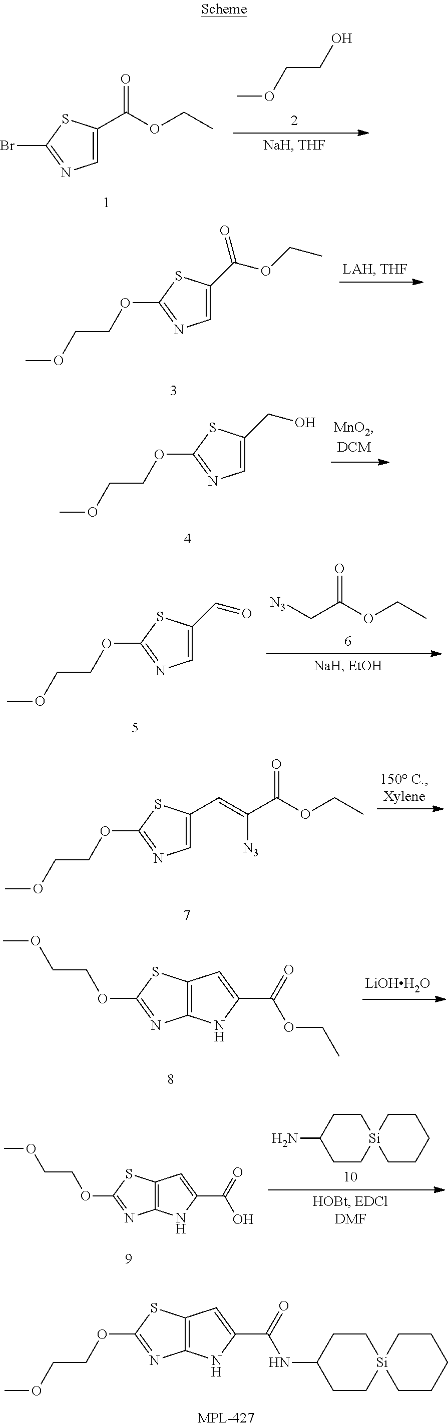

### Step 1. Synthesis of ethyl 2-(2-methoxyethoxy)thiazole-5-carboxylate

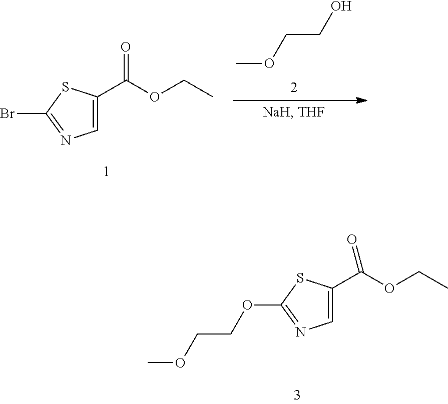

To a solution of 2-methoxyethanol (16.12 g, 211.79 mmol, 10 eq) in THF (60 mL) was added NaH (4.24 g, 105.89 mmol, 60% purity, 5 eq) in batches at 0° C. The mixture was stirred at this temperature for 1 hr, ethyl 2-bromothiazole-5-carboxylate (5 g, 21.18 mmol, 1 eq) was then added. The mixture was stirred at 20° C. for 1 hr. The reaction mixture was adjusted to pH 6 with aqueous HCl (1 N, 150 mL), and then extracted with EtOAc (60 mL×2). The combined organic layer was washed with brine (50 mL), dried over Na₂SO₄, and then ltered and concentrated under reduced pressure. Compound ethyl 2-(2-methoxyethoxy)thiazole-5-carboxylate (6.4 g, crude) was obtained as a yellow oil. The crude product was used for the next step without further purification.

LCMS (ESI) m/z: 232.2 [M+H]⁺

### Step 2. Synthesis of [2-(2-methoxyethoxy)thiazol-5-yl]methanol

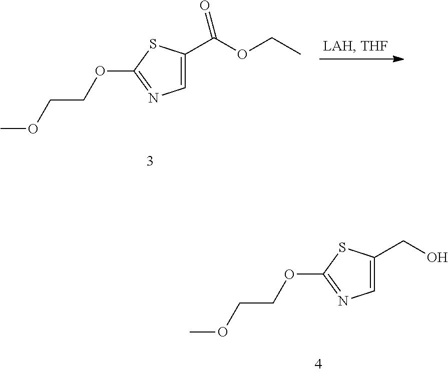

To an ice-cooled solution of ethyl 2-(2-methoxyethoxy) thiazole-5-carboxylate (1 g, 4.32 mmol, 1 eq) in dried THF (10 mL) was added LAH (246.17 mg, 6.49 mmol, 1.5 eq) in batches. The mixture was stirred at 0-20° C. for 1 hr. TLC (Petroleum ether: Ethyl acetate=5:1) indicated the starting material was consumed completely and one new spot formed. The reaction was quenched with water (0.246 mL), NaOH (15% in water, 0.246 mL), follow by water (0.738 mL). The mixture was filtered. The filter cake was washed with EtOAc (20 mL×2). The combined filtrate was concentrated under reduced pressure. Compound [2-(2-methoxyethoxy)thiazol-5-yl]methanol (686 mg, 2.74 mmol, 63.42% yield, 75.65% purity) was obtained as a yellow oil. The crude product was used for the next step without further purification.

LCMS m/z: 190.1 [M+1]⁺

### Step 3. Synthesis of 2-(2-methoxyethoxy)thiazole-5-carbaldehyde

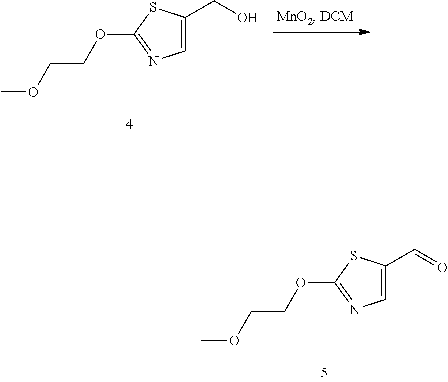

To a solution of [2-(2-methoxyethoxy)thiazol-5-yl]methanol (686 mg, 3.63 mmol, 1 eq) in DCM (6 mL) was added MnO₂ (3.15 g, 36.25 mmol, 10 eq). The mixture was stirred at 25° C. for 12 hr. TLC (Petroleum ether: Ethyl acetate=1:1) indicated the starting material was consumed and one new spot formed. The reaction mixture was filtered and concentrated under reduced pressure to give a residue which was purified by column chromatography (SiO₂, 0-14% Ethyl acetate in petroleum ether). Compound 2-(2-methoxyethoxy)thiazole-5-carbaldehyde (314 mg, 1.59 mmol, 43.95% yield, 95% purity) was obtained as a yellow solid. ¹H NMR was recorded.

### Step 4. Synthesis of ethyl (Z)-2-azido-3-[2-(2-methoxyethoxy)thiazol-5-yl]prop-2-enoate

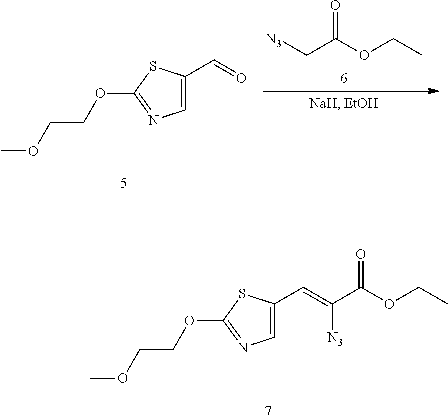

NaH (201.26 mg, 5.03 mmol, 60% purity, 3 eq) was added to EtOH (5 mL) in batches. The mixture was stirred at 20° C. until a clear solution formed, and then cooled to −10° C. A solution of 2-(2-methoxyethoxy)thiazole-5-carbaldehyde (314 mg, 1.68 mmol, 1 eq) and ethyl 2-azidoacetate (649.67 mg, 5.03 mmol, 706.16 uL, 3 eq) in THF (5 mL) was then added to the mixture dropwise. The mixture was stirred at −10° C.˜0° C. for 2 hr. LCMS showed desired mass. The reaction was quenched with saturated NH₄Cl (40 mL), and then extracted with EtOAc (20 mL×2). The combined organic layer was washed with brine (20 mL×2), dried over Na₂SO₄, filtered and concentrated under reduced pressure to give a residue which was purified by column chromatography (SiO₂, 0-20% Ethyl acetate in petroleum ether). Compound ethyl (Z)-2-azido-3-[2-(2-methoxyethoxy)thiazol-5-yl]prop-2-enoate (99 mg, 331.86 umol, 19.79% yield,) was obtained as a yellow solid.

LCMS (ESI) m/z: 299.1 [M+H]⁺

### Step 5. Synthesis of ethyl 2-(2-methoxyethoxy)-4H-pyrrolo[2,3-d] thiazole-5-carboxylate

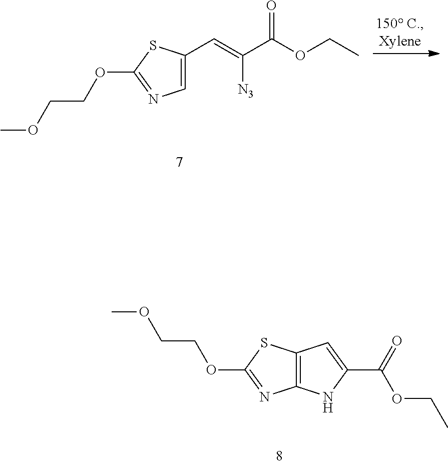

A solution of ethyl (Z)-2-azido-3-[2-(2-methoxyethoxy)thiazol-5-yl]prop-2-enoate (99 mg, 331.86 umol, 1 eq) in xylene (2 mL) was stirred at 150° C. for 30 min. LCMS showed desired mass was detected. The reaction mixture was concentrated under reduced pressure to remove solvent. The residue was purified by column chromatography (SiO₂, 0-22% Ethyl acetate in petroleum ether). Compound ethyl 2-(2-methoxyethoxy)-4H-pyrrolo[2,3-d] thiazole-5-carboxylate (91 mg, 319.82 umol, 96.37% yield, 95% purity) was obtained as a white solid.

LCMS (ESI) m/z: 271.1 [M+H]⁺; ¹H NMR was recorded.

### Step 6. Synthesis of 2-(2-methoxyethoxy)-4H-pyrrolo[2,3-d]thiazole-5-carboxylic acid

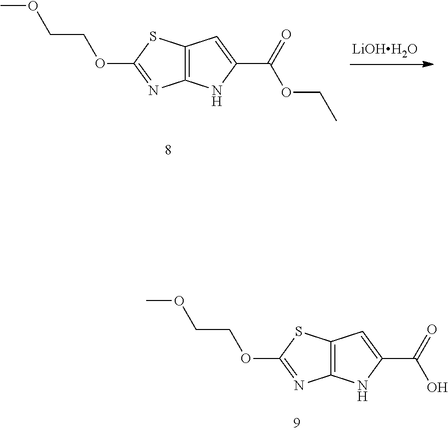

To a solution of ethyl 2-(2-methoxyethoxy)-4H-pyrrolo[2,3-d]thiazole-5-carboxylate (91 mg, 336.66 umol, 1 eq) in THF (2 mL) was added a solution of LiOH.H₂O (84.76 mg, 2.02 mmol, 6 eq) in H₂O (2 mL). The mixture was stirred at 80° C. for 6 hr. TLC (Petroleum ether: Ethyl acetate=3:1) indicated the reactant was consumed completely and one new spot formed. The reaction mixture was concentrated under reduced pressure to remove THF. The aqueous solution was acidified to pH 3-4 with HCl (1 N in water) and filtered. The cake was washed with petroleum ether and then dried under reduced pressure. Compound 2-(2-methoxyethoxy)-4H-pyrrolo[2,3-d] thiazole-5-carboxylic acid (41 mg, 160.78 umol, 47.76% yield, 95% purity) was obtained as a brown solid.

¹H NMR (400 MHz, DMSO-d₆) δ=12.53-12.18 (m, 2H), 6.93 (d, J=2.0 Hz, 1H), 4.61-4.51 (m, 2H), 3.72-3.66 (m, 2H), 3.30 (s, 3H).

### Step 7. Synthesis of 2-(2-methoxyethoxy)-N-(6-silaspiro[5.5]undecan-3-yl)-4H-pyrrolo[2,3-d] thiazole-5-carboxamide

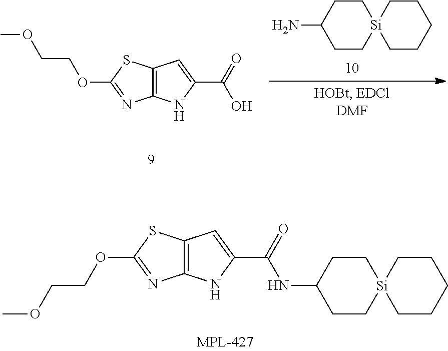 [SMILES: COCCOc1nc2[nH]c(C(=O)O)cc2s1 ; NC1CC[Si]2(CCCCC2)CC1 ; COCCOc1nc2[nH]c(C(=O)NC3CC[Si]4(CCCCC4)CC3)cc2s1]

To a solution of 2-(2-methoxyethoxy)-4H-pyrrolo[2,3-d]thiazole-5-carboxylic acid (41 mg, 169.25 umol, 1 eq) and 6-silaspiro[5.5]undecan-3-amine (44.65 mg, 203.09 umol, 1.2 eq, HCl salt) in DMF (2 mL) was added a solution of EDCI (97.33 mg, 507.74 umol, 3 eq) and HOBt (68.61 mg, 507.74 umol, 3 eq) in DMF (0.5 mL), followed by TEA (102.75 mg, 1.02 mmol, 141.34 uL, 6 eq). The mixture was stirred at 25° C. for 1 hr. LC-MS showed desired mass was detected. The reaction mixture was filtered to obtain filtrate, which was purified by prep-HPLC (column: YMC-Actus Triart C18 150*30 mm*5 um; mobile phase: A: 0.2% formic acid in water, B: CH₃CN; gradient: 61%-91% B over 11 min). Compound 2-(2-methoxyethoxy)-N-(6-silaspiro [5.5]undecan-3-yl)-4H-pyrrolo[2,3-d]thiazole-5-carboxamide (6.7 mg, 16.44 umol, 9.71% yield, 100% purity) was obtained as a yellow solid.

LCMS (ESI) m/z: 408.1 [M+H]⁺; ¹H NMR (400 MHz, DMSO-d₆) δ=12.08 (br s, 1H), 7.76 (br d, J=8.1 Hz, 1H), 6.96 (s, 1H), 4.59-4.47 (m, 2H), 3.74-3.58 (m, 3H), 3.29 (s, 3H), 2.02-1.91 (m, 2H), 1.72-1.44 (m, 6H), 1.38 (br s, 2H), 0.87 (br d, J=14.7 Hz, 2H), 0.75-0.64 (m, 2H), 0.63-0.48 (m, 4H).

### Example 40, MPL-429

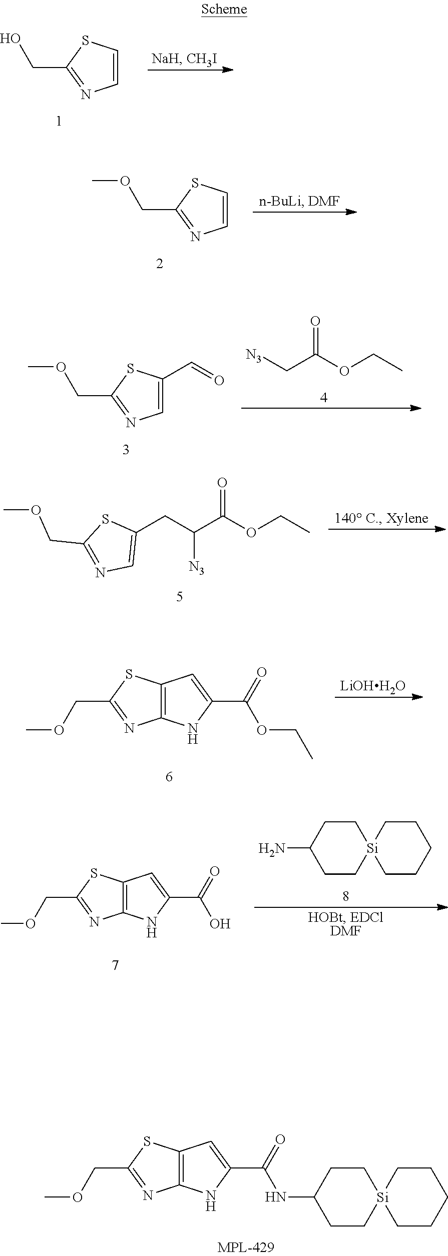

### Step 1. Synthesis of 2-(methoxymethyl) thiazole

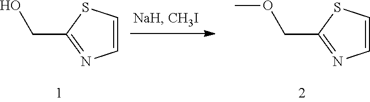

To a solution of thiazol-2-ylmethanol (4.5 g, 39.08 mmol, 1 eq) in THF (50 mL) was added NaH (2.03 g, 50.80 mmol, 60% purity, 1.3 eq) at 0° C. The mixture was stirred at 0° C. for 20 min. Then CH₃I (12.76 g, 89.88 mmol, 5.60 mL, 2.3 eq) (14.15 g) was added into the mixture at 0° C. The reaction mixture was stirred at 0° C. for 2 hr. TLC (Petroleum ether: Ethyl acetate=3:1) indicated the reactant was consumed completely and one new spot formed. The reaction mixture was quenched by addition of saturated NH₄Cl (100 mL) at 25° C., and then extracted with EtOAc (60 mL×2). The combined organic layer was washed with brine (60 mL), dried over Na₂SO₄, filtered and concentrated under reduced pressure to give a residue which was purified by column chromatography (SiO2, 0-4% Ethyl acetate in petroleum ether). Compound 2-(methoxymethyl) thiazole (2.8 g, 20.59 mmol, 52.69% yield, 95% purity) was obtained as a colorless oil. ¹H NMR was recorded.

### Step 2. Synthesis of 2-(methoxymethyl) thiazole-5-carbaldehyde

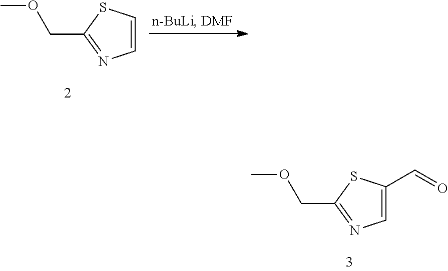

To a solution of 2-(methoxymethyl) thiazole (2.8 g, 21.68 mmol, 1 eq) in THF (10 mL) was added n-BuLi (2.5 M in n-hexane, 13.01 mL, 1.5 eq) dropwise at −78° C. under N₂. After stirring at −78° C. for 1 hr, DMF (3.17 g) was added dropwise at −78° C. The reaction mixture was stirred at −78° C. for another 2 hr. TLC (Petroleum ether: Ethyl acetate=3:1) indicated the reactant was consumed completely and many new spots formed. The reaction mixture was quenched with saturated NH₄Cl (100 mL) at 25° C., and then extracted with EtOAc (60 mL×2). The combined organic layer was washed with brine (80 mL), dried over Na₂SO₄, and then filtered and concentrated under reduced pressure to give a residue which was purified by column chromatography (SiO₂, 0-13% Ethyl acetate in petroleum ether). Compound 2-(methoxymethyl) thiazole-5-carbaldehyde (1.7 g, 10.27 mmol, 47.40% yield, 95% purity) was obtained as a yellow oil. ¹H NMR was recorded.

### Step 3. Synthesis of ethyl (Z)-2-azido-3-[2-(methoxymethyl) thiazol-5-yl] prop-2-enoate

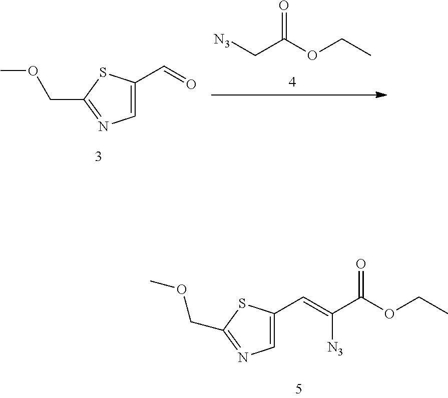

NaH (1.30 g, 32.44 mmol, 60% purity, 3 eq) was added to EtOH (10 mL) in batches. The mixture was stirred at 20° C. until a clear solution formed, and then cooled to −10° C. A solution of 2-(methoxymethyl) thiazole-5-carbaldehyde (1.7 g, 10.81 mmol, 1 eq) and ethyl 2-azidoacetate (4.19 g, 32.44 mmol, 4.55 mL, 3 eq) in THF (10 mL) was added to the mixture dropwise. The mixture was stirred at −10° C.˜0° C. for 2 hr. TLC (Petroleum ether: Ethyl acetate=1:1) indicated the reactant was consumed completely and new spots formed. The reaction was quenched with saturated NH₄Cl (50 mL), and then extracted with EtOAc (50 mL×2). The combined organic layer was washed with brine (60 mL×2), dried over Na₂SO₄, filtered and concentrated under reduced pressure to give a residue which was purified by column chromatography (SiO₂, 0-20% Ethyl acetate in petroleum ether). Compound ethyl (Z)-2-azido-3-[2-(methoxymethyl) thiazol-5-yl] prop-2-enoate (349 mg, 1.26 mmol, 11.69% yield, 97.185% purity) was obtained as a yellow solid.

LCMS (ESI) m/z: 269.2 [M+H]⁺

### Step 4. Synthesis of ethyl 2-(methoxymethyl)-4H-pyrrolo [2,3-d] thiazole-5-carboxylate

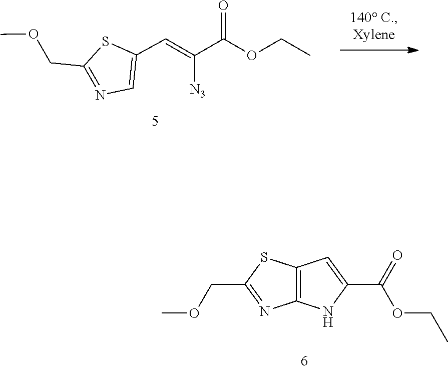

A solution of ethyl (Z)-2-azido-3-[2-(methoxymethyl) thiazol-5-yl]prop-2-enoate (349 mg, 1.30 mmol, 1 eq) in xylene (2 mL) was stirred at 140° C. for 30 min. LC-MS showed desired mass was detected. The reaction mixture was concentrated under reduced pressure to remove solvent. The residue was purified by column chromatography (SiO2, 0-33% ethyl acetate in petroleum ether). Compound ethyl 2-(methoxymethyl)-4H-pyrrolo [2,3-d] thiazole-5-carboxylate (262 mg, 1.04 mmol, 79.63% yield, 95% purity) was obtained as a yellow solid.

LCMS (ESI) m/z: 241.2 [M+H]⁺; ¹H NMR was recorded.

### Step 5. Synthesis of 2-(methoxymethyl)-4H-pyrrolo[2,3-d]thiazole-5-carboxylic acid

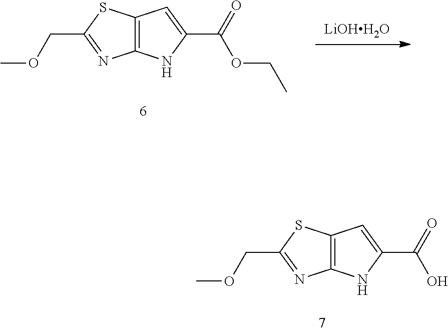

To a solution of ethyl 2-(methoxymethyl)-4H-pyrrolo[2,3-d]thiazole-5-carboxylate (262 mg, 547.49 umol, 50.210% purity, 1 eq) in THF (3 mL) was added a solution of LiOH.H₂O (137.85 mg, 3.28 mmol, 6 eq) in H₂O (3 mL). The mixture was stirred at 80° C. for 12 hr. LCMS showed desired mass was detected. The reaction mixture was concentrated under reduced pressure to remove THF. The aqueous solution was acidified to pH 3-4 with HCl (6 N in water) and filtered. The cake was washed with petroleum ether (30 mL) and dried under reduced pressure. Compound 2-(methoxymethyl)-4H-pyrrolo[2,3-d]thiazole-5-carboxylic acid (186 mg, crude) was obtained as a brown solid.

LCMS (ESI) m/z: 213.1 [M+H]⁺; ¹H NMR (400 MHz, DMSO-d₆) δ=12.91-12.47 (m, 2H), 7.05 (d, J=2.0 Hz, 1H), 4.74 (s, 2H), 3.40 (s, 3H).

### Step 6. Synthesis of 2-(methoxymethyl)-N-(6-silaspiro[5.5]undecan-3-yl)-4H-pyrrolo[2,3-d]thiazole-5-carboxamide

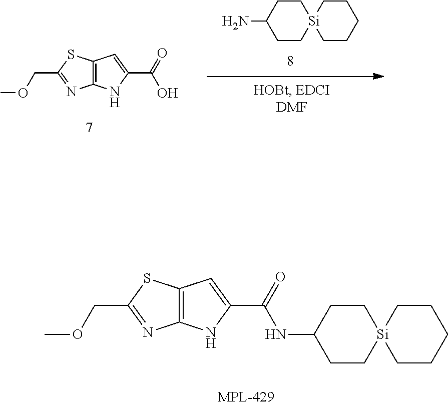 [SMILES: NC1CC[Si]2(CCCCC2)CC1 ; COCc1nc2[nH]c(C(=O)O)cc2s1 ; COCc1nc2[nH]c(C(=O)NC3CC[Si]4(CCCCC4)CC3)cc2s1]

To a solution of 2-(methoxymethyl)-4H-pyrrolo[2,3-d]thiazole-5-carboxylic acid (50 mg, 235.60 umol, 1 eq) and 6-silaspiro[5.5]undecan-3-amine (51.79 mg, 235.60 umol, 1 eq, HCl salt) in DMF (1.5 mL) was added a solution of EDCI (135.49 mg, 706.80 umol, 3 eq) and HOBt (95.50 mg, 706.80 umol, 3 eq) in DMF (1 mL), followed by TEA (119.20 mg, 1.18 mmol, 163.96 uL, 5 eq). The mixture was stirred at 25° C. for 1 hr. LCMS showed desired mass was detected. The reaction mixture was filtered to obtain filtrate, which was purified by prep-HPLC (column: Phenomenex Synergi C18 150*30 mm*4 um; mobile phase: A: 0.225% formic acid in water, B: CH₃CN; gradient: 62%-91% B over 11 min). Compound 2-(methoxymethyl)-N-(6-silaspiro[5.5] undecan-3-yl) -4H-pyrrolo [2,3-d] thiazole-5-carboxamide (12.9 mg, 34.17 umol, 14.50% yield, 100% purity) was obtained as a yellow solid.

LCMS (ESI) m/z: 378.2 [M+H]⁺; ¹H NMR (500 MHz, DMSO-d₆) δ=12.30 (br s, 1H), 7.96 (d, J=8.1 Hz, 1H), 7.08 (s, 1H), 4.82-4.62 (m, 2H), 3.74-3.65 (m, 1H), 3.38 (s, 3H), 2.03-1.94 (m, 2H), 1.70-1.49 (m, 6H), 1.38 (br s, 2H), 0.89 (br d, J=14.5 Hz, 2H), 0.72-0.65 (m, 2H), 0.63-0.52 (m, 4H).

### Example 41, MPL-431

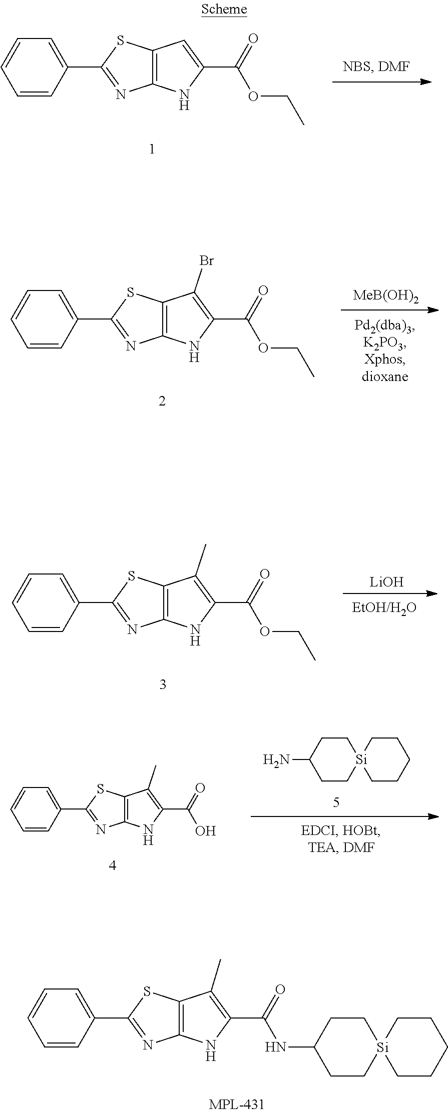

### Step 1. Synthesis of ethyl 6-bromo-2-phenyl-4H-pyrrolo[2,3-d]thiazole-5-carboxylate

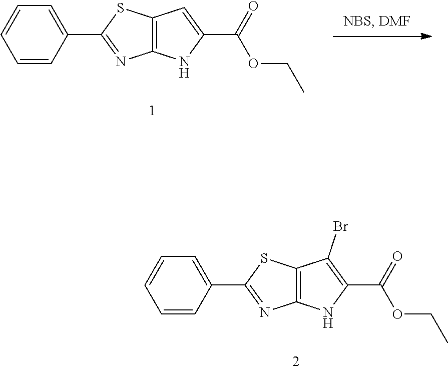 [SMILES: CCOC(=O)c1cc2sc(-c3ccccc3)nc2[nH]1 ; CCOC(=O)c1[nH]c2nc(-c3ccccc3)sc2c1Br]

To a solution of ethyl 2-phenyl-4H-pyrrolo[2,3-d]thiazole-5-carboxylate (200 mg, 734.42 umol, 1 eq) in DMF (3 mL) was added NBS (143.79 mg, 807.87 umol, 1.1 eq). The mixture was stirred at 30° C. for 30 min. LCMS showed that desired product was detected. The mixture was poured into saturated LiCl (20 mL), and then extracted with EtOAc (20 mL×2). The combined organic layer was dried over by Na₂SO₄ and filtered and concentrated under reduce pressure. The residue was purified by column chromatography (SiO₂, 0-20% Ethyl acetate in petroleum ether). Compound ethyl 6-bromo-2-phenyl-4H-pyrrolo[2,3-d]thiazole-5-carboxylate (130 mg, 333.13 umol, 45.36% yield, 90% purity) was obtained as a yellow solid.

LCMS (ESI) m/z: 290.7 [M+1]⁺; ¹H NMR was recorded.

### Step 2. Synthesis of ethyl 6-methyl-2-phenyl-4H-pyrrolo[2,3-d]thiazole-5-carboxylate

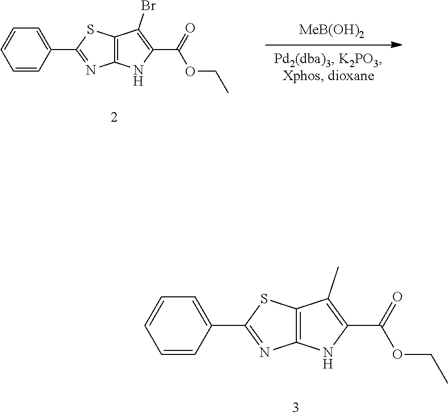 [SMILES: "MeB(OH)2" ; CCOC(=O)c1[nH]c2nc(-c3ccccc3)sc2c1Br ; CCOC(=O)c1[nH]c2nc(-c3ccccc3)sc2c1C]

A mixture of ethyl 6-bromo-2-phenyl-4H-pyrrolo[2,3-d]thiazole-5-carboxylate (130 mg, 370.14 umol, 1 eq), K₃PO₄ (235.70 mg, 1.11 mmol, 3 eq) and methylboronic acid (110.78 mg, 1.85 mmol, 5 eq) in dioxane (2 mL) was degassed under N₂ atmosphere. Pd₂(dba)₃ (50 mg, 54.60 umol, 1.48e-1 eq) and XPhos (24.70 mg, 51.82 umol, 0.14 eq) were then added. The suspension was degassed and purged with N₂ for 3 times, and then stirred under N₂ at 120° C. for 12 hr. LC-MS showed desired mass. EtOAc (50 mL) was added. The mixture was filtered to remove insoluble materials. The filtrate was concentrated in vacuo. The resulting residue was purified by column chromatography (SiO₂, 0-10% Ethyl acetate in petroleum ether). Compound ethyl 6-methyl-2-phenyl-4H-pyrrolo[2,3-d]thiazole-5-carboxylate (100 mg, 209.54 umol, 56.61% yield, 60% purity) was obtained as a white solid.

LCMS (ESI) m/z: 287.1 [M+H]⁺; ¹H NMR was recorded.

### Step 3. Synthesis of 6-methyl-2-phenyl-4H-pyrrolo[2,3-d]thiazole-5-carboxylic acid

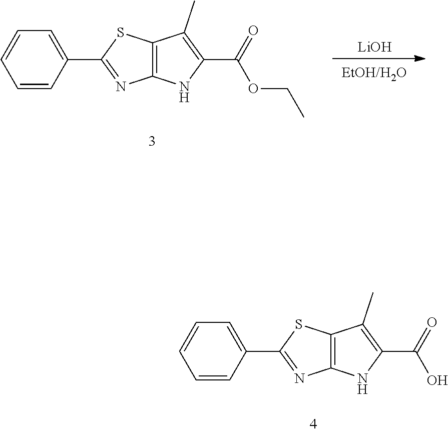 [SMILES: CCOC(=O)c1[nH]c2nc(-c3ccccc3)sc2c1C ; "LiOH" ; Cc1c(C(=O)O)[nH]c2nc(-c3ccccc3)sc12]

To a solution of ethyl 6-methyl-2-phenyl-4H-pyrrolo[2,3-d]thiazole-5-carboxylate (600 mg, 2.10 mmol, 1 eq) in EtOH (10 mL) was added a solution of LiOH.H₂O (1.76 g, 41.91 mmol, 20 eq) in H₂O (5 mL). The mixture was stirred at 80° C. for 1 hr. LCMS showed that desired mass and reactant 3 was consumed. The mixture was concentrated under reduced pressure to remove EtOH (10 mL). The residue was diluted with water (20 mL) and acidified to pH 4 with 1 N HCl in water, and then extracted with EtOAc (30 mL×2). The combined organic layer was dried over Na₂SO₄ and concentrated under reduced pressure to afford 6-methyl-2-phenyl-4H-pyrrolo[2,3-d]thiazole-5-carboxylic acid (500 mg, 1.84 mmol, 87.77% yield, 95% purity) was obtained as a yellow solid.

LCMS (ESI) m/z: 259.1 [M+H]⁺; ¹H NMR (400 MHz, DMSO-d₆) δ=7.91 (dd, J=1.3, 7.7 Hz, 2H), 7.62-7.38 (m, 3H), 1.87 (s, 3H).

### Step 4. Synthesis of 6-methyl-2-phenyl-N-(6-silaspiro[5.5]undecan-3-yl)-4H-pyrrolo[2,3-d]thiazole-5-carboxamide

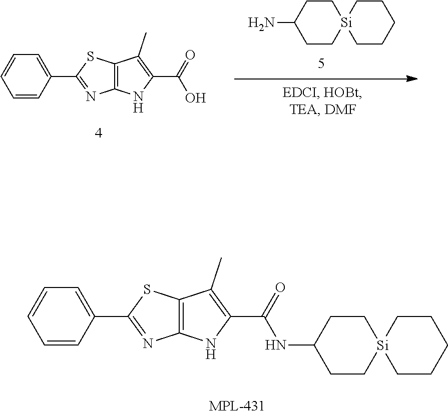 [SMILES: Cc1c(C(=O)O)[nH]c2nc(-c3ccccc3)sc12 ; NC1CC[Si]2(CCCCC2)CC1 ; Cc1c(C(=O)NC2CC[Si]3(CCCCC3)CC2)[nH]c2nc(-c3ccccc3)sc12]

To a solution of 6-methyl-2-phenyl-4H-pyrrolo[2,3-d]thiazole-5-carboxylic acid (80 mg, 309.72 umol, 1 eq) and 6-silaspiro[5.5]undecan-3-amine (68.09 mg, 309.72 umol, 1 eq, HCl salt) in DMF (0.5 mL) was added a solution of HOBt (125.55 mg, 929.17 umol, 3 eq) and EDCI (178.12 mg, 929.17 umol, 3 eq) in DMF (0.5 mL), followed by TEA (188.04 mg, 1.86 mmol, 258.66 uL, 6 eq). The mixture was stirred at 25° C. for 1 hr. LC-MS showed that the desired compound was detected. The reaction mixture was filtered and the filtrate was purified by prep-HPLC (column: YMC-Actus Triart C18 150*30 mm*5 um; mobile phase: A: 0.225% formic acid in water, B: CH₃CN; gradient: 85%-100% B over 11 min). 6-methyl-2-phenyl-N-(6-silaspiro[5.5]undecan-3-yl)-4H-pyrrolo[2,3-d]thiazole-5-carboxamide (11.3 mg, 26.05 umol, 8.41% yield, 97.67% purity) was obtained as a white solid.

LCMS (ESI) m/z: 424.2 [M+H]⁺; ¹H NMR (400 MHz, METHANOL-d₄) δ=7.97 (dd, J=1.6, 7.8 Hz, 2H), 7.50-7.42 (m, 3H), 3.79 (br t, J=11.3 Hz, 1H), 2.52 (s, 3H),2.18 (br d, J=12.1 Hz, 2H), 1.79-1.61 (m, 6H), 1.46 (br s, 2H), 0.99 (br s, 1H), 0.95 (br s, 1H), 0.81-0.76 (m, 2H), 0.74-0.64 (m, 4H).

### Example 42, MPL-433, MPL-433A and MPL-433B

### Synthesis of N-(4-methylcyclohexyl)-2-phenyl-4H-pyrrolo[2,3-d]thiazole-5-carboxamide, cis-N-(4-methylcyclohexyl)-2-phenyl-4H-pyrrolo[2,3-d]thiazole-5-carboxamide and trans-N-(4-methylcyclohexyl)-2-phenyl-4H-pyrrolo[2,3-d]thiazole-5-carboxamide

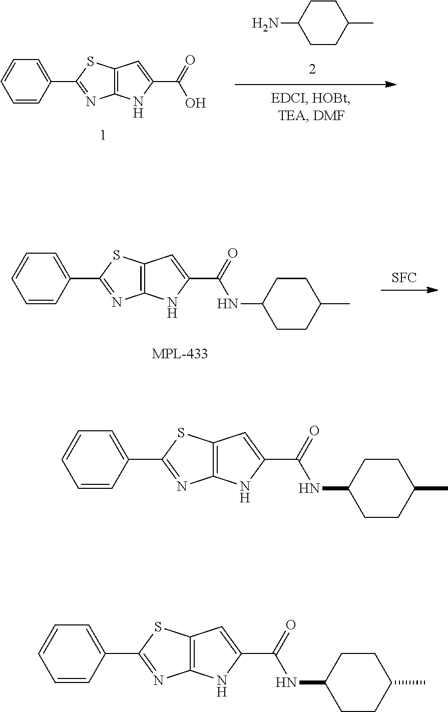

To a solution of 2-phenyl-4H-pyrrolo[2,3-d]thiazole-5-carboxylic acid (58 mg, 237.44 umol, 1 eq), 4-methylcyclohexanamine (29.57 mg, 261.19 umol, 34.58 uL, 1.1 eq) in DMF (0.5 mL) was added a solution of EDCI (136.56 mg, 712.33 umol, 3 eq) and HOBt (96.25 mg, 712.33 umol, 3 eq) in DMF (0.5 mL), followed by TEA (144.16 mg, 1.42 mmol, 198.29 uL, 6 eq). The mixture was stirred at 25° C. for 1 hr. LC-MS showed the desired compound was detected. The reaction mixture was filtered. The filtrate was purified by prep-HPLC (column: YMC-Actus Triart C18 150*30 mm*5 um; mobile phase: A: 0.225% formic acid in water, B: CH₃CN; gradient: 65%-88% B over 11 min). Compound N-(4-methylcyclohexyl)-2-phenyl-4H-pyrrolo[2,3-d]thiazole-5-carboxamide (40 mg, 94.27 umol, 39.70% yield, 80% purity) was obtained as a white solid.

LCMS m/z: 340.2 [M+1]⁺; ¹H NMR was recorded.

MPL-433 was separated by prep-SFC (Berger MG II; column: DAICEL CHIRALPAK; AS(250 mm*30 mm,10 um); mobile phase: A: 0.1%NH₃H₂O in MeOH, B CO₂; 35%B isocratic; flow rate: 60 mL/min) to afford two peaks (cis- and trans-isomers)

Peak 1 was assigned as MPL-433 A: 2.7 mg, 7.95 umol, 6.75% yield, 100% purity, white solid.

LCMS m/z: 340.1 [M+1]⁺; ¹H NMR (400 MHz, METHANOL-d₄) δ=7.98 (d, J=6.9 Hz, 2H), 7.51-7.41 (m, 3H), 7.09 (s, 1H), 3.82 (br t, J=12.0 Hz, 1H), 1.98 (br d, J=11.7 Hz, 2H), 1.84-1.72 (m, 2H), 1.43-1.38 (m, 2H), 1.31-1.27 (m, 1H), 1.16-1.06 (m, 2H), 0.95 (d, J=6.6 Hz, 3H).

Peak 2 was assigned as MPL-433B: 13.3 mg, 39.18 umol, 33.25% yield, 100% purity white solid.

LCMS m/z: 340.0 [M+1]⁺; ¹H NMR (400 MHz, METHANOL-d₄) δ=7.98 (d, J=6.7 Hz, 2H), 7.51-7.41 (m, 3H), 7.14 (s, 1H), 4.07-3.97 (m, 1H), 1.82-1.75 (m, 2H), 1.74-1.61 (m, 5H), 1.52-1.43 (m, 2H), 1.02 (d, J=6.7 Hz, 3H).

### Example 43, MPL-456

### Synthesis of N-(4,4-dimethylcyclohexyl)-2-methoxy-4H-pyrrolo[2,3-d]thiazole-5-carboxamide

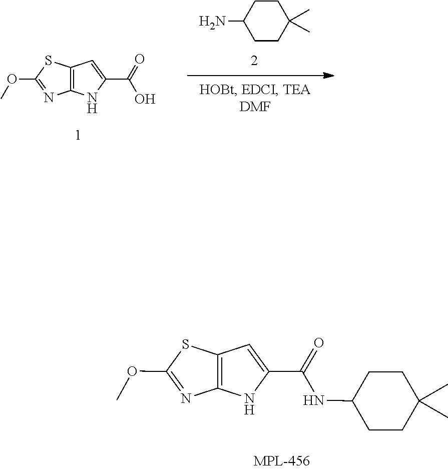

To a solution of 2-methoxy-4H-pyrrolo[2,3-d]thiazole-5-carboxylic acid (50 mg, 252.27 umol, 1 eq) and 4,4-dimethylcyclohexanamine (35.31 mg, 277.50 umol, 1.1 eq) in DMF (1 mL) at 25° C. was added a solution of HOBt (102.26 mg, 756.82 umol, 3 eq) and EDCI (145.08 mg, 756.82 umol, 3 eq) in DMF (1 mL), followed by TEA (127.64 mg, 1.26 mmol, 175.56 uL, 5 eq). The mixture was stirred at 25° C. for 2 hr. LC-MS showed desired compound was detected. The mixture was purified by prep-HPLC (column: Phenomenex Synergi C18 150*30 mm*4 um; mobile phase: A: 0.225% formic acid in water, B: CH₃CN; gradient: 45%-75% B over 11 min). Compound N-(4,4-dimethylcyclohexyl)-2-methoxy-4H-pyrrolo[2,3-d]thiazole-5-carboxamide (18.3 mg, 59.53 umol, 23.60% yield, 100% purity) was obtained as a brown solid.

### LCMS (ESI) m/z 308.1 [M+H]⁺; ¹H NMR (500 MHz, DMSO-d₆) δ=12.09 (s, 1H), 7.75 (d, J=8.1 Hz, 1H), 7.02-6.92 (m, 1H), 4.13-3.99 (m, 3H), 3.73-3.58 (m, 1H), 1.63 (br dd, J=3.4, 13.2 Hz, 2H), 1.54-1.43 (m, 2H), 1.39 (br d, J=12.8 Hz, 2H), 1.31-1.20 (m, 2H), 0.92 (d, J=9.2 Hz, 6H).

### Example 44, MPL-457

### Synthesis of N-[(1R,2R,3S,5R)-2-hydroxy-2,6,6-trimethyl-norpinan-3-yl]-2-methoxy-4H-pyrrolo[2,3-d]thiazole-5-carboxamide

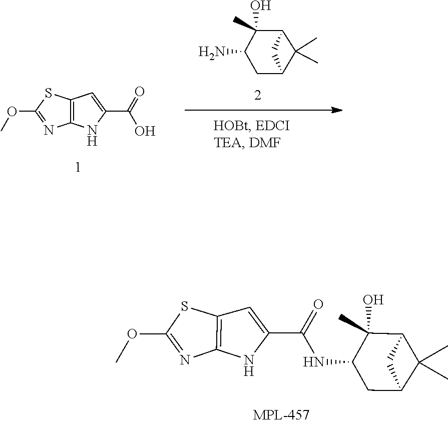

To a solution of 2-methoxy-4H-pyrrolo[2,3-d]thiazole-5-carboxylic acid (50 mg, 252.27 umol, 1 eq) and (1R,2R,3S,5R)-3-amino-2,6,6-trimethyl-norpinan-2-ol (57.09 mg, 277.50 umol, 1.1 eq, HCl) in DMF (1 mL) at 25° C. was added a solution of HOBt (102.26 mg, 756.81 umol, 3 eq) and EDCI (145.08 mg, 756.81 umol, 3 eq), followed by TEA (127.64 mg, 1.26 mmol, 175.56 uL, 5 eq). The mixture was stirred at 25° C. for 2 hr. LC-MS showed desired compound was detected. The mixture was purified by prep-HPLC (column: Phenomenex Synergi C18 150*30 mm*4 um; mobile phase: A: 0.225% formic acid in water, B: CH₃CN; gradient: 45%-71% B over 11 min). Compound N-[(1R,2R,3S,5R)-2-hydroxy-2,6,6-trimethyl-norpinan-3-yl]-2-methoxy-4H-pyrrolo[2,3-d]thiazole-5-carboxamide (13.7 mg, 39.20 umol, 15.54% yield, 100% purity) was obtained as a brown solid.

LCMS (ESI) m/z 350.2 [M+H]⁺; ¹H NMR (500 MHz, DMSO-d₆) δ=12.19 (s, 1H), 7.44 (d, J=9.0 Hz, 1H), 6.98 (d, J=1.4 Hz, 1H), 4.51 (s, 1H), 4.46 (q, J=9.1 Hz, 1H), 4.12-4.05 (m, 3H), 2.31-2.21 (m, 1H), 2.17-2.04 (m, 1H), 1.89 (d, J=6.0 Hz, 2H), 1.63-1.53 (m, 2H), 1.31-1.23 (m, 3H), 1.18 (s, 3H), 1.06 (s, 3H).

### Example 45, MPL-458

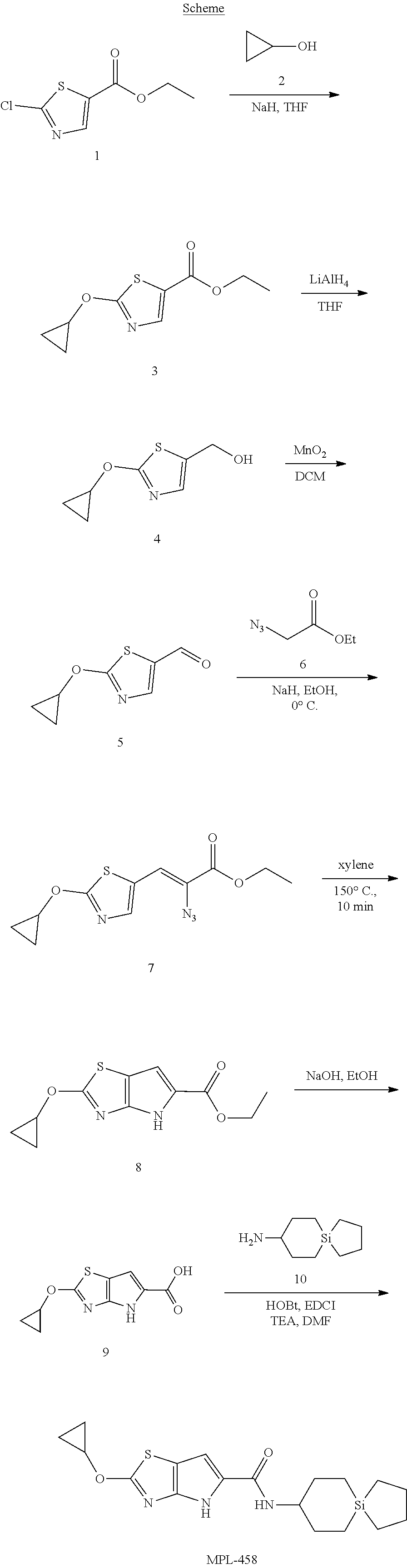

### Step 1. Synthesis of ethyl 2-(cyclopropoxy)thiazole-5-carboxylate

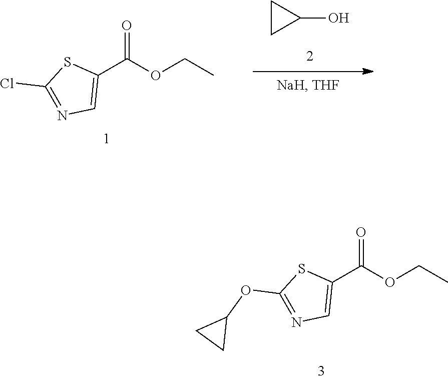

To an ice-cooled solution of cyclopropanol (227.38 mg, 3.92 mmol, 1.5 eq) in THF (10 mL) was added NaH (156.53 mg, 3.92 mmol, 60% purity, 1.5 eq). The mixture was stirred at 0-5° C. for 10 min. Then a solution of ethyl 2-chlorothiazole-5-carboxylate (500 mg, 2.61 mmol, 1 eq) in THF (2 mL) was added. The mixture was stirred at 0-5° C. for 30 min. LC-MS showed desired compound was detected. The reaction mixture was adjusted to pH 6 with aqueous HCl (2 M), and then extracted with EtOAc (50 mL×2). The combined organic layer was washed with brine (50 mL×2), dried over Na₂SO₄, and filtered and concentrated under reduced pressure to afford ethyl 2-(cyclopropoxy)thiazole-5-carboxylate (530 mg, 1.74 mmol, 66.66% yield, 70% purity) as a yellow oil. The crude product was used for the next step without further purification.

LCMS (ESI) m/z 214.1 [M+H]⁺; ¹H NMR was recorded.

### Step 2. Synthesis of [2-(cyclopropoxy)thiazol-5-yl]methanol

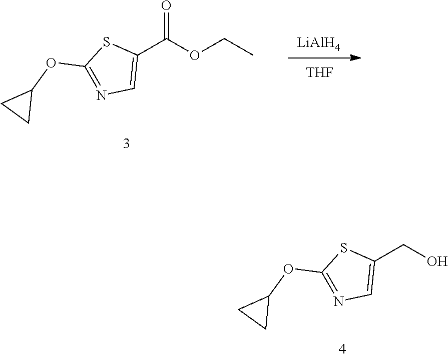

To an ice-cooled solution of ethyl 2-(cyclopropoxy)thiazole-5-carboxylate (530 mg, 2.49 mmol, 1 eq) in dried THF (10 mL) was added LiAlH₄ (140 mg, 3.69 mmol, 1.48 eq) in batches, the mixture was stirred at 0-10° C. for 30 min. LC-MS showed desired compound was detected. The reaction was quenched by addition of water (0.14 mL), NaOH (15%, 0.14 mL) and water (0.42 mL). The mixture was filtered. The filter cake was washed with EtOAc (50 mL×3). The combined filtrate was dried over Na₂SO₄, and then filtered and concentrated under reduced pressure. Compound [2 (cyclopropoxy) thiazol-5-yl]methanol (340 mg, 1.39 mmol, 55.93% yield, 70% purity) was obtained as a yellow oil. The crude product was used for the next step without further purification.

LCMS (ESI) m/z 172.0 [M+H]⁺; ¹H NMR was recorded.

### Step 3. Synthesis of 2-(cyclopropoxy)thiazole-5-carbaldehyde

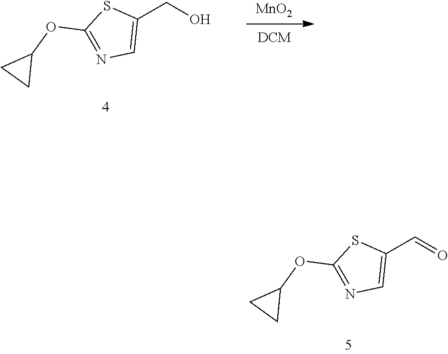

To a solution of [2-(cyclopropoxy)thiazol-5-yl]methanol (340 mg, 1.99 mmol, 1 eq) in DCM (5 mL) was added MnO₂ (1.73 g, 19.86 mmol, 10 eq). The mixture was stirred at 25° C. for 2 hr. LC-MS showed desired compound was detected. The mixture was filtered. The filter cake was washed with EtOAc (10 mL×2). The combined organic layer was concentrated under reduced pressure to give a residue which was purified by column chromatography (SiO₂, 0-10% Ethyl acetate in petroleum ether). Compound 2-(cyclopropoxy)thiazole-5-carbaldehyde (233 mg, 1.10 mmol, 55.48% yield, 80% purity) was obtained as a yellow oil.

LCMS (ESI) m/z 170.0 [M+H]⁺; ¹H NMR was recorded.

### Step 4. Synthesis of ethyl (Z)-2-azido-3-[2-(cyclopropoxy)thiazol-5-yl]prop-2-enoate

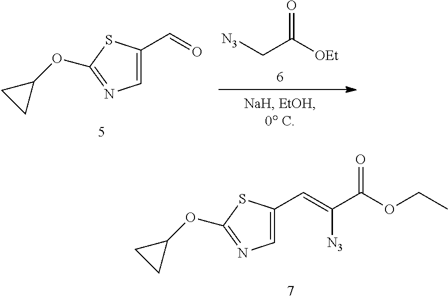

NaH (275.39 mg, 6.89 mmol, 60% purity, 5 eq) was added to EtOH (5 mL) in batches. The mixture was stirred at 30° C. until a clear solution formed, and then cooled to −10° C. A solution of 2-(cyclopropoxy)thiazole-5-carbaldehyde (233 mg, 1.38 mmol, 1 eq) and ethyl 2-azidoacetate (889.01 mg, 6.89 mmol, 966.31 uL, 5 eq) in THF (2 mL) was added to the mixture dropwise. The mixture was stirred at −10° C.˜0° C. for 2 hr. LC-MS showed desired compound was detected. The mixture was adjusted to pH 6 with aqueous HCl (2 M), and then extracted with EtOAc (50 mL×2). The combined organic layer was washed with brine (50 mL×2), dried over Na₂SO₄, filtered and concentrated under reduced pressure to give a residue which was purified by column chromatography (SiO₂, 0-10% Ethyl acetate in petroleum ether). Compound ethyl (Z)-2-azido-3-[2-(cyclopropoxy)thiazol-5-yl]prop-2-enoate (350 mg, 874.06 umol, 63.47% yield, 70% purity) was obtained as a yellow oil.

LCMS (ESI) m/z 281.0 [M+H]⁺; ¹H NMR was recorded.

### Step 5. Synthesis of ethyl 2-(cyclopropoxy)-4H-pyrrolo[2,3-d]thiazole-5-carboxylate

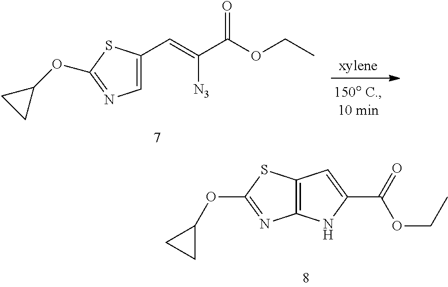 [SMILES: CCOC(=O)/C(=C/c1cnc(OC2CC2)s1)N=[N+]=[N-] ; CCOC(=O)c1cc2sc(OC3CC3)nc2[nH]1]

Ethyl (Z)-2-azido-3-[2-(cyclopropoxy)thiazol-5-yl]prop-2-enoate (350 mg, 1.25 mmol, 1 eq) in xylene (5 mL) was stirred at 150° C. for 10 min. LC-MS showed desired compound was detected. The reaction mixture was concentrated under reduced pressure to afford ethyl 2-(cyclopropoxy)-4H-pyrrolo[2,3-d]thiazole-5-carboxylate (300 mg, 832.38 umol, 66.66% yield, 70% purity) as a yellow oil. The crude product was used for the next step without further purification.

LCMS (ESI) m/z 253.0 [M+H]⁺; ¹H NMR was recorded.

### Step 6. Synthesis of 2-(cyclopropoxy)-4H-pyrrolo[2,3-d]thiazole-5-carboxylic acid

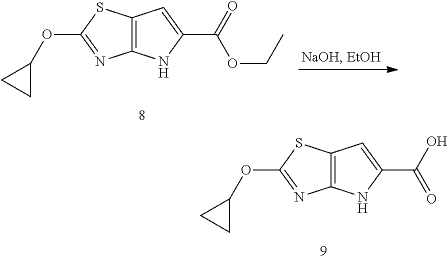 [SMILES: CCOC(=O)c1cc2sc(OC3CC3)nc2[nH]1 ; O=C(O)c1cc2sc(OC3CC3)nc2[nH]1]

To a solution of ethyl 2-(cyclopropoxy)-4H-pyrrolo[2,3-d]thiazole-5-carboxylate (300 mg, 1.19 mmol, 1 eq) in EtOH (2 mL) was added NaOH (2 M, 2 mL, 3.36 eq). The mixture was stirred at 80° C. for 2 hr. LC-MS showed desired compound was detected. The reaction mixture was concentrated under reduced pressure to remove EtOH. The residue was diluted with water (20 mL), acidified to pH 2 with HCl (2M in water), and extracted with EtOAc (20 mL×2). The combined organic layer was washed with brine (20 mL×2), dried over Na₂SO₄, and then filtered and concentrated under reduced pressure. Compound 2-(cyclopropoxy)-4H-pyrrolo[2,3-d]thiazole-5-carboxylic acid (190 mg, 847.32 umol, 71.26% yield) was obtained as a brown oil. The crude product was used for next step without further purification.

LCMS (ESI) m/z 225.0 [M+H]⁺; ¹H NMR (500 MHz, DMSO-d₆) δ=12.38 (br s, 1H), 7.01-6.90 (m, 1H), 1.99 (s, 1H), 1.17 (br t, J=7.1 Hz, 2H), 0.90-0.86 (m, 2H).

### Step 7. Synthesis of 2-(cyclopropoxy)-N-(5-silaspiro[4.5]decan-8-yl)-4H-pyrrolo[2,3-d]thiazole-5-carboxamide

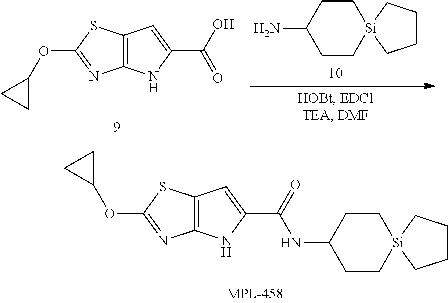 [SMILES: O=C(O)c1cc2sc(OC3CC3)nc2[nH]1 ; NC1CC[Si]2(CCCC2)CC1 ; O=C(NC1CC[Si]2(CCCC2)CC1)c1cc2sc(OC3CC3)nc2[nH]1]

To a solution of 2-(cyclopropoxy)-4H-pyrrolo[2,3-d]thiazole-5-carboxylic acid (190 mg, 847.32 umol, 1 eq) and 5-silaspiro[4.5]decan-8-amine (191.82 mg, 932.05 umol, 1.1 eq, HCl salt) in DMF (1 mL) at 25° C. was added a solution of HOBt (343.48 mg, 2.54 mmol, 3 eq) and EDCI (487.30 mg, 2.54 mmol, 3 eq) in DMF (1 mL), followed by TEA (428.70 mg, 4.24 mmol, 589.68 uL, 5 eq). The reaction was stirred at 25° C. for 1 hr. LC-MS showed desired compound was detected. The mixture was purified by prep-HPLC (column: Phenomenex Synergi C18 150*30 mm*4 um; mobile phase: A: 0.225% formic acid in water, B: CH₃CN; gradient: 60%-90% B over 11 min). Compound 2-(cyclopropoxy)-N-(5-silaspiro[4.5]decan-8-yl)-4H-pyrrolo[2,3-d]thiazole-5-carboxamide (5 mg, 13.31 umol, 1.57% yield, 100% purity) was obtained as a brown solid.

LCMS (ESI) m/z 376.2 [M+H]⁺; ¹H NMR (500 MHz, DMSO-d₆) δ=12.07 (br s, 1H), 7.82-7.70 (m, 1H), 7.04-6.91 (m, 1H), 4.34 (tt, J=3.1, 5.9 Hz, 1H), 3.78-3.62 (m, 1H), 2.17-1.81 (m, 2H), 1.71-1.38 (m, 6H), 0.86-0.51 (m, 10H).

### Example 46, MPL-459

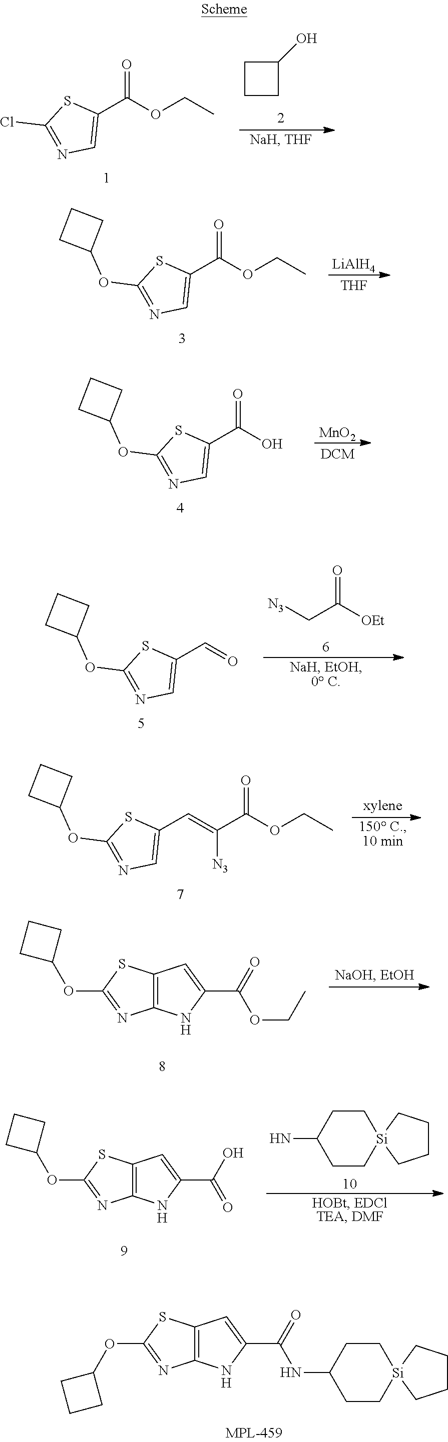

### Step 1. Synthesis of ethyl 2-(cyclobutoxy)thiazole-5-carboxylate

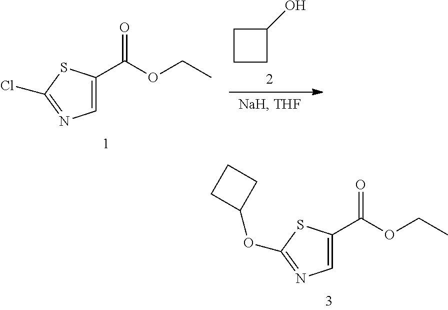

To an ice-cooled solution of cyclobutanol (188.20 mg, 2.61 mmol, 1 eq) in THF (10 mL) was added NaH (156.58 mg, 3.92 mmol, 60% purity, 1.5 eq) and stirred at 0-5° C. for 10 min. Then a solution of ethyl 2-chlorothiazole-5-carboxylate (500 mg, 2.61 mmol, 1 eq) in THF (2 mL) was added. The mixture was stirred at 0-5° C. for 30 min. LC-MS showed desired compound was detected. The mixture was adjusted to pH 6 using aqueous HCl (2 M), and then extracted with EtOAc (50 mL×2). The combined organic layer was washed with brine (50 mL×2), dried over Na₂SO₄, filtered and concentrated under reduced pressure. Compound ethyl 2-(cyclobutoxy)thiazole-5-carboxylate (590 mg, 1.30 mmol, 49.73% yield, 50% purity) was obtained as a yellow oil. The crude product was used for the next step without further purification.

LCMS (ESI) m/z 228.0 [M+H]⁺; ¹H NMR was recorded.

### Step 2. Synthesis of [2-(cyclobutoxy)thiazol-5-yl]methanol

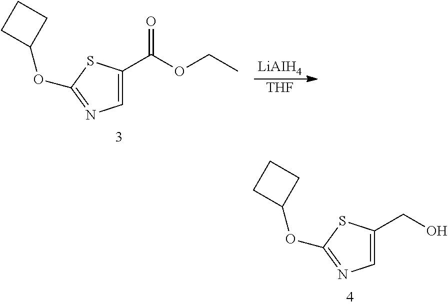

To an ice-cooled solution of ethyl 2-(cyclobutoxy)thiazole-5-carboxylate (590 mg, 2.60 mmol, 1 eq) in dried THF (5 mL) was added LiAlH₄ (150 mg, 3.95 mmol, 1.52 eq) in batches. The mixture was stirred at 0-5° C. for 30 min. LC-MS showed desired compound was detected. The reaction was quenched with water (0.15 mL), NaOH (15%, 0.15 mL) and water (0.45 mL). The mixture was then filtered. The filter cake was washed with EtOAc (50 mL×3). The combined filtrate was dried over Na₂SO₄, and then filtered and concentrated under reduced pressure. Compound [2-(cyclobutoxy)thiazol-5-yl]methanol (367 mg, 1.58 mmol, 61.06% yield, 80% purity) was obtained as a yellow oil. The crude product was used for the next step without further purification.

LCMS (ESI) m/z 186.1 [M+H]⁺; ¹H NMR was recorded.

### Step 3. Synthesis of 2-(cyclobutoxy)thiazole-5-carbaldehyde

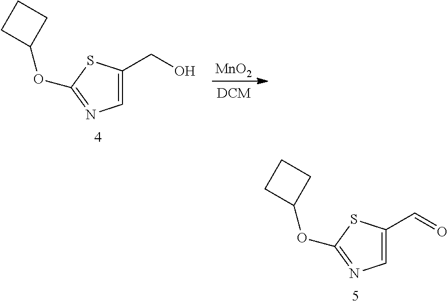

To a solution of [2-(cyclobutoxy)thiazol-5-yl]methanol (367 mg, 1.98 mmol, 1 eq) in DCM (5 mL) was added MnO₂ (1.72 g, 19.81 mmol, 10 eq). The mixture was stirred at 25° C. for 1 hr. LC-MS showed desired compound was detected. The mixture was filtered. The filter cake was washed with EtOAc (10 mL×2). The combined organic layer was concentrated under reduced pressure to give a residue which was purified by column chromatography (SiO₂, 0-10% Ethyl acetate in petroleum ether). Compound 2-(cyclobutoxy)thiazole-5-carbaldehyde (230 mg, 1.13 mmol, 57.02% yield, 90% purity) was obtained as a yellow oil.

LCMS (ESI) m/z 184.0 [M+H]⁺; ¹H NMR was recorded.

### Step 4. Synthesis of ethyl (Z)-2-azido-3-[2-(cyclobutoxy)thiazol-5-yl]prop-2-enoate

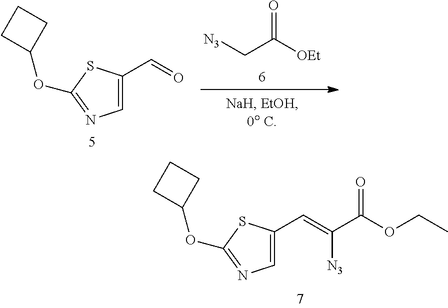 [SMILES: O=Cc1cnc(OC2CCC2)s1 ; CCOC(=O)CN=[N+]=[N-] ; CCOC(=O)/C(=C/c1cnc(OC2CCC2)s1)N=[N+]=[N-]]

NaH (251.03 mg, 6.28 mmol, 60% purity, 5 eq) was added to EtOH (5 mL) in batches. The mixture was stirred at 30° C. until a clear solution formed, and then cooled to −10° C. A solution of 2-(cyclobutoxy)thiazole-5-carbaldehyde (230 mg, 1.26 mmol, 1 eq) and ethyl 2-azidoacetate (810.39 mg, 6.28 mmol, 880.85 uL, 5 eq) in THF (2 mL) was added to the mixture dropwise. The mixture was stirred at −10° C.˜0° C. for 2 hr. LC-MS showed desired compound was detected. The reaction mixture was adjusted to pH 6 using aqueous HCl (2 M), and then extracted with EtOAc (50 mL×2). The combined organic layer was washed with brine (50 mL×2), dried over Na₂SO₄, filtered and concentrated under reduced pressure to give a residue which was purified by column chromatography (SiO₂, 0-10% Ethyl acetate in petroleum ether). Compound ethyl (Z)-2-azido-3-[2-(cyclobutoxy)thiazol-5-yl]prop-2-enoate (350 mg, 832.40 umol, 66.31% yield, 70% purity) was obtained as a yellow oil.

LCMS (ESI) m/z 295.1 [M+H]⁺; ¹H NMR was recorded.

### Step 5. Synthesis of ethyl 2-(cyclobutoxy)-4H-pyrrolo[2,3-d]thiazole-5-carboxylate

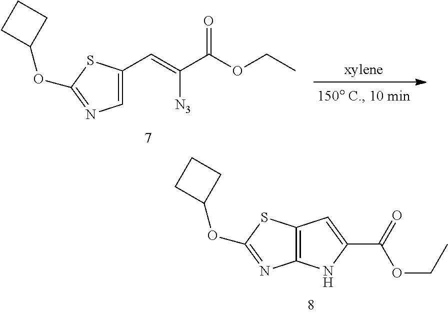 [SMILES: CCOC(=O)/C(=C/c1cnc(OC2CCC2)s1)N=[N+]=[N-] ; CCOC(=O)c1cc2sc(OC3CCC3)nc2[nH]1]

Ethyl (Z)-2-azido-3-[2-(cyclobutoxy)thiazol-5-yl]prop-2-enoate (350 mg, 1.19 mmol, 1 eq) in xylene (5 mL) was stirred at 150° C. for 10 min. LC-MS showed desired compound was detected. The reaction mixture was concentrated under reduced pressure to afford ethyl 2-(cyclobutoxy)-4H-pyrrolo[2,3-d]thiazole-5-carboxylate (300 mg, 788.54 umol, 66.31% yield, 70% purity) as a yellow oil. The crude product was used for the next step without further purification.

LCMS (ESI) m/z 267.1 [M+H]⁺; ¹H NMR was recorded.

### Step 6. Synthesis of 2-(cyclobutoxy)-4H-pyrrolo[2,3-d]thiazole-5-carboxylic acid

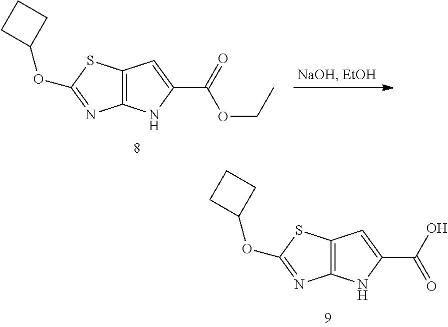 [SMILES: CCOC(=O)c1cc2sc(OC3CCC3)nc2[nH]1 ; O=C(O)c1cc2sc(OC3CCC3)nc2[nH]1]

To a solution of ethyl 2-(cyclobutoxy)-4H-pyrrolo[2,3-d]thiazole-5-carboxylate (300 mg, 1.13 mmol, 1 eq) in EtOH (2 mL) was added NaOH (2 M, 2 mL, 3.55 eq). The mixture was stirred at 80° C. for 2 hr. LC-MS showed desired compound was detected. The reaction mixture was concentrated under reduced pressure to remove EtOH. The residue was diluted with water (20 mL), acidified to pH 2 with HCl (2M in water), and then extracted with EtOAc (20 mL×2). The combined organic layer was washed with brine (20 mL×2), dried over Na₂SO₄, and filtered and concentrated under reduced pressure. Compound 2-(cyclobutoxy)-4H-pyrrolo[2,3-d]thiazole-5-carboxylic acid (219 mg, 735.32 umol, 65.28% yield, 80% purity) was obtained as a brown oil. The crude product was used for the next step without further purification.

LCMS (ESI) m/z 239.1 [M+H]⁺; ¹H NMR (500 MHz, DMSO-d₆) δ=12.35 (br s, 1H), 6.92 (d, J=1.8 Hz, 1H), 5.24-5.14 (m, 1H), 2.20-2.14 (m, 1H), 2.14-2.14 (m, 1H), 1.89-1.52 (m, 4H).

### Step 7. Synthesis of 2-(cyclobutoxy)-N-(5-silaspiro[4.5]decan-8-yl)-4H-pyrrolo[2,3-d]thiazole-5-carboxamide

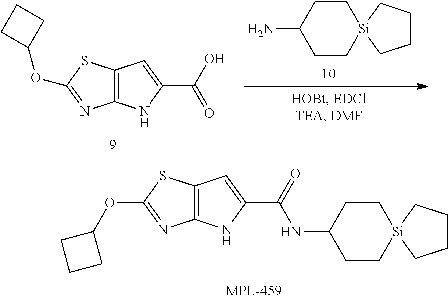 [SMILES: O=C(O)c1cc2sc(OC3CCC3)nc2[nH]1 ; NC1CC[Si]2(CCCC2)CC1 ; O=C(NC1CC[Si]2(CCCC2)CC1)c1cc2sc(OC3CCC3)nc2[nH]1]

To a solution of 2-(cyclobutoxy)-4H-pyrrolo[2,3-d]thiazole-5-carboxylic acid (219 mg, 919.15 umol, 1 eq) and 5-silaspiro[4.5]decan-8-amine (208.08 mg, 1.01 mmol, 1.1 eq, HCl salt) in DMF (1 mL) at 25° C. was added a solution of HOBt (372.60 mg, 2.76 mmol, 3 eq) and EDCI (528.61 mg, 2.76 mmol, 3 eq) in DMF (1 mL), followed by TEA (465.04 mg, 4.60 mmol, 639.67 uL, 5 eq). The mixture was stirred at 25° C. for 1 hr. LC-MS showed desired compound was detected. The mixture was purified by prep-HPLC (column: Phenomenex Synergi C18 150*30 mm*4 um; mobile phase: A: 0.225% formic acid in water, B: CH₃CN; gradient: 65%-95% B over 11 min). Compound 2-(cyclobutoxy)-N-(5 -silaspiro[4.5]decan-8-yl)-4H-pyrrolo [2,3 -d]thiazole-5 -carboxamide (7.5 mg, 19.25 umol, 2.09% yield, 100% purity) was obtained as a brown solid.

LCMS (ESI) m/z 390.2 [M+H]⁺; ¹H NMR (500 MHz, DMSO-d₆) δ=12.04 (br s, 1H), 7.73 (d, J=8.1 Hz, 1H), 6.95 (s, 1H), 5.15 (quin, J=7.2 Hz, 1H), 3.69 (dt, J=8.1, 11.0 Hz, 1H), 2.46-2.38 (m, 2H), 2.25-1.96 (m, 4H), 1.89-1.38 (m, 8H), 0.83-0.46 (m, 8H).

### Example 47, MPL-472

### Synthesis of 2-(cyclopropoxy)-N-(1,1-dimethylsilepan-4-yl)-4H-pyrrolo[2,3-d]thiazole-5-carboxamide

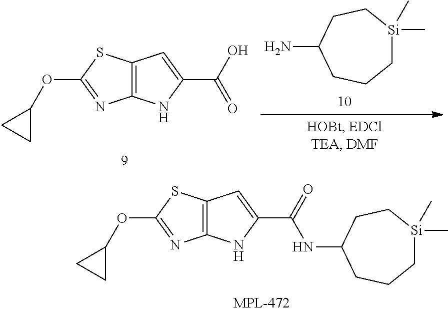

To a solution of 2-(cyclopropoxy)-4H-pyrrolo[2,3-d]thiazole-5-carboxylic acid (50 mg, 222.98 umol, 1 eq) and 1,1-dimethylsilepan-4-amine (51.85 mg, 267.58 umol, 1.2 eq, HCl salt) in DMF (1 mL) at 25° C. was added a solution of HOBt (90.39 mg, 668.94 umol, 3 eq) and EDCI (128.24 mg, 668.94 umol, 3 eq) in DMF (1 mL), followed by TEA (112.82 mg, 1.11 mmol, 155.18 uL, 5 eq). The mixture was stirred at 25° C. for 2 hr. LC-MS showed desired compound was detected. The mixture was purified by prep-HPLC (column: Phenomenex Synergi C18 150*30 mm*4 um; mobile phase: A: 0.225% formic acid in water, B: CH₃CN; gradient:55%-85% B over 11 min). Compound 2-(cyclopropoxy)-N-(1,1-dimethylsilepan-4-yl)-4H-pyrrolo[2,3-d]thiazole-5-carboxamide (28.8 mg, 79.22 umol, 35.53% yield, 100% purity) was obtained as a white solid.

LCMS (ESI) m/z 364.2 [M+H]⁺; ¹H NMR (500 MHz, DMSO-d₆) δ=12.04 (br s, 1H), 7.78 (d, J=8.1 Hz, 1H), 6.99 (s, 1H), 4.34 (tt, J=3.1, 6.0 Hz, 1H), 3.82 (br s, 1H), 1.95-1.37 (m, 6H), 0.93-0.52 (m, 8H), 0.02 (d, J=7.5 Hz, 6H).

### Example 48, MPL-474

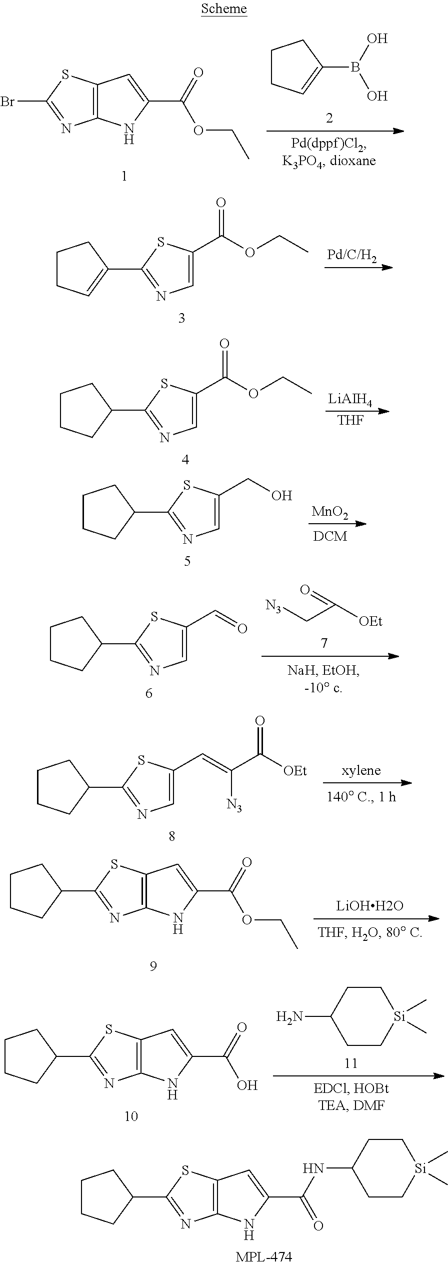

### Step 1. Synthesis of ethyl 2-(cyclopenten-1-yl)thiazole-5-carboxylate

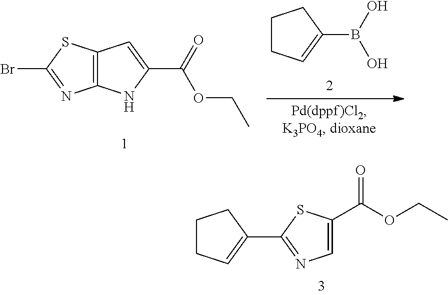

To a mixture of ethyl 2-bromothiazole-5-carboxylate (3 g, 12.71 mmol, 1 eq), cyclopenten-1-ylboronic acid (2.13 g, 19.06 mmol, 1.5 eq) and K₃PO₄ (10.79 g, 50.83 mmol, 4 eq) in dioxane (40 mL) was added Pd(dppf)Cl₂ (929.80 mg, 1.27 mmol, 0.1 eq) under N₂. The mixture was heated at 110° C. for 12 hr. TLC (Petroleum ether: Ethyl acetate=5:1) indicated compound 1 was consumed completely and new spots formed. The reaction mixture was filtered and concentrated under reduced pressure to give a residue which was purified by column chromatography (SiO₂, 0-10% Ethyl acetate in petroleum ether). Compound ethyl 2-(cyclopenten-1-yl)thiazole-5-carboxylate (2.26 g, 9.62 mmol, 75.67% yield, 95% purity) was obtained as a brown solid. ¹H NMR was recorded.

### Step 2. Synthesis of 2-cyclopentylthiazole-5-carboxylate

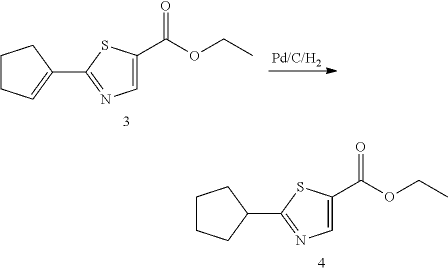

To a solution of ethyl 2-(cyclopenten-1-yl)thiazole-5-carboxylate (2.2 g, 9.85 mmol, 1 eq) in MeOH (20 mL) was added Pd/C (200 mg, 9.85 mmol, 10% purity, 1.00 eq) under H₂ atmosphere. The suspension was degassed and purged with H₂ for 3 times, and then stirred under H₂ (15 Psi) at 25° C. for 12 hr. LCMS showed desired mass was detected. The reaction mixture was filtered and concentrated under reduced pressure to give a residue which was purified by column chromatography (SiO₂, 0-10% Ethyl acetate in petroleum ether). Compound ethyl 2-cyclopentylthiazole-5-carboxylate (2.2 g, 8.79 mmol, 89.19% yield, 90% purity) was obtained as a white solid.

LCMS (ESI) m/z: 226.1 [M+H]⁺; ¹H NMR was recorded.

### Step 3. Synthesis of (2-cyclopentylthiazol-5-yl)methanol

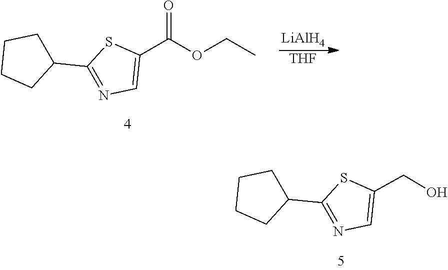

To an ice-cooled solution of ethyl 2-cyclopentylthiazole-5-carboxylate (2.2 g, 9.76 mmol, 1 eq) in dried THF (20 mL) was added LAH (555.91 mg, 14.65 mmol, 1.5 eq) in batches. The mixture was stirred at 0° C. for 1 hr. TLC (Petroleum ether: Ethyl acetate=5:1) indicated the reactant was consumed completely and one new spot formed. The reaction was quenched with water (0.555 mL), NaOH (15%, 0.555 mL) and water (1.665 mL), and then Na₂SO₄ was added. The mixture was filtered. The filtrate was concentrated under reduced pressure. The resulting residue was purified by column chromatography (SiO₂, 0-40% Ethyl acetate in petroleum ether). Compound (2-cyclopentylthiazol-5-yl)methanol (1.45 g, 7.12 mmol, 72.92% yield, 90% purity) was obtained as a colorless oil. ¹H NMR was recorded.

### Step 4. Synthesis of 2-cyclopentylthiazole-5-carbaldehyde

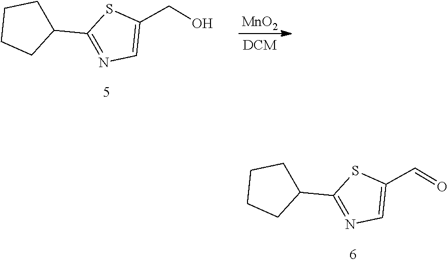

To a solution of (2-cyclopentylthiazol-5-yl)methanol (1.45 g, 7.91 mmol, 1 eq) in DCM (20 mL) was added MnO₂ (6.88 g, 79.12 mmol, 10 eq). The mixture was stirred at 25° C. for 12 hr. TLC (Petroleum ether: Ethyl acetate=5:1) indicated one major new spot formed. The reaction mixture was filtered and concentrated under reduced pressure to give a residue which was purified by column chromatography (SiO₂, 0-20% Ethyl acetate in petroleum ether). Compound 2-cyclopentylthiazole-5-carbaldehyde (1.2 g, 5.96 mmol, 75.31% yield, 90% purity) was obtained as a yellow oil. ¹H NMR was recorded.

### Step 5. Synthesis of ethyl (Z)-2-azido-3-(2-cyclopentylthiazol-5-yl) prop-2-enoate

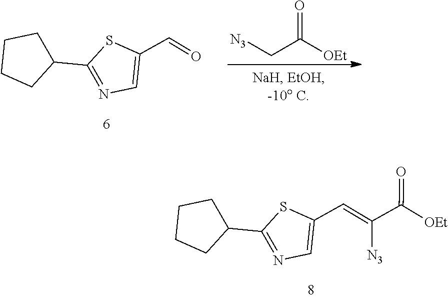 [SMILES: O=Cc1cnc(C2CCCC2)s1 ; CCOC(=O)CN=[N+]=[N-] ; CCOC(=O)/C(=C/c1cnc(C2CCCC2)s1)N=[N+]=[N-]]

NaH (794.38 mg, 19.86 mmol, 60% purity, 3 eq) was added to EtOH (10 mL) in batches. The mixture was stirred at 20° C. until a clear solution formed, and then cooled to −10° C. A solution of 2-cyclopentylthiazole-5-carbaldehyde (1.2 g, 6.62 mmol, 1 eq) and ethyl 2-azidoacetate (2.56 g, 19.86 mmol, 2.79 mL, 3 eq) in THF (10 mL) was added to the mixture dropwise. The mixture was stirred at −10° C.˜0° C. for 2 hr. TLC (Petroleum ether: Ethyl acetate=5:1) indicated the aldehyde was consumed completely and new spots formed. The reaction was quenched with saturated NH₄Cl (60 mL), and then extracted with EtOAc (50 mL×2). The combined organic layer was washed with brine (60 mL×2), dried over Na₂SO₄, filtered and concentrated under reduced pressure to give a residue which was purified by column chromatography (SiO₂, 0-6% Ethyl acetate in petroleum ether). Compound ethyl (Z)-2-azido-3-(2-cyclopentylthiazol-5-yl) prop-2-enoate (1.1 g, crude) was obtained as a yellow oil.

LCMS (ESI) m/z: 293.1 [M+1]⁺

### Step 6. Synthesis of ethyl 2-cyclopentyl-4H-pyrrolo[2,3-d]thiazole-5-carboxylate

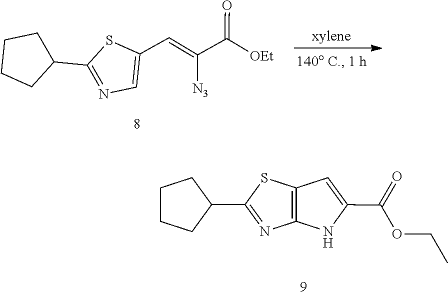 [SMILES: CCOC(=O)/C(=C/c1cnc(C2CCCC2)s1)N=[N+]=[N-] ; CCOC(=O)c1cc2sc(C3CCCC3)nc2[nH]1]

A solution of ethyl (Z)-2-azido-3-(2-cyclopentylthiazol-5-yl)prop-2-enoate (1.1 g, 3.76 mmol, 1 eq) in xylene (5 mL) was stirred at 140° C. for 30 min. TLC (Petroleum ether: Ethyl acetate=5:1) indicated the reactant was consumed completely and new spots formed. The reaction mixture was was purified by column chromatography (SiO₂, 0-20% Ethyl acetate in petroleum ether). Compound ethyl 2-cyclopentyl-4H-pyrrolo[2,3-d]thiazole-5-carboxylate (834 mg, 2.84 mmol, 75.47% yield, 90% purity) was obtained as a white solid. ¹H NMR was recorded.

### Step 7. Synthesis of 2-cyclopentyl-4H-pyrrolo[2,3-d]thiazole-5-carboxylic acid

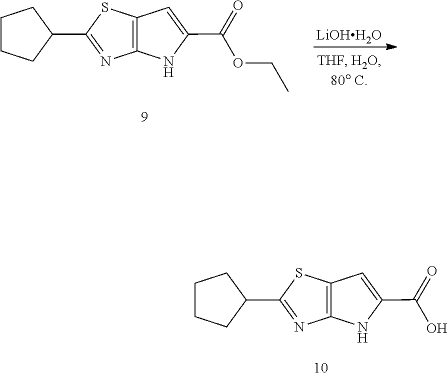

To a solution of ethyl 2-cyclopentyl-4H-pyrrolo[2,3-d]thiazole-5-carboxylate (834 mg, 3.15 mmol, 1 eq) in THF (10 mL) was added a solution of LiOH.H₂O (794.37 mg, 18.93 mmol, 6 eq) in H₂O (10 mL). The mixture was stirred at 80° C. for 6 hr. TLC (Petroleum ether: Ethyl acetate=5:1) indicated the reactant was consumed completely and one new spot formed. The reaction mixture was concentrated under reduced pressure to remove THF, and then acidified to pH=3-4 with HCl (1 N, in water) and filtered. The filter cake was washed with petroleum ether (15 mL) and dried under reduced pressure. Compound 2-cyclopentyl-4H-pyrrolo[2,3-d]thiazole-5-carboxylic acid (690 mg, 2.63 mmol, 83.30% yield, 90% purity) was obtained as a brown solid. The crude product was used for the next step without further purification.

¹H NMR (500 MHz, DMSO-d₆) δ=12.74-12.25 (m, 2H), 7.00 (d, J=1.8 Hz, 1H), 3.52-3.41 (m, 1H), 2.18-2.06 (m, 2H), 1.86-1.71 (m, 4H), 1.71-1.61 (m, 2H).

### Step 8. Synthesis of 2-cyclopentyl-N-(1,1-dimethylsilinan-4-yl)-4H-pyrrolo[2,3-d] thiazole-5-carboxamide

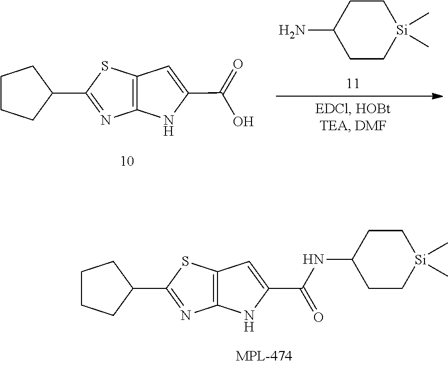

To a solution of 2-cyclopentyl-4H-pyrrolo[2,3-d]thiazole-5-carboxylic acid (50 mg, 211.60 umol, 1 eq) and 1,1-dimethylsilinan-4-amine (45.65 mg, 253.93 umol, 1.2 eq, HCl salt) in DMF (1 mL) was added a solution of EDCI (121.69 mg, 634.81 umol, 3 eq) and HOBt (85.78 mg, 634.81 umol, 3 eq) in DMF (1 mL), followed by TEA (107.06 mg, 1.06 mmol, 147.26 uL, 5 eq). The mixture was stirred at 25° C. for 1 hr. LC-MS showed desired mass was detected. The reaction mixture was filtered to obtain filtrate, which was purified by prep-HPLC (column: Phenomenex Synergi C18 150*30 mm*4 um; mobile phase: A: 0.225% formic acid in water, B: CH₃CN; gradient: 60%-90% B over 11 min). Compound 2-cyclopentyl-N-(1,1-dimethylsilinan-4-yl)-4H-pyrrolo[2,3-d] thiazole-5-carboxamide (57.4 mg, 158.75 umol, 75.02% yield, 100% purity) was obtained as a white solid.

LCMS (ESI) m/z: 362.2 [M+H]⁺; ¹H NMR (400 MHz, DMSO-d₆) δ=12.16 (s, 1H), 7.87 (d, J=8.2 Hz, 1H), 7.15-6.92 (m, 1H), 3.73-3.61 (m, 1H), 3.44 (quin, J=7.8 Hz, 1H), 2.16-2.05 (m, 2H), 1.97 (br d, J=9.8 Hz, 2H), 1.87-1.49 (m, 8H), 0.81-0.76 (m, 2H), 0.65-0.53 (m, 2H), 0.14-0.03 (m, 6H).

### Example 49, MPL-475

### Synthesis of 2-(cyclobutoxy)-N-(1,1-dimethylsilepan-4-yl)-4H-pyrrolo[2,3-d]thiazole-5-carboxamide

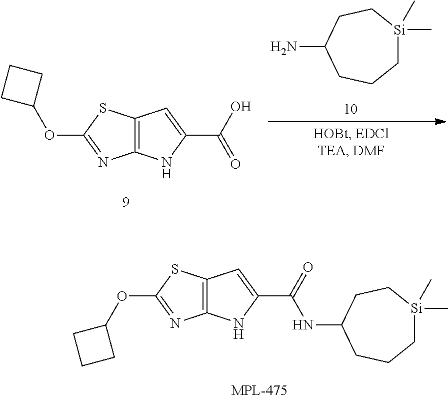

To a solution of 2-(cyclobutoxy)-4H-pyrrolo[2,3-d]thiazole-5-carboxylic acid (50 mg, 209.85 umol, 1 eq) and 1,1-dimethylsilepan-4-amine (48.80 mg, 251.82 umol, 1.2 eq, HCl salt) in DMF (1 mL) at 25° C., a solution of HOBt (85.07 mg, 629.56 umol, 3 eq) and EDCI (120.69 mg, 629.56 umol, 3 eq) in DMF (1 mL) was added, followed by TEA (106.17 mg, 1.05 mmol, 146.04 uL, 5 eq). The reaction mixture was stirred at 25° C. for 2 hr. LC-MS showed desired compound was detected. The mixture was purified by prep-HPLC (column: Phenomenex Synergi C18 150*30 mm*4 um; mobile phase: A: 0.225% formic acid in water, B: CH₃CN; gradient: 62%-92% B over 11 min). Compound 2-(cyclobutoxy)-N-(1,1-dimethylsilepan-4-yl)-4H-pyrrolo[2,3-d]thiazole-5-carboxamide (26.6 mg, 70.45 umol, 33.57% yield, 100% purity) was obtained as a white solid.

LCMS (ESI) m/z 378.3 [M+H]⁺; ¹H NMR (500 MHz, DMSO-d₆) δ=12.02 (br s, 1H), 7.75 (d, J=7.9 Hz, 1H), 6.96 (s, 1H), 5.15 (quin, J=7.3 Hz, 1H), 3.82 (br d, J=8.4 Hz, 1H), 2.47-2.38 (m, 2H), 2.24-2.09 (m, 2H), 1.93-1.38 (m, 8H), 0.82-0.52 (m, 4H), 0.02 (d, J=7.3 Hz, 6H).

### Example 50, MPL-478

### Synthesis of 2-(cyclopentoxy)-N-(1,1-dimethylsilepan-4-yl)-4H-pyrrolo[2,3-d]thiazole-5-carboxamide

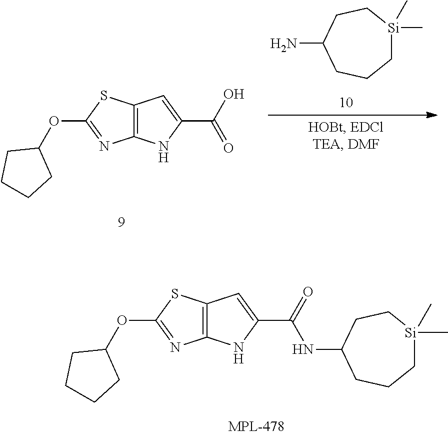

To a solution of 2-(cyclopentoxy)-4H-pyrrolo[2,3-d]thiazole-5-carboxylic acid (50 mg, 198.19 umol, 1 eq) and 1,1-dimethylsilepan-4-amine (46.09 mg, 237.82 umol, 1.2 eq, HCl salt) in DMF (1 mL) was added a solution of HOBt (80.34 mg, 594.56 umol, 3 eq) and EDCI (113.98 mg, 594.56 umol, 3 eq) in DMF (1 mL), followed by TEA (100.27 mg, 990.93 umol, 137.93 uL, 5 eq). The reaction was stirred at 25° C. for 2 hr. LC-MS showed desired compound was detected. The mixture was purified by prep-HPLC (column: Phenomenex Synergi C18 150*30 mm*4 um; mobile phase: A: 0.225% formic acid in water, B: CH₃CN; gradient: 67%-97% B over 11 min). Compound 2-(cyclopentoxy)-N-(1,1-dimethylsilepan-4-yl)-4H-pyrrolo[2,3-d]thiazole-5-carboxamide (26.6 mg, 67.93 umol, 34.27% yield, 100% purity) was obtained as a white solid.

LCMS (ESI) m/z 392.3 [M+H]⁺; ¹H NMR (500 MHz, DMSO-d₆) δ=12.01 (br s, 1H), 7.75 (br d, J=7.8 Hz, 1H), 6.95 (s, 1H), 5.41-5.29 (m, 1H), 3.83 (br s, 1H), 2.04-1.36 (m, 14H), 0.82-0.51 (m, 4H), 0.02 (d, J=7.3 Hz, 6H).

### Example 51, MPL-311

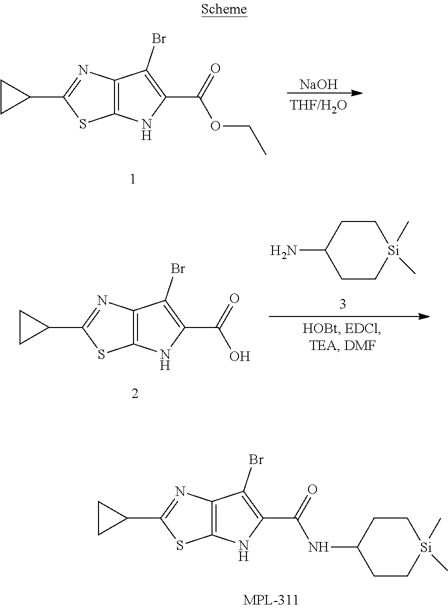

### Step 1. Synthesis of 6-bromo-2-cyclopropyl-4H-pyrrolo[3,2-d]thiazole-5-carboxylic acid

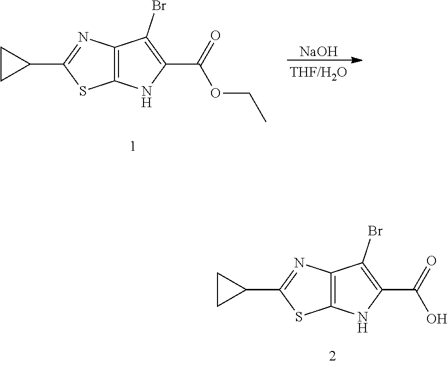 [SMILES: CCOC(=O)c1[nH]c2sc(C3CC3)nc2c1Br ; "NaOH" ; O=C(O)c1[nH]c2sc(C3CC3)nc2c1Br]

To a solution of ethyl 6-bromo-2-cyclopropyl-4H-pyrrolo[3,2-d]thiazole-5-carboxylate (100 mg, 317.27 umol, 1 eq) in THF (2 mL) was added NaOH (2 M in water, 2 mL, 12.61 eq). The mixture was stirred at 50° C. for 16 hr. LCMS indicated that the desired mass was detected. The reaction mixture was adjusted to pH to 3 by 1 M HCl (6 ml) and extracted with EtOAc 6 mL (2 mL×3). The combined organic layer was dried over Na₂SO₄ and concentrated under reduced pressure. Compound 6-bromo-2-cyclopropyl-4H-pyrrolo[3,2-d]thiazole-5-carboxylic acid (90 mg, crude) was obtained as a brown solid. The crude product was used for the next step without purification.

LCMS m/z: 288.8 [M+H]⁺

### Step 2. Synthesis of 6-bromo-2-cyclopropyl-N-(1,1-dimethylsilinan-4-yl)-4H-pyrrolo[3,2-d]thiazole-5-carboxamide

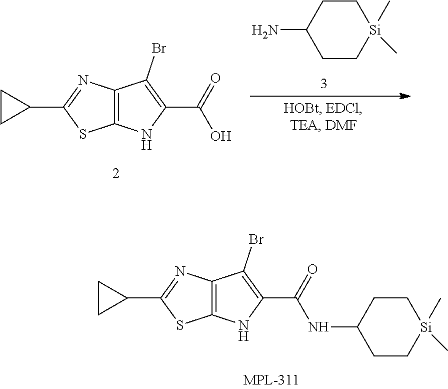

To a solution of 6-bromo-2-cyclopropyl-4H-pyrrolo[3,2-d]thiazole-5-carboxylic acid (90 mg, 313.44 umol, 1 eq) and 1,1-dimethylsilinan-4-amine (44.92 mg, 313.44 umol, 1 eq) in DMF (2 mL) was added HOBt (127.06 mg, 940.33 umol, 3 eq) and EDCI (180.26 mg, 940.33 umol, 3eq), followed by TEA (190.30 mg, 1.88 mmol, 261.77 uL, 6 eq). The mixture was stirred at 30° C. for 1 hr. LC-MS showed desired compound was detected. The reaction mixture was diluted with H₂O (10 mL) and extracted with EtOAC (10 mL×2). The combined organic layer was washed with 5% LiCl in water (10 mL×2), dried over Na₂SO₄, and filtered and concentrated under reduced pressure. The residue was purified by prep-HPLC (column: YMC-Actus Triart C18 150×30 mm×5 um; mobile phase: A: 0.225% formic acid in water, B: CH₃CN; gradient:70%-100% B over 11 min). Compound 6-bromo-2-cyclopropyl-N-(1,1-dimethylsilinan −4-yl)-4H-pyrrolo[3,2-d]thiazole-5-carboxamide (21.6 mg, 51.40 umol, 16.40% yield, 98.14% purity) was obtained as a white solid.

LCMS m/z: 411.9 [M+1]⁺; ¹H NMR (400 MHz, METHANOL-d₄) δ=4.03-3.79 (m, 1H), 2.33 (tt, J=4.9, 8.3 Hz, 1H), 2.18-2.05 (m, 2H), 1.84-1.67 (m, 2H), 1.25-1.15 (m, 2H), 1.13-1.05 (m, 2H), 0.89-0.79 (m,2H), 0.78-0.63 (m, 2H), 0.09 (d, J=17.4 Hz, 6H).

### Example 52, MPL-312

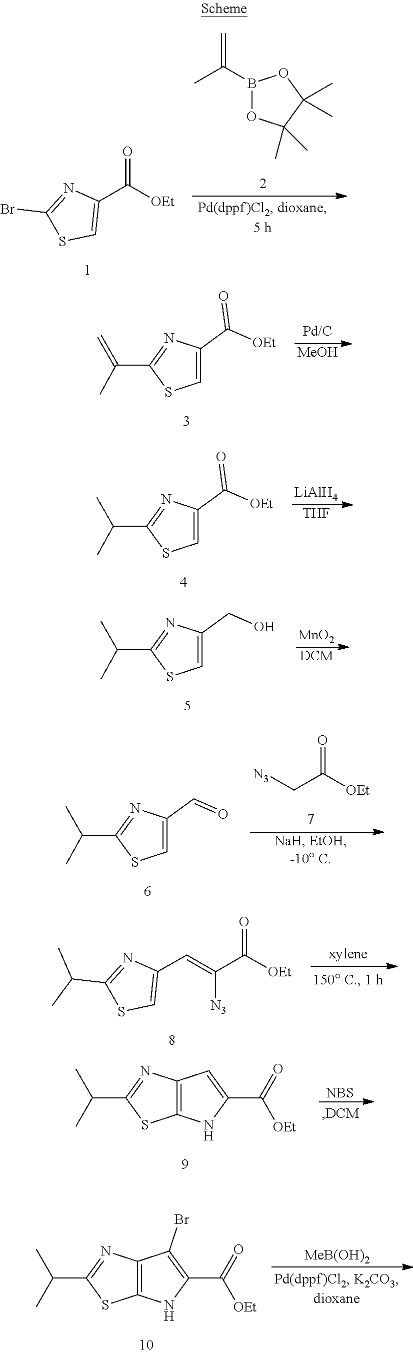

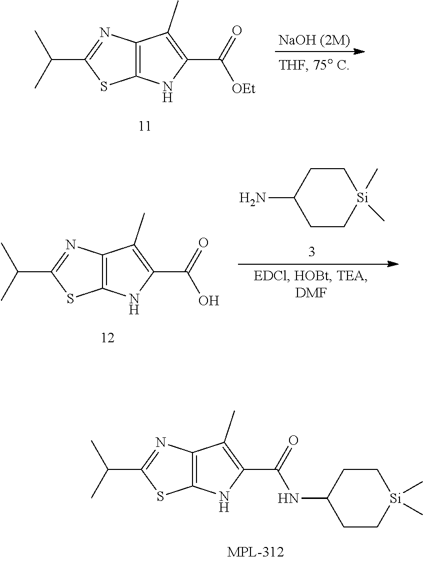

### Step 1. Synthesis of ethyl 2-isopropenylthiazole-4-carboxylate

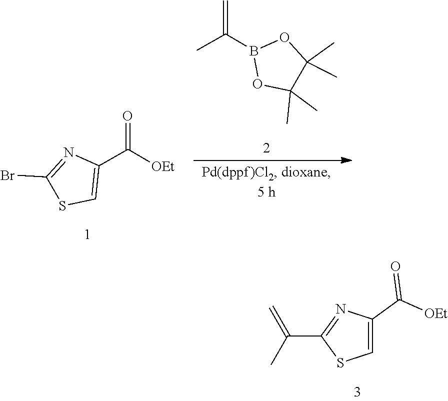

To a mixture of ethyl 2-bromothiazole-4-carboxylate (16 g, 67.77 mmol, 1 eq) and 2-isopropenyl-4,4,5,5-tetramethyl-1,3,2-dioxaborolane (34.17 g, 203.32 mmol, 3 eq) in dioxane (160 mL) was added K₃PO₄ (21.58 g, 101.66 mmol, 1.5 eq) and cyclopentyl(diphenyl)phosphane;dichloropalladium;iron (495.89 mg, 677.72 umol, 0.01 eq) under N₂. The mixture was stirred at 100° C. for 5 hr under N₂. LCMS showed desired mass was detected. The reaction mixture was concentrated under reduced pressure. The residue was diluted with H₂O (160 mL) and extracted with EtOAc (160 mL×2). The combined organic layer was dried over Na₂SO₄, and then filtered and concentrated under reduced pressure to give a residue which was purified by flash silica gel chromatography (0-30% Ethyl acetate in petroleum ether). Compound ethyl 2-isopropenylthiazole-4-carboxylate (3.3 g, 16.73 mmol, 23.29% yield, 100% purity) was obtained as a yellow liquid.

LCMS (ESI) m/z 198.0 [M+H]⁺; ¹H NMR was recorded.

### Step 2. Synthesis of ethyl 2-isopropylthiazole-4-carboxylate

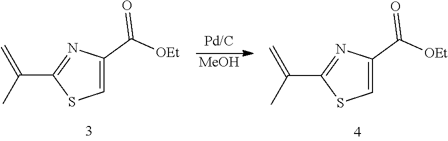

A mixture of ethyl 2-isopropenylthiazole-4-carboxylate (3.30 g, 16.73 mmol, 1 eq) and Pd/C (0.5 g, 10% purity) in MeOH (50 mL) was degassed and purged with H₂ for 3 times, and then stirred at 25° C. for 16 hr under H₂ atmosphere. LCMS indicated desired mass was detected. The reaction mixture was filtered and concentrated under reduced pressure. Compound ethyl 2-isopropylthiazole-4-carboxylate (3.2 g, 14.45 mmol, 86.39% yield, 90% purity) was obtained as a red oil.

LCMS (ESI) m/z 200.0 [M+H]⁺; ¹H NMR was recorded.

### Step 3. Synthesis of ethyl (2-isopropylthiazol-4-yl)methanol

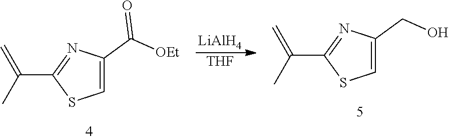

To the mixture of ethyl 2-isopropylthiazole-4-carboxylate (2.2 g, 11.04 mmol, 1 eq) in THF (25 mL) at 0° C. under N₂ was added LiAlH₄ (440.00 mg, 11.59 mmol, 1.05 eq). The mixture was stirred at 0° C. for 10 min. TLC showed the reactant was consumed completely. The reaction was quenched by addition of H₂O (0.44 mL), NaOH (15% in water, 0.44 mL) and H₂O (1.32 mL), and then filtered. The filter cake was washed with EtOAC (50 mL). The filtrate was dried over by Na₂SO₄ and concentrated in vacuo. Ethyl (2-isopropylthiazol-4-yl)methanol (1.30 g, crude) was obtained as a yellow oil. The crude product was used for the next step without purification.

### Step 4. Synthesis of 2-isopropylthiazole-4-carbaldehyde

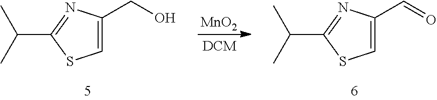

To a solution of (2-isopropylthiazol-4-yl)methanol (1.3 g, 8.27 mmol, 1 eq) in DCM (20 mL) was added MnO₂ (7.19 g, 82.68 mmol, 10 eq). The mixture was stirred at 25° C. for 6 hr. TLC showed the reactant was consumed and a new spot formed. The mixture was filtered. The filter cake was washed with DCM (40 mL). The combined filtrate was concentrated under reduce pressure. The residue was purified by column chromatography (SiO₂, 0-20% Ethyl acetate in petroleum ether) to afford 2-isopropylthiazole-4-carbaldehyde (800 mg, 5.15 mmol, 62.34% yield, 100% purity) as a yellow oil.

### Step 5. Synthesis of ethyl (Z)-2-azido-3-(2-isopropylthiazol-4-yl)prop-2-enoate

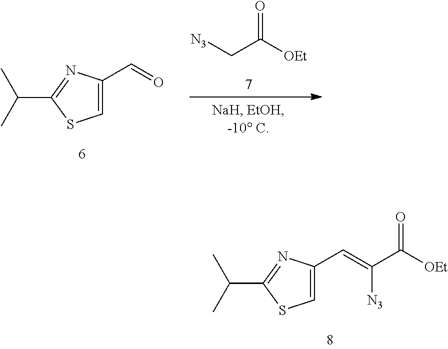

NaH (1.26 g, 31.57 mmol, 60% purity, 5 eq) was added into EtOH (5 mL) and the mixture was stirred at −10° C. for 0.1 h. Then a mixture of 2-isopropylthiazole-4-carbaldehyde (980 mg, 6.31 mmol, 1 eq) and ethyl 2-azidoacetate (2.45 g, 18.94 mmol, 2.66 mL, 3 eq) in EtOH (5 mL) was added dropwise at −10° C. The reaction mixture was stirred for 2.9 h at the same temperature. TLC indicated reactant 6 was consumed completely, and one new spot formed. The reaction mixture was concentrated under reduce pressure. The residue was purified by column chromatography (SiO2, 0-20% Ethyl acetate in petroleum ether) to afford ethyl (Z)-2-azido-3-(2-isopropylthiazol-4-yl)prop-2-enoate (680 mg, 2.43 mmol, 38.42% yield, 95% purity) as a yellow oil.

### Step 6. Synthesis of ethyl 2-isopropyl-4H-pyrrolo[3,2-d]thiazole-5-carboxylate

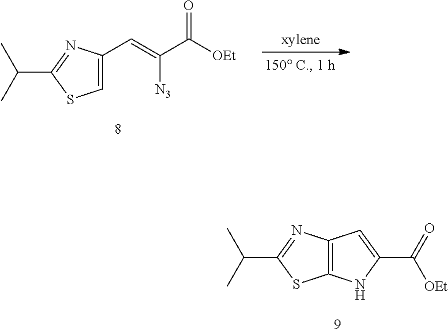 [SMILES: CCOC(=O)/C(=C/c1csc(C(C)C)n1)N=[N+]=[N-] ; CCOC(=O)c1cc2nc(C(C)C)sc2[nH]1]

A solution of ethyl (Z)-2-azido-3-(2-isopropylthiazol-4-yl)prop-2-enoate (680 mg, 2.55 mmol, 1 eq) in xylene (5 mL) was stirred at 150° C. for 1 hr. TLC showed the reactant was consumed completely and one new spot formed. The mixture was concentrated under reduce pressure. Compound ethyl 2-isopropyl-4H-pyrrolo[3,2-d]thiazole-5-carboxylate (380 mg, 1.51 mmol, 52.27% yield, 95% purity) was obtained as a yellow solid. ¹H NMR was recorded.

### Step 7. Synthesis of ethyl 6-bromo-2-isopropyl-4H-pyrrolo[3,2-d]thiazole-5-carboxylate

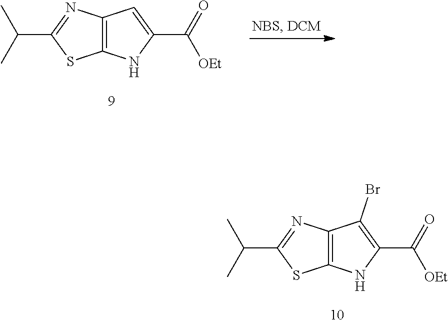 [SMILES: CCOC(=O)c1cc2nc(C(C)C)sc2[nH]1 ; CCOC(=O)c1[nH]c2sc(C(C)C)nc2c1Br]

To a solution of ethyl 2-isopropyl-4H-pyrrolo[3,2-d]thiazole-5-carboxylate (330 mg, 1.38 mmol, 1 eq) in DCM (5 mL) was added NBS (492.94 mg, 2.77 mmol, 2 eq). The reaction mixture was stirred at 0° C. for 30 min. LCMS showed desired compound was detected. The reaction mixture was quenched by saturated sodium sulfite (10 mL) and stirred for 15 min. Then the mixture was diluted with DCM (10 mL). The organic layer was separated and dried over Na₂SO₄, filtered and concentrated under reduced pressure. The residue was purified by column chromatography (SiO2, 0-20% Ethyl acetate in petroleum ether). Compound ethyl 6-bromo-2-isopropyl-4H-pyrrolo[3,2-d]thiazole-5-carboxylate (350 mg, 1.05 mmol, 65.82% yield, 95% purity) was obtained as a white solid.

LCMS (ESI) m/z 317.0 [M+H]⁺; ¹H NMR (400 MHz, METHANOL-d₄) δ=4.34 (q, J=7.1 Hz, 2H), 3.36-3.30 (m, 1H), 1.47-1.30 (m, 9H).

### Step 8. Synthesis of ethyl 2-isopropyl-6-methyl-4H-pyrrolo[3,2-d]thiazole-5-carboxylate

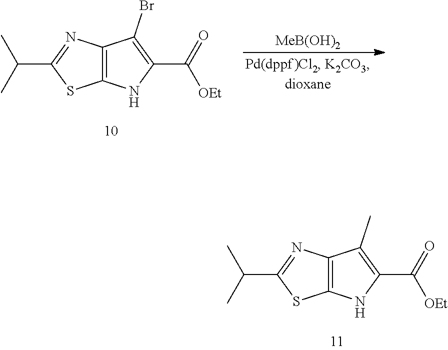 [SMILES: "MeB(OH)2" ; CCOC(=O)c1[nH]c2sc(C(C)C)nc2c1Br ; CCOC(=O)c1[nH]c2sc(C(C)C)nc2c1C]

To a mixture of ethyl 6-bromo-2-isopropyl-4H-pyrrolo[3,2-d]thiazole-5-carboxylate (260 mg, 819.67 umol, 1 eq), methylboronic acid (490.65 mg, 8.20 mmol, 10 eq) and K₂CO₃ (453.15 mg, 3.28 mmol, 4 eq) was added dioxane (10 mL). Then Pd(dppf)Cl₂ (299.88 mg, 409.83 umol, 0.5 eq) was added under N₂. The mixture was stirred at 110° C. for 12 hr under N₂. LCMS showed the desired mass was detected. The mixture was filtered. The filter cake was washed with EtOAc (5 mL×2). The combined filtrate was evaporated. The residue was purified by column chromatography (SiO₂, 0-20% Ethyl acetate in petroleum ether). Compound ethyl 2-isopropyl-6-methyl-4H-pyrrolo[3,2-d]thiazole-5-carboxylate (98 mg, 343.24 umol, 41.88% yield, 88.38% purity) was obtained as a yellow solid.

LCMS (ESI) m/z 253.0 [M+H]⁺

### Step 9. Synthesis of 2-isopropyl-6-methyl-4H-pyrrolo[3,2-d]thiazole-5-carboxylic acid

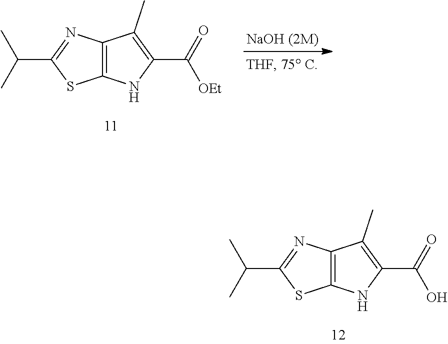 [SMILES: CCOC(=O)c1[nH]c2sc(C(C)C)nc2c1C ; Cc1c(C(=O)O)[nH]c2sc(C(C)C)nc12]

To a solution of ethyl 2-isopropyl-6-methyl-4H-pyrrolo[3,2-d]thiazole-5-carboxylate (116 mg, 459.71 umol, 1 eq) in THF (2 mL) was added NaOH (2 M, 2 mL, 8.70 eq) (in water). The mixture was stirred at 75° C. for 1 hr. LCMS showed the desired compound was detected. The reaction mixture was adjusted to pH to 3 with 1 M HCl (6 ml) and then extracted with EtOAc 6 mL (2 mL×3). The combined organic layer was dried over Na₂SO₄ and concentrated under reduced pressure. Compound 2-isopropyl-6-methyl-4H-pyrrolo[3,2-d]thiazole-5-carboxylic acid (70 mg, crude) was obtained as a brown solid. The crude product was used for the next step without purification.

LCMS (ESI) m/z 224.9 [M+H]⁺

### Step 10. Synthesis of N-(1,1-dimethylsilinan-4-yl)-2-isopropyl-6-methyl-4H-pyrrolo[3,2-d]thiazole-5-carboxamide

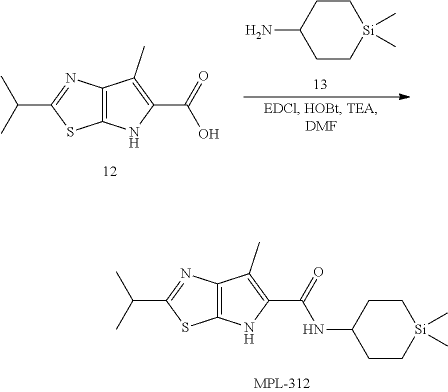

To a solution of 2-isopropyl-6-methyl-4H-pyrrolo[3,2-d]thiazole-5-carboxylic acid (70 mg, 312.11 umol, 1 eq), 1,1-dimethylsilinan-4-amine (67.33 mg, 374.53 umol, 1.2 eq, HCl) in DMF (1 mL) was added HOBt (63.26 mg, 468.17 umol, 1.5 eq), EDCI (89.75 mg, 468.17 umol, 1.5 eq) and TEA (94.75 mg, 936.33 umol, 130.33 uL, 3 eq). The mixture was stirred at 25° C. for 1 hr. LCMS showed desired compound was detected. The reaction mixture was diluted with CH₃OH (2 mL) and filtered. The filtrate was purified by prep-HPLC (column: YMC-Actus Triart C18 150×30 mm×5 um; mobile phase: A: 0.225% formic acid in water, B: CH₃CN; gradient: 59%-89% B over 11 min). Compound N-(1,1-dimethylsilinan-4-yl)-2-isopropyl-6-methyl-4H-pyrrolo[3,2-d]thiazole-5-carboxamide (16.4 mg, 46.92 umol, 15.03% yield, 100% purity) was obtained as a white solid.

LCMS (ESI) m/z 350.3 [M+H]⁺; ¹H NMR (400 MHz, METHANOL-d₄) δ=7.21 (br d, J=7.4 Hz, 1H), 3.82-3.71 (m, 1H), 3.37-3.33 (m, 1H), 2.55 (s, 3H), 2.19-2.10 (m,2H), 1.74-1.61 (m, 2H), 1.42 (d, J=6.7 Hz, 6H), 0.87-0.79 (m, 2H), 0.77-0.65 (m, 2H), 0.12 (s, 3H), 0.05 (s, 3H).

### Example 53, MPL-313

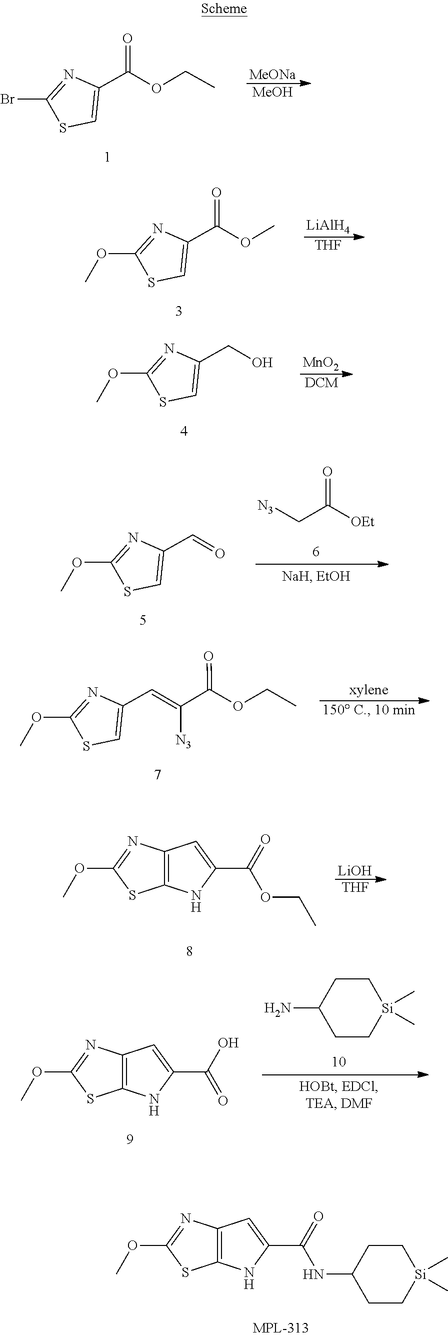

### Step 1. Synthesis of methyl 2-methoxythiazole-4-carboxylate

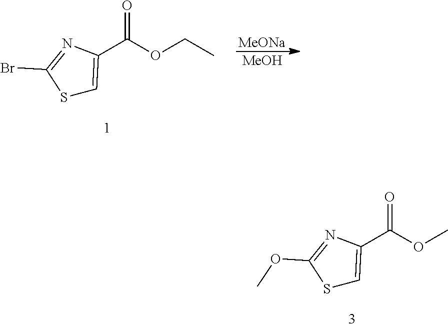

To a solution of ethyl 2-bromothiazole-4-carboxylate (10 g, 42.36 mmol, 1 eq) in MeOH (100 mL) was added NaOMe (38.14 g, 211.79 mmol, 30% in MeOH, 5 eq). The mixture was stirred at 70° C. for 3 hr. TLC indicated reactant 1 was consumed completely. The reaction was quenched by addition of aqueous HCl (6 M) until pH 2. The mixture was diluted with water (100 mL), and then extracted with EtOAc (100 mL×2). The combined organic layer was dried over Na₂SO₄, and then filtered and concentrated in vacuo. Compound methyl 2-methoxythiazole-4-carboxylate (7.78 g, crude) was obtained as a brown oil. The crude product was used for the next step without purification. ¹H NMR was recorded.

### Step 2. Synthesis of (2-methoxythiazol-4-yl)methanol

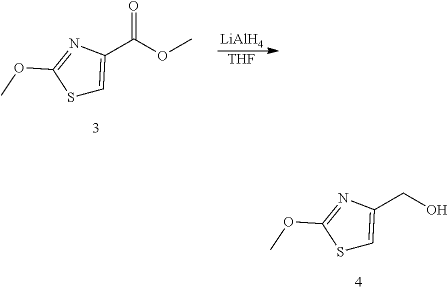

To an ice-cooled solution of methyl 2-methoxythiazole-4-carboxylate (7.78 g, 44.90 mmol, 1 eq) in dried THF (100 mL) was added LiAlH₄ (2.5 g, 65.88 mmol, 1.47 eq) in batches. The mixture was stirred at 0-20° C. for 1 hr. TLC indicated reactant 3 was consumed completely. The reaction was quenched with water (2.5 mL), NaOH (15%, 2.5 mL) and water (7.5 mL). The mixture was then filtered. The filter cake was washed with DCM (100 mL×10). The combined filtrate was dried over Na₂SO₄, and then filtered and concentrated under reduced pressure. The residue was purified by column chromatography (SiO₂, 0-66% Ethyl acetate in petroleum ether). Compound (2-methoxythiazol-4-yl)methanol (1.06 g, 6.55 mmol, 14.58% yield, 90% purity) was obtained as a yellow oil. ¹H NMR was recorded.

### Step 3. Synthesis of 2-methoxythiazole-4-carbaldehyde

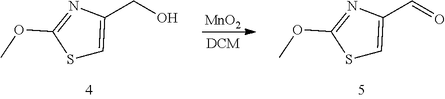

To a solution of (2-methoxythiazol-4-yl)methanol (1.15 g, 7.92 mmol, 1 eq) in DCM (10 mL) was added MnO₂ (6.89 g, 79.21 mmol, 10 eq). The mixture was stirred at 25° C. for 5 hr. TLC (Petroleum ether: EtOAc=3:1) showed reactant 4 was consumed completely, and one major new spot with lower polarity formed. The mixture was filtered. The filter cake was washed with EtOAc (10 mL×5). The combined filtrate was dried over Na₂SO₄, and then filtered and concentrated under reduced pressure. The residue was purified by column chromatography (SiO₂, 0-25% Ethyl acetate in petroleum ether). Compound 2-methoxythiazole-4-carbaldehyde (914 mg, 5.75 mmol, 72.54% yield, 90% purity) was obtained as a yellow solid. ¹H NMR was recorded.

### Step 4. Synthesis of ethyl (Z)-2-azido-3-(2-methoxythiazol-4-yl)prop-2-enoate

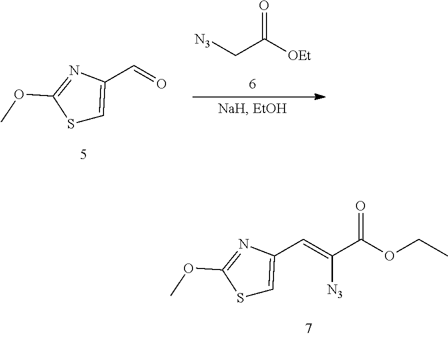

NaH (1.28 g, 31.92 mmol, 60% purity, 5 eq) was added to EtOH (10 mL) in batches. The mixture was stirred at 30° C. until a clear solution formed, and then cooled to −10° C. Then a solution of 2-methoxythiazole-4-carbaldehyde (914 mg, 6.38 mmol, 1 eq) and ethyl 2-azidoacetate (4.12 g, 31.92 mmol, 4.48 mL, 5 eq) in EtOH (10 mL) was added to the mixture dropwise. The mixture was stirred at −10° C.˜0° C. for 2 hr. LCMS showed reactant 5 was consumed completely and desired mass was detected. The reaction was quenched with HCl (3 M in water, about 5 eq) until pH turned to 6, concentrated under reduced pressure until ⅕ of the original volume left, and then extracted with EtOAc (50 mL×2). The combined organic layer was washed with brine (50 mL×2), dried over Na₂SO₄, filtered and concentrated under reduced pressure to give a residue which was purified by column chromatography (SiO₂, 0-30% Ethyl acetate in petroleum ether). Compound ethyl (Z)-2-azido-3-(2-methoxythiazol-4-yl)prop-2-enoate (500 mg, crude) was obtained as a yellow oil. ¹H NMR was recorded.

### Step 5. Synthesis of ethyl 2-methoxy-4H-pyrrolo[3,2-d]thiazole-5-carboxylate

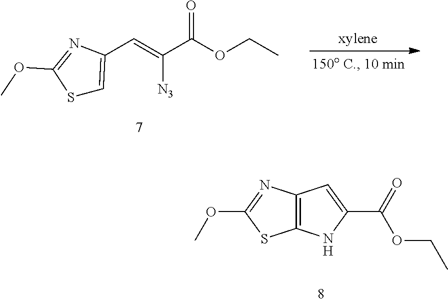

Ethyl (Z)-2-azido-3-(2-methoxythiazol-4-yl)prop-2-enoate (500 mg, 1.97 mmol, 1 eq) in xylene (2 mL) was stirred at 150° C. for 10 min. TLC (Petroleum ether: EtOAc=3:1) indicated reactant 7 was consumed completely. The mixture was filtered. The cake was washed with petroleum ether (5 mL×2) and collected. Compound ethyl 2-methoxy-4H-pyrrolo[3,2-d]thiazole-5-carboxylate (440 mg, 1.94 mmol, 98.90% yield, 100% purity) was obtained as a yellow solid. ¹H NMR was recorded.

### Step 6. Synthesis of 2-methoxy-4H-pyrrolo[3,2-d]thiazole-5-carboxylic acid

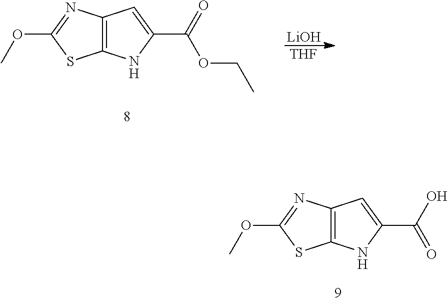

To a solution of ethyl 2-methoxy-4H-pyrrolo[3,2-d]thiazole-5-carboxylate (80 mg, 353.59 umol, 1 eq) in THF (1 mL) was added a solution of LiOH.H₂O (59.35 mg, 1.41 mmol, 4 eq) in H₂O (1 mL). The mixture was stirred at 70° C. for 2 hr. LCMS showed the starting material remained. The mixture was stirred at 70° C. for additional 12 hr. LCMS showed reactant 8 was consumed completely and desired mass was detected. The reaction mixture was concentrated under reduced pressure to remove THF, and then acidified with HCl (6 M in water) to pH 3, and then extracted with EtOAc (10 mL×3). The combined organic layer was washed with brine (10 mL×2), dried over Na₂SO₄, and then filtered and concentrated under reduced pressure. The resulting residue was diluted with CH₃CN (5 mL) and water (5 mL) and then lyophilized. Compound 2-methoxy-4H-pyrrolo[3,2-d]thiazole-5-carboxylic acid (20 mg, 95.86 umol, 27.11% yield, 95% purity) was obtained as a brown solid.

¹H NMR (500 MHz, DMSO-d₆) δ=12.54 (br, s, 1H), 12.03 (s, 1H), 6.89 (s, 1H), 4.05 (s, 3H).

### Step 7. N-(1,1-dimethylsilinan-4-yl)-2-methoxy-4H-pyrrolo[3,2-d]thiazole-5-carboxamide

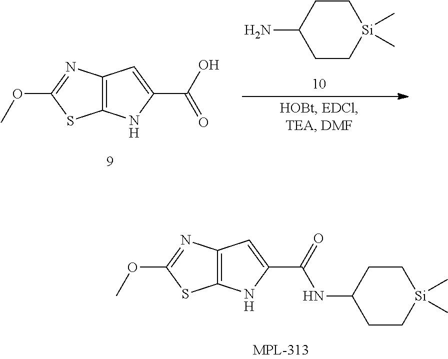

To a solution of 2-methoxy-4H-pyrrolo[3,2-d]thiazole-5-carboxylic acid (20 mg, 100.91 umol, 1 eq) and 1,1-dimethylsilinan-4-amine (21.77 mg, 121.09 umol, 1.2 eq, HCl salt) in DMF (1 mL) was added a solution of HOBt (40.91 mg, 302.73 umol, 3 eq) and EDCI (58.03 mg, 302.73 umol, 3 eq) in DMF (1 mL), followed by TEA (51.05 mg, 504.54 umol, 70.23 uL, 5 eq). The reaction was stirred at 25° C. for 12 hr. LCMS showed the starting material remained. Additional 1,1-dimethylsilinan-4-amine HCl salt (5.44 mg, 0.3 eq) was added. The reaction mixture was stirred at 25° C. for 12 h. LCMS showed the starting material remained. HOBt (13.64 mg, 1 eq) and EDCI (19.34 mg, 1 eq) were added, and the reaction mixture was stirred at 25° C. for 3 h. LCMS showed the reaction completed. The mixture was purified by prep-HPLC (column: YMC-Actus Triart C18 150*30 mm*5 um; mobile phase: A: 0.225% formic acid in water, B: CH₃CN; gradient:35%-65% B over 11 min). Compound N-(1,1-dimethylsilinan-4-yl)-2-methoxy-4H-pyrrolo[3,2-d]thiazole-5-carboxamide (11.8 mg, 36.24 umol, 35.91% yield, 99.34% purity) was obtained as a white solid.

LCMS (ESI) m/z 324.1 [M+H]⁺; ¹H NMR (500 MHz, DMSO-d₆) δ=11.66 (s, 1H), 7.85 (d, J=8.2 Hz, 1H), 6.99 (s, 1H), 4.04 (s, 3H), 3.65 (dt, J=8.5, 11.3 Hz, 1H), 1.99-1.93 (m, 2H), 1.62-1.47 (m, 2H), 0.75 (br d, J=14.6 Hz, 2H), 0.59 (dt, J=4.7, 14.2 Hz, 2H), 0.08 (s, 3H), 0.02 (s, 3H).

### Example 54: Biological Experiments

MIC (Minimum Inhibitory Concentration) determination of anti-tuberculosis drugs. The antituberculosis activity of each compound against M. tb H37Rv was measured by the green fluorescent protein reporter assay (Collins 1998). Briefly, the compound was initially dissolved in dimethylsulfoxide (DMSO), and two fold dilutions were made in DMSO. The same amount of each dilution of compound solution was added to 7H9 broth in microplates. The initial inoculum of 2×10⁵ CFU/ml of Mtb H37Rv-GFP that was grown in Middlebrook 7H9 media was exposed to the compound for 10 days. The fluorescence was measured in a Fluostar Optima microplate fluorometer (BMG Labtech, Germany), and the MIC was defined as the lowest concentration of compounds that inhibited fluorescence by 90% comparing to the fluorescence of bacteria only wells. CFU=colony forming units. Column 1 of Table 1 shows anti Mycobacterium tuberculosis activity of representative compounds of the invention. The procedures described in Collins 1997 were used to generate the data shown in column 1.

Column 2 of Table 1 shows anti Mycobacterium abscessus activity of representative compounds of the invention. The procedure described in Franz 2017 was used to generate the data shown in column 2 of Table 1.

TABLE 1

| | 1 | 2 |
|---|---|---|
| | M. tb H37Rv: MIC- | M. ab_ATCC: MIC |
| Compound | MABA (μg/mL) | MHII: MIC (μg/mL) |
| MPL-273 | 0.48 | >64 |
| MPL-272 | 0.96 | >64 |
| MPL-224 | 0.39 | >64 |
| MPL-240 | 0.46 | 0.5 |
| MPL-228 | 0.061 | >64 |
| MPL-150 | 0.3 | >64 |
| MPL-142 | 0.93 | >64 |
| MPL-205 | 0.18 | >64 |
| MPL-144 | 1.5 | >64 |
| MPL-042 | 0.35 | >64 |
| MPL-025 | 0.71 | 4 |
| MPL-020 | 0.53 | 2 |
| MPL-026 | 3.1 | nd |
| MPL-021 | 1.3 | >64 |
| MPL-269 | >1.0 | >64 |
| MPL-268 | >1.0 | >64 |
| MPL-288 | 0.72 | >64 |
| MPL-206 | >1.6 | >64 |
| MPL-091 | >3.2 | >64 |
| MPL-090 | 3.2 | >64 |
| MPL-291 | 0.372 | >64 |
| MPL-293 | 0.062 | >64 |
| MPL-297 | 0.982 | >64 |
| MPL-304 | 0.291 | >64 |
| MPL-308 | 0.007 | >64 |
| MPL-309 | 0.088 | 0.625 |
| MPL-311 | 0.457 | >64 |
| MPL-312 | 0.498 | >64 |
| MPL-313 | 0.254 | >64 |
| MPL-357 | 0.012 | >64 |
| MPL-357A | 0.007 | nd |
| MPL-357B | 0.110 | nd |
| MPL-358 | 0.033 | >64 |
| MPL-359 | 0.014 | >64 |
| MPL-364 | 0.423 | >64 |
| MPL-365 | 0.125 | >64 |
| MPL-369 | 0.031 | >64 |
| MPL-370 | 0.574 | >64 |
| MPL-371 | 0.041 | >64 |
| MPL-372 | 0.138 | >64 |
| MPL-373 | 0.084 | 6 |
| MPL-393 | 0.062 | >64 |
| MPL-394 | 0.040 | >64 |
| MPL-395 | 0.047 | 1 |
| MPL-395A | 0.030 | 0.5 |
| MPL-395B | 0.999 | >64 |
| MPL-396 | 0.309 | >64 |
| MPL-403 | 0.809 | 4 |
| MPL-404 | 0.083 | 64 |
| MPL-426 | 0.120 | nd |
| MPL-427 | 0.036 | >64 |
| MPL-429 | 0.058 | 64 |
| MPL-431 | 0.021 | >64 |
| MPL-433A | >1 | nd |
| MPL-433B | 0.69 | nd |
| MPL-456 | 0.317 | 1 |
| MPL-457 | >1 | nd |
| MPL-458 | 0.124 | >64 |
| MPL-459 | 0.0593 | >64 |
| MPL-472 | 0.060 | >64 |
| MPL-474 | 0.061 | >64 |
| MPL-475 | 0.046 | 16 |
| MPL-478 | 0.020 | >64 |

Key: MIC: Minimum Inhibitory Concentration; MABA: microplate-based Alamar Blue assay; Mab: Mycobacterium abscessus; ATCC: American Type Culture Collection.

In summary, the compounds of the invention exhibit potent anti-Mycobacterium activity (against Mycobacterium tuberculosis and non-tuberculosis Mycobacterium infections).

### Example 55

Compounds are prepared having the structures shown below:

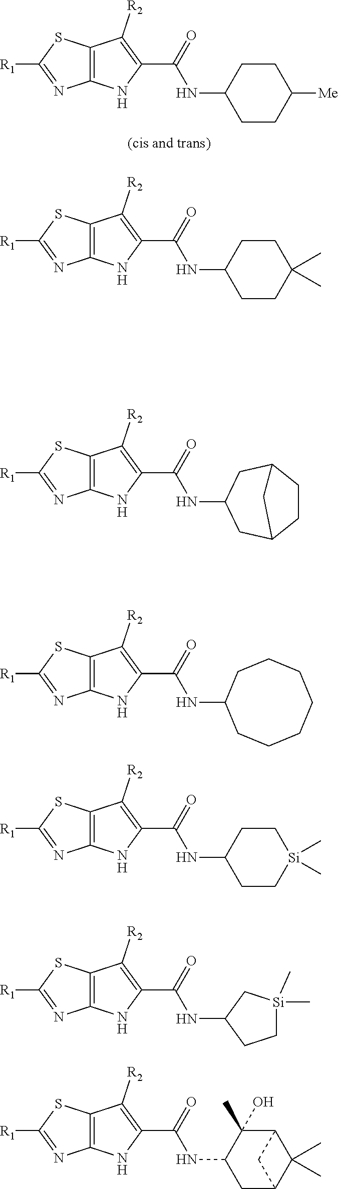

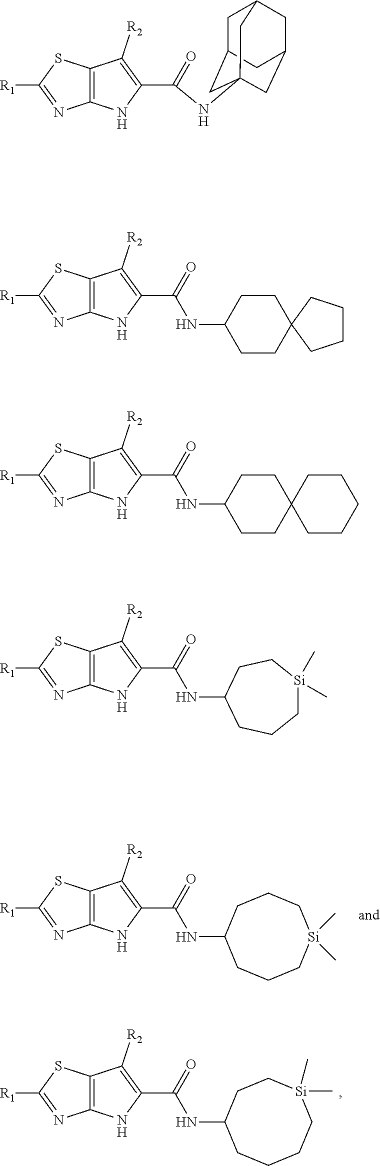

wherein R₁ may be hydrogen, (C₁-C₁₁)alkyl, cycloalkyl, aryl, heteroaryl, alkoxy, or cycloalkoxy; and R2 may be hydrogen, alkyl, cycloalkyl, CN, or halogen.

It is contemplated that these compounds will show anti-Mycobacterium activity against Mycobacterium tuberculosis and non-tuberculosis Mycobacterium infections.

### Example 56

Compounds are prepared having the structures shown below:

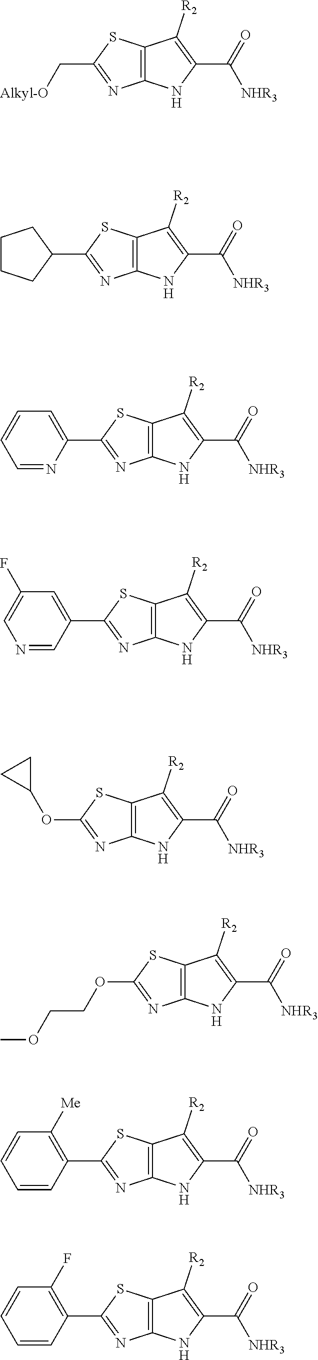

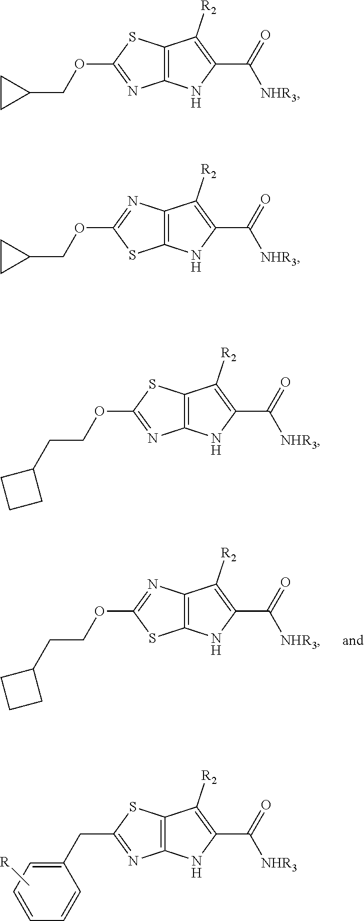

wherein R₂ may be hydrogen, alkyl, cycloalkyl, CN, or halogen, and R₃NH may be (C₄-C₆)alkyl-NH or (C₄-C₇)alkyl-NH; (C₅-C₁₀)cycloalkyl-NH; —CH₂-(C₅-C₇)cycloalkyl-NH; spiro(C₈-C₁₁)cycloalkyl-NH; phenyl-NH;

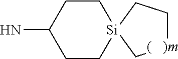

wherein m is 1 or 2; or

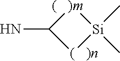

wherein m is 1, 2 or 3 and n is 1, 2, 3, or 4.

It is contemplated that these compounds will show anti Mycobacterium activity against Mycobacterium tuberculosis and non-tuberculosis Mycobacterium infections.

### REFERENCES

Collins, L. Torrero M., and Franzblau, S. Antimicrob. Agents Chemother. 1998, 42, 344-347.

Collins, L., and S. G. Franzblau. “Microplate Alamar Blue Assay versus BACTEC 460 System for High-Throughput Screening of Compounds against Mycobacterium Tuberculosis and Mycobacterium Avium.” Antimicrobial Agents and Chemotherapy, vol. 41, no. 5, May 1997, pp. 1004-09.

Falzari, Kanakeshwari, et al. “In Vitro and in Vivo Activities of Macrolide Derivatives against Mycobacterium Tuberculosis.” Antimicrobial Agents and Chemotherapy, vol. 49, no. 4, April 2005, pp. 1447-54. PubMed, doi:10.1128/AAC.49.4.1447-1454.2005.

Franz, Nicholas D., et al. “Design, Synthesis and Evaluation of Indole-2-Carboxamides with Pan Anti-Mycobacterial Activity.” Bioorganic & Medicinal Chemistry, vol. 25, no. 14, 15 2017, pp. 3746-55. PubMed, doi:10.1016/j.bmc.2017.05.015.

It is to be understood that the invention is not limited to the particular embodiments of the invention described above, as variations of the particular embodiments may be made and still fall within the scope of the appended claims.

The invention will be further described, without limitation, by the following numbered paragraphs:

1. A compound of Formula (I) or Formula (II):

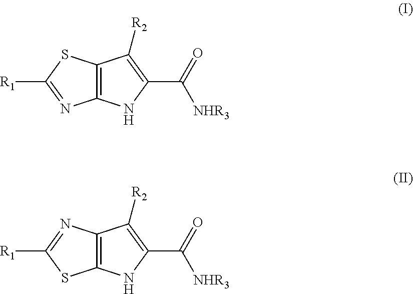

wherein

R₁ is hydrogen, (C₁-C₁₁)alkyl, cycloalkyl, aryl, heteroaryl, alkoxy, or cycloalkoxy;

R2 is hydrogen, alkyl, cycloalkyl, CN, or halogen; R₃NH is
(i) (C₄-C₆)alkyl-NH or (C₄-C₇)alkyl-NH;
(ii) (C₅-C₁₀)cycloalkyl-NH;
(iii) —CH₂-(C₅-C₇)cycloalkyl-NH;
(iv) spiro(C₈-C₁₁)cycloalkyl-NH;
(v) phenyl-NH;
(vi)

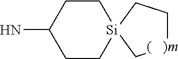

wherein m is 1 or 2; or
(vii)

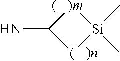

wherein m is 1, 2 or 3 and n is 1, 2, 3, or 4, or a pharmaceutically acceptable salt thereof.

2. The compound according to paragraph 1, or a pharmaceutically acceptable salt thereof, wherein R₁ is hydrogen, (C₁-C₆)alkyl, (C₇-C₁₀)ara-alkyl, (C₆-C₉)heteroara-alkyl, fluoro-substituted (C₁-C₆)alkyl, or alkoxy-substituted (C₁-C₆)alkyl.

3. The compound according to paragraph 1, or a pharmaceutically acceptable salt thereof, wherein R₁ is hydrogen, methyl, cyclopropyl, pyridinyl, or phenyl.

4. The compound according to paragraph 1, or a pharmaceutically acceptable salt thereof, wherein R₁ is (C₁-C₁₀)alkyl; —OCH₂CH₂OCH₃; or —CH₂OCH₃.

5. The compound according to paragraph 1, or a pharmaceutically acceptable salt thereof, wherein R₁ is
(i) (C₁-C₁₁)alkyl substituted with one to four substituents each independently selected from an alkoxy, halogen, CN, or aryl;
(ii) aryl substituted with one to four substituents each independently selected from an alkyl, halogen, alkoxy, and trifluoromethyl;
(iii) phenyl substituted with one to four substituents each independently selected from an alkyl, halogen, alkoxy, and trifluoromethyl; or
(iv) alkoxy substituted with an alkoxy.

6. The compound according to any one of paragraphs 1-5 , or a pharmaceutically acceptable salt thereof, wherein R₂ is hydrogen, (C₁-C₃)alkyl, chloro, or bromo.

7. The compound according to paragraph 6, or a pharmaceutically acceptable salt thereof, wherein R₂ is hydrogen or methyl.

8. The compound according to any one of paragraphs 1-5, or a pharmaceutically acceptable salt thereof, wherein R₂ is lower alkyl, CH₂F, CHF₂, or CF₃.

9. The compound according to any one of paragraphs 1-8, or a pharmaceutically acceptable salt thereof, wherein R₃NH is (C₅-C₉)cycloalkyl-NH; or bridged cycloalkyl-NH.

10. The compound according to any one of paragraphs 1-8, or a pharmaceutically acceptable salt thereof, wherein R₃NH is
(i) a bridged cycloalkyl-NH substituted with one to four substituents selected from lower alkyl and hydroxyl;
(ii) (C₄-C₆)alkyl-NH substituted with one or two substituents each independently selected from (C₁-C₄)alkyl, fluoro substituted (C₁-C₄)alkyl, methoxy, hydroxy(C₁-C₄)alkyl, methoxy(C₁-C₄)alkyl, ethynyl, cyano, halo, hydroxy and hydroxyl;
(iii) (C₅-C₉)cycloalkyl-NH substituted with one to two substituents each independently selected from (C₁-C₄)alkyl, fluoro-substituted (C₁-C₄)alkyl, methoxy, and hydroxyl;
(iv) —CH₂-(C₅-C₇)cycloalkyl-NH, wherein said (C₅-C₇)cycloalkyl is substituted with one to two substituents each independently selected from (C₁-C₄)alkyl, fluoro-substituted (C₁-C₄)alkyl, methoxy and hydroxyl;
(v) spiro(C₈-C₁₁)cycloalkyl-NH; or phenyl-NH substituted with one to two substituents each independently selected from (C₁-C₄)alkyl, fluoro substituted (C₁-C₄)alkyl, methoxy, hydroxy(C₁-C₄)alkyl, methoxy(C₁-C₄)alkyl, ethynyl, cyano, halo, or hydroxyl.

11. The compound according to any one of paragraphs 1-8, or a pharmaceutically acceptable salt thereof, wherein R₃NH is

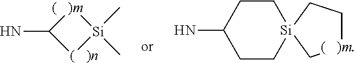

12. The compound according to any one of paragraphs 1-8, or a pharmaceutically acceptable salt thereof, wherein R₃NH is

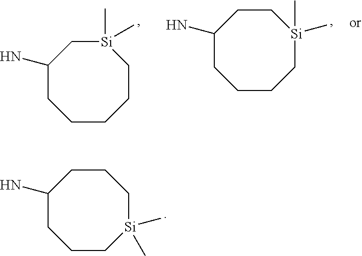

13. The compound according to any one of paragraphs 1-8, or a pharmaceutically acceptable salt thereof, wherein R₃NH is

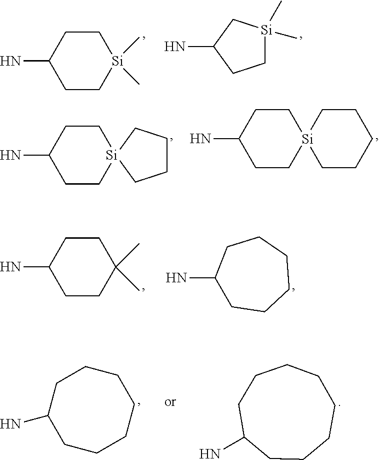

14. The compound according to any one of paragraphs 1-8, or a pharmaceutically acceptable salt thereof, wherein NHR₃ is

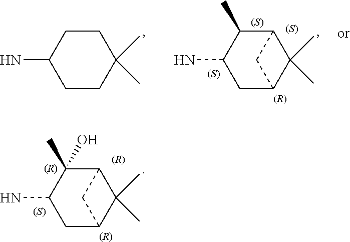

15. The compound according to any one of paragraphs 1-14, or a pharmaceutically acceptable salt thereof, which has Formula (I).

16. The compound according to any one of paragraphs 1-14, or a pharmaceutically acceptable salt thereof, which has Formula (II).

17. A compound according to paragraph 1 which is:

N-(4,4-dimethylcyclohexyl)-2-methyl-4H-pyrrolo[2,3-d]thiazole-5-carboxamide;

N-(4,4-dimethylcyclohexyl)-4H-pyrrolo[2,3-d]thiazole-5-carboxamide;

2-methyl-N-[(1S,2S,3 S, 5R)-2, 6, 6-trimethylnorpinan-3-yl]-4H-pyrrolo[2,3 -d]thiazole-5-carboxamide;

N-[(1S,2S,3S,5R)-2,6,6-trimethylnorpinan-3-yl]-4H-pyrrolo[2,3-d]thiazole-5-carboxamide;

N-cyclooctyl-2-methyl-4H-pyrrolo[2,3-d]thiazole-5-carboxamide;

2-methyl-N-((1 S,2 S,3 S,5R)-2,6,6-trimethylbicyclo [3.1.1]heptan-3-yl)-4H-pyrrolo [3,2-d]thiazole-5-carboxamide;

N-cyclooctyl-2-methyl-4H-pyrrolo[3,2-d]thiazole-5-carboxamide;

N-cyclooctyl-2-cyclopropyl-4H-pyrrolo[2,3-d]thiazole-5-carboxamide;

2-cyclopropyl-N-((1 S,2 S,3 S, 5R)-2, 6, 6-trimethylbicyclo [3.1.1]heptan-3-yl)-4H-pyrrolo [2,3 -d]thiazole-5-carboxamide;

2-cyclopropyl-6-methyl-N-((1S,2S,3 S, 5R)-2, 6, 6-trimethylbicyclo [3.1.1]heptan-3-yl)-4H-pyrrolo [2,3 -d]thiazole-5-carboxamide;

N-(1,1-dimethylsilinan-4-yl)-2-methyl-4H-pyrrolo [2,3-d]thiazole-5-carboxamide;

N-(1,1-dimethylsilinan-4-yl)-2-methyl-4H-pyrrolo[3,2-d]thiazole-5-carboxamide; N-[(1R,2R,3 S,5R)-2-hydroxy-2,6,6-trimethyl-norpinan-3-yl]-2-methyl-4H-pyrrolo [2,3-d]thiazole-5-carboxamide;

2-cyclopropyl-N-(1,1-dimethylsilinan-4-yl)-6-methyl-4H-pyrrolo [2,3 -d]thiazole-5-carboxamide;

2-cyclopropyl-N-(1,1-dimethylsilinan-4-yl)-4H-pyrrolo [2,3 -d]thiazole-5-carboxamide;

N-cyclooctyl-2-cyclopropyl-6-methyl-4H-pyrrolo [2,3 -d]thiazole-5-carboxamide;

2-cyclopropyl-N-(1,1-dimethylsilocan-4-yl)-6-methyl-4H-pyrrolo [2,3 -d]thiazole-5-carboxamide;

2-cyclopropyl-N-(1,1-dimethylsilolan-3-yl)-4H-pyrrolo [2,3 -d]thiazole-5-carboxamide;

2-cyclopropyl-N-(1,1-dimethylsilocan-5-yl) -4H-pyrrolo [2,3 -d]thiazole-5-carboxamide;

N-(1,1-dimethylsilinan-4-yl)-2-phenyl-4H-pyrrolo [2,3 -d]thiazole-5-carboxamide;

N-(1,1-dimethylsilinan-4-yl)-2-methoxy-4H-pyrrolo [2,3 -d]thiazole-5-carboxamide;

N-(1,1-dimethylsilepan-4-yl)-2-phenyl-4H-pyrrolo [2,3 -d]thiazole-5-carboxamide;

(R)-N-(1,1-dimethylsilepan-4-yl)-2-phenyl-4H-pyrrolo[2,3-d]thiazole-5-carboxamide; (S)-N-(1,1-dimethylsilepan-4-yl)-2-phenyl-4H-pyrrolo[2,3-d]thiazole-5-carboxamide 2-phenyl-N-(5-silaspiro [4.5] decan-8-yl)-4H-pyrrolo [2,3 -d]thiazole-5-carboxamide;

2-phenyl-N-(6-silaspiro[5.5]undecan-3-yl)-4H-pyrrolo[2,3-d]thiazole-5-carboxamide;

N-(1,1-dimethylsilepan-4-yl)-2-(2-pyridyl)-4H-pyrrolo[2,3-d]thiazole-5-carboxamide;

N-(1,1-dimethylsilepan-4-yl)-2-(3-pyridyl)-4H-pyrrolo[2,3-d]thiazole-5-carboxamide;

N-(1,1-dimethylsilocan-5-yl)-2-phenyl-4H-pyrrolo [2,3 -d]thiazole-5-carboxamide;

N-(1,1-dimethylsilinan-4-yl)-2-(4-pyridyl)-4H-pyrrolo[2,3-d]thiazole-5-carboxamide;

N-(1,1-dimethylsilinan-4-yl)-2-(o-tolyl)-4H-pyrrolo [2,3 -d]thiazole-5-carboxamide;

N-(1,1-dimethylsilinan-4-yl)-2-(2-methoxyphenyl)-4H-pyrrolo[2,3-d]thiazole-5-carboxamide;

N-(1,1-dimethylsilinan-4-yl)-2-(2-fluorophenyl)-4H-pyrrolo [2,3 -d]thiazole-5-carboxamide;

2-methoxy-N-(5-silaspiro [4.5] decan-8-yl)-4H-pyrrolo [2,3 -d]thiazole-5-carboxamide;

2-methoxy-N-(6-silaspiro [5.5]undecan-3-yl)-4H-pyrrolo [2,3 -d]thiazole-5-carboxamide;

N-(1,1-dimethylsilepan-4-yl)-2-methoxy-4H-pyrrolo[2,3-d]thiazole-5-carboxamide;

(R)-N-(1,1-dimethylsilepan-4-yl)-2-methoxy-4H-pyrrolo[2,3-d]thiazole-5-carboxamide;

(S)-N-(1,1-dimethylsilepan-4-yl)-2-methoxy-4H-pyrrolo[2,3-d]thiazole-5-carboxamide;

N-(1,1-dimethylsilocan-5-yl)-2-methoxy-4H-pyrrolo[2,3-d]thiazole-5-carboxamide;

N-(1,1-dimethylsilolan-3-yl)-2-methoxy-4H-pyrrolo[2,3-d]thiazole-5-carboxamide;

N-(1,1-dimethylsilolan-3-yl)-2-phenyl-4H-pyrrolo[2,3-d]thiazole-5-carboxamide;

2-(4-tert-butylphenyl)-N-(6-silaspiro[5.5]undecan-3-yl)-4H-pyrrolo[2,3-d]thiazole-5-carboxamide;

2-(2-methoxyethoxy)-N-(6-silaspiro[5.5]undecan-3-yl)-4H-pyrrolo[2,3-d] thiazole-5-carboxamide;

2-(methoxymethyl)-N-(6-silaspiro[5.5]undecan-3-yl)-4H-pyrrolo[2,3-d] thiazole-5-carboxamide;

6-methyl-2-phenyl-N-(6-silaspiro[5.5]undecan-3-yl)-4H-pyrrolo[2,3-d]thiazole-5-carboxamide;

N-(4-methylcyclohexyl)-2-phenyl-4H-pyrrolo[2,3-d]thiazole-5-carboxamide;

cis-N-(4-methylcyclohexyl)-2-phenyl-4H-pyrrolo[2,3-d]thiazole-5-carboxamide;

trans-N-(4-methylcyclohexyl)-2-phenyl-4H-pyrrolo[2,3-d]thiazole-5-carboxamide;

N-(4,4-dimethylcyclohexyl)-2-methoxy-4H-pyrrolo[2,3-d]thiazole-5-carboxamide;

N-[(1R,2R,3 S, 5R)-2-hydroxy-2, 6, 6-trimethyl-norpinan-3 -yl] -2-methoxy-4H-pyrrolo[2,3 -d]thiazole-5-carboxamide;

2-(cyclopropoxy)-N-(5-silaspiro[4.5]decan-8-yl)-4H-pyrrolo[2,3-d]thiazole-5-carboxamide;

2-(cyclobutoxy)-N-(5-silaspiro[4.5]decan-8-yl)-4H-pyrrolo[2,3-d]thiazole-5-carboxamide;

2-(cyclopropoxy)-N-(1,1-dimethylsilepan-4-yl)-4H-pyrrolo[2,3-d]thiazole-5-carboxamide;

2-cyclopentyl-N-(1,1-dimethylsilinan-4-yl)-4H-pyrrolo[2,3-d] thiazole-5-carboxamide;

2-(cyclobutoxy)-N-(1,1-dimethylsilepan-4-yl)-4H-pyrrolo[2,3-d]thiazole-5-carboxamide;

2-(cyclopentoxy)-N-(1,1-dimethylsilepan-4-yl)-4H-pyrrolo[2,3-d]thiazole-5-carboxamide;

6-bromo-2-cyclopropyl-N-(1,1-dimethylsilinan-4-yl)-4H-pyrrolo[3,2-d]thiazole-5-carboxamide;

N-(1,1-dimethylsilinan-4-yl)-2-isopropyl-6-methyl-4H-pyrrolo[3,2-d]thiazole-5-carboxamide; or

N-(1,1-dimethylsilinan-4-yl)-2-methoxy-4H-pyrrolo[3,2-d]thiazole-5-carboxamide;

or a pharmaceutically acceptable salt thereof.

18. A pharmaceutical composition, comprising a compound according to any one of paragraphs 1-17, or a pharmaceutically acceptable salt thereof, and one or more pharmaceutically acceptable carriers and/or additives.

19. The pharmaceutical composition according to paragraph 18, further comprising one or more additional anti-infective agents.

20. The pharmaceutical composition according to paragraph 19, wherein said additional anti-infective agent is rifampicin, rifabutin, rifapentene, isoniazid, ethambutol, kanamycin, amikacin, capreomycin, clofazimine, cycloserine, para-aminosalicylic acid, linezolid, sutezolid, bedaquiline, delamanid, pretomanid, moxifloxacin or levofloxacin, or combinations thereof.

21. A method of treating a mycobacterial infection, comprising the step of administering a therapeutically effective amount of a compound according to any one of paragraphs 1-17, or a pharmaceutically acceptable salt thereof, to a patient in need thereof.

22. The method according to paragraph 21, wherein the mycobacterial infection is caused by Mycobacterium tuberculosis, Mycobacterium avium, Mycobacterium kansasii, Mycobacterium abscessus or Mycobacterium chelonae.

23. The method according to paragraph 21, wherein the mycobacterial infection is caused by Mycobacterium tuberculosis.

24. The method according to any one of paragraphs 21-23, wherein the patient is afflicted with tuberculosis (TB), multi-drug-resistant tuberculosis (MDR-TB), pre-extensively drug resistant (Pre-XDR-TB) or extensively drug-resistant tuberculosis (XDR-TB).

It is to be understood that the invention is not limited to the particular embodiments of the invention described above, as variations of the particular embodiments may be made and still fall within the scope of the appended claims.

What is claimed is:

1. A compound of Formula (I) or Formula (II): 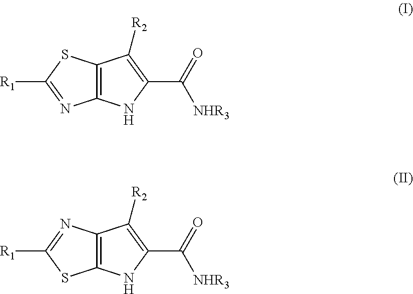
wherein
R₁ is hydrogen, (C₁-C₁₁)alkyl, cycloalkyl, aryl, heteroaryl, alkoxy, or cycloalkoxy;
R₂ is hydrogen, alkyl, cycloalkyl, CN, or halogen;
R₃NH is
(i) (C₄-C₆)alkyl-NH or (C₄-C₇)alkyl-NH;
(ii) (C₅-C₁₀)cycloalkyl-NH;
(iii) —CH₂-(C₅-C₇)cycloalkyl-NH;
(iv) spiro(C₈-C₁₁)cycloalkyl-NH;
(v) phenyl-NH;
(vi) 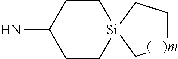
wherein m is 1 or 2; or
(vii) 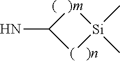
wherein m is 1, 2 or 3 and n is 1, 2, 3, or 4, or a pharmaceutically acceptable salt thereof.

2. The compound according to claim 1, or a pharmaceutically acceptable salt thereof, wherein R₁ is hydrogen, (C₁-C₆)alkyl, (C₇-C₁₀)ara-alkyl, (C₆-C₉)heteroara-alkyl, fluoro-substituted (C₁-C₆)alkyl, or alkoxy-substituted (C₁-C₆)alkyl.

3. The compound according to claim 1, or a pharmaceutically acceptable salt thereof, wherein R₁ is hydrogen, methyl, cyclopropyl, pyridinyl, or phenyl.

4. The compound according to claim 1, or a pharmaceutically acceptable salt thereof, wherein R₁ is (C₁-C₁₀)alkyl; —OCH₂CH₂OCH₃; or —CH₂OCH₃.

5. The compound according to claim 1, or a pharmaceutically acceptable salt thereof, wherein R₁ is
(i) (C₁-C₁₁)alkyl substituted with one to four substituents each independently selected from an alkoxy, halogen, CN, or aryl;
(ii) aryl substituted with one to four substituents each independently selected from an alkyl, halogen, alkoxy, and trifluoromethyl;
(iii) phenyl substituted with one to four substituents each independently selected from an alkyl, halogen, alkoxy, and trifluoromethyl; or
(iv) alkoxy substituted with an alkoxy.

6. The compound according to claim 1, or a pharmaceutically acceptable salt thereof, wherein R₂ is hydrogen, (C₁-C₃)alkyl, chloro, or bromo.

7. The compound according to claim 4, or a pharmaceutically acceptable salt thereof, wherein R₂ is hydrogen or methyl.

8. The compound according to claim 1, or a pharmaceutically acceptable salt thereof, wherein R₂ is lower alkyl, CH₂F, CHF₂, or CF₃.

9. The compound according to claim 1, or a pharmaceutically acceptable salt thereof, wherein R₃NH is (C₅-C₉)cycloalkyl-NH; or bridged cycloalkyl-NH.

10. The compound according to claim 1, or a pharmaceutically acceptable salt thereof, wherein R₃NH is
(i) a bridged cycloalkyl-NH substituted with one to four substituents selected from lower alkyl and hydroxyl;
(ii) (C₄-C₆)alkyl-NH substituted with one or two substituents each independently selected from (C₁-C₄)alkyl, fluoro substituted (C₁-C₄)alkyl, methoxy, hydroxy(C₁-C₄)alkyl, methoxy(C₁-C₄)alkyl, ethynyl, cyano, halo, hydroxy and hydroxyl;
(iii) (C₅-C₉)cycloalkyl-NH substituted with one to two substituents each independently selected from (C₁-C₄)alkyl, fluoro-substituted (C₁-C₄)alkyl, methoxy, and hydroxyl;
(iv) —CH₂-(C₅-C₇)cycloalkyl-NH, wherein said (C₅-C₇)cycloalkyl is substituted with one to two substituents each independently selected from (C₁-C₄)alkyl, fluoro-substituted (C₁-C₄)alkyl, methoxy and hydroxyl;
(v) spiro(C₈-C₁₁)cycloalkyl-NH; or phenyl-NH substituted with one to two substituents each independently selected from (C₁-C₄)alkyl, fluoro substituted (C₁-C₄)alkyl, methoxy, hydroxy(C₁-C₄)alkyl, methoxy(C₁-C₄)alkyl, ethynyl, cyano, halo, or hydroxyl.

11. The compound according to claim 1, or a pharmaceutically acceptable salt thereof, wherein R₃NH is

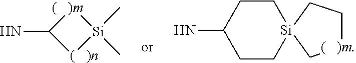

12. The compound according to claim 1, or a pharmaceutically acceptable salt thereof, wherein R₃NH is

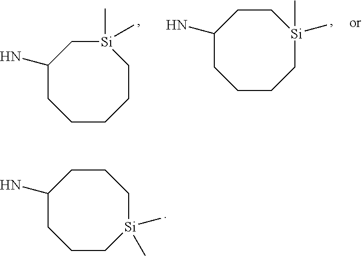

13. The compound according to claim 1, or a pharmaceutically acceptable salt thereof, wherein R₃NH is

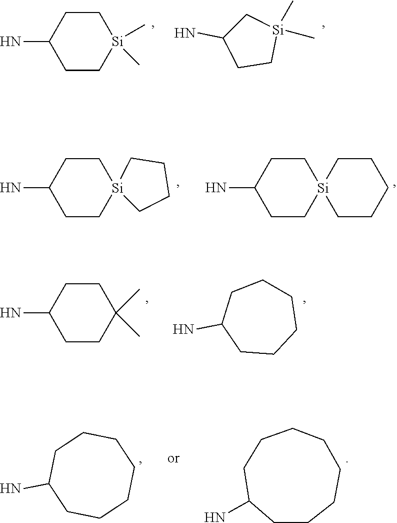

14. The compound according to claim 1, or a pharmaceutically acceptable salt thereof, wherein NHR₃ is

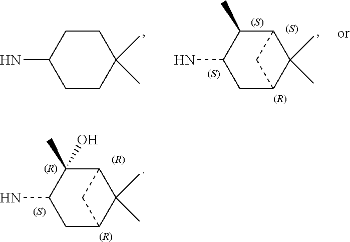

15. The compound according to claim 1, or a pharmaceutically acceptable salt thereof, which has Formula (I).

16. The compound according to claim 1, or a pharmaceutically acceptable salt thereof, which has Formula (II).

17. A compound which is:
N-(4,4-dimethylcyclohexyl)-2-methyl-4H-pyrrolo[2,3-d]thiazole-5-carboxamide;
N-(4,4-dimethylcyclohexyl)-4H-pyrrolo[2,3-d]thiazole-5-carboxamide;
2-methyl-N-[(1S,2S,3 S, 5R)-2, 6, 6-trimethylnorpinan-3 -yl]-4H-pyrrolo [2,3 -d]thiazole-5-carboxamide;
N-[(1S,2S,3S,5R)-2,6,6-trimethylnorpinan-3-yl]-4H-pyrrolo[2,3-d]thiazole-5-carboxamide;
N-cyclooctyl-2-methyl-4H-pyrrolo[2,3-d]thiazole-5-carboxamide;
2-methyl-N-((1 S,2 S,3 S,5R)-2,6,6-trimethylbicyclo [3.1.1]heptan-3-yl)-4H-pyrrolo [3,2-d]thiazole-5-carboxamide;
N-cyclooctyl-2-methyl-4H-pyrrolo[3,2-d]thiazole-5-carboxamide;
N-cyclooctyl-2-cyclopropyl-4H-pyrrolo[2,3-d]thiazole-5-carboxamide;
2-cyclopropyl-N-((1 S,2 S,3 S, 5R)-2, 6, 6-trimethylbicyclo [3.1.1]heptan-3 -yl)-4H-pyrrolo [2,3 -d]thiazole-5-carboxamide;
2-cyclopropyl-6-methyl-N-((1S,2S,3 S, 5R)-2, 6, 6-trimethylbicyclo [3.1.1]heptan-3-yl)-4H-pyrrolo[2,3-d]thiazole-5-carboxamide;
N-(1,1-dimethylsilinan-4-yl)-2-methyl-4H-pyrrolo [2,3-d]thiazole-5-carboxamide;
N-(1,1-dimethylsilinan-4-yl)-2-methyl-4H-pyrrolo[3,2-d]thiazole-5-carboxamide;
N-[(1R,2R,3S,5R)-2-hydroxy-2,6,6-trimethyl-norpinan-3-yl]-2-methyl-4H-pyrrolo[2,3-d]thiazole-5-carboxamide;
2-cyclopropyl-N-(1,1-dimethylsilinan-4-yl)-6-methyl-4H-pyrrolo[2,3-d]thiazole-5-carboxamide;
2-cyclopropyl-N-(1,1-dimethylsilinan-4-yl)-4H-pyrrolo[2,3-d]thiazole-5-carboxamide;
N-cyclooctyl-2-cyclopropyl-6-methyl-4H-pyrrolo[2,3-d]thiazole-5-carboxamide;
2-cyclopropyl-N-(1,1-dimethylsilocan-4-yl)-6-methyl-4H-pyrrolo[2,3-d]thiazole-5-carboxamide;
2-cyclopropyl-N-(1,1-dimethylsilolan-3-yl)-4H-pyrrolo[2,3-d]thiazole-5-carboxamide;
2-cyclopropyl-N-(1,1-dimethylsilocan-5-yl) -4H-pyrrolo[2,3-d]thiazole-5-carboxamide;
N-(1,1-dimethylsilinan-4-yl)-2-phenyl-4H-pyrrolo[2,3-d]thiazole-5-carboxamide;
N-(1,1-dimethylsilinan-4-yl)-2-methoxy-4H-pyrrolo[2,3-d]thiazole-5-carboxamide;
N-(1,1-dimethylsilepan-4-yl)-2-phenyl-4H-pyrrolo[2,3-d]thiazole-5-carboxamide;
(R)-N-(1,1-dimethylsilepan-4-yl)-2-phenyl-4H-pyrrolo[2,3-d]thiazole-5-carboxamide;
(S)-N-(1,1-dimethylsilepan-4-yl)-2-phenyl-4H-pyrrolo[2,3-d]thiazole-5-carboxamide 2-phenyl-N-(5-silaspiro[4.5]decan-8-yl)-4H-pyrrolo[2,3-d]thiazole-5-carboxamide;
2-phenyl-N-(6-silaspiro[5.5]undecan-3-yl)-4H-pyrrolo[2,3-d]thiazole-5-carboxamide;
N-(1,1-dimethylsilepan-4-yl)-2-(2-pyridyl)-4H-pyrrolo[2,3-d]thiazole-5-carboxamide;
N-(1,1-dimethylsilepan-4-yl)-2-(3-pyridyl)-4H-pyrrolo[2,3-d]thiazole-5-carboxamide;
N-(1,1-dimethylsilocan-5-yl)-2-phenyl-4H-pyrrolo[2,3-d]thiazole-5-carboxamide;
N-(1,1-dimethylsilinan-4-yl)-2-(4-pyridyl)-4H-pyrrolo[2,3-d]thiazole-5-carboxamide;
N-(1,1-dimethylsilinan-4-yl)-2-(o-tolyl)-4H-pyrrolo[2,3-d]thiazole-5-carboxamide;
N-(1,1-dimethylsilinan-4-yl)-2-(2-methoxyphenyl)-4H-pyrrolo[2,3-d]thiazole-5-carboxamide;
N-(1,1-dimethylsilinan-4-yl)-2-(2-fluorophenyl)-4H-pyrrolo[2,3-d]thiazole-5-carboxamide;
2-methoxy-N-(5-silaspiro[4.5]decan-8-yl)-4H-pyrrolo[2,3-d]thiazole-5-carboxamide;
2-methoxy-N-(6-silaspiro[5.5]undecan-3-yl)-4H-pyrrolo[2,3-d]thiazole-5-carboxamide;
N-(1,1-dimethylsilepan-4-yl)-2-methoxy-4H-pyrrolo[2,3-d]thiazole-5-carboxamide;
(R)-N-(1,1-dimethylsilepan-4-yl)-2-methoxy-4H-pyrrolo[2,3-d]thiazole-5-carboxamide;
(S)-N-(1,1-dimethylsilepan-4-yl)-2-methoxy-4H-pyrrolo[2,3-d]thiazole-5-carboxamide;
N-(1,1-dimethylsilocan-5-yl)-2-methoxy-4H-pyrrolo[2,3-d]thiazole-5-carboxamide;
N-(1,1-dimethylsilolan-3-yl)-2-methoxy-4H-pyrrolo[2,3-d]thiazole-5-carboxamide;
N-(1,1-dimethylsilolan-3-yl)-2-phenyl-4H-pyrrolo[2,3-d]thiazole-5-carboxamide;
2-(4-tert-butylphenyl)-N-(6-silaspiro[5.5]undecan-3-yl)-4H-pyrrolo[2,3-d]thiazole-5-carboxamide;
2-(2-methoxyethoxy)-N-(6-silaspiro[5.5]undecan-3-yl)-4H-pyrrolo[2,3-d] thiazole-5-carboxamide;
2-(methoxymethyl)-N-(6-silaspiro[5.5]undecan-3-yl)-4H-pyrrolo[2,3-d] thiazole-5-carboxamide;
6-methyl-2-phenyl-N-(6-silaspiro[5.5]undecan-3-yl)-4H-pyrrolo[2,3-d]thiazole-5-carboxamide;
N-(4-methylcyclohexyl)-2-phenyl-4H-pyrrolo[2,3-d]thiazole-5-carboxamide;
cis-N-(4-methylcyclohexyl)-2-phenyl-4H-pyrrolo[2,3-d]thiazole-5-carboxamide;
trans-N-(4-methylcyclohexyl)-2-phenyl-4H-pyrrolo[2,3-d]thiazole-5-carboxamide;
N-(4,4-dimethylcyclohexyl)-2-methoxy-4H-pyrrolo[2,3-d]thiazole-5-carboxamide;
N-[(1R,2R,3 S, 5R)-2-hydroxy-2, 6, 6-trimethyl-norpinan-3 -yl] -2-methoxy-4H-pyrrolo [2,3-d]thiazole-5-carboxamide;
2-(cyclopropoxy)-N-(5-silaspiro[4.5]decan-8-yl)-4H-pyrrolo[2,3-d]thiazole-5-carboxamide;
2-(cyclobutoxy)-N-(5-silaspiro[4.5]decan-8-yl)-4H-pyrrolo[2,3-d]thiazole-5-carboxamide;
2-(cyclopropoxy)-N-(1,1-dimethylsilepan-4-yl)-4H-pyrrolo[2,3-d]thiazole-5-carboxamide;
2-cyclopentyl-N-(1,1-dimethylsilinan-4-yl)-4H-pyrrolo[2,3-d] thiazole-5-carboxamide;
2-(cyclobutoxy)-N-(1,1-dimethylsilepan-4-yl)-4H-pyrrolo[2,3-d]thiazole-5-carboxamide;
2-(cyclopentoxy)-N-(1,1-dimethylsilepan-4-yl)-4H-pyrrolo[2,3-d]thiazole-5-carboxamide;
6-bromo-2-cyclopropyl-N-(1,1-dimethylsilinan-4-yl)-4H-pyrrolo[3,2-d]thiazole-5-carboxamide;
N-(1,1-dimethylsilinan-4-yl)-2-isopropyl-6-methyl-4H-pyrrolo[3,2-d]thiazole-5-carboxamide; or
N-(1,1-dimethylsilinan-4-yl)-2-methoxy-4H-pyrrolo[3,2-d]thiazole-5-carboxamide;
or a pharmaceutically acceptable salt thereof.

18. A pharmaceutical composition, comprising a compound according to claim 1, or a pharmaceutically acceptable salt thereof, and one or more pharmaceutically acceptable carriers and/or additives.

19. The pharmaceutical composition according to claim 18, further comprising one or more additional anti-infective agents.

20. The pharmaceutical composition according to claim 19, wherein said additional anti-infective agent is rifampicin, rifabutin, rifapentene, isoniazid, ethambutol, kanamycin, amikacin, capreomycin, clofazimine, cycloserine, para-aminosalicylic acid, linezolid, sutezolid, bedaquiline, delamanid, pretomanid, moxifloxacin or levofloxacin, or combinations thereof.

21. A method of treating a mycobacterial infection, comprising the step of administering a therapeutically effective amount of a compound according to claim 1, or a pharmaceutically acceptable salt thereof, to a patient in need thereof.

22. The method according to claim 21, wherein the mycobacterial infection is caused by Mycobacterium tuberculosis, Mycobacterium avium, Mycobacterium kansasii, Mycobacterium abscessus or Mycobacterium chelonae.

23. The method according to claim 21, wherein the mycobacterial infection is caused by Mycobacterium tuberculosis.

24. The method according to claim 21, wherein the patient is afflicted with tuberculosis (TB), multi-drug-resistant tuberculosis (MDR-TB), pre-extensively drug resistant (Pre-XDR-TB) or extensively drug-resistant tuberculosis (XDR-TB).
